(19) United States
(12) Patent Application Publication
(10) Pub. No.: US 2026/0158126 A1
(43) Pub. Date: Jun. 11, 2026

(54) RUMINAL AND METHANOGEN VACCINES AND USES THEREOF

(71) Applicant: HELIX NANOTECHNOLOGIES, INC., Boston, MA (US)

(72) Inventors: Hannu Rajaniemi, Corte Madera, CA (US); Nikolai Eroshenko, Boston, MA (US); Marianna Keaveney, Walpole, MA (US); Rachel Smith, Cambridge, MA (US); Everett Webster, Boston, MA (US); Taylor Gill, Cambridge, MA (US); Kemo Jammeh, Brighton, MA (US); Nikhil Dhar, Boston, MA (US); Kyle Backman, South San Francisco, CA (US); Dario De Jessus Davila Pasillas, Boston, MA (US); Justin Sean Huang, Pine Brook, NJ (US); Katherine Rowe, Boston, MA (US); Arianna Lizeth Lopez Sauceda, Boston, MA (US); Nikolai Paul Radzinski, Melrose, MA (US); Tao Qin, North Reading, MA (US)

(21) Appl. No.: 19/126,716

(22) PCT Filed: Nov. 3, 2023
(86) PCT No.: PCT/US2023/036773

§ 371 Date: May 2, 2025

Related U.S. Application Data
(60) Provisional application No. 63/422,312, filed on Nov. 3, 2022.

(51) Int. Cl. A61K 39/02; A61K 39/00; A61P 31/04; C07K 14/195; C12N 15/62

(52) U.S. Cl. CPC A61K 39/02; A61K 39/0001; A61P 31/04; C07K 14/195; C12N 15/625; A61K 2039/53; A61K 2039/545; A61K 2039/552; A61K 2039/555; A61K 2039/70

(57) ABSTRACT

Disclosed herein are polynucleotides encoding one or more ruminal-associated antigens, and polypeptides encoded by said polynucleotides. Also provided herein are compositions comprising the same, and method of making and using said compositions.

### CROSS REFERENCE TO RELATED APPLICATIONS

This application claims priority to and the benefit of U.S. Provisional Patent Application No. 63/422,312, filed on Nov. 3, 2022, the entire contents of which are hereby incorporated by reference in its entirety.

### BACKGROUND

Controlling methane emissions from animals, e.g., ruminants, is a new and emerging field.

### SUMMARY

The present disclosure identifies certain challenges with increases in global surface temperature due to increased levels of greenhouse gases, namely methane (CH4). For example, the present disclosure identifies that increase in methane from agriculture represents a major source of greenhouse gas emissions. The present disclosure also identifies that reducing methane emissions from animals, e.g., ruminants, will be important to reduce greenhouse gas emissions.

Among other things, the present disclosure provides technologies for reducing methane emissions from animals, e.g., ruminants, by providing compositions for vaccinating animals, e.g., ruminants, against ruminal-associated antigens. In some embodiments, a composition comprising ruminal-associated antigens can further comprise one or more chemokines and/or cytokines. Without wishing to be bound by theory, the present disclosure proposes that a composition comprising a ruminal-associated antigen (e.g., a ruminal antigen and/or a methanogen antigen), can reduce methane emissions and/or reduce the abundance of microorganisms (e.g., methanogens) in the digestive tract of animals. In some embodiments, reducing methane emissions and/or reducing abundance of methanogens in the digestive tract of animals (e.g., ruminants) can increase the energy efficiency of animals.

This disclosure provides an isolated polynucleotide encoding one or more ruminal-associated antigens, fragments thereof, variants thereof, or variant fragments thereof.

In some embodiments, one or more ruminal-associated antigens comprises one or more ruminal antigens.

In some embodiments, one or more ruminal antigens are derived from: a polypeptide that is involved in attachment to fermenting bacteria, or a fragment or variant or variant fragment thereof.

In some embodiments, a polynucleotide comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 ruminal antigens.

In some embodiments, one or more ruminal-associated antigens comprises one or more methanogen antigens.

In some embodiments, one or more methanogen antigens are derived from a polypeptide found on a cell surface of a wild-type methanogen, or a fragment or variant or variant fragment thereof.

In some embodiments, a polynucleotide comprises at least 2, at least 3, at least 4, at least 5, at least 6, at least 7, at least 8, at least 9, at least 10, at least 15, or at least 20 methanogen antigens.

In some embodiments, a polynucleotide comprises about 2 to about 20, about 2 to about 15, about 2 to about 10, about 2 to about 9, about 2 to about 8, about 2 to about 7, about 2 to about 6, about 2 to about 5, about 2 to about 5, about 2 to about 4, about 2 to about 3, about 3 to about 20, about 4 to about 20, about 5 to about 20, about 6 to about 20, about 7 to about 20, about 8 to about 20, about 9 to about 20, about 10 to about 20, or about 15 to about 20 methanogen antigens.

In some embodiments, one or more methanogen antigens are the same, e.g., having the same sequence.

In some embodiments, one or more methanogen antigens are different, e.g., having different sequences.

In some embodiments, one or more methanogen antigens comprise:
(i) one or more peptides having at least 80% sequence identity to a methanogen protein;
(ii) one or more secreted antigens comprising a signal peptide;
(iii) a plurality of peptides having at least 80% sequence identity to each other,
(iv) a plurality of peptides associated with at least 2 different, at least 3 different, at least 4 different or at least 5 different methanogen species;
(v) one or more peptides having at least 80% sequence identity to a polypeptide having signal peptidase activity;
(vi) one or more peptides having at least 80% sequence identity to an AglB polypeptide or substantially similar function to an AglB polypeptide;
(vii) one or more peptides having at least 80% sequence identity to an Ig-like domain-containing polypeptide or substantially similar function to an Ig-like domain-containing polypeptide; or
(viii) or any combination thereof.

In some embodiments, one or more methanogen antigens comprise:
(i) one or more peptides having at least 80% sequence identity to a methanogen protein,
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to a polypeptide having signal peptidase activity;
(vi) or any combination thereof.

In some embodiments, a polynucleotide comprises three methanogen antigens. In some embodiments, the three methanogen antigens are associated with at least three different methanogen species.

In some embodiments, one or more methanogen antigens comprise:
(i) one or more peptides having at least 80% sequence identity to a methanogen protein;
   (ii) one or more secreted antigens comprising a signal peptide;
   (iii) a plurality of peptides associated with at least 2 different, at least 3 different, at least 4 different or at least 5 different methanogen species;
   (iv) one or more peptides having at least 80% sequence identity to an AglB polypeptide or substantially similar function to an AglB polypeptide; or
(v) any combination thereof.

In some embodiments, a polynucleotide comprises five methanogen antigens. In some embodiments, five methanogen antigens are associated with at least five different methanogen species.

In some embodiments, one or more methanogen antigens comprise
(i) one or more peptides having at least 80% sequence identity to a methanogen protein;
   (ii) one or more secreted antigens comprising a signal peptide;
   (iii) a plurality of peptides having at least 80% sequence identity to each other;
(iv) a plurality of peptides associated with at least 2 different, at least 3 different, at least 4 different or at least 5 different methanogen species;
   (v) one or more peptides having at least 80% sequence identity to an Ig-like domain-containing polypeptide or substantially similar function to an Ig-like domain-containing polypeptide; or
(vi) any combination thereof.

In some embodiments, a polynucleotide comprises eight methanogen antigens. In some embodiments, eight methanogen antigens are associated with at least three different methanogen species.

In some embodiments, one or more methanogen antigens comprise a plurality of peptides associated with at least 2 different, at least 3 different, at least 4 different, at least 5 different, at least 6 different, at least 7 different, at least 8 different, at least 9 different, at least 10 different, at least 11 different, at least 12 different, at least 15 different or at least 20 different methanogen species.

In some embodiments, one or more methanogen antigens comprise a plurality of peptides associated with about 2 different, about 3 different, about 4 different, about 5 different, about 6 different, about 7 different, about 8 different, about 9 different, about 10 different, about 11 different, about 12 different, about 15 different or about 20 different methanogen species.

In some embodiments, one or more methanogen antigens are derived from a polypeptide found on the cell surface of a wild-type methanogen, or a fragment or variant thereof or variant fragment thereof.

In some embodiments, one or more methanogen antigens is secreted.

In some embodiments, a methanogen antigen comprises a peptide that is involved in adhesion, attachment, or mobility, or a fragment or variant of a peptide that is involved in adhesion, attachment, mobility.

In some embodiments, a secreted methanogen antigen comprises a signal peptide. In some embodiments, a signal peptide can be predicted using a prediction algorithm and optionally wherein the prediction score is at least 0.5.

In some embodiments, one or more methanogen antigens comprise one or more peptides having at least 80% sequence identity to a methanogen protein.

In some embodiments, a polynucleotide comprises a plurality of methanogen antigens each having at least 80% sequence identity to each other.

In some embodiments, a polynucleotide comprises a plurality of methanogen antigens wherein each methanogen antigen in the plurality is associated with a different methanogen species. In some embodiments, a polynucleotide comprises a plurality of methanogen antigens, and wherein the methanogen antigens in the plurality are associated with at least 2 different, at least 3 different, at least 4 different or at least 5 different methanogen species. In some embodiments, a polynucleotide comprises a plurality of methanogen antigens and wherein each methanogen antigen in the plurality is associated with the same methanogen species. In some embodiments, a methanogen species comprises: Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoream, Methanosphaera stadtmanae, Methanomicrobium mobile, Methanosarcina mazei, Methanobrevibacter thaueri, Methanobrevibacter sp. UBA188, or Methanosarcina soligelidi. In some embodiments, a methanogen species comprises: Methanobrevibacter ruminantium, Methanobrevibacter smithii, or Methanobrevibacter oralis. In some embodiments, a methanogen species comprises: Methanobrevibacter ruminantium Methanobrevibacter smithii Methanobrevibacter oralis Methanomicrobium mobile or Methanosphaera stadtmanae.

In some embodiments, a polynucleotide comprises one or more methanogen antigens comprising one or more peptides having at least 80% sequence identity to a polypeptide having signal peptidase activity. In some embodiments, a polypeptide having signal peptidase activity: (i) is membrane bound; (ii) has the ability to cleave one or more signal peptides; (iii) plays a role in converting a secretory protein to a mature form (e.g., from a non-secretory form to a secretory form); and/or (iv) or any combination thereof.

In some embodiments, a polynucleotide comprises one or more methanogen antigens comprising one or more peptides having: (1) at least 80% sequence identity to an Immunoglobulin (Ig)-like polypeptide, or (2) substantially similar function to an Ig-like domain-containing polypeptide. In some embodiments, an an Ig-like domain-containing polypeptide comprises a polypeptide characterized as a cell-surface protein that facilitates binding to other cells and/or cell surfaces.

In some embodiments, a polynucleotide comprises one or more methanogen antigens comprising an adhesin or fragment or variant thereof, a pilli protein or a fragment or variant thereof, or a flagellin protein or a fragment or a variant thereof.

In some embodiments, a polynucleotide comprises a sequence encoding one or more methanogen antigens comprising an antigen provided in Table 1 or a sequence with at least 85%, at least 90%, at least 91%, at least 92%, at least 93%, at least 94%, at least 95%, at least 96%, at least 97%, at least 98%, or at least 99% identity thereto.

In some embodiments, a polynucleotide comprises a sequence encoding one or more methanogen antigens comprising an antigen sequence provided in Table 2 or a sequence with at least 85%, at least 90%, at least 91%, at least 92%, at least 93%, at least 94%, at least 95%, at least 96%, at least 97%, at least 98%, or at least 99% identity thereto. In some embodiments, a polynucleotide comprises a sequence encoding one or more methanogen antigens comprising a fragment, a variant, or a variant fragment of an antigen sequence provided in Table 2. Exemplary antigen sequences provided in Table 2 are marked with no bolding or italicizing.

In some embodiments, a polynucleotide comprises an antigen nucleic acid sequence provided in Table 2 or a sequence with at least 85%, at least 90%, at least 91%, at least 92%, at least 93%, at least 94%, at least 95%, at least 96%, at least 97%, at least 98%, or at least 99% identity thereto.

In some embodiments, a polynucleotide comprises a sequence encoding a polypeptide having at least 85%, at least 90%, at least 91%, at least 92%, at least 93%, at least 94%, at least 95%, at least 96%, at least 97%, at least 98%, or at least 99% identity to a polypeptide sequence provided in Table 2.

In some embodiments, a polynucleotide comprises one or more methanogen antigens comprising one or more peptides having: (1) at least 80% sequence identity to an archaeal oligosaccharyltransferase (AglB) polypeptide, or (2) substantially similar function to an AglB polypeptide. In some embodiments, an AglB polypeptide is characterized as having N-glycosylation activity.

In some embodiments, a methanogen antigen comprises a peptide that is involved in adhesion, attachment, or mobility, or a fragment or variant of a peptide that is involved in adhesion, attachment, mobility.

In some embodiments, a methanogen antigen comprises an adhesin or fragment or variant thereof. In some embodiments, a methanogen antigen comprises an adhesin protein provided in Table 1 or a sequence with at least 85% identity thereto. In some embodiments, a methanogen antigen comprises mru1499, or a fragment or variant thereof.

In some embodiments, a methanogen antigen comprises an antigen sequence in Table 2, or a variant or a fragment or a variant fragment thereof. In some embodiments, a methanogen antigen comprises an antigen sequence in Table 2 or a sequence having at least 85%, at least 90%, at least 91%, at least 92%, at least 93%, at least 94%, at least 95%, at least 96%, at least 97%, at least 98%, or at least 99% identity thereto. Exemplary antigen sequences provided in Table 2 are marked with no bolding or italicizing.

In some embodiments, a methanogen antigen comprises an antigen sequence encoded by a nucleotide sequence provided in SEQ ID NO: 1, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 2, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 5, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 6, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 7, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 8, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 16, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 15, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 18, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 17, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 20, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 19, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 22, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 21, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 24, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 23, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 26, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 25, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 28, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 27, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 30, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 29, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 32, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 31, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 34, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 33, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 36, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 35, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 38, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 37, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 40, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 39, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 42, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 41, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 44, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 43, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 46, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 45, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 48, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 47, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 50, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 49, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 52, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 51, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 54, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 53, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 56, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 55, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 58, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 57, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 60, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 59, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 62, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 61, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 64, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 63, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 66, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 65, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 68, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 67, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 70, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 69, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 72, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 71, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 74, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 73, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 76, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100/6 identity thereto. In some embodiments, a methanogen antigen comprises an antigen sequence provided in SEQ ID NO: 75, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 78, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 77, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 80, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 79, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 82, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 81, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 84, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 83, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 86, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 85, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 88, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 87, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 90, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 89, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 92, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto. In some embodiments, a methanogen antigen comprises an antigen sequence provided in SEQ ID NO: 91, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence encoded by a nucleotide sequence provided in SEQ ID NO: 94, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least or 100% identity thereto. In some embodiments, a methanogen antigen comprises an antigen sequence provided in SEQ ID NO: 93, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises a pili protein or fragment or variant thereof.

In some embodiments, a methanogen antigen comprises a flagellin protein or fragment or variant thereof.

In some embodiments, a polynucleotide comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 methanogen antigens.

In some embodiments, a polynucleotide comprises a signal peptide. In some embodiments, a signal peptide has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% homology to an archaeal signal peptide.

In some embodiments, a polynucleotide comprises at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% homology to a bacterial signal peptide.

In some embodiments, a methanogen antigen comprises an antigen sequence provided in SEQ ID NO: 97, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 98, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 99, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 100, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 101, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 102, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 103, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%4, at least or 100% identity thereto.

In some embodiments, a methanogen antigen comprises an antigen sequence provided in SEQ ID NO: 104, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 105, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 106, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%1, at least or 100% identity thereto.

In some embodiments, a methanogen antigen comprises an antigen sequence provided in SEQ ID NO: 107, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 108, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 109, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 110, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%1c, at least or 100% identity thereto.

In some embodiments, a methanogen antigen comprises an antigen sequence provided in SEQ ID NO: 111, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 112, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 113, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 114, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 115, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 116, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 117, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 118, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 119, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 120, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a methanogen antigen comprises an antigen sequence provided in SEQ ID NO: 121, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%1, at least or 100% identity thereto.

In some embodiments, a methanogen antigen comprises an antigen sequence provided in SEQ ID NO: 122, or a sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a signal peptide is or comprises a PPA2 signal peptide, or a fragment or variant thereof.

In some embodiments, signal peptide is or comprises an SSP signal peptide, or a fragment or variant thereof.

In some embodiments, a signal peptide is or comprises a SARS-CoV-2 Spike secretion signal, or a fragment or variant thereof.

In some embodiments, a signal peptide is situated at the N terminal of the ruminal-associated antigen sequence.

In some embodiments, a polynucleotide comprises: (i) a first nucleotide sequence encoding one or more ruminal antigens, and (ii) a second nucleotide sequence encoding one or more methanogen antigens.

In some embodiments, a polynucleotide comprises; (i) a first nucleotide sequence encoding one or more ruminal antigens, (ii) a second nucleotide sequence encoding one or more methanogen antigens; and (iii) a third nucleotide sequence encoding a chemokine and/or cytokine.

In some embodiments, a polynucleotide comprises: (i) a first nucleotide sequence encoding one or more ruminal antigens, (ii) a second nucleotide sequence encoding a chemokine and/or cytokine.

In some embodiments, a polynucleotide comprises: (i) a first nucleotide sequence encoding one or more methanogen antigens, and (ii) a second nucleotide sequence encoding a chemokine and/or cytokine.

In some embodiments, a polynucleotide comprises a nucleotide encoding a chemokine and/or a cytokine. In some embodiments, a chemokine and/or cytokine are chosen from APRIL, VIP and/or CXCL10.

In some embodiments, a polynucleotide comprises a sequence encoding a chemokine or cytokine provided in SEQ ID NO: 10 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a polynucleotide comprises a chemokine or cytokine nucleotide sequence provided in SEQ ID NO: 9 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a polynucleotide comprises a sequence encoding a chemokine or cytokine provided in SEQ ID NO: 12 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a polynucleotide comprises a chemokine or cytokine nucleotide sequence provided in SEQ ID NO: 11 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a polynucleotide comprises a sequence encoding a chemokine or cytokine provided in SEQ ID NO: 14 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a polynucleotide comprises a chemokine or cytokine nucleotide sequence provided in SEQ ID NO: 13 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least or 100% identity thereto.

In some embodiments, a nucleotide sequence encoding one or more ruminal antigens comprises at least 2, at least 3, at least 4, at least 5, at least 6, at least 7, at least 8, at least 9, or at least 10 ruminal antigens.

In some embodiments, a nucleotide sequence encoding one or more methanogen antigens comprises at least 2, at least 3, at least 4, at least 5, at least 6, at least 7, at least 8, at least 9, or at least 10 methanogen antigens.

In some embodiments, a first nucleotide sequence, a second nucleotide sequence and/or a third nucleotide sequence are situated on one polynucleotide.

In some embodiments, a first nucleotide sequence, a second nucleotide sequence and/or a third nucleotide sequence are situated on different polynucleotides.

In some embodiments, one or more ruminal antigens and/or one or more methanogen antigens are each situated on a separate nucleotide sequence.

In some embodiments, a polynucleotide comprises at least 2 methanogen antigens and the at least 2 methanogen antigens are situated on separate nucleotide sequences.

In some embodiments, a polynucleotide comprises at least 3 methanogen antigens and the at least 3 methanogen antigens are situated on separate nucleotide sequences.

In some embodiments, a polynucleotide comprises at least 4 methanogen antigens and the at least 4 methanogen antigens are situated on separate nucleotide sequences.

In some embodiments, a polynucleotide comprises at least 5 methanogen antigens and the at least 5 methanogen antigens are situated on separate nucleotide sequences.

In some embodiments, a polynucleotide comprises at least 6 methanogen antigens and the at least 6 methanogen antigens are situated on separate nucleotide sequences.

In some embodiments, a polynucleotide comprises at least 7 methanogen antigens and the at least 7 methanogen antigens are situated on separate nucleotide sequences.

In some embodiments, a polynucleotide comprises at least 8 methanogen antigens and the at least 8 methanogen antigens are situated on separate nucleotide sequences.

In some embodiments, a polynucleotide comprises at least 9 methanogen antigens and the at least 9 methanogen antigens are situated on separate nucleotide sequences.

In some embodiments, a polynucleotide comprises at least 10 methanogen antigens and the at least 10 methanogen antigens are situated on separate nucleotide sequences.

In some embodiments, one or more ruminal antigens and/or one or more methanogen antigens are situated on the same nucleotide sequence.

In some embodiments, a polynucleotide comprises a transmembrane domain.

In some embodiments, a polynucleotide further comprises a complement C3d-binding polypeptide from an immunoglobulin-binding protein (Sbi) of Staphylococcus aureus. In some embodiments, a complement C3d-binding polypeptide is or comprises one or both of domain III and domain IV of the Sbi of Staphylococcus aureus, or a functional fragment or a variant thereof.

In some embodiments, a polynucleotide is or comprises DNA.

In some embodiments, a polynucleotide is or comprises RNA. In some embodiments, a RNA comprises a 5′ cap. In some embodiments, a RNA comprises a polyA tail.

In some embodiments, a polynucleotide sequence comprises one or more ribonucleotides comprising a nucleoside comprising an acetyl group, wherein the nucleoside is N4-acetylcytidine and the modified ribonucleotide has a structure of

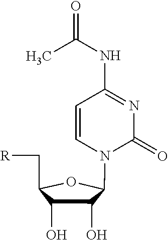

wherein R is a 5′ monophosphate, a 5′ diphosphate, or a 5′ triphosphate.

In some embodiments, a polyribonucleotide further comprises one or more modified ribonucleotides other than N4-acetylcytidine, optionally wherein the nucleoside is chosen from: an adenosine, an inosine, a guanosine, a cytidine or a uridine, or any combination thereof.

In some embodiments, a nucleoside of the one or more modified ribonucleotides is 5-hydroxymethyluridine, and the modified ribonucleotide has a structure of

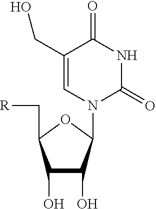

wherein R is a 5′ monophosphate, a 5′ diphosphate, or a 5′ triphosphate.

In some embodiments, a polynucleotide sequence comprises one or more ribonucleotides comprising a nucleoside comprising a hydroxymethyl group, wherein the nucleoside is 5-hydroxymethyluridine and the modified ribonucleotide has a structure of

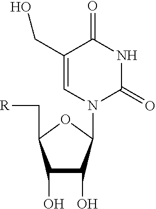

wherein R is a 5′ monophosphate, a 5′ diphosphate, or a 5′ triphosphate.

In some embodiments, a polyribonucleotide further comprises one or more modified ribonucleotides other than 5-hydroxymethyluridine, wherein the one or more modified ribonucleotides comprises a nucleoside chosen from: an adenosine, an inosine, a guanosine, a cytidine or a uridine, or any combination thereof.

In some embodiments, a nucleoside of the one or more modified ribonucleotides is N4-acetylcytidine and the modified ribonucleotide has a structure of

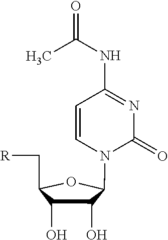

wherein R is a 5′ monophosphate, a 5′ diphosphate, or a 5′ triphosphate.

Also provided herein is a polypeptide encoded by a polynucleotide disclosed herein.

This disclosure further provides a composition comprising one or more polyribonucleotides disclosed herein or a polypeptide disclosed herein.

In some embodiments, a composition comprises a plurality of polyribonucleotides disclosed herein. In some embodiments, each of the plurality of polyribonucleotides comprises one or more methanogen antigens. In some embodiments, each of the plurality of polyribonucleotides encodes a different methanogen antigen. In some embodiments, each of the plurality of polyribonucleotides encodes the same methanogen antigen.

In some embodiments of a composition comprising a plurality of polyribonucleotides, the plurality of polyribonucleotides comprises a mixture of polyribonucleotides, e.g., a portion that encodes the same methanogen antigen and a portion that encodes a different methanogen antigen.

In some embodiments, a composition disclosed herein comprises one or more polynucleotides comprising one or more ruminal-associated antigens, e.g., methanogen antigens. In some embodiments, a composition (e.g., a multi-component composition, e.g., a multi-component vaccine composition) comprises a plurality of polynucleotides (e.g., a plurality of RNA) each comprising an antigen. In some embodiments, a composition (e.g., a multi-component composition, a multi-component vaccine composition) comprises a plurality of polynucleotides (e.g., a plurality of RNA) each comprising one or more antigens (e.g., the same or different antigens).

In some embodiments, a composition (e.g., a multi-component composition, e.g., a multi-component vaccine composition), comprising a plurality of polynucleotides comprises about 1% of a first polynucleotide and about 99% of one or more additional polynucleotides. In some embodiments, a composition (e.g., a multi-component composition, e.g., a multi-component vaccine composition), comprising a plurality of polynucleotides comprises about 5% of a first polynucleotide and about 95% of one or more additional polynucleotides. In some embodiments, a composition (e.g., a multi-component composition, e.g., a multi-component vaccine composition), comprising a plurality of polynucleotides comprises about 10% of a first polynucleotide and about 90% of one or more additional polynucleotides. In some embodiments, a composition (e.g., a multi-component composition, e.g., a multi-component vaccine composition), comprising a plurality of polynucleotides comprises about 20% of a first polynucleotide and about 80% of one or more additional polynucleotides. In some embodiments, a composition (e.g., a multi-component composition, e.g., a multi-component vaccine composition), comprising a plurality of polynucleotides comprises about 30% of a first polynucleotide and about 70% of one or more additional polynucleotides. In some embodiments, a composition (e.g., a multi-component composition, e.g., a multi-component vaccine composition), comprising a plurality of polynucleotides comprises about 40% of a first polynucleotide and about 60% of one or more additional polynucleotides. In some embodiments, a composition (e.g., a multi-component composition, e.g., a multi-component vaccine composition), comprising a plurality of polynucleotides comprises about 50% of a first polynucleotide and about 50% of one or more additional polynucleotides. In some embodiments, a composition (e.g., a multi-component composition, e.g., a multi-component vaccine composition), comprising a plurality of polynucleotides comprises about 60% of a first polynucleotide and about 40% of one or more additional polynucleotides. In some embodiments, a composition (e.g., a multi-component composition, e.g., a multi-component vaccine composition), comprising a plurality of polynucleotides comprises about 70% of a first polynucleotide and about 30% of one or more additional polynucleotides. In some embodiments, a composition (e.g., a multi-component composition, e.g., a multi-component vaccine composition), comprising a plurality of polynucleotides comprises about 80% of a first polynucleotide and about 20% of one or more additional polynucleotides. In some embodiments, a composition (e.g., a multi-component composition, e.g., a multi-component vaccine composition), comprising a plurality of polynucleotides comprises about 90% of a first polynucleotide and about 10% of one or more additional polynucleotides. In some embodiments, a composition (e.g., a multi-component composition, e.g., a multi-component vaccine composition), comprising a plurality of polynucleotides comprises about 99% of a first polynucleotide and about 1% of one or more additional polynucleotides.

In some embodiments, a composition (e.g., a multi-component composition, e.g., a multi-component vaccine composition), comprising a plurality of polynucleotides comprises about 33% of a first polynucleotide, about 33% of a second polynucleotides and about 33% of a third polynucleotide.

In some embodiments, a composition is a pharmaceutical composition.

In some embodiments, a composition is an immunogenic composition.

In some embodiments, a composition is a vaccine composition.

In some embodiments, a composition is formulated for delivery with a carrier. In some embodiments, a carrier is a lipid nanoparticle, a cationic lipid, a polymeric particle.

In some embodiments, a composition is formulated for delivery without a carrier.

This disclosure provides, a method comprising administering a composition disclosed herein, to a cell, tissue or animal, e.g., a ruminant.

In some embodiments, a method is a vaccination method.

In some embodiments, an animal is a ruminant, e.g., cattle, sheep, goats, buffalo, moose, antelope, caribou, or deer.

In some embodiments, an animal is a domestic animal.

In some embodiments, a vaccine reduces methane emissions from an animal as compared to an animal not administered a vaccine or administered a different vaccine.

In some embodiments, a composition is characterized in that administration of a composition to an animal reduces methane emissions from an animal as compared to an otherwise comparable animal not administered the composition or administered a different composition. In some embodiments, a reduction in methane emissions is at least 5%, at least 10%, at least 20%, at least 30%, at least 40%, at least 50?%, at least 60%, at least 70%, at least 80%, or at least 90% as compared to an otherwise comparable animal not administered the composition or administered a different composition.

In some embodiments, a composition is characterized in that administration of the composition to the animal reduces a population of microorganisms in the animal, as compared to an otherwise comparable animal not administered the composition or administered a different composition. In some embodiments, microorganisms are methanogens. In some embodiments, methanogen comprises a methanogen from one or more of the following clades: Methanobrevibacter, Methanosphaera, Methanobacterium, Methanosarcinales, Methanomicrobiales, Methanothermobacter, Candidatus Methanomethylophilus, Thermoplasmatales.

In some embodiments, a methanogen comprises Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

In some embodiments, a composition is characterized in that administration of the composition to the animal increases a growth rate of the animal as compared to the growth rate of an otherwise comparable animal not administered the composition or administered a different composition. In some embodiments, an increase in growth rate comprises a daily increase in weight of the animal, optionally wherein the daily increase in weight of the animal is an increase of at least 5%, at least 10%, at least 20%, at least 30%, at least 40%, at least 50%, at least 60%, at least 70%, at least 80%, or at least 90% of weight as compared to an otherwise comparable animal not administered the composition or administered a different composition.

In some embodiments, a method disclosed herein comprises administering one dose of the vaccine composition to the animal.

In some embodiments, a method disclosed herein comprises administering a plurality of doses of the vaccine composition to the animal.

In some embodiments, an animal is administered a first dose of the composition followed by one or more subsequent doses of the composition.

In some embodiments, a first dose and the one or more subsequent doses of the composition comprise the same methanogen antigens and/or ruminal antigens.

In some embodiments, a first dose and the one or more subsequent doses of the composition comprise different methanogen antigens and/or ruminal antigens.

In some embodiments, a composition is administered 1, 2, 3, 4, 5, 6, 7, 8, 9, 10, 20 or 30 times to an animal.

In some embodiments, a composition is administered once every 1 week, 2 weeks, 3 weeks, 1 month, 2 months, 3 months, 4 months, 5 months or 6 months.

In some embodiments, a composition is administered in combination with one or more additional agents.

In some embodiments, a one or more additional agents comprises a chemical additive, a biological feed additive.

In some embodiments, a composition is administered in combination with one or more additional compositions.

In some embodiments, an additional composition immunizes an animal from a disease, e.g., an infectious disease.

Also disclosed herein is a composition comprising an isolated polynucleotide disclosed herein, or a pharmaceutical composition disclosed herein, for use in administration to (e.g., vaccination of) an animal.

This disclosure also provides use of a composition comprising an isolated polynucleotide disclosed herein, or a pharmaceutical composition disclosed herein, in the preparation of a medicament for administration to (e.g., vaccination of) an animal.

In some embodiments of any of the composition for use, or use disclosed herein an isolated polynucleotide or pharmaceutical composition is administered to the animal.

In some embodiments of any of the composition for use, or use disclosed herein administration of the isolated polynucleotide or pharmaceutical composition results in:
(i) reduced methane emissions;
(ii) reduced abundance of one or more microorganisms (e.g., methanogens) in a digestive tract of the animal; and/or
(iii) increased growth rate of the animal,
as compared to an otherwise comparable animal not administered the composition, or administered a different composition.

In some embodiments of any of the composition for use, or use disclosed herein the animal is a ruminant or a domestic animal.

In some embodiments of any of the composition for use, or use disclosed herein a methanogen comprises Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methancaldoococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

### BRIEF DESCRIPTION OF THE DRAWINGS

FIGS. 1A-1B describe antigen-specific IgG titers from serum, for an exemplary multi-component RNA vaccine formulation. Experimental groups comprised of calves receiving 1 Dose (Day 20, n=5), 2 Dose (Day 20 & 34, n=5), or no dose/untreated (UTD, n=5) of the exemplary RNA vaccine formulation: 0.5 mg total of 50% OVA_HNadj (85% HNmod1, wherein HNmod1 corresponds to Ac4C and 50% mru1499 HNadj (85% HNmod1). Serum was collected at Day 20, 34, and 90. ELISAs were run for each antigen of the multi-component vaccine, respectively (OVA, mru1499). Geometric mean reciprocal titers are provided for 1 Dose and 2 Dose groups, for each timepoint, and for each timepoint to measure OVA-IgG (FIG. 1A) and mru1499-IgG (FIG. 1B) response to the exemplary RNA vaccine in cattle FIG. 2 describes normalized relative abundance of methanogen species in rumen fluid, for an exemplary multi-component RNA vaccine formulation. Experimental groups comprised of calves receiving 1 Dose (Day 20, n=5), 2 Dose (Day 20 & 34, n=5), or no dose/untreated (UTD, n=5) of the exemplary RNA vaccine formulation: 0.5 mg total of 50% OVA_HNadj (85% HNmod1) and 50% mru1499_HNadj (85% HNmod1). Rumen fluid was collected at Day 0, 20, 34, and 90. Samples were sequenced, resulting in relative abundance measurements of methanogen Methanobrevibacter (Mbb.) ruminantium M1 for 1 Dose and 2 Dose groups, for each timepoint in response to the exemplary RNA vaccine in cattle. Each timepoint value is a delta from that group's Day 0 value. The y-axis is normalized to 1.0 with the cohort's average pre-study M1/Archaea.

FIGS. 3A-3B describe normalized relative abundance of methanogen and archaeal species in rumen fluid, for exemplary RNA vaccine formulations targeting methanogen proteins and with various secretion signals. Experimental groups comprised of calf groups (n=4) receiving prime/boost doses of the specified RNA vaccine formulations (0.5 mg, 100% HNmod1). Rumen fluid was collected at Day 0, 20, 34, and 90, and processed into a pellet. Samples were sequenced, resulting in relative abundance measurements of methanogen Methanohrevibacter (Mbh.) ruminanlium M1 (FIG. 3A) and total Archaea (FIG. 3B) for each formulation group in response to the exemplary RNA vaccine in cattle. Each timepoint value is a delta from that group's Day 0 value. The y-axis is normalized to 1.0 with the cohort's average pre-study M1/Archaea.

FIG. 4 describes normalized relative abundance of methanogen and archaeal species in rumen fluid, for exemplary RNA vaccine formulations with cytokines and with double chem mods (HNmod1/HNmod2, wherein HNMod1 corresponds to Ac4C and HNMOD2 corresponds to 5hmU). Experimental groups comprised of calf groups (n=4) receiving prime/boost doses of the specified RNA vaccine formulations (0.5 mg; respective composition described in Figure). Experimental setup is similar to FIG. 3; however, RNA vaccine formulations include cytokine and double chem mod (HNmod1/HNmod2) features. Note n=1 for F8.

FIG. 5 describes methane emissions per feed intake, for exemplary RNA vaccine formulations targeting methanogen proteins and with various secretion signals. Experimental groups comprised of calf groups (n=4) receiving prime/boost doses of specified RNA vaccine formulations (0.5 mg, 100% HNmod1; respective composition described in Figure). Enteric methane emissions were measured by animal, as average daily CH4 (g/d) per total daily feed intake (lbs/d), and then averaged across an 8-day measurement timeframe.

FIG. 6 describes methane emissions per feed intake, for exemplary RNA vaccine formulations with cytokines and with single chem mods (HNmod1) or double chem mods (HNmod1, HNmod2). Experimental groups comprised of calf groups (n=4) receiving prime/boost doses of specified RNA vaccine formulations (0.5 mg; respective composition described in Figure). Experimental setup is similar to FIG. 5; however, RNA vaccine formulations include cytokine features. Note n=3 for group F5 and n=2 for group F7, due to calf non-adherence to GreenFeed Systems.

FIGS. 7A-7B describe normalized relative abundance of all archaeal species in rumen fluid, for exemplary multi-component RNA vaccine formulations targeting multiple methanogen proteins. Experimental groups comprised of calf groups (n=10) receiving three doses of specified RNA vaccine formulations (0.55 mg, 100% Hnmod1; respective composition described in Table 3). Rumen fluid was collected at Day 90, 109 and 122, and processed into a pellet, in which Day 90 represents a pre-injection (prior to the third injection) sample (“PRE”, 14 days prior to boost), Day 109 represents a 5-day post-injection (after the third injection) sample (“+5d POST”), and Day 122 represents an 18-day post-injection (after the third injection) sample (“+18d POST”). In this study, rumen collections were not taken after the first and second injections, to minimize disturbance to the rumen biome. Samples were sequenced, resulting in relative abundance measurements (e.g., % of total sequence population) for all bacterial and archaeal species in response to RNA vaccine formulations CNT(OVA) and F3 The bacterial and archaeal species tested included M. wolinui, M. stadtmanae. M. oralis, M. smithii, and M. rumiiantlium. FIG. 7A shows the relative abundance levels of all archaeal species identified at time points PRE. +5-d POST and +18-d POST. The “Other” category comprises of species with a relative abundance of <0.01%, and include M. mazei, M. soligelidi, M. arboriphilus, Thermoplasmatales sp., M. formicicum, M. boviskoreani, M. mobile, Methanothermobacter sp., Candidatus Methanomethylophilus alvus, and Nitrososphaera sp. FIG. 7B shows the % change of each archaeal species relative to the PRE timepoint and normalized to the CNT(OVA).

FIGS. 8A-8B describe raw methane emissions and methane emissions per feed intake, for exemplary multi-component RNA vaccine formulations targeting multiple Methanogen proteins. Experimental groups comprised of calf groups (n=10) receiving prime/boost doses of specified RNA vaccine formulations (0.55 mg, 100% Hnmod1; respective composition described in Table 3). Enteric methane emissions were measured by animal, as average daily CH4 (g/d) per total daily feed intake (lbs/d), and then averaged across a 14-day timeframe post prime injection, T1 (D0-D14), and the pre-injection baseline, T0 (D-8-D-2). Percent Delta Methane (FIG. 8A) and Methane/Intake (FIG. 8B) are determined by dividing raw methane or methane/intake values of the later timeframe (T 1) by that of the baseline (TO), and then normalized by measuring the difference in this percentage for each treatment group versus that of the control group (CNT-OVA). Error bars reflect standard error.

FIG. 9 describes growth efficiency measurements for exemplary RNA vaccine formulations targeting Methanogen proteins and with various secretion signals, with cytokines, and with single chem mods (HNmod1) or double chem mods (HNmod1, HNmod2). Experimental groups comprised of calf groups (n=4) receiving prime/boost doses of specified RNA vaccine formulations (0.5 mg; compositions correspond to those in FIGS. 5 and 6). Average Daily Gain (ADG, lb/day), a reflection of growth efficiency where a larger value reflects increased efficiency, was calculated by determining the linear regression of weight measurements between time periods. Specifically, “Pre-Injection Baseline” is the average weight gained per day from D-32 to D0 (e.g., 32-day period pre-injection), “DO-D20” is the average weight gained per day from D0 to D20 (e.g., 20-day period post prime injection), and “D20-D90” is the average weight gained per day from D20 to D90 (e.g., 70-day period post boost injection). Delta ADG represents the difference between ADG at a specified timeframe and the Pre-injection Baseline per treatment group, and is normalized by then finding the difference in this delta and that of the control group (UTD). Not displayed is vaccine condition F8 (n=1), which had a ADG change of +1.70 lb/day at D20 and +0.91 lb/day at D90.

FIGS. 10A-10B describe growth efficiency measurements for exemplary multi-component RNA vaccine formulations targeting multiple Methanogen proteins. Experimental groups comprised of calf groups (n=10) receiving prime/boost doses of specified RNA vaccine formulations (0.55 mg; 100% Hnmod1; respective composition described in Table 3). Average Daily Gain (ADG, lb/day), a reflection of growth efficiency where a larger value reflects increased efficiency, was calculated by determining the linear regression of weight measurements between time periods (FIG. 10A). Specifically, “Pre-Injection Baseline” is the average weight gained per day from D-49 to D0 (e.g., 50-day period pre-injection), “D25” is the average weight gained per day from D0 to D25 (e.g. e.g., 25-day period post prime injection), and “D90” is the average weight gained per day from D-25 to D90 (e.g., 65-day period post boost injection). FIG. 10B shows Delta ADG in a pound per day unit (lb/d), representing the difference between ADG at a specified timeframe and the Pre-Injection Baseline per treatment group, then normalized by finding the difference in this delta and that of the control group (CNT-OVA). The right panel shows Delta ADG in a percentage unit (%), representing the ratio of the later time by the earlier time, then normalized by finding the difference in this percentage and that of the control group (CNT-OVA).

### CERTAIN DEFINITIONS

About or approximately: As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to an animal to achieve delivery of an agent that is, or is included in, the composition. Those of ordinary skill in the art will be aware of a variety of routes that may, in appropriate circumstances, be utilized for administration to an animal, e.g., a ruminant. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments. administration may involve continuous dosing (e.g., perfusion) for at least a selected period of time.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen comprises at least one epitope of a target protein. In some embodiments, an epitope may be a linear epitope. In some embodiments, an epitope may be a conformational epitope. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Delivery/contacting: As used interchangeably herein, the term “delivery,” “delivering,” or “contacting” refers to introduction of a fusion polynucleotide (e.g., as described herein) or a fusion polypeptide (e.g., as described herein) into a target cell. A target cell can be cultured in vitro or ex vivo or be present in an animal (in vivo), Methods of introducing a fusion polynucleotide (e.g., as described herein) or a fusion polypeptide (e.g., as described herein) into a target cell can vary with in vitro, ex vivo, or in vivo applications. In some embodiments, a fusion polynucleotide (e.g., as described herein) or a fusion polypeptide (e.g., as described herein) can be introduced into a target cell in a cell culture by in vitro transfection. In some embodiments, a fusion polynucleotide (e.g., as described herein) or a fusion polypeptide (e.g., as described herein) can be introduced into a target cell via delivery vehicles (e.g., nanoparticles, liposomes, and/or complexation with a cell-penetrating agent). In some embodiments, a fusion polynucleotide (e.g., as described herein) or a fusion polypeptide (e.g., as described herein) can be introduced into a target cell in an animal by administering a fusion polynucleotide (e.g., as described herein) or a fusion polypeptide (e.g., as described herein) to an animal.

Functional: As used herein, the term “functional” is used to refer to a form or fragment of an entity that exhibits a particular property and/or activity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a fragment comprises a polynucleotide fragment. In some embodiments, a fragment comprises a polypeptide fragment. In some embodiments, a polynucleotide fragment or a polypeptide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polynucleotide or whole polypeptide. In some embodiments, a polynucleotide fragment or a polypeptide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polynucleotide or whole polypeptide. The whole polypeptide or whole polynucleotide may in some embodiments be referred to as the “parent” of the polynucleotide fragment or polypeptide fragment.

Methanogen: As used herein, a “methanogen” is a microorganism that produces methane. In some embodiments, a methanogen can inhabit a digestive tract of an animal and participate in a fermentation process. In some embodiments, a methanogen is an organism that conserves energy for ATP synthesis by producing methane gas. In some embodiments, a methanogen can inhabit a digestive tract of a ruminant. In some embodiments, a methanogen can inhabit a digestive tract of a non-ruminant. Exemplary methanogens are provided in Buan N. R. (2018) Emerg Top life Sci 2(4): pp. 629-646, the entire contents of which are hereby incorporated by reference. In some embodiments, a methanogen comprises: one or more methanogens from one or more of the following clades: Methanobrevibacter, Methanosphaera, Methanobacterium, Methanosarcinales, Methanomicrobiales, Methanothermobacter, Candidatus Methanomethylophilus, Thermoplasmatales. In some embodiments, a methanogen comprises: Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

Nucleic acid/Oligonucleotide/Polynucleotide: As used herein, the terms “nucleic acid” and “polynucleotide” and “oligonucleotide” are used interchangeably, and refer to a polymer of 3 nucleotides or more. In some embodiments, a nucleic acid comprises DNA. In some embodiments, a nucleic acid comprises RNA. In some embodiments, a nucleic acid comprises messenger RNA (mRNA). In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long. When a number of nucleotides is used as an indication of size. e.g., of a fusion polynucleotide, a certain number of nucleotides refers to the number of nucleotides on a single strand, e.g., of a fusion polynucleotide.

Polypeptide. The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

Polyribonucleotide: As used herein, the term “polyribonucleotide” refers to a polymer of 3 ribonucleotides or more. In some embodiments, a polyribonucleotide is single stranded. In some embodiments, a polyribonucleotide is double stranded. In some embodiments, a polyribonucleotide comprises both single and double stranded portions. In some embodiments, a polyribonucleotide can comprise a backbone structure as described in the definition of “Nucleic acid “Oligonucleotide” above. A polyribonucleotide can be a regulatory RNA (e.g., siRNA, microRNA, etc.), or a messenger RNA (mRNA) oligonucleotide. In some embodiments, a polyribonucleotide typically comprises at its 3′ end a poly(A) region. In some embodiments, a polyribonucleotide typically comprises at its 5′ end an art-recognized cap structure, e.g., for recognizing and attachment of an RNA to a ribosome to initiate translation. In some embodiments, a polyribonucleotide comprises an RNA oligonucleotide. When a number of ribonucleotides is used as an indication of size, e.g., for a polyribonucleotide, a certain number of nucleotides refers to the number of ribonucleotides on a single strand.

Ruminal-associated antigen: As used herein, a “ruminal associated antigen” is an antigen that is expressed in a ruminant. In some embodiments, a ruminal-associated antigen is a “ruminal antigen”, e.g., an antigen encoded by a nucleotide sequence naturally occurring in a ruminant's genome. In some embodiments, a ruminal-associated antigen is encoded by a nucleotide sequence that is not naturally occurring in a ruminant's genome, e.g., has been introduced into a ruminant's genome, or is part of a microorganism that can inhabit a digestive tract, e.g., a rumen, of a ruminant. In some embodiments, a ruminal-associated antigen comprises: a methanogen antigen, an antigen from a rumen ciliate symbiotic with methanogens; an antigen from a hydrogen-producing organism; an antigen from a taxa associated with low feed efficiency, or any combination thereof. In some embodiments, a ruminal-associated antigen is or comprises a methanogen antigen.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide.

Standard techniques may be used for recombinant DNA, oligonucleotide synthesis, e.g., RNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2nd ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present disclosure identifies that increase in methane from agriculture represents a major source of greenhouse gas emissions. The present disclosure also identifies that reducing methane emissions from animals, e.g., ruminants, will be important to reduce greenhouse gas emissions.

Methane is produced in the rumen of ruminant animals by methanogens, a subgroup of the Archaea domain, Methanogens can reduce the efficiency of nutrients and/or energy available to a ruminant as methane production by methanogens can account for 2-12% of ingested energy (See Leahy S C et al., (2010) PlosOne; volume 5, issue 1; e8926, the entire contents of which are incorporated by reference). Accordingly, the present disclosure identifies that reducing methane emissions from a ruminant can be beneficial in increasing ruminant energy efficiency and reducing methane emissions into the atmosphere.

Among other things, the present disclosure provides technologies for reducing methane emissions from animals, e.g., ruminants, by providing compositions for administering to animals, e.g., ruminants, ruminal-associated antigens (e.g., vaccinating animals (e.g., ruminants) against ruminal-associated antigens). The present disclosure also provides technologies for increasing energy efficiency in animals, e.g., ruminants, for administering to animals, e.g., ruminants, ruminal-associated antigens (e.g., vaccinating animals (e.g., ruminants) against ruminal-associated antigens), thus reducing the abundance of one or more methanogens in the rumen of an animal, e.g., ruminant.

Without wishing to be bound by theory, the present disclosure proposes that a composition comprising a ruminal-associated antigen (e.g., a ruminal antigen and/or a methanogen antigen), can reduce methane emissions and/or reduce the abundance of microorganisms (e.g., methanogens) in the digestive tract of animals. In some embodiments, reducing methane emissions and/or reducing abundance of methanogens in the digestive tract of animals (e.g., ruminants) can increase the energy efficiency of animals. In turn, animals (e.g., ruminants) can require less food, experience an increase in muscle mass, and/or have improved overall health. The benefits can lead to lower costs for raising animals and/or increased revenue from the sale of animals or meat obtained from such animals.

### Ruminal-Associated Antigens

A ruminal-associated antigen as described herein comprises a ruminal antigen and/or a methanogen antigen. In some embodiments, a ruminal-associated antigen is endogenous to a ruminant, e.g., encoded by a nucleotide sequence in a ruminant genome. In some embodiments, a ruminal-associated antigen is not endogenous to a ruminant, e.g., encoded by a nucleotide sequence that is introduced to a ruminant, or encoded by a nucleotide sequence from a microorganism present in or introduced into a ruminant.

In some embodiments, a ruminant is chosen from a cattle, sheep, goat, buffalo, moose, antelope, caribou, or deer, or combinations thereof.

Exemplary ruminal-associated antigens include peptides that are involved in attachment to bacteria, e.g., fermenting bacteria, and fragments, or variants thereof.

In some embodiments, a polynucleotide comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 ruminal antigens.

In some embodiments, a polynucleotide comprises about 2-100, about 3-100, about 4-100, about 5-100, about 6-100, about 7-100, about 8-100, about 9-100, about 10-100, about 20-100, about 30-100, about 40-100, about 50-100, about 60-100, about 70-100, about 80-100, about 90-100, about 2-90, about 2-80, about 2-70, about 2-60, about 2-50, about 2-40, about 2-30, about 2-20, about 2-10, about 2-15, about 2-14, about 2-13, about 2-12, about 2-11, about 2-10, about 2-9, about 2-8, about 2-7, about 2-6, about 2-5, about 2-4, about 2-3 ruminal antigens.

### Methanogens and Methanogen Antigens

A methanogen is an anaerobic archaea characterized by the ability to conserve energy for ATP synthesis by producing methane gas, as described in Buan N. R. (2018). Methanogens can also inhabit the digestive tract of animals, e.g., ruminants and humans. Methanogens can reduce the efficiency of nutrients and/or energy available to a ruminant as methane production by methanogens can account for 2-12% of ingested energy (See Leahy S C et al., (2010) PlosOne; volume 5, issue 1; e8926, the entire contents of which are incorporated by reference). Accordingly, reducing methane emissions from a ruminant can be beneficial in increasing ruminant energy efficiency and/or reducing methane emissions into the atmosphere.

In some embodiments, a methanogen comprises a methanogen from one or more of the following clades: Methanobrevibacter, Methanosphaera, Methanobacterium, Methanosarcinales, Methanomicrobiales, Methanothermobacter, Candidatus Methanomethylophilus, Thermoplasmatales.

In some embodiments, a methanogen comprises a methanogen from a Methanobrevibacter clade. In some embodiments, a methanogen from a Methanobrevibacter clade comprises: Methanobrevibacter ruminantium, Methanobrevibacter smithii, Methanobrevibacter oralis, Methanobrevibacter thaueri, Methanobrevibacter gonschalkii, Methanobrevibacter millerae, Methanobrevibacter woesei, Methanobrevibacter arboriphilus. Methanobrevibacter wolinii, Methanobrevibacter olleyae, Methanobrevibacter boviskoreani, or combinations thereof.

In some embodiments, a methanogen comprises a methanogen from a Methanosphaera clade. In some embodiments, a methanogen from a Methanosphaera clade comprises: Methanosphaera stadtmanae.

In some embodiments, a methanogen comprises a methanogen from a Methanobacterium clade. In some embodiments, a methanogen from a Methanobacterium clade comprises: Methanobacterium bryantii or Methanobacterium formicicum.

In some embodiments, a methanogen comprises a methanogen from a Methanosarcinales clade. In some embodiments, a methanogen from a Methanosarcinales clade comprises Methanosarcina bakeri, Methanosarcina marei, or Methanosarcina soligelidi

In some embodiments, a methanogen comprises a methanogen from a Methanomicrobiales clade. In some embodiments, a methanogen from a Methanomicrobiales clade comprises Methanofollis liminatans, Methanospirillum hungatei, Methanolacinia payteri, Methanocullens marisngigri, or Methanoculleis sp.

In some embodiments, a methanogen comprises a methanogen from a Methanomicrobum clade. In some embodiments, a methanogen from a Methanomicrobium clade comprises Methanomicrobium mobile.

In some embodiments, a methanogen comprises a methanogen from a Thermoplasmatales clade. In some embodiments, a methanogen from a Thermoplasmatales clade comprises Thermoplasma volcanium or Thermoplasma acidophium.

In some embodiments, a methanogen comprises: Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

A methanogen antigen comprises any protein expressed or produced in any methanogen clade, e.g., as described herein. Exemplary methanogen antigens are provided in Table 1. In some embodiments, a methanogen antigen is an antigen provided in Table 1, or a fragment thereof, or a variant thereof. In some embodiments, a methanogen antigen has a sequence with at least 85% identity to a methanogen antigen provided in Table 1. In some embodiments, a methanogen antigen is an adhesin, e.g., mru 1499.

In some embodiments, a methanogen antigen is a peptide involved in adhesion, attachment, mobility, or any combination thereof. In some embodiments, a methanogen antigen is an adhesin, a pili protein, a flagellin protein, or any combination thereof.

In some embodiments, a polynucleotide comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 methanogen antigens.

In some embodiments, a polynucleotide comprises about 2-100, about 3-100, about 4-100, about 5-100, about 6-100, about 7-100, about 8-100, about 9-100, about 10-100, about 20-100, about 30-100, about 40-100, about 50-100, about 60-100, about 70-100, about 80-100, about 90-100, about 2-90, about 2-80, about 2-70, about 2-60, about 2-50, about 2-40, about 2-30, about 2-20, about 2-10, about 2-15, about 2-14, about 2-13, about 2-12, about 2-11, about 2-10, about 2-9, about 2-8, about 2-7, about 2-6, about 2-5, about 2-4, about 2-3 methanogen antigens.

In some embodiments, a polynucleotide comprises one or more methanogen antigens from one or more methanogens.

In some embodiments, a methanogen antigen comprises an antigen sequence provided in Table 2 or a fragment, or a variant or a variant fragment thereof. In some embodiments, a methanogen antigen comprises an antigen sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identity to a sequence provided in Table 2. Exemplary antigen sequences provided in Table 2 are marked with no bolding or italicizing.

In some embodiments, a methanogen antigen comprises an antigen sequence provided in any one of SEQ ID NOs: 2, 6, 8, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 97, 98, 99, 100, 101, 102, 103, 14, 105, 106, 107, 108, 109, 110, 111, 112, 113, 114, 115, 116, 117, 118, 119, 120, 121, 122 or a sequence with at least 85%, at least 90%, at least 91%, at least 92%, at least 93%, at least 94%, at least 95%, at least 96%, at least 97%, at least 98%, or at least 99% identity thereto. Antigen sequences are unmarked (no bolding or italicizing) in SEQ ID NOs: 2, 6, 8, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 97, 98, 99, 100, 101, 102, 103, 14, 105, 106, 107, 108, 109,110, 111, 112, 113, 114, 115, 116, 117, 118, 119, 120, 121, 122.

In some embodiments, a methanogen antigen comprises an antigen encoded by an antigen nucleotide sequence provided in any one of SEQ ID NOs: 1, 5, 7, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, or 94 or a sequence with at least 85%, at least 90%, at least 91%, at least 92%, at least 93%, at least 94%, at least 95%, at least 96%, at least 97%, at least 98%, or at least 99% identity thereto

TABLE 1
Exemplary methanogen antigens.

| Locus | Protein annotation | Size (bp) |
|---|---|---|
| mru0019 | adhesin-like protein | 1220 |
| mru0327 | adhesin-like protein | 2090 |
| mru0687 | adhesin-like protein | 2963 |
| mru1210 | adhesin-like protein | 7250 |
| mru1222 | adhesin-like protein | 4055 |
| mru1506 | adhesin-like protein | 857 |
| mru2053 | adhesin-like protein | 3494 |
| mru2134 | adhesin-like protein | 17957 |
| mru2147 | adhesin-like protein | 16955 |
| mru2178 | adhesin-like protein | 9239 |
| mru0031 | adhesin-like protein | 4415 |
| mru0704 | adhesin-like protein | 2858 |
| mru0963 | adhesin-like protein | 8159 |
| mru0976/0977 | adhesin-like protein | 4775 |
| mru0020 | adhesin-like protein with | 6014 |
| | cysteine protease domain | |
| mru0064 | adhesin-like protein | 3536 |
| mru0072 | adhesin-like protein | 2918 |
| mru0076 | adhesin-like protein | 6605 |
| mru0077 | adhesin-like protein | 9161 |
| mru0079 | adhesin-like protein | 3560 |
| mru0083 | adhesin-like protein | 839 |
| mru0084 | adhesin-like protein | 14477 |
| mru0085 | adhesin-like protein | 8030 |
| mru0086 | adhesin-like protein | 10175 |
| mru0143 | adhesin-like protein with | 3284 |
| | cysteine protease domain | |
| mru0160 | adhesin-like protein | 3176 |
| mru0222 | adhesin-like protein with | 3302 |
| | cysteine protease domain | |
| mru0327 | adhesin-like protein | 2060 |
| mru0338 | adhesin-like protein | 6929 |
| mru0417/0418 | adhesin-like protein | 1391 |
| mru0419 | adhesin-like protein | 4175 |
| mru0727 | adhesin-like protein with | 3788 |
| | cysteine protease domain | |
| mru0772 | adhesin-like protein with | 3281 |
| | cysteine protease domain | |
| mru0839 | adhesin-like protein with | 8639 |
| | cysteine protease domain | |
| mru0842 | adhesin-like protein with | 3977 |
| | cysteine protease domain | |
| mru0978 | adhesin-like protein | 6606 |
| mru0979 | adhesin-like protein | 8753 |
| mru1076 | adhesin-like protein | 2681 |
| mru1077 | adhesin-like protein | 2273 |
| mru1246 | adhesin-like protein | 4619 |
| mru1247 | adhesin-like protein | 5060 |
| mru1465 | adhesin-like protein | 2882 |
| mru1513 | adhesin-like protein | 1853 |
| mru1650 | adhesin-like protein | 9161 |
| mru1726 | adhesin-like protein | 6767 |
| mru1971 | adhesin-like protein | 1937 |
| mru1996 | adhesin-like protein | 4496 |
| mru2043 | adhesin-like protein | 9530 |
| mru2048 | adhesin-like protein | 5417 |
| mru2049 | adhesin-like protein | 10355 |
| mru2052 | adhesin-like protein | 4112 |
| mru2054 | adhesin-like protein | 5054 |
| mru2055 | adhesin-like protein | 8906 |
| mru2059 | adhesin-like protein | 4415 |
| mru2090 | adhesin-like protein | 15200 |
| mru0004 | adhesin-like protein | 2237 |
| mru0331 | adhesin-like protein | 1622 |
| mru0843 | adhesin-like protein with | 6197 |
| | cysteine protease domain | |
| mru0015 | adhesin-like protein with | 3845 |
| | cysteine protease domain | |
| mru0090 | adhesin-like protein | 2063 |
| mru0255 | adhesin-like protein | 4187 |
| mru0450 | adhesin-like protein | 803 |
| mru0723 | adhesin-like protein | 7727 |
| mru0962 | adhesin-like protein | 14789 |
| mru0970 | adhesin-like protein | 2483 |
| mru1263 | adhesin-like protein | 2585 |
| mru1358 | adhesin-like protein | 2243 |
| mru1386 | adhesin-like protein | 1841 |
| mru1387 | adhesin-like protein with | 2957 |
| | cysteine protease domain | |
| mru1424 | adhesin-like protein | 1445 |
| mru1500 | adhesin-like protein | 3896 |
| mru0493 | adhesin-like protein | 2447 |
| mru0824 | adhesin-like protein with | 2027 |
| | transglutaminase domain | |
| mru1499 | adhesin-like protein with | 3032 |
| | transglutaminase domain | |
| mru1604 | adhesin-like protein with | 2996 |
| | transglutaminase domain | |

Antigens from Hydrogen-Producing Symbiotes

In some embodiments, a ruminal-associated antigen comprises an antigen from a hydrogen-producing symbiote. In some embodiments, hydrogen-producing symbiotes are microbes that produce rumen hydrogen in a way that reduces cow efficiency similar to methanogens.

In some embodiments, a ruminal-associated antigen comprises an antigen from a rumen ciliate. In some embodiments, rumen ciliates are symbiotic with methanogens. In some embodiments, rumen ciliates comprises: Diplodinium dentatum (syn. Diplodinium denticilatum), Diploplastron affine (syn. Eudiplodinium affine), Enoploplastron triloricatum (syn. Ostracodinium triloricalum), Entodinium simplex, Entodinium caudatum, Entodinium longinucleatum, Epidinium ecaudatum, Eremoplastron bovis (syn. Eudiplodinium neglectum), Eudiplodinium maggii, Ostracodnium obtusum, Polyplastron multivesiculatum, or combinations thereof.

In some embodiments, hydrogen-producing symbiotes supply nutrients and/or energy (e.g., hydrogen) to methanogens. In some embodiments, hydrogen-producing symbiotes which supply nutrients and/or energy (e.g., hydrogen) to methanogens comprise Butyrivibrio proteoclasticus, Bacteroides thetaiotaomicron, Ruminococcus flavefaciens, Rumninococcus albus, or combinations thereof.

In some embodiments, hydrogen-producing symbiotes comprise taxa associated with low feed efficiency. In some embodiments, hydrogen-producing symbiotes comprising taxa associated with low feed efficiency, comprise: Veilonellaceace, Preivotellaceae, Lachnospiraceae, Succinivibrioniceae, Fibrobacteraceae, Anaerovibrio, Clostridiales, Prevotella, Ruminococcaceae.

### Exemplary Multi-Component Compositions Comprising Ruminal-Associated Antigens

Disclosed herein are polynucleotides (e.g., RNA) comprising one or more ruminal-associated methanogens, e.g., ruminal antigens and/or methanogen antigens. Also disclosed herein are compositions comprising the same, as well as methods of making and using the same for administration to (e.g., vaccination of) an animal, e.g., a ruminant. In some embodiments, a polynucleotide disclosed herein comprises a plurality of ruminal-associated methanogens, e.g., a plurality of ruminal antigens and/or a plurality of methanogen antigens. In some embodiments, a composition comprising such a polynucleotide is also referred to as a multi-component vaccine composition or a multiplexed vaccine composition.

In some embodiments, a polynucleotide is or comprises RNA. In some embodiments, an RNA comprises a 5′ cap, e.g., a 5′-5′ triphosphate linked guanosine. In some embodiments, an RNA comprises a polyA tail. In some embodiments, an RNA comprises one or more untranslated regions, e.g., a 5′ UTR and/or a 3′ UTR.

In some embodiments, a polynucleotide is an RNA comprising: (i) a 5′ cap, (ii) a 5′ UTR region; (iii) a sequence encoding one or more payloads (e.g., one or more ruminal-associated antigens); (iv) a polyA tail, and (v) a 3′ UTR region. In some embodiments, a polynucleotide further comprises one or more elements, such as one or more sequences encoding an additional payload, or one or more sequences that can form a secondary structure (e.g., hairpin, or an IRES), etc. In some embodiments, a polynucleotide is a messenger RNA.

In some embodiments of an isolated polynucleotide disclosed herein, a composition comprising the same, or methods of making or using a polynucleotide disclosed herein, a polynucleotide comprises one or more methanogen antigens. In some embodiments, a polynucleotide comprises at least 2, at least 3, at least 4, at least 5, at least 6, at least 7, at least 8, at least 9, at least 10, at least 15, or at least 20 methanogen antigens. In some embodiments, one or more methanogen antigens are the same, e.g., antigens having the same sequence, structure and/or function. In some embodiments, one or more methanogen antigens are different, e.g., antigens having different sequence, structure and/or function. In some embodiments, one or more methanogen antigens include a mixture of antigens that are the same and antigens that are different. In some embodiments, one or more methanogen antigens are associated with one or more methanogen species. In some embodiments, one or more methanogen antigens are associated with a single methanogen species.

In some embodiments, one or more methanogen antigens comprise one or more, or all, or any combination of: (i) one or more peptides having at least 80% sequence identity to a methanogen protein: (ii) one or more secreted antigens, e.g., each comprising a signal peptide; (iii) a plurality of peptides having at least 80% sequence identity to each other; (iv) a plurality of peptides associated with at least 2 different, at least 3 different, at least 4 different or at least 5 different methanogen species, (v) one or more peptides having at least 80% sequence identity to a polypeptide having signal peptidase activity; (vi) one or more peptides having at least 80% sequence identity to an AglB polypeptide or substantially similar function to an AglB polypeptide; (vii) one or more peptides having at least 80% sequence identity to an Ig-like domain-containing polypeptide or substantially similar function to an Ig-like domain-containing polypeptide; or (viii) one or more methanogen antigens provided in Table 1 or sequences with at least 85% identity thereto. In some embodiments, a polynucleotide comprises one or more methanogen antigens from each of (i)-(viii).

In some embodiments, one or more methanogen antigens comprise one or more peptides having at least 80%, at least 85%, at least 90%, at least 95%, or at least 99% sequence identity to a methanogen protein. In some embodiments, a methanogen protein comprises a polypeptide encoded from a genome of a methanogen, or a variant or fragment thereof.

In some embodiments, one or more methanogen antigens comprise one or more secreted antigens, e.g., one or more methanogen antigens comprise a signal peptide. In some embodiments, a secreted antigen is or comprises an antigen that is a surface associated protein, or a protein that is secreted out of a cell. In some embodiments, a secreted antigen comprises a signal peptide. In some embodiments, a signal peptide can be predicted using an algorithm. In some embodiments, a prediction score of a signal peptide is at least 0.5.

In some embodiments, one or more methanogen antigens comprise a plurality of peptides having at least 80%, at least 85%, at least 90%, at least 95%, or at least 99% sequence identity to each other. In some embodiments, one or more methanogen antigens comprising at least 80% identity to each other are also referred to as a cluster homology.

In some embodiments, one or more methanogen antigens comprise a plurality of peptides associated with at least 2 different, at least 3 different, at least 4 different, at least 5 different, at least 6 different, at least 7 different, at least 8 different, at least 9 different, at least 10 different, at least 1 l different, at least 12 different, at least 15 different or at least 20 different methanogen species.

In some embodiments, one or more methanogen antigens comprise a plurality of peptides associated with about 2 different, about 3 different, about 4 different, about 5 different, about 6 different, about 7 different, about 8 different, about 9 different, about 10 different, about 11 different, about 12 different, about 15 different or about 20 different methanogen species.

In some embodiments, one or more methanogen antigens comprise a plurality of peptides associated with no more than 2 different, no more than 3 different, no more than 4 different, no more than 5 different, no more than 6 different, no more than 7 different, no more than 8 different, no more than 9 different, no more than 10 different, no more than 11 different, no more than 12 different, no more than 15 different or no more than 20 different methanogen species.

In some embodiments, one or more methanogen antigens comprise a plurality of peptides associated with about 2 to about 20, about 2 to about 15, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 20, about 4 to about 20, about 5 to about 20, about 6 to about 20, about 7 to about 20, about 8 to about 20, about 9 to about 20, about 10 to about 20, about 11 to about 20, about 12 to about 20, or about 15 to about 20 different methanogen species.

In some embodiments, one or more methanogen antigens that are associated with a methanogen species refer to antigens that can be found in a methanogen species. For example, a methanogen antigen associated with a particular methanogen species comprises an antigen encoded by a nucleotide sequence naturally occurring in said methanogen species.

In some embodiments, one or more methanogen antigens comprise a methanogen from one, or more, or all of the following clades: Methanobrevibacter, Methanosphaera, Methanobacterium, Methanosarcinales, Methanomicrobiales, Methanothermobacter, Candidatus Methanomethylophilus, Thermoplasmatales.

In some embodiments, a methanogen species is chosen from: Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thennautotrophicus, Methanococcus aeolicus, Methancaldoococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, or Mathanosarcina acetivorans.

In some embodiments, a methanogen species is chosen from Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188 or Methanosarcina soligelidi.

In some embodiments, a methanogen species is chosen from Methanobrevibacter ruminantium, Methanobrevibacter smithii, or Methanobrevibacter oralis.

In some embodiments, a methanogen species is chosen from Methanobrevibacter ruminantium Methanobrevibacter smithii Methanobrevibacter oralis Methanomicrobium mobile or Methanosphaera stadtmanae.

In some embodiments, one or more methanogen antigens comprise a plurality of methanogen antigens each of which is associated with the same methanogen species.

In some embodiments, one or more methanogen antigens comprise one or more peptides having at least 80%, at least 85%, at least 90%, at least 95%, or at least 99% NO sequence identity to a polypeptide having signal peptidase activity. In some embodiments, a polypeptide having signal peptidase activity has one or more (or all) of the following characteristics. (i) is membrane bound; (ii) has the ability to cleave one or more signal peptides; and/or (iii) plays a role in converting a secretory protein to a mature form (e.g., from a non-secretory form to a secretory form).

In some embodiments, one or more methanogen antigens comprise one or more peptides having at least 80%, at least 85%, at least 90%, at least 95%, or at least 99% sequence identity to an AglB polypeptide or substantially similar function to an AglB polypeptide. In some embodiments, an AglB polypeptide is characterized as having N-glycosylation activity.

In some embodiments, one or more methanogen antigens comprise one or more peptides having at least 80%, at least 85%, at least 90%, at least 95%, or at least 99% sequence identity to an Ig-like domain-containing polypeptide or substantially similar function to an Ig-like domain-containing polypeptide. In some embodiments, an Ig-like domain-containing polypeptide comprises a polypeptide characterized as a cell-surface protein that facilitates binding to other cells and/or cell surfaces.

An Ig-like domain containing polypeptide described herein can include: (i) transglutaminase domain-containing protein or a fragment, variant or variant fragment thereof, (ii) a pseudomurein-binding repeat-containing protein or a fragment, variant or variant fragment thereof, (iii) aright-handed parallel beta-helix repeat-containing protein or a fragment, variant or variant fragment thereof, (iv) a succinylglutamate desuccinylase/aspartoacylase family protein or a fragment, variant or variant fragment thereof, or (v) any combination of (i)-(iv).

In some embodiments, one or more methanogen antigens comprise one or more antigens provided in Table 1 or sequences with at least 80%, at least 85%, at least 90%, at least 95%, or at least 99% identity thereto. In some embodiments, one or more methanogen antigens comprise an adhesin or a fragment or a variant thereof. In some embodiments, one or more methanogen antigens comprise a pilli protein or a fragment or a variant thereof. In some embodiments, one or more methanogen antigens comprise a flagellin protein or a fragment or a variant thereof.

In some embodiments of an isolated polynucleotide disclosed herein, a composition comprising the same, or methods of making or using a polynucleotide disclosed herein, a polynucleotide comprises one or more methanogen antigens. In some embodiments, a polynucleotide comprises one or more methanogen antigens chosen from, one or more, or all, or any combination of (i) one or more peptides having at least 80% sequence identity to a methanogen protein; (ii) one or more secreted antigens comprising a signal peptide; (iii) a plurality of peptides having at least 80% sequence identity to each other; (iv) a plurality of peptides associated with at least 2 different, at least 3 different, at least 4 different or at least 5 different methanogen species; or (v) one or more peptides having at least 80% sequence identity to a polypeptide having signal peptidase activity. In some embodiments, a polynucleotide comprises one or more methanogen antigens from each of (i)-(v). In some embodiments, a polynucleotide comprises three methanogen antigens. In some embodiments, three methanogen antigens are associated with at least three different methanogen species, e.g., Methanobrevibacter ruminantium, Methanobrevibacter smithii, Methanobrevibacter oralis. Exemplary multi-component compositions are described in Table 3.

In some embodiments of an isolated polynucleotide disclosed herein, a composition comprising the same, or methods of making or using a polynucleotide disclosed herein, a polynucleotide comprises one or more methanogen antigens. In some embodiments, a polynucleotide comprises one or more methanogen antigens chosen from, one or more, or all, or any combination of: (i) one or more peptides having at least 80% sequence identity to a methanogen protein; (ii) one or more secreted antigens comprising a signal peptide; (iii) a plurality of peptides associated with at least 2 different, at least 3 different, at least 4 different or at least 5 different methanogen species; or (iv) one or more peptides having at least 80% sequence identity to an AglB polypeptide or substantially similar function to an AglB polypeptide. In some embodiments, a polynucleotide comprises one or more methanogen antigens from each of (i)-(iv). In some embodiments, a polynucleotide comprises five methanogen antigens. In some embodiments, five methanogen antigens are associated with at least five different methanogen species, e.g., Methanobrevibacter ruminantium Methanobrevibacter smithii Methanobrevibacter oralis Methanomicrobium mobile Methanosphaera stadtmanae. Exemplary multi-component compositions are described in Table 3.

In some embodiments of an isolated polynucleotide disclosed herein, a composition comprising the same, or methods of making or using a polynucleotide disclosed herein, a polynucleotide comprises one or more methanogen antigens. In some embodiments, a polynucleotide comprises one or more methanogen antigens chosen from, one or more, or all, or any combination of: (i) one or more peptides having at least 80% sequence identity to a methanogen protein; (ii) one or more secreted antigens comprising a signal peptide; (iii) a plurality of peptides having at least 80% sequence identity to each other; (iv) a plurality of peptides associated with at least 2 different, at least 3 different, at least 4 different or at least 5 different methanogen species; or (v) one or more peptides having at least 80% sequence identity to an Ig-like domain-containing polypeptide or substantially similar function to an Ig-like domain-containing polypeptide. In some embodiments, a polynucleotide comprises one or more methanogen antigens from each of (i)-(v). In some embodiments, a polynucleotide comprises eight methanogen antigens. In some embodiments, eight methanogen antigens are associated with at least three different methanogen species, e.g., Methanobrevibacter ruminantium, Methanobrevibacter smithii, Methanobrevibacter oralis. Exemplary multi-component compositions are described in Table 3.

In some embodiments of an isolated polynucleotide disclosed herein, a composition comprising the same, or methods of making or using a polynucleotide disclosed herein, a polynucleotide comprises one or more methanogen antigens. In some embodiments, a polynucleotide comprises one or more methanogen antigens chosen from, one or more, or all, or any combination of: (i) one or more peptides having at least 80% sequence identity to a methanogen protein. (ii) one or more secreted antigens comprising a signal peptide; (iii) a plurality of peptides having at least 80% sequence identity to each other; (iv) a plurality of peptides associated with at least 2 different, at least 3 different, at least 4 different or at least 5 different methanogen species; or (v) one or more peptides having at least 80% sequence identity to an Ig-like domain-containing polypeptide or substantially similar function to an Ig-like domain-containing polypeptide. In some embodiments, a polynucleotide comprises one or more methanogen antigens from each of (i)-(v). In some embodiments, a polynucleotide comprises 34 methanogen antigens. In some embodiments, one or more methanogen antigens are associated with at least five different methanogen species, e.g., Methanobrevibacter ruminantium, Methanobrevibacter smithii, Methanobrevibacler oralis, Methanobrevibacter thaueri, Methaniobrevibacter sp. UBA188. An exemplary multi-component composition is described in Table 5.

In some embodiments, a composition disclosed herein comprises one or more polynucleotides comprising one or more ruminal-associated antigens, e.g., methanogen antigens. In some embodiments, a composition (e.g., a multi-component composition, e.g., a multi-component vaccine composition) comprises a plurality of polynucleotides (e.g., a plurality of RNA) each comprising an antigen. In some embodiments, a composition (e.g., a multi-component composition, a multi-component vaccine composition) comprises a plurality of polynucleotides (e.g., a plurality of RNA) each comprising one or more antigens (e.g., the same or different antigens).

In some embodiments, a composition (e.g., a multi-component composition, e.g., a multi-component vaccine composition), comprising a plurality of polynucleotides comprises about 1% of a first polynucleotide and about 99% of one or more additional polynucleotides. In some embodiments, a composition (e.g., a multi-component composition, e.g., a multi-component vaccine composition), comprising a plurality of polynucleotides comprises about 5% of a first polynucleotide and about 95% of one or more additional polynucleotides. In some embodiments, a composition (e.g., a multi-component composition, e.g., a multi-component vaccine composition), comprising a plurality of polynucleotides comprises about 10% of a first polynucleotide and about 90% of one or more additional polynucleotides. In some embodiments, a composition (e.g., a multi-component composition, e.g., a multi-component vaccine composition), comprising a plurality of polynucleotides comprises about 20% of a first polynucleotide and about 80% of one or more additional polynucleotides. In some embodiments, a composition (e.g., a multi-component composition, e.g., a multi-component vaccine composition), comprising a plurality of polynucleotides comprises about 30% of a first polynucleotide and about 70% of one or more additional polynucleotides. In some embodiments, a composition (e.g., a multi-component composition, e.g., a multi-component vaccine composition), comprising a plurality of polynucleotides comprises about 40% of a first polynucleotide and about 60% of one or more additional polynucleotides. In some embodiments, a composition (e.g., a multi-component composition, e.g., a multi-component vaccine composition), comprising a plurality of polynucleotides comprises about 50% of a first polynucleotide and about 50% of one or more additional polynucleotides. In some embodiments, a composition (e.g., a multi-component composition, e.g., a multi-component vaccine composition), comprising a plurality of polynucleotides comprises about 60% of a first polynucleotide and about 40%, of one or more additional polynucleotides. In some embodiments, a composition (e.g., a multi-component composition, e.g., a multi-component vaccine composition), comprising a plurality of polynucleotides comprises about 70% of a first polynucleotide and about 300% of one or more additional polynucleotides. In some embodiments, a composition (e.g., a multi-component composition, e.g., a multi-component vaccine composition), comprising a plurality of polynucleotides comprises about 80% of a first polynucleotide and about 20% of one or more additional polynucleotides. In some embodiments, a composition (e.g., a multi-component composition, e.g., a multi-component vaccine composition), comprising a plurality of polynucleotides comprises about 90% of a first polynucleotide and about 10% of one or more additional polynucleotides. In some embodiments, a composition (e.g., a multi-component composition, e.g., a multi-component vaccine composition), comprising a plurality of polynucleotides comprises about 99% of a first polynucleotide and about 1% of one or more additional polynucleotides.

In some embodiments, a composition (e.g., a multi-component composition, e.g., a multi-component vaccine composition), comprising a plurality of polynucleotides comprises about 33% of a first polynucleotide, about 33% of a second polynucleotides and about 33% of a third polynucleotide.

Exemplary methanogen antigens disclosed herein can be obtained, e.g., from sequencing samples from a rumen of an animal, e.g., a ruminant. Sequencing samples from a rumen of an animal, e.g., a ruminant, can provide information about specific protein targets and/or specific species, e.g., methanogen species. Additionally, methanogen antigens can also be obtained by using a list, e.g., a defined list, of target species, e.g, methanogens, as would readily be ascertainable by one with knowledge in the pertinent field Additionally or alternatively, methanogen antigens can also be obtained by using algorithms or language models such as those known in the field, to generate de novo proteins or variants of related sequence or structure.

Accordingly, also provided herein is a method of identifying one or more methanogen antigens for use in a polynucleotide disclosed herein or a composition comprising the same, e.g., for vaccinating an animal, e.g., a ruminant.

### Acetylated Nucleotides

Among other things, provided herein are polyribonucleotides comprising one or more modified ribonucleotides including a nucleoside comprising an acetyl group. In some embodiments, a nucleoside of a modified ribonucleotide is N4-acetylcytidine and the modified ribonucleotide has. a 5′ monophosphate, a 5′ diphosphate or a 5′ triphosphate. Additional details about polyribonucleotides comprising one or more modified ribonucleotides including a nucleoside comprising an acetyl group is provided in International Patent Application PCT/US22/27721 filed on Jul. 18, 202, the entire contents of which are hereby incorporated by reference.

In some embodiments, a nucleoside of a modified ribonucleotide is N4-acetylcytidine and the modified ribonucleotide has a structure of

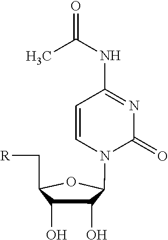

wherein R is a 5′ monophosphate, a 5′ diphosphate, or a 5′ triphosphate.

In some embodiments, a polyribonucleotide disclosed herein comprises cytidine residues. In some embodiments, at least 5% of cytidine residues in a polyribonucleotide comprise N4-acetylcytidine. In some embodiments, less than 100% of cytidine residues in a polyribonucleotide comprise N4-acetylcytidine.

In some embodiments, a polyribonucleotide disclosed herein comprises cytidine residues and at least 5% of cytidine residues in a polyribonucleotide comprises N4-acetylcytidine.

In some embodiments, a polyribonucleotide disclosed herein comprises cytidine residues and at least 10% of cytidine residues in a polyribonucleotide comprises N4-acetylcytidine.

In some embodiments, a polyribonucleotide disclosed herein comprises cytidine residues and at least 15% of cytidine residues in a polyribonucleotide comprises N4-acetylcytidine.

In some embodiments, a polyribonucleotide disclosed herein comprises cytidine residues and at least 20% of cytidine residues in a polyribonucleotide comprises N4-acetylcytidine.

In some embodiments, a polyribonucleotide disclosed herein comprises cytidine residues and at least 25% of cytidine residues in a polyribonucleotide comprises N4-acetylcytidine.

In some embodiments, a polyribonucleotide disclosed herein comprises cytidine residues and at least 30% of cytidine residues in a polyribonucleotide comprises N4-acetylcytidine.

In some embodiments, a polyribonucleotide disclosed herein comprises cytidine residues and at least 35% of cytidine residues in a polyribonucleotide comprises N4-acetylcytidine.

In some embodiments, a polyribonucleotide disclosed herein comprises cytidine residues and at least 40% of cytidine residues in a polyribonucleotide comprises N4-acetylcytidine.

In some embodiments, a polyribonucleotide disclosed herein comprises cytidine residues and at least 45% of cytidine residues in a polyribonucleotide comprises N4-acetylcytidine.

In some embodiments, a polyribonucleotide disclosed herein comprises cytidine residues and at least 50% of cytidine residues in a polyribonucleotide comprises N4-acetylcytidine.

In some embodiments, a polyribonucleotide disclosed herein comprises cytidine residues and at least 55% of cytidine residues in a polyribonucleotide comprises N4-acetylcytidine.

In some embodiments, a polyribonucleotide disclosed herein comprises cytidine residues and at least 60% of cytidine residues in a polyribonucleotide comprises N4-acetylcytidine.

In some embodiments, a polyribonucleotide disclosed herein comprises cytidine residues and at least 65% of cytidine residues in a polyribonucleotide comprises N4-acetylcytidine.

In some embodiments, a polyribonucleotide disclosed herein comprises cytidine residues and at least 70% of cytidine residues in a polyribonucleotide comprises N4-acetylcytidine.

In some embodiments, a polyribonucleotide disclosed herein comprises cytidine residues and at least 75% of cytidine residues in a polyribonucleotide comprises N4-acetylcytidine.

In some embodiments, a polyribonucleotide disclosed herein comprises cytidine residues and at least 80% of cytidine residues in a polyribonucleotide comprises N4-acetylcytidine.

In some embodiments, a polyribonucleotide disclosed herein comprises cytidine residues and at least 85% of cytidine residues in a polyribonucleotide comprises N4-acetylcytidine.

In some embodiments, a polyribonucleotide disclosed herein comprises cytidine residues and at least 90% of cytidine residues in a polyribonucleotide comprises N4-acetylcytidine.

In some embodiments, a polyribonucleotide disclosed herein comprises cytidine residues and at least 95% of cytidine residues in a polyribonucleotide comprises N4-acetylcytidine.

In some embodiments, a polyribonucleotide disclosed herein comprises cytidine residues. In some embodiments, about 5% to 99%, about 5% to 95%, about 5% to 90%, about 5% to 85%, about 5% to 80%, about 5% to 75%, about 5% to 70%, about 5% to 65%, about 5% to 60%, about 5% to 55%, about 5% to 50%, about 5% to 45%, about 5% to 40%, about 5% to 35%, about 5% to 30%, about 5% to 25%, about 5% to 20%, about 5% to 15%, about 5% to 10%, about 10% to 99%, about 15% to 99%, about 20% to 99%, about 25% to 99%, about 30% to 99%, about 35% to 99%, about 40% to 99%, about 45% to 99%, about 50% to 99%, about 55% to 99%, about 60% to 99%, about 65% to 99%, about 70% to 99%, about 80% to 99%, about 85% to 99%, about 90% to 99%, or about 95% to 99% of cytidine residues in a polyribonucleotide comprises N4-acetylcytidine.

In some embodiments, a polyribonucleotide disclosed herein comprises cytidine residues and more than about 60% of cytidine residues in a polyribonucleotide comprises N4-acetylcytidine.

In some embodiments, a polyribonucleotide disclosed herein comprises cytidine residues and more than about 65% of cytidine residues in a polyribonucleotide comprises N4-acetylcytidine.

In some embodiments, a polyribonucleotide disclosed herein comprises cytidine residues and more than about 70% of cytidine residues in a polyribonucleotide comprises N4-acetylcytidine.

In some embodiments, a polyribonucleotide disclosed herein comprises cytidine residues and more than about 75% of cytidine residues in a polyribonucleotide comprises N4-acetylcytidine.

In some embodiments, a polyribonucleotide disclosed herein comprises cytidine residues and more than about 80% of cytidine residues in a polyribonucleotide comprises N4-acetylcytidine.

In some embodiments, a polyribonucleotide disclosed herein comprises cytidine residues and more than about 85% of cytidine residues in a polyribonucleotide comprises N4-acetylcytidine.

In some embodiments, a polyribonucleotide disclosed herein comprises cytidine residues and more than about 90% of cytidine residues in a polyribonucleotide comprises N4-acetylcytidine.

In some embodiments, a polyribonucleotide disclosed herein comprises cytidine residues and more than about 95% of cytidine residues in a polyribonucleotide comprises N4-acetylcytidine.

In some embodiments, a polyribonucleotide disclosed herein comprises cytidine residues and more than about 99%6 of cytidine residues in a polyribonucleotide comprises N4-acetylcytidine.

In some embodiments, a polyribonucleotide disclosed herein comprises cytidine residues and about 5% of cytidine residues in a polyribonucleotide comprises N4-acetylcytidine.

In some embodiments, a polyribonucleotide disclosed herein comprises cytidine residues and about 10% of cytidine residues in a polyribonucleotide comprises N4-acetylcytidine.

In some embodiments, a polyribonucleotide disclosed herein comprises cytidine residues and about 15% of cytidine residues in a polyribonucleotide comprises N4-acetylcytidine.

In some embodiments, a polyribonucleotide disclosed herein comprises cytidine residues and about 20% of cytidine residues in a polyribonucleotide comprises N4-acetylcytidine.

In some embodiments, a polyribonucleotide disclosed herein comprises cytidine residues and about 25% of cytidine residues in a polyribonucleotide comprises N4-acetylcytidine.

In some embodiments, a polyribonucleotide disclosed herein comprises cytidine residues and about 30% of cytidine residues in a polyribonucleotide comprises N4-acetylcytidine.

In some embodiments, a polyribonucleotide disclosed herein comprises cytidine residues and about 35% of cytidine residues in a polyribonucleotide comprises N4-acetylcytidine.

In some embodiments, a polyribonucleotide disclosed herein comprises cytidine residues and about 40% of cytidine residues in a polyribonucleotide comprises N4-acetylcytidine.

In some embodiments, a polyribonucleotide disclosed herein comprises cytidine residues and about 45% of cytidine residues in a polyribonucleotide comprises N4-acetylcytidine.

In some embodiments, a polyribonucleotide disclosed herein comprises cytidine residues and about 50% of cytidine residues in a polyribonucleotide comprises N4-acetylcytidine.

In some embodiments, a polyribonucleotide disclosed herein comprises cytidine residues and about 55% of cytidine residues in a polyribonucleotide comprises N4-acetylcytidine.

In some embodiments, a polyribonucleotide disclosed herein comprises cytidine residues and about 60% of cytidine residues in a polyribonucleotide comprises N4-acetylcytidine.

In some embodiments, a polyribonucleotide disclosed herein comprises cytidine residues and about 65% of cytidine residues in a polyribonucleotide comprises N4-acetylcytidine.

In some embodiments, a polyribonucleotide disclosed herein comprises cytidine residues and about 75% of cytidine residues in a polyribonucleotide comprises N4-acetylcytidine.

In some embodiments, a polyribonucleotide disclosed herein comprises cytidine residues and about 80% of cytidine residues in a polyribonucleotide comprises N4-acetylcytidine.

In some embodiments, a polyribonucleotide disclosed herein comprises cytidine residues and about 85% of cytidine residues in a polyribonucleotide comprises N4-acetylcytidine.

In some embodiments, a polyribonucleotide disclosed herein comprises cytidine residues and about 90% of cytidine residues in a polyribonucleotide comprises N4-acetylcytidine.

In some embodiments, a polyribonucleotide disclosed herein comprises cytidine residues and about 95% of cytidine residues in a polyribonucleotide comprises N4-acetylcytidine.

In some embodiments, a polyribonucleotide disclosed herein comprises cytidine residues and about 99% of cytidine residues in a polyribonucleotide comprises N4-acetylcytidine.

In some embodiments, a polyribonucleotide disclosed herein comprises cytidine residues and 100% of cytidine residues in a polyribonucleotide comprise N4-acetylcytidine.

In some embodiments, a polyribonucleotide disclosed herein (e.g., a polyribonucleotide comprising cytidine residues with about 5%-100% cytidine residues comprising N4-aceytlcytidine) comprises one or more additional modified ribonucleotides. In some embodiments, one or more additional modified ribonucleotides comprises a nucleoside chosen from: an adenosine, an inosine, a guanosine, a cytidine or a uridine, or any combination thereof. In some embodiments, one or more additional modified ribonucleotides comprises a 5-hydroxymethyl group. In some embodiments, one or more additional modified ribonucleotides comprises 5-hydroxymethyluridine. In some embodiments 5%-100% of uridine residues in a polyribonucleotide comprising uridine are 5-hydroxymethyluridine.

In some embodiments, a polyribonucleotide can have a length of at least 5 nucleotides or longer. In some embodiments, a polyribonucleotide can have a length of at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, at least 200 nucleotides, at least 300 nucleotides, at least 400 nucleotides, at least 500 nucleotides, at least 1000 nucleotides, at least 2000 nucleotides, at least 5000 nucleotides or longer.

In some embodiments, a polyribonucleotide can have a length of about 5 nucleotides to about 200,000 nucleotides, about 5 nucleotides to about 150,000 nucleotides, about 5 nucleotides to about 100,000 nucleotides, about 5 nucleotides to about 50,000 nucleotides, about 5 nucleotides to about 10,000 nucleotides, about 5 nucleotides to about 5000 nucleotides, about 5 nucleotides to about 1000 nucleotides, about 5 nucleotides to about 500 nucleotides, about 5 nucleotides to about 400 nucleotides, about 5 nucleotides to about 300 nucleotides, about 5 nucleotides to about 200 nucleotides, about 5 nucleotides to about 100 nucleotides, about 5 nucleotides to about 90 nucleotides, about 5 nucleotides to about 85 nucleotides, about 5 nucleotides to about 80 nucleotides, about 5 nucleotides to about 75 nucleotides, about 5 nucleotides to about 70 nucleotides, about 5 nucleotides to about 65 nucleotides, about 5 nucleotides to about 60 nucleotides, about 5 nucleotides to about 55 nucleotides, about 5 nucleotides to about 50 nucleotides, about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides.

In some embodiments, a polyribonucleotide can have a length of about 5 nucleotides to about 200,000 nucleotides, about 10 nucleotides to about 200.000 nucleotides, 15 nucleotides to about 200,000 nucleotides, about 20 nucleotides to about 200,000 nucleotides, about 30 nucleotides to about 200,000 nucleotides, about 40 nucleotides to about 200,000 nucleotides, about 50 nucleotides to about 200,000 nucleotides, about 100 nucleotides to about 200,000 nucleotides, about 200 nucleotides to about 200,000 nucleotides, about 300 nucleotides to about 200,000 nucleotides, about 400 nucleotides to about 200,000 nucleotides, about 500 nucleotides to about 200,000 nucleotides, about 1000 nucleotides to about 200,000 nucleotides, about 2000 nucleotides to about 200,000 nucleotides, about 3000 nucleotides to about 200,000 nucleotides, about 4000 nucleotides to about 200,000 nucleotides, about 5000 nucleotides to about 200,000 nucleotides, about 10,000 nucleotides to about 200,000 nucleotides, about 20,000 nucleotides to about 200,000 nucleotides, about 30,000 nucleotides to about 200,000 nucleotides, about 40,000 nucleotides to about 200,000 nucleotides, about 50,000 nucleotides to about 200,000 nucleotides, about 100,000 nucleotides to about 200,000 nucleotides, about 150,000 nucleotides to about 200,000 nucleotides.

In some embodiments, a polyribonucleotide can have a length of no more than 200.000 nucleotides, no more than 150,000 nucleotides, no more than 100,000 nucleotides, or no more than 50,000 nucleotides.

### 5-Hydroxymethyl Modified Nucleotides

Among other things, provided herein are polyribonucleotides comprising one or more modified ribonucleotides including a nucleoside comprising a 5-hydroxymethyl group. In some embodiments, a nucleoside of a modified ribonucleotide is 5-hydroxymethyluridine and the modified ribonucleotide has: a 5′ monophosphate, a 5′ diphosphate or a 5′ triphosphate. Additional details about ribonucleotides including a nucleoside comprising a 5-hydroxymethyl group is provided in International Patent Application PCT/US22/27721 filed on Jul. 18, 202, the entire contents of which are hereby incorporated by reference.

In some embodiments, a nucleoside of a modified ribonucleotide is 5-hydroxymethyluridine and the modified ribonucleotide has a structure of

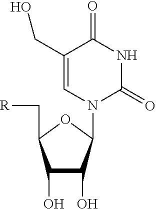

wherein R is a 5′ monophosphate, a 5′ diphosphate, or a 5′ triphosphate.

In some embodiments, a polyribonucleotide disclosed herein comprises uridine residues. In some embodiments, at least 5% of uridine residues in a polyribonucleotide comprise 5-hydroxymethyluridine. In some embodiments, less than 100% of uridine residues in a polyribonucleotide comprise 5-hydroxymethyluridine.

In some embodiments, a polyribonucleotide disclosed herein comprises uridine residues and at least 5% of uridine residues in a polyribonucleotide comprises 5-hydroxymethyluridine.

In some embodiments, a polyribonucleotide disclosed herein comprises uridine residues and at least 10% of uridine residues in a polyribonucleotide comprises 5-hydroxymethyluridine.

In some embodiments, a polyribonucleotide disclosed herein comprises uridine residues and at least 15% of uridine residues in a polyribonucleotide comprises 5-hydroxymethyluridine.

In some embodiments, a polyribonucleotide disclosed herein comprises uridine residues and at least 20% of uridine residues in a polyribonucleotide comprises 5-hydroxymethyluridine.

In some embodiments, a polyribonucleotide disclosed herein comprises uridine residues and at least 25% of uridine residues in a polyribonucleotide comprises 5-hydroxymethyluridine.

In some embodiments, a polyribonucleotide disclosed herein comprises uridine residues and at least 30% of uridine residues in a polyribonucleotide comprises 5-hydroxymethyluridine.

In some embodiments, a polyribonucleotide disclosed herein comprises uridine residues and at least 35% of uridine residues in a polyribonucleotide comprises 5-hydroxymethyluridine.

In some embodiments, a polyribonucleotide disclosed herein comprises uridine residues and at least 40% of uridine residues in a polyribonucleotide comprises 5-hydroxymethyluridine.

In some embodiments, a polyribonucleotide disclosed herein comprises uridine residues and at least 45% of uridine residues in a polyribonucleotide comprises 5-hydroxymethyluridine.

In some embodiments, a polyribonucleotide disclosed herein comprises uridine residues and at least 50% of uridine residues in a polyribonucleotide comprises 5-hydroxymethyluridine.

In some embodiments, a polyribonucleotide disclosed herein comprises uridine residues and at least 55% of uridine residues in a polyribonucleotide comprises 5-hydroxymethyluridine.

In some embodiments, a polyribonucleotide disclosed herein comprises uridine residues and at least 60% of uridine residues in a polyribonucleotide comprises 5-hydroxymethyluridine.

In some embodiments, a polyribonucleotide disclosed herein comprises uridine residues and at least 65% of uridine residues in a polyribonucleotide comprises 5-hydroxymethyluridine.

In some embodiments, a polyribonucleotide disclosed herein comprises uridine residues and at least 70% of uridine residues in a polyribonucleotide comprises 5-hydroxymethyluridine.

In some embodiments, a polyribonucleotide disclosed herein comprises uridine residues and at least 75% of uridine residues in a polyribonucleotide comprises 5-hydroxymethyluridine.

In some embodiments, a polyribonucleotide disclosed herein comprises uridine residues and at least 80% of uridine residues in a polyribonucleotide comprises 5-hydroxymethyluridine.

In some embodiments, a polyribonucleotide disclosed herein comprises uridine residues and at least 85% of uridine residues in a polyribonucleotide comprises 5-hydroxymethyluridine.

In some embodiments, a polyribonucleotide disclosed herein comprises uridine residues and at least 90% of uridine residues in a polyribonucleotide comprises 5-hydroxymethyluridine.

In some embodiments, a polyribonucleotide disclosed herein comprises uridine residues and at least 95% of uridine residues in a polyribonucleotide comprises 5-hydroxymethyluridine.

In some embodiments, a polyribonucleotide disclosed herein comprises uridine residues and at least 99% of uridine residues in a polyribonucleotide comprises 5-hydroxymethyluridine.

In some embodiments, a polyribonucleotide disclosed herein comprises uridine residues. In some embodiments, about 5% to 99%, about 5% to 95%, about 5% to 90%, about 5% to 85%, about 5% to 80%, about 5% to 75%, about 5% to 70%, about 5% to 65%, about 5% to 60%, about 5% to 55%, about 5% to 50%, about 5% to 45%, about 5% to 40%, about 5% to 35%, about 5% to 30%, about 5% to 25%, about 5% to 20%, about 5% to 15%, about 5% to 10%, about 10% to 99%, about 15% to 99%, about 20% to 99%, about 25% to 99%, about 30% to 99%, about 35% to 99%, about 40% to 99%, about 45% to 99%/, about 50% to 99%, about 55% to 99%, about 60% to 99%, about 65% to 99%, about 70% to 99%, about 80% to 99%, about 85% to 99%, about 90% to 99%, or about 95% to 99% of uridine residues in a polyribonucleotide comprises 5-hydroxymethyluridine.

In some embodiments, a polyribonucleotide disclosed herein comprises uridine residues and more than 60% of uridine residues in a polyribonucleotide comprises 5-hydroxymethyluridine.

In some embodiments, a polyribonucleotide disclosed herein comprises uridine residues and more than 65% of uridine residues in a polyribonucleotide comprises 5-hydroxymethyluridine.

In some embodiments, a polyribonucleotide disclosed herein comprises uridine residues and more than 70% of uridine residues in a polyribonucleotide comprises 5-hydroxymethyluridine.

In some embodiments, a polyribonucleotide disclosed herein comprises uridine residues and more than 75% of uridine residues in a polyribonucleotide comprises 5-hydroxymethyluridine.

In some embodiments, a polyribonucleotide disclosed herein comprises uridine residues and more than 80% of uridine residues in a polyribonucleotide comprises 5-hydroxymethyluridine.

In some embodiments, a polyribonucleotide disclosed herein comprises uridine residues and more than 85% of uridine residues in a polyribonucleotide comprises 5-hydroxymethyluridine.

In some embodiments, a polyribonucleotide disclosed herein comprises uridine residues and more than 90% of uridine residues in a polyribonucleotide comprises 5-hydroxymethyluridine.

In some embodiments, a polyribonucleotide disclosed herein comprises uridine residues and more than 95% of uridine residues in a polyribonucleotide comprises 5-hydroxymethyluridine.

In some embodiments, a polyribonucleotide disclosed herein comprises uridine residues and more than 99% of uridine residues in a polyribonucleotide comprises 5-hydroxymethyluridine.

In some embodiments, a polyribonucleotide disclosed herein comprises uridine residues and about 5% of uridine residues in a polyribonucleotide comprises 5-hydroxymethyluridine.

In some embodiments, a polyribonucleotide disclosed herein comprises uridine residues and about 10% of uridine residues in a polyribonucleotide comprises 5-hydroxymethyluridine.

In some embodiments, a polyribonucleotide disclosed herein comprises uridine residues and about 15% of uridine residues in a polyribonucleotide comprises 5-hydroxymethyluridine.

In some embodiments, a polyribonucleotide disclosed herein comprises uridine residues and about 20% of uridine residues in a polyribonucleotide comprises 5-hydroxymethyluridine.

In some embodiments, a polyribonucleotide disclosed herein comprises uridine residues and about 25% of uridine residues in a polyribonucleotide comprises 5-hydroxymethyluridine.

In some embodiments, a polyribonucleotide disclosed herein comprises uridine residues and about 30% of uridine residues in a polyribonucleotide comprises 5-hydroxymethyluridine.

In some embodiments, a polyribonucleotide disclosed herein comprises uridine residues and about 35% of uridine residues in a polyribonucleotide comprises 5-hydroxymethyluridine.

In some embodiments, a polyribonucleotide disclosed herein comprises uridine residues and about 40% of uridine residues in a polyribonucleotide comprises 5-hydroxymethyluridine.

In some embodiments, a polyribonucleotide disclosed herein comprises uridine residues and about 45% of uridine residues in a polyribonucleotide comprises 5-hydroxymethyluridine.

In some embodiments, a polyribonucleotide disclosed herein comprises uridine residues and about 50% of uridine residues in a polyribonucleotide comprises 5-hydroxymethyluridine.

In some embodiments, a polyribonucleotide disclosed herein comprises uridine residues and about 55% of uridine residues in a polyribonucleotide comprises 5-hydroxymethyluridine.

In some embodiments, a polyribonucleotide disclosed herein comprises uridine residues and about 60% of uridine residues in a polyribonucleotide comprises 5-hydroxymethyluridine.

In some embodiments, a polyribonucleotide disclosed herein comprises uridine residues and about 65% of uridine residues in a polyribonucleotide comprises 5-hydroxymethyluridine.

In some embodiments, a polyribonucleotide disclosed herein comprises uridine residues and about 75% of uridine residues in a polyribonucleotide comprises 5-hydroxymethyluridine.

In some embodiments, a polyribonucleotide disclosed herein comprises uridine residues and about 80% of uridine residues in a polyribonucleotide comprises 5-hydroxymethyluridine.

In some embodiments, a polyribonucleotide disclosed herein comprises uridine residues and about 85% of uridine residues in a polyribonucleotide comprises 5-hydroxymethyluridine.

In some embodiments, a polyribonucleotide disclosed herein comprises uridine residues and about 90% of uridine residues in a polyribonucleotide comprises 5-hydroxymethyluridine.

In some embodiments, a polyribonucleotide disclosed herein comprises uridine residues and about 95% of uridine residues in a polyribonucleotide comprises 5-hydroxymethyluridine.

In some embodiments, a polyribonucleotide disclosed herein comprises uridine residues and about 99% of uridine residues in a polyribonucleotide comprises 5-hydroxymethyluridine.

In some embodiments, a polyribonucleotide disclosed herein comprises uridine residues and 100% of uridine residues in a polyribonucleotide comprises 5-hydroxymethyluridine.

In some embodiments, a polyribonucleotide disclosed herein (e.g., a polyribonucleotide comprising uridine residues with about 5%-100% uridine residues comprising 5-hydroxymethyluridine) comprises one or more additional modified ribonucleotides other than 5-hydroxymethyluridine. In some embodiments, one or more additional modified ribonucleotides comprises a nucleoside chosen from: an adenosine, an inosine, a guanosine, a cytidine or a uridine, or any combination thereof. In some embodiments, one or more additional modified ribonucleotides comprises an acetyl group. In some embodiments, one or more additional modified ribonucleotides comprises N4-aceytlcytidine. In some embodiments 5%-100% of cytidine residues in a polyribonucleotide comprising cytidine are N4-aceytlcytidine.

In some embodiments, a polyribonucleotide can have a length of at least 5 nucleotides or longer. In some embodiments, a polyribonucleotide can have a length of at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, at least 200 nucleotides, at least 300 nucleotides, at least 400 nucleotides, at least 500 nucleotides, at least 1000 nucleotides, at least 2000 nucleotides, at least 5000 nucleotides or longer.

In some embodiments, a polyribonucleotide can have a length of about 5 nucleotides to about 200,000 nucleotides, about 5 nucleotides to about 150,000 nucleotides, about 5 nucleotides to about 100,000 nucleotides, about 5 nucleotides to about 50,000 nucleotides, about 5 nucleotides to about 10,000 nucleotides, about 5 nucleotides to about 5000 nucleotides, about 5 nucleotides to about 1000 nucleotides, about 5 nucleotides to about 500 nucleotides, about 5 nucleotides to about 400 nucleotides, about 5 nucleotides to about 300 nucleotides, about 5 nucleotides to about 200 nucleotides, about 5 nucleotides to about 100 nucleotides, about 5 nucleotides to about 90 nucleotides, about 5 nucleotides to about 85 nucleotides, about 5 nucleotides to about 80 nucleotides, about 5 nucleotides to about 75 nucleotides, about 5 nucleotides to about 70 nucleotides, about 5 nucleotides to about 65 nucleotides, about 5 nucleotides to about 60 nucleotides, about 5 nucleotides to about 55 nucleotides, about 5 nucleotides to about 50 nucleotides, about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides.

In some embodiments, a polyribonucleotide can have a length of about 5 nucleotides to about 200,000 nucleotides, about 10 nucleotides to about 200,000 nucleotides, 15 nucleotides to about 200,000 nucleotides, about 20 nucleotides to about 200,000 nucleotides, about 30 nucleotides to about 200,000 nucleotides, about 40 nucleotides to about 200,000 nucleotides, about 50 nucleotides to about 200,000 nucleotides, about 100 nucleotides to about 200,000 nucleotides, about 200 nucleotides to about 200,000 nucleotides, about 300 nucleotides to about 200,000 nucleotides, about 400 nucleotides to about 200,000 nucleotides, about 500 nucleotides to about 200,000 nucleotides, about 1000 nucleotides to about 200,000 nucleotides, about 2000 nucleotides to about 200,000 nucleotides, about 3000 nucleotides to about 200,000 nucleotides, about 4000 nucleotides to about 200,000 nucleotides, about 5000 nucleotides to about 200,000 nucleotides, about 10,000 nucleotides to about 200,000 nucleotides, about 20,000 nucleotides to about 200,000 nucleotides, about 30,000 nucleotides to about 200,000 nucleotides, about 40,000 nucleotides to about 200,000 nucleotides, about 50,000 nucleotides to about 200,000 nucleotides, about 100,000 nucleotides to about 200,000 nucleotides, about 150,000 nucleotides to about 200,000 nucleotides.

In some embodiments, a polyribonucleotide can have a length of no more than 200,000 nucleotides, no more than 150,000 nucleotides, no more than 100,000 nucleotides, or no more than 50,000 nucleotides.

### SBI Adjuvant

Polynucleotides disclosed herein encoding one or more ruminal-associated antigens can further comprise a sequence encoding an adjuvant.

In some embodiments, an adjuvant disclosed herein can be used to elicit and/or modulate an immune response elicited by an antigen (e.g., fragment antigen or antigen variant) described herein. In some embodiments, an adjuvant disclosed herein comprises a complement binding polypeptide. In some embodiments, a complement binding polypeptide comprises a complement C3d binding polypeptide. An exemplary C3d binding polypeptide is an immunoglobulin-binding protein (Sbi) of Staphylococcus aureus.

As disclosed herein. S. aureus binder of immunoglobulin (Sbi) is an exemplary polypeptide which can bind complement C3d (as described in Clark et al. (2011) Mol Immunol. 48(4): 452-462, the entire contents of which is incorporated herein by reference). Sbi comprises two immunoglobulin binding domains (Domains I and II) and two complement C3d binding domains (Domains III and IV). Sbi domains III and IV can bind C3d (in native C3, iC3b and C3dg) and can result in fluid phase consumption of C3 via activation of the alternative pathway (see Clark et al 2011). It has also been shown that Sbi can be secreted and is involved in S. aureus immune evasion (Burman et al., 2008 J. Biol. Chem; 283:17579-17593).

Without wishing to be bound by theory, it is believed that in some embodiments, a complement C3d-binding polypeptide from Sbi of S. aureus can be used as an adjuvant to enhance and/or modulate an immune response from an antigen described herein. In some embodiments, the immune response is elicited by a fragment antigen or antigen variant disclosed herein. In some embodiments, the immune response is elicited by a component of Sbi of S. aureus.

Exemplary Sbi adjuvants and compositions comprising the same are disclosed in International Patent Application PCT/US2022/018610 filed on Mar. 3, 2022, the entire contents of which are hereby incorporated by reference.

### Compositions

Among other things, the present disclosure provides compositions comprising one or more ruminal-associated antigens, e.g., one or more ruminal antigens and/or one or more methanogen antigens. Compositions disclosed herein may also include polynucleotides encoding the same.

In some embodiments, a composition disclosed herein comprises a polynucleotide comprising a first nucleotide sequence encoding one or more ruminal antigens, and a second nucleotide sequence encoding one or more methanogen antigens.

In some embodiments, a first nucleotide sequence comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 ruminal antigens.

In some embodiments, a first nucleotide sequence comprises about 2-100, about 3-100, about 4-100, about 5-100, about 6-100, about 7-100, about 8-100, about 9-100, about 10-100, about 20-100, about 30-100, about 40-100, about 50-100, about 60-100, about 70-100, about 80-100, about 90-100, about 2-90, about 2-80, about 2-70, about 2-60, about 2-50, about 2-40, about 2-30, about 2-20, about 2-10, about 2-15, about 2-14, about 2-13, about 2-12, about 2-11, about 2-10, about 2-9, about 2-8, about 2-7, about 2-6, about 2-5, about 2-4, about 2-3 ruminal antigens.

In some embodiments, a second nucleotide sequence comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 methanogen antigens.

In some embodiments, a second nucleotide sequence comprises about 2-100, about 3-100, about 4-100, about 5-100, about 6-100, about 7-100, about 8-100, about 9-100, about 10-100, about 20-100, about 30-100, about 40-100, about 50-100, about 60-100, about 70-100, about 80-100, about 90-100, about 2-90, about 2-80, about 2-70, about 2-60, about 2-50, about 2-40, about 2-30, about 2-20, about 2-10, about 2-15, about 2-14, about 2-13, about 2-12, about 2-11, about 2-10, about 2-9, about 2-8, about 2-7, about 2-6, about 2-5, about 2-4, about 2-3 methanogen antigens.

In some embodiments, a composition disclosed herein comprises a polynucleotide comprising one or more ruminal-associated antigens and a polynucleotide comprising one or more cytokines and/or chemokines. In some embodiments, a cytokine and/or chemokine is one expressed in a ruminant. In some embodiments, a cytokine and/or chemokine is or comprises VIP, CXCL10, APRIL, or any combination thereof.

In some embodiments, a cytokine and/or chemokine comprises a sequence provided in any one of SEQ ID NOs: 9, 11, or 13, or a sequence with at least 85% identity thereto. Cytokine/chemokine sequences are underlined in SEQ ID NOs: 9, 11 or 13.

In some embodiments, a cytokine and/or chemokine comprises a polypeptide encoded by a cytokine and/or chemokine nucleotide sequence provided in any one of SEQ ID NOs: 8, 10 or 12, or a sequence with at least 85% identity thereto. Cytokine and/or chemokine sequences are underlined in SEQ ID NOs: 8, 10 and 12

In some embodiments, a composition disclosed herein is a pharmaceutical composition.

In some embodiments, a composition disclosed herein is an immunogenic composition.

In some embodiments, a composition disclosed herein is a vaccine composition.

In some embodiments, a composition disclosed herein is administered at a dose of about 5 ng to about 1000 ng, about 5 ng to about 900 ng, about 5 ng to about 800 ng, about 5 ng to about 700 ng, about 5 ng to about 600 ng, about 5 ng to about 500 ng, about 5 ng to about 400 ng, about 5 ng to about 300 ng, about 5 ng to about 200 ng, about 5 ng to about 100 ng, about 5 ng to about 90 ng, about 5 ng to about 80 ng, about 5 ng to about 70 ng, about 5 ng to about 60 ng, about 5 ng to about 50 ng, about 5 ng to about 40 ng, about 5 ng to about 30 ng, about 5 ng to about 20 ng, or about 5 ng to about 10 ng. In some embodiments, a composition disclosed herein is administered at a dose of about 10 ng to about 1000 ng, about 20 ng to about 1000 ng, about 30 ng to about 1000 ng, about 40 ng to about 1000 ng, about 50 ng to about 100 ng, about 60 ng to about 1000 ng, about 70 ng to about 1000 ng, about 80 ng to about 1000 ng, about 90 ng to about 1000 ng, about 100 ng to about 1000 ng, about 200 ng to about 1000 ng, about 300 ng to about 1000 ng, about 40 ng to about 1000 ng, about 50 ng to about 1000 ng, about 60 ng to about 1000 ng, about 700 ng to about 1000 ng, about 800 ng to about 1000 ng, or about 900 ng to about 1000 ng.

In some embodiments, a composition disclosed herein is administered at a dose of about 5 ng, about 10 ng, about 20 ng, about 30 ng, about 40 ng, about 50 ng, about 60 ng, about 70 ng, about 80 ng, about 90 ng, about 100 ng, 150 ng, about 200 ng, about 250 ng, about 300 ng, about 350 ng, about 400 ng, about 450 ng, about 500 ng, about 550 ng, about 600 ng, about 650 ng, about 700 ng, about 750 ng, about 800 ng, about 850 ng, about 900 ng, about 950 ng, or about 1000 ng.

In some embodiments, a composition disclosed herein is administered at a dose of at least 5 ng, at least 10 ng, at least 20 ng, at least 30 ng, at least 40 ng, at least 50 ng, at least 60 ng, at least 70 ng, at least 80 ng, at least 90 ng, at least 100 ng, at least 150 ng, at least 200 ng, at least 250 ng, at least 300 ng, at least 350 ng, at least 400 ng, at least 450 ng, at least 500 ng, at least 550 ng, at least 600 ng, at least 650 ng, at least 700 ng, at least 750 ng, at least 800 ng, at least 850 ng, at least 900 ng, at least 950 ng, or at least 1000 ng.

### Pharmaceutical Compositions

In some embodiments, a composition comprising one or more ruminal-associated antigens is a pharmaceutical composition. In some embodiments, a pharmaceutical composition further comprises a pharmaceutically acceptable excipient. Pharmaceutical compositions of the present disclosure may comprise a polypeptide disclosed herein, a polynucleotide disclosed herein, or an expression vector comprising a polynucleotide disclosed herein.

In some embodiments, a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc, as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration is typically a solution in sterile isotonic aqueous buffer Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of a pharmaceutical composition described herein.

### RNA Formulations

Among other things, provided herein are compositions comprising polyribonucleotides comprising ruminal-associated antigens, and formulations thereof. In some embodiments, a composition comprising a polyribonucleotide disclosed herein is formulated in a lipid nanoparticle (LNP) formulation.

In some embodiments, a polyribonucleotide disclosed herein encodes for a polypeptide. In some embodiments, a polyribonucleotide disclosed herein is or comprises a messenger RNA. In some embodiments, a composition comprising a polyribonucleotide comprising a messenger RNA is formulated in a lipid nanoparticle (LNP) formulation.

In some embodiments, the disclosure provides an LNP formulation comprising a polyribonucleotide disclosed herein for use in a pharmaceutical composition, e.g., an immunogenic composition.

### Methods of Using Compositions Disclosed Herein

The disclosure provides, among other things, methods for using a polyribonucleotide disclosed herein, or a composition comprising the same.

In some embodiments, provided herein is a method of administering a polyribonucleotide disclosed herein or a composition comprising a polyribonucleotide disclosed herein to a cell, tissue or an animal, e.g., a ruminant.

In some embodiments, provided herein is an administration method, e.g., a vaccination method. comprising administering a polyribonucleotide disclosed herein or a composition comprising a polyribonucleotide disclosed herein to a cell, tissue or animal, e.g., a ruminant. In some embodiments, a ruminant comprises a cattle, sheep, goat, buffalo, moose, antelope, caribou, or deer, or combinations thereof.

In some embodiments, a composition disclosed herein is characterized in that when administered to an animal, a composition reduces methane emissions from an animal as compared to an otherwise comparable animal not administered a composition or administered a different composition. In some embodiments, a reduction in methane emissions is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to methane emissions in an otherwise comparable animal not administered a composition or administered a different composition. In some embodiments, a reduction in methane emissions is at least 5%.

In some embodiments, a reduction in methane emissions is about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to methane emissions in an otherwise comparable animal not administered the composition or administered a different composition.

In some embodiments, a reduction in methane emissions about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 95%, about 15% to about 95%, about 20% to about 95%, about 25% to about 95%, about 30% to about 95%, about 35% to about 95%, about 40% to about 95%, about 45% to about 95%, about 50% to about 95%, about 55% to about 95%, about 60% to about 95%, about 65% to about 95%, about 70% to about 95%, about 75% to about 95%, about 80% to about 95%, about 85% to about 95%, about 90% to about 95% as compared to methane emissions in an otherwise comparable animal not administered the composition or administered a different composition.

In some embodiments, a composition disclosed herein is characterized in that when administered to an animal, a composition reduces a population of microorganisms in an animal, as compared to an animal not administered a composition or administered a different composition. In some embodiments, microorganisms comprise methanogens. In some embodiments, microorganisms are methanogens from: Methanobrevibacter, Methanosphaera, Methanobacterium, Methanosarcinales, Methanomicrobiales, Thermoplasmatales, or a combination thereof. In some embodiments, a methanogen comprises Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielli, Methanococcus maripaludis, Methanopyrus kandleri, Methanocorpusculum labreanum, Methanococcoides burtonii, Methanosaete thermophila, Methanoregula boonei, Methanosphaerula palustris, Methanoculleus marisnigri, Methanospirillim hungatei, Mathanosarcina acetivorans, or any combination thereof. In some embodiments, a methanogen abundance is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to abundance of a methanogen in an otherwise comparable animal not administered a composition or administered a different composition. In some embodiments, a methanogen abundance is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to abundance of a methanogen in an otherwise comparable animal not administered the composition or administered a different composition.

In some embodiments, a composition disclosed herein is characterized in that when administered to an animal, a composition reduces abundance of all or substantially all methanogens (e.g., total methanogen abundance) in an animal as compared to total methanogen abundance in an otherwise comparable animal not administered a composition or administered a different composition In some embodiments, methanogen abundance, e.g., total methanogen abundance, is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to methanogen abundance in an otherwise comparable animal not administered a composition or administered a different composition. In some embodiments, methanogen abundance, e.g., total methanogen abundance, is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to methanogen abundance in an otherwise comparable animal not administered a composition or administered a different composition.

In some embodiments, a composition disclosed herein is characterized in that when administered to an animal, a composition reduces abundance of at least one methanogen species in an animal as compared to abundance of the same methanogen species in an otherwise comparable animal not administered a composition or administered a different composition. In some embodiments, at least one methanogen species is reduced by at least 5%, at least 10%, at least 15%, at least 20%, at least 25%, at least 30%, at least 35%, at least 40%, at least 45%, at least 50%, at least 55%, at least 60%, at least 65%, at least 70%/o, at least 75%, at least 80%, at least 85%, or at least 90% as compared to abundance of the same methanogen species in an otherwise comparable animal not administered a composition or administered a different composition. In some embodiments, at least one methanogen species is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to abundance of the same methanogen species in an otherwise comparable animal not administered a composition or administered a different composition. In some embodiments, abundance of at least 1, at least 2, at least 3, at least 4, at least 5, at least 6, at least 7, at least 8, at least 9, at least 10, at least 11, at least 12, at least 13, at least 14, at least 15, at least 16, at least 18, at least 19, at least 20 methanogen species is reduced.

In some embodiments, a composition disclosed herein is characterized in that when administered to an animal, a composition increases a growth rate of the animal as compared to a growth rate of an otherwise comparable animal not administered a composition or administered a different composition In some embodiments, growth rate is assessed by measuring a weight of an animal at one or more timepoints. In some embodiments, growth rate is assessed by measuring a weight of an animal at one or more timepoints. In some embodiments, the weight of an animal is measured daily. In some embodiments, a growth rate of an animal increases over a period of time. In some embodiments, a period of time comprises at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months or at least 12 months. In some embodiments, an increase in growth rate comprises a daily increase in weight of an animal. In some embodiments, a daily increase in weight of an animal is an increase of at least 5%, at least 10%, at least 15%, at least 20%, at least 25%, at least 30%, at least 35%, at least 40%, at least 45%, at least 50%, at least 55%, at least 600, at least 65%, at least 70%, at least 75%, at least 80%, at least 85%, or at least 90% as compared to weight in an otherwise comparable animal not administered the composition or administered a different composition. In some embodiments, a daily increase in weight of an animal is an increase of about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to weight in an otherwise comparable animal not administered a composition or administered a different composition.

In some embodiments, a composition disclosed herein is characterized in that when administered to an animal, the composition increases energy efficiency in an animal as compared to energy efficiency in an otherwise comparable animal not administered a composition or administered a different composition. In some embodiments, increasing energy efficiency comprises increasing digestion efficiency. In some embodiments, digestion efficiency comprises one or more, or all of the following parameters: (i) rate of weight gain, (ii) weight to intake relationship, or (iii) milk quality. In some embodiments, milk quality can be assessed by: weight gain metrics (e.g., Average Daily Gain (ADG) of cattle), intake to weight metrics (e.g., Food Conversation Ratio, or Residual Feed Intake), and/or milk composition metrics. In some embodiments, increasing digestion efficiency comprises decreasing digestion-related disorders in animals, e.g., ruminants.

In some embodiments, a composition disclosed herein is characterized in that when administered to an animal, the composition modifies output of cellulose fermentation.

In some embodiments, a composition disclosed herein is administered in one dose. In some embodiments, a composition is administered in a plurality of doses. In some embodiments, a composition is administered as a first dose of a composition followed by one or more subsequent doses of a composition. In some embodiments, a first dose and one or more subsequent doses of a composition comprise the same methanogen antigens and/or ruminal antigens. In some embodiments, a first dose and one or more subsequent doses of a composition comprise different methanogen antigens and/or ruminal antigens.

In some embodiments, one or more methanogen antigens and/or one or more ruminal antigens are specific to an animal. In some embodiments, one or more methanogen antigens and/or one or more ruminal antigens that are specific to an animal are obtained by a method comprising (i) identifying one or more methanogen antigens and/or one or more ruminal antigens that are expressed in an animal and (ii) responsive to the identification, selecting one or more methanogen antigens and/or one or more ruminal antigens to be included in a composition. In some embodiments, one or more methanogen antigens and/or one or more ruminal antigens that are specific to an animal is different from one or more methanogen antigens and/or one or more ruminal antigens that are specific to a different animal. In some embodiments, a different animals comprises any one or all of (i) a different species of animal, (ii) an animal of a different sex, (iii) an animal of a different age, or (iv) an animal located in a different geographical location, or (v) the same animal but at a different timepoint.

In some embodiments, a composition is administered 1, 2, 3, 4, 5, 6, 7, 8, 9, 10, 20 or 30 times to an animal. In some embodiments, a composition is administered once every 1 week, 2 weeks, 3 weeks, 1 month, 2 months, 3 months, 4 months, 5 months or 6 months.

In some embodiments, a composition is administered in combination with one or more additional agents. In some embodiments, one or more additional agents comprises a chemical additive, a biological feed additive. In some embodiments, a composition is administered in combination with one or more additional compositions. In some embodiments, the additional composition immunizes the animal from a disease, e.g., an infectious disease

### Kits

Another aspect of the present disclosure further provides a pharmaceutical pack or kit. In some embodiments, a kit can comprise a polyribonucleotide or a composition described herein, e.g., comprising a polynucleotide comprising a nucleotide sequence encoding a ruminal-associated antigen and/or a nucleotide sequence encoding a chemokine and/or cytokine. In some embodiment, kits may be used in any applicable method, e.g., methods as described herein.

### Sequences

TABLE 2
Exemplary antigen sequences

| | | | SEQ ID |
|---|---|---|---|
| Cluster | Description | Sequence | NO. |
| M1.1 | mru1499_sbi, | cttgttctttttgcagaagctcagaataaacgctcaactttggccaccATGCTCCTG | SEQ ID |
| | nucleotide | GCGGTCTTGCTGATGGGTTTTGTGCTCATTAGTAGCGTCTCTGCTATCGATATAGAT | NO.: 1 |
| | | GAGGCATCAAGTAGCAGCGATTTGTCCGATTCTAGTATCTCTAATGACTATCTTGTT | |
| | | GCTAACAGCGGCGATGACTCTGTCGCGAGCTCATCAGCCTCCTCTTCTATTGCCGCG | |
| | | GATGACTCAGACCTCTCTAATAATGCTTCTTCATCTAATGTCAATTTCGAGAACGAA | |
| | | GTGTTGTCTACTAACAACAACGAAGACACAGAAAGCGAAATCGTTAAGGATTCTAAA | |
| | | AATCAACTTTCCAGCAGCTCATTGCAGGCTTCTACCAAGACAAAGACGACCCTGAAG | |
| | | GGTAGCGGCTCATCTGTCTATAGGGGCAACCCTTACTATGTTACACTCACGGACAGC | |
| | | AACGGGAAGGTTCTGGCTTCACAGAAAGTTACGTTCAATATCCTCGGAAAAAAT | |
| | | TATACACGAACAACTGATTCTAAGGGGGTCGCCTCTATCAACATAAACTTGGCG | |
| | | AAGGGGAAGTATAATATCGCCTGTCTGTATGCTGGTACAGAGAATTATGCCAGT | |
| | | TCCAAACTTTCTGTGGCGCTTACAGTCAACTTGATGTCTACAAAAATTAACACTG | |
| | | GAGGCTCAACGGTCAAAAAAGGGAATGCATACAGTGTGACTCTTACGGATGGA | |
| | | AACGGTAAAGCCTTGAGCAGTCAAAAAGTGACACTGAACATACTGGGTAAAAA | |
| | | TTATACAAGGACGACGGATAGCAAGGGCGTTGCGAGCATAGCAATAAATCTCGC | |
| | | TGCAGGAAAAAAATTCACACTTACAGCCAGTTACGCAGGGTCCGCAAATTACCT | |
| | | CAGCAGCAAAGTGTCAGCTACCGTGACGGTTCAGAAAGGGGACACATCAATCA | |
| | | AGCCTAGCGGGACCTCAATTGTTAAAGGTAATTCCTATTCCTTTACACTCGTTGA | |
| | | CGGGTCTGGAAAGGGACTTGCAAACCAAAAGGTGGCCATCAAGATCTCTGGAA | |
| | | AGTCATACTCCCGCACAACAAACAGTAATGGCGTCGCATCCATAGCTATCAATT | |
| | | TGGCAGCCGGGAAGAAATATAGTATTGTGTGTTCTTATGCAGGTTCCTCTAATTA | |
| | | TAAAGCGAGCTCCTCAACGGTGTCCCTCTCCGTGACAAATCCGTCAACTAATTCT | |
| | | AAAACATTCTCCATTGCGAAGATTGAGGCCGCAGCGACAAATCTCAAAGCTTAT | |
| | | GTCAACAAGAATAAGGCTGTCCCTACAACGGTTTCTGTTGGCGGCACTAATCTG | |
| | | AAGATTTCCGAGTTCTCCTATCTGATGTCTAAGGCCATAGTGAATCTGAACTCTA | |
| | | ACAACACCAACGCGATCACTCTGCCGAGTGGCATTTATAATGGCGCTTCCGCAT | |
| | | CCAACTCCCTTAATGCCACTGTTTACAAGGCCCAATATGTTGATTTGTCCAAACG | |
| | | GGTGTATAACTATATAGACAAGAATAAGGTTCCTGCCGCTTATGGTACGGTGTA | |
| | | CAATGCCAATGGAGCGTCACTGGGCAACGCAGGGTTTAACCTCTACACATTCGC | |
| | | GTTCGCTAAGATTCTTGACTTCCACAAAACAAATAAATATCTTCCAAATTATTGT | |
| | | TCCTTCGATAGCTCTGTTTTCAAGGCATCCAATGGCAGTAGCAGTAGCAATAGTA | |
| | | GTAGCTCCACTAACTCATCATCATCTACTAATTCCAGCAGTGGAAGTAGCAATTC | |
| | | AAGCGGTTCTGGCTCTAGTACCCCTGCGGTCACAGTTAAAGCTACCAGTTTGAA | |
| | | GGCAGCCAGCACAAGTGTCATAAGGGGGGACGACTATAGCGTGACGCTCACAG | |
| | | ATTCCAGCGGTAATGCCCTCGCCAACCAGAAAATAACGTTCGCGCTTTCCTCTAG | |
| | | TAGTTATACCCGGACAACGAACAGCAAGGGGGTCGCTAGCTTGACCCTGAACCT | |
| | | CGCAGGAGGCAAATACTCCATCACCACTTCCTACGCCGGAACGTCTGCCTACAA | |
| | | AGCAAGCAAACTTACTAACACCGTTACGATATCTAACTCATCATCCAGGTTTTTT | |
| | | TTGAACGATATAGAAACCGCTGCGGAAAACGTTAAAACGTATGTGACTAAAAAC | |
| | | AAAGCACTGCCTAATACTGTCACTGTCGCCGGAACCCAACTGACCCTTTCTCAGT | |
| | | TTAGTTACGTCATGGCAAAGGCGATCCACAATATTAACGCTTCCAATTCAAACTA | |
| | | TATTTCTCTGAAGTCTGTCGCTTCCAGTAACTCTACTGGTGACTATTTGGATACC | |
| | | ACCGTGTACCGGGCACAGTACATGAACTTGACAAATCGAGTGATAAGCTTCGTT | |
| | | GAATCTGATAAGATCACACCTACTTTCGCAACGGTGTATAATTCTAATGGTAAA | |
| | | AGCGTCGGTAAAGCCGAATTCAAACTGTATACATTCGCATTTGCTAAAATACTC | |
| | | GCTTTTTACAAGACTAATAATTATTTGCCGACCTATTGCACCTTCCAGTCAAGTG | |
| | | CCATCGGGGTTGTCCCAGACGTCGCCACCAACGTGACGATAAATAGCAAGATAA | |
| | | ATGCCAATATGAATCAATTCAAAGTCGGGTTGAACGAAAAGAATACTGTTTCTA | |
| | | ATTTGTCTGCATATCTGGTTGGTACAGGACAGTCCACGATCACGACTAATATAAA | |
| | | AAACGTCGCGGCGCAACTGACGAAAGGTCTCAATTCCACCGCCACAAAGGCGCT | |
| | | CGCGATCTATAACTTTGTGAGAGACGACATTTCCTATTCCTATTATAGTGATTCA | |
| | | CGGAAAGGGGCAGATGGGACGCTCTCTTCCGGTAGCGGGAATTGCGTGGATCAA | |
| | | GCTAGCCTTGTCGTGGCATTGTGCAGGGCTGCCGGTATTCCTGCAAGGTACAGCC | |
| | | ATGCTCAGGGGTGCACATTTTCCTCTGGCCTGGTCACAGGCCACGTGTGGGCTCA | |
| | | AATTTTGGTTGATGGAGTTTGGTACTCTGCGGACGCAACGTCAGTTCGGAACTCC | |
| | | CTTGGTAACATAGTTAATTGGAACACCAATAGCTATCACAGCATGAAACAGTAT | |
| | | GCCGCTGTTCCATTTGGCGGCGGCGGTTCTGGAGGCGGGGGTTCCGGGGGCGGTG | |
| | | GCTCCATCGAAAACGCTGACAAGGCCATTAAGGATTTCCAGGATAATAAAGCGCCACA | |
| | | TGATAAGTCTGCGGCCTATGAAGCTAATAGTAAACTGCCGAAAGATCTGCGGGACAAG | |
| | | AATAACCGCTTTGTTGAAAAGGTGTCCATCGAAAAGGCAATAGTCCGCCACGATGAAA | |
| | | GAGTGAAATCAGCTAACGATGCCATAAGTAAATTGAACGAGAAGGATTCTATCGAGAAT | |
| | | CGACGGTTGGCACAGAGAGAGGTGAACAAAGCTCCAATGGATGTCAAGGAGCACCTT | |
| | | CAGAAACAACTCGACtaatgatagaccagcctcaagaacacccgaatggagtctctaagc | |
| | | tacataataccaacttacactttacaaaatgttgtcccccaaaatgtagccattcgtatc | |
| | | tgctcctaataaaaagaaagtttcttcacattct | |
| M1.1 | mru1499_sbi, | MLLAVLLMGFVLISSVSAIDIDEASSSSDLSDSSISNDYLVANSGDDSVASSSASSSIA | SEQ ID |
| | amino acid | ADDSDLSNNASSSNVNFENEVLSTNNNEDTESEIVKDSKNQLSSSSLQASTKTKTTL | NO.: 2 |
| | | KGSGSSVYRGNPYYVTLTDSNGKVLASQKVTFNILGKNYTRTTDSKGVASININLAK | |
| | | GKYNIACLYAGTENYASSKLSVALTVNLMSTKINTGGSTVKKGNAYSVTLTDGNGK | |
| | | ALSSQKVTLNILGKNYTRTTDSKGVASIAINLAAGKKFTLTASYAGSANYLSSKVSA | |
| | | TVTVQKGDTSIKPSGTSIVKGNSYSFTLVDGSGKGLANQKVAIKISGKSYSRTTNSNG | |
| | | VASIAINLAAGKKYSIVCSYAGSSNYKASSSTVSLSVTNPSTNSKTFSIAKIEAAATNL | |
| | | KAYVNKNKAVPTTVSVGGTNLKISEFSYLMSKAIVNLNSNNTNAITLPSGIYNGASA | |
| | | SNSLNATVYKAQYVDLSKRVYNYIDKNKVPAAYGTVYNANGASLGNAGFNLYTFA | |
| | | FAKILDFHKTNKYLPNYCSFDSSVFKASNGSSSSNSSSSTNSSSSTNSSSGSSNSSGSGS | |
| | | STPAVTVKATSLKAASTSVIRGDDYSVTLTDSSGNALANQKITFALSSSSYTRTTNSK | |
| | | GVASLTLNLAGGKYSITTSYAGTSAYKASKLTNTVTISNSSSRFFLNDIETAAENVKT | |
| | | YVTKNKALPNTVTVAGTQLTLSQFSYVMAKAIHNINASNSNYISLKSVASSNSTGDY | |
| | | LDTTVYRAQYMNLTNRVISFVESDKITPTFATVYNSNGKSVGKAEFKLYTFAFAKIL | |
| | | AFYKTNNYLPTYCTFQSSAIGVVPDVATNVTINSKINANMNQFKVGLNEKNTVSNLS | |
| | | AYLVGTGQSTITTNIKNVAAQLTKGLNSTATKALAIYNFVRDDISYSYYSDSRKGAD | |
| | | GTLSSGSGNCVDQASLVVALCRAAGIPARYSHAQGCTFSSGLVTGHVWAQILVDGV | |
| | | WYSADATSVRNSLGNIVNWNTNSYHSMKQYAAVPFGGGGSGGGGSGGGGSIENAD | |
| | | KAIKDFQDNKAPHDKSAAYEANSKLPKDLRDKNNRFVEKVSIEKAIVRHDERVKSANDAI | |
| | | SKLNEKDSIENRRLAQREVNKAPMDVKEHLQKQLD | |
| M1.2 | OVA_sbi, | cttgttctttttgcagaagctcagaataaacgctcaactttggccaccATGGGGTCCATA | SEQ ID |
| | nucleotide | GGGGCCGCGTCAATGGAGTTTTGCTTCGACGTCTTCAAAGAGTTGAAGGTCCACCACGCG | NO.: 3 |
| | | AATGAAAATATCTTCTATTGTCCTATTGCAATTATGAGTGCCCTTGCAATGGTTTACCTT | |
| | | GGGGCAAAAGACAGTACAAGGACACAAATCAACAAAGTGGTCCGATTTGATAAATTGC | |
| | | CGGGATTTGGGGATAGTATCGAAGCTCAGTGTGGGACGAGTGTCAACGTGCATT | |
| | | CTTCTCTTCGCGATATCCTCAATCAGATCACCAAGCCTAACGACGTGTACTCCTT | |
| | | TTCCCTCGCTTCTCGACTTTATGCGGAAGAGCGGTATCCTATATTGCCTGAGTAT | |
| | | CTTCAGTGCGTTAAAGAGCTGTATCGCGGCGGTCTCGAACCAATCAATTTTCAAA | |
| | | CGGCGGCCGACCAAGCTCGAGAGCTGATTAATTCTTGGGTGGAATCTCAGACCA | |
| | | ACGGGATTATCCGCAACGTTCTTCAGCCGTCTTCTGTCGACTCACAGACCGCTAT | |
| | | GGTTTTGGTGAACGCAATCGTCTTTAAAGGGTTGTGGGAGAAAGCGTTTAAGGA | |
| | | CGAAGATACTCAGGCAATGCCATTTAGAGTTACAGAACAAGAATCCAAACCTGT | |
| | | CCAGATGATGTACCAAATTGGCTTGTTCAGGGTGGCGTCAATGGCTTCAGAGAA | |
| | | AATGAAGATCTTGGAGCTTCCTTTTGCTTCCGGTACTATGTCCATGCTGGTCCTG | |
| | | CTCCCGGATGAAGTCTCAGGTCTGGAACAACTGGAGAGCATTATAAATTTTGAG | |
| | | AAACTCACGGAATGGACCTCCTCAAATGTTATGGAAGAAAGAAAGATCAAGGTC | |
| | | TATCTGCCGCGGATGAAGATGGAGGAAAAATACAACCTTACTTCTGTGTTGATG | |
| | | GCTATGGGCATAACTGATGTGTTCAGCAGTTCTGCTAATCTTAGCGGGATATCAA | |
| | | GCGCAGAATCTTTGAAGATCTCTCAAGCTGTTCACGCTGCTCACGCTGAAATAA | |
| | | ACGAGGCTGGGCGAGAAGTGGTCGGTTCCGCGGAGGCCGGGGTGGATGCGGCG | |
| | | TCCGTTTCAGAAGAGTTCCGAGCGGACCATCCTTTCCTGTTTTGTATCAAGCATA | |
| | | TTGCGACTAATGCTGTGCTCTTTTTCGGGCGCTGCGTGTCTCCTGGCGGCGGCGG | |
| | | TTCTGGAGGCGGGGGTTCCGGGGGCGGTGGCTCCATCGAAAACGCTGACAAGGCCA | |
| | | TTAAGGATTTCCAGGATAATAAAGCGCCACATGATAAGTCTGCGGCCTATGAAGCTAAT | |
| | | AGTAAACTGCCGAAAGATCTGCGGGACAAGAATAACCGCTTTGTTGAAAAGGTGTCCA | |
| | | TCGAAAAGGCAATAGTCCGCCACGATGAAAGAGTGAAATCAGCTAACGATGCCATAAG | |
| | | TAAATTGAACGAGAAGGATTCTATCGAGAATCGACGGTTGGCACAGAGAGAGGTGAAC | |
| | | AAAGCTCCAATGGATGTCAAGGAGCACCTTCAGAAACAACTCGACtaatgatagaccagc | |
| | | ctcaagaacacccgaatggagtctctaagctacataataccaacttacactttacaaaat | |
| | | gtgtcccccaaaatgtagccattcgtatctgctcctaataaaaagaaagtttcttcacat | |
| | | tct | |
| M1.2 | OVA_sbi, | MGSIGAASMEFCFDVFKELKVHHANENIFYCPIAIMSALAMVYLGAKDSTRTQINKV | SEQ ID |
| | amino acid | VRFDKLPGFGDSIEAQCGTSVNVHSSLRDILNQITKPNDVYSFSLASRLYAEERYPILP | NO.: 4 |
| | | EYLQCVKELYRGGLEPINFQTAADQARELINSWVESQINGIIRNVLQPSSVDSQTAM | |
| | | VLVNAIVFKGLWEKAFKDEDTQAMPFRVTEQESKPVQMMYQIGLFRVASMASEKM | |
| | | KILELPFASGTMSMLVLLPDEVSGLEQLESIINFEKLTEWTSSNVMEERKIKVYLPRM | |
| | | KMEEKYNLTSVLMAMGITDVFSSSANLSGISSAESLKISQAVHAAHAEINEAGREVV | |
| | | GSAEAGVDAASVSEEFRADHPFLFCIKHIATNAVLFFGRCVSPGGGGSGGGGSGGGG | |
| | | SIENADKAIKDFQDNKAPHDKSAAYEANSKLPKDLRDKNNRFVEKVSIEKAIVRHDERVK | |
| | | SANDAISKLNEKDSIENRRLAQREVNKAPMDVKEHLQKQLD* | |
| M2.1 | ssp_mru1499_ | cttgttctttttgcagaagctcagaataaacgctcaactttggccaccATGTTCGTGTTC | SEQ ID |
| | sbi, | CTGGTGCTCCTGCCTCTGGTGTCATCAGCCGCCATCGACATAGATGAAGCTTCCTCCTCC | NO.: 5 |
| | nucleotide | AGCGATCTGTCCGATTCTAGCATCAGCAATGACTACCTGGTCGCTAACAGCGGGGACGAC | |
| | | TCAGTCGCTTCCAGTAGTGCAAGCTCCTCCATAGCAGCTGACGACAGCGATCTC | |
| | | AGCAATAATGCCAGCTCCAGCAATGTGAATTTCGAGAACGAGGTCCTCAGCACC | |
| | | AACAATAACGAGGACACAGAGAGTGAAATCGTGAAAGATAGCAAGAACCAATT | |
| | | GAGCAGCTCCAGTCTGCAGGCCAGCACAAAGACTAAGACCACTTTGAAGGGGTC | |
| | | CGGAAGCAGCGTCTACCGGGGGAACCCATACTACGTCACCCTGACCGATTCAAA | |
| | | TGGAAAGGTTCTGGCCTCCCAGAAAGTGACTTTCAACATCCTGGGCAAAAATTA | |
| | | CACCCGGACCACTGATAGTAAAGGCGTGGCCTCCATCAACATCAATCTGGCCAA | |
| | | AGGCAAATATAATATCGCTTGTCTGTACGCCGGTACAGAGAATTACGCATCATC | |
| | | TAAGTTGAGTGTGGCTCTGACCGTCAACCTTATGTCCACCAAAATTAATACCGGT | |
| | | GGATCTACCGTCAAGAAGGGTAATGCATATTCCGTTACCCTGACCGATGGAAAC | |
| | | GGAAAGGCCCTCTCCAGCCAGAAGGTCACCTTGAACATCCTCGGTAAAAACTAT | |
| | | ACCCGCACTACCGACTCTAAAGGCGTGGCCAGCATCGCCATCAATCTTGCTGCA | |
| | | GGCAAGAAATTCACACTGACTGCTAGCTATGCAGGCTCTGCAAACTATCTTAGT | |
| | | AGTAAGGTGTCTGCCACAGTGACCGTCCAGAAGGGCGATACTAGCATCAAACCA | |
| | | AGCGGAACCTCCATCGTGAAAGGAAACTCATATAGCTTCACCTTGGTGGACGGC | |
| | | AGTGGGAAGGGCCTCGCAAATCAAAAGGTCGCCATCAAAATCAGCGGTAAGAG | |
| | | CTACTCCAGGACCACCAACTCTAACGGTGTGGCCAGTATCGCCATCAACCTGGC | |
| | | CGCTGGCAAGAAGTACTCTATCGTGTGTTCCTATGCCGGCAGCAGCAACTACAA | |
| | | GGCTTCCTCTAGCACCGTTAGTCTGAGTGTCACCAACCCATCCACTAATAGCAAG | |
| | | ACCTTCTCCATAGCCAAAATTGAAGCTGCTGCCACTAACCTCAAAGCCTATGTGA | |
| | | ACAAGAACAAAGCAGTGCCAACAACCGTGAGCGTCGGAGGCACTAACTTGAAG | |
| | | ATCAGTGAGTTTTCATACTTGATGTCTAAGGCCATTGTGAATCTGAATAGCAATA | |
| | | ATACCAACGCAATCACCCTGCCTTCAGGTATCTACAATGGGGCAAGCGCATCTA | |
| | | ACTCACTGAACGCAACTGTCTATAAAGCACAGTACGTCGACCTGTCAAAGAGAG | |
| | | TTTACAATTATATAGATAAAAACAAGGTGCCAGCCGCTTACGGAACCGTTTATA | |
| | | ACGCTAACGGTGCCTCCCTGGGGAACGCTGGCTTTAATCTGTATACCTTTGCCTT | |
| | | TGCCAAGATCCTGGACTTCCACAAAACAAACAAGTACCTGCCGAACTACTGTTC | |
| | | CTTCGACAGTTCCGTCTTTAAGGCCTCTAATGGCTCAAGTAGCTCCAATAGTTCA | |
| | | AGTTCCACTAACTCTAGTAGTTCTACCAATTCAAGCTCCGGCTCCAGCAACTCTA | |
| | | GTGGCTCTGGGTCCTCTACACCGGCCGTCACCGTGAAGGCTACCTCACTGAAAG | |
| | | CCGCCTCAACCAGCGTTATCCGGGGCGACGACTACTCTGTTACTCTGACTGACTC | |
| | | CAGCGGGAATGCCCTTGCTAATCAGAAAATTACCTTCGCACTGTCCAGCAGCTC | |
| | | ATATACACGCACCACAAACAGCAAGGGCGTGGCTTCACTCACACTCAATCTCGC | |
| | | TGGAGGGAAGTATTCAATCACAACCAGCTACGCTGGAACATCCGCTTACAAGGC | |
| | | TTCCAAGCTTACCAACACCGTGACTATCAGCAATTCTTCTTCCAGATTCTTTTTGA | |
| | | ACGACATTGAGACAGCAGCTGAGAATGTTAAGACTTATGTCACAAAAAACAAG | |
| | | GCACTTCCAAATACTGTCACCGTGGCTGGAACCCAGCTGACACTGTCACAGTTCT | |
| | | CTTACGTCATGGCAAAAGCCATTCACAACATAAACGCCAGTAATAGCAACTATA | |
| | | TATCCCTGAAAAGCGTGGCTAGCTCTAACTCTACTGGAGACTACCTTGACACCAC | |
| | | CGTGTACCGGGCCCAGTACATGAATCTCACAAACCGAGTGATTTCTTTTGTGGAA | |
| | | AGCGATAAGATTACACCTACCTTTGCAACCGTTTATAACAGCAATGGCAAGAGC | |
| | | GTGGGCAAGGCTGAGTTCAAGCTGTACACCTTCGCCTTTGCCAAAATTCTGGCCT | |
| | | TTTACAAGACCAATAACTACCTGCCTACCTATTGCACATTCCAGTCTAGCGCCAT | |
| | | TGGGGTGGTGCCTGACGTGGCCACCAACGTGACTATCAACTCCAAGATTAACGC | |
| | | TAACATGAATCAGTTCAAGGTGGGGCTGAATGAGAAGAACACCGTGTCAAACTT | |
| | | GTCCGCCTACCTGGTGGGCACAGGCCAATCCACCATAACAACCAATATCAAGAA | |
| | | CGTTGCTGCACAGCTCACCAAAGGCCTGAATTCCACCGCAACCAAGGCCCTCGC | |
| | | TATATACAACTTTGTGAGAGACGACATCTCCTACTCATACTACAGTGACTCTAGG | |
| | | AAGGGTGCAGACGGTACCCTGTCCAGTGGAAGTGGTAACTGTGTTGACCAGGCC | |
| | | AGTCTCGTCGTGGCTCTGTGCCGCGCCGCCGGGATCCCAGCTCGATATAGTCACG | |
| | | CACAGGGATGTACATTTTCAAGCGGGCTGGTTACAGGGCATGTGTGGGCTCAAA | |
| | | TTCTGGTCGATGGCGTCTGGTACTCAGCCGACGCAACATCCGTGCGGAATTCACT | |
| | | GGGGAACATTGTGAATTGGAACACCAACTCCTACCATAGCATGAAACAGTATGC | |
| | | TGCTGTCCCTTTTGGTGGCGGCGGTTCTGGGGGAGGCGGCAGCGGAGGTGGCGGC | |
| | | TCTATCGAAAACGCTGATAAGGCCATCAAGGATTTCCAAGATAATAAGGCCCCGCATG | |
| | | ACAAGTCCGCTGCCTACGAGGCCAACTCCAAACTGCCGAAGGACCTCCGGGACAAGA | |
| | | ATAACCGCTTTGTGGAGAAAGTGTCCATTGAAAAGGCCATCGTCCGCCACGACGAGA | |
| | | GGGTCAAGAGCGCCAACGATGCTATTTCCAAGCTTAACGAAAAGGACAGCATTGAGAA | |
| | | CCGCCGGCTGGCACAGCGAGAAGTGAATAAAGCACCTATGGATGTGAAGGAGCACCT | |
| | | GCAGAAACAACTGGACtaatgatagaccagcctcaagaacacccgaatggagtctctaag | |
| | | ctacataataccaacttacactttacaaaatgttgtcccccaaaatgtagccattcgtat | |
| | | ctgctcctaataaaaagaaagtttcttcacattct | |
| M2.1 | ssp_mru1499, | MFVFLVLLPLVSSAAIDIDEASSSSDLSDSSISNDYLVANSGDDSVASSSASSSIAAD | SEQ ID |
| | amino acid | DSDLSNNASSSNVNFENEVLSTNNNEDTESEIVKDSKNQLSSSSLQASTKTKTTLKGS | NO.: 6 |
| | | GSSVYRGNPYYVTLTDSNGKVLASQKVTFNILGKNYTRTTDSKGVASININLAKGK | |
| | | YNIACLYAGTENYASSKLSVALTVNLMSTKINTGGSTVKKGNAYSVTLTDGNGKAL | |
| | | SSQKVTLNILGKNYTRTTDSKGVASIAINLAAGKKFTLTASYAGSANYLSSKVSATV | |
| | | TVQKGDTSIKPSGTSIVKGNSYSFTLVDGSGKGLANQKVAIKISGKSYSRTTNSNGV | |
| | | ASIAINLAAGKKYSIVCSYAGSSNYKASSSTVSLSVTNPSTNSKTFSIAKIEAAATNLK | |
| | | AYVNKNKAVPTTVSVGGTNLKISEFSYLMSKAIVNLNSNNTNAITLPSGIYNGASAS | |
| | | NSLNATVYKAQYVDLSKRVYNYIDKNKVPAAYGTVYNANGASLGNAGFNLYTFAF | |
| | | AKILDFHKTNKYLPNYCSFDSSVFKASNGSSSSNSSSSTNSSSSTNSSSGSSNSSGSGSS | |
| | | TPAVTVKATSLKAASTSVIRGDDYSVTLTDSSGNALANQKITFALSSSSYTRTTNSKG | |
| | | VASLTLNLAGGKYSITTSYAGTSAYKASKLTNTVTISNSSSRFFLNDIETAAENVKTY | |
| | | VTKNKALPNTVTVAGTQLTLSQFSYVMAKAIHNINASNSNYISLKSVASSNSTGDYL | |
| | | DTTVYRAQYMNLINRVISFVESDKITPTFATVYNSNGKSVGKAEFKLYTFAFAKILA | |
| | | FYKTNNYLPTYCTFQSSAIGVVPDVATNVTINSKINANMNQFKVGLNEKNTVSNLSA | |
| | | YLVGTGQSTITTNIKNVAAQLTKGLNSTATKALAIYNFVRDDISYSYYSDSRKGADG | |
| | | TLSSGSGNCVDQASLVVALCRAAGIPARYSHAQGCTFSSGLVTGHVWAQILVDGV | |
| | | WYSADATSVRNSLGNIVNWNTNSYHSMKQYAAVPFGGGGSGGGGSGGGGSIENAD | |
| | | KAIKDFQDNKAPHDKSAAYEANSKLPKDLRDKNNRFVEKVSIEKAIVRHDERVKSANDAI | |
| | | SKLNEKDSIENRRLAQREVNKAPMDVKEHLQKQLD* | |
| M2.2 | ppa2_ | cttgttctttttgcagaagctcagaataaacgctcaactttggccaccATGCGGTTCCCT | SEQ ID |
| | mru1499_sbi, | TCTATTTTCACCGCAGTCTTGTTCGCTGCTAGTAGCGCTTTGGCTATGCCACTGAGCGGA | NO.: 7 |
| | nucleotide | GGCGGATCTGGGGGGTCAGGCTCTATGCCTCTGAACACTACCACAGAGGACGAGA | |
| | | CTGCACAGATACCTGCAGAGGCAGTCATCGGGTACCTCGACCTCGAGGGTG | |
| | | ACTTTGATGTGGCCGTTTTGCCTTTCTCTAATTCCACTAACAACGGCCTGCT | |
| | | GTTCATCAACACCACCATAGCCAGTATCGCCGCAAAAGAAGAGGGAGTCAG | |
| | | CCGGAGGTTGTTGCGGAGGTTGCTGAGAAGGTTGCTGGCCGCAATCGACAT | |
| | | AGATGAAGCTTCCTCCTCCAGCGATCTGTCCGATTCTAGCATCAGCAATGACTAC | |
| | | CTGGTCGCTAACAGCGGGGACGACTCAGTCGCTTCCAGTAGTGCAAGCTCCTCC | |
| | | ATAGCAGCTGACGACAGCGATCTCAGCAATAATGCCAGCTCCAGCAATGTGAAT | |
| | | TTCGAGAACGAGGTCCTCAGCACCAACAATAACGAGGACACAGAGAGTGAAAT | |
| | | CGTGAAAGATAGCAAGAACCAATTGAGCAGCTCCAGTCTGCAGGCCAGCACAA | |
| | | AGACTAAGACCACTTTGAAGGGGTCCGGAAGCAGCGTCTACCGGGGGAACCCAT | |
| | | ACTACGTCACCCTGACCGATTCAAATGGAAAGGTTCTGGCCTCCCAGAAAGTGA | |
| | | CTTTCAACATCCTGGGCAAAAATTACACCCGGACCACTGATAGTAAAGGCGTGG | |
| | | CCTCCATCAACATCAATCTGGCCAAAGGCAAATATAATATCGCTTGTCTGTACGC | |
| | | CGGTACAGAGAATTACGCATCATCTAAGTTGAGTGTGGCTCTGACCGTCAACCTT | |
| | | ATGTCCACCAAAATTAATACCGGTGGATCTACCGTCAAGAAGGGTAATGCATAT | |
| | | TCCGTTACCCTGACCGATGGAAACGGAAAGGCCCTCTCCAGCCAGAAGGTCACC | |
| | | TTGAACATCCTCGGTAAAAACTATACCCGCACTACCGACTCTAAAGGCGTGGCC | |
| | | AGCATCGCCATCAATCTTGCTGCAGGCAAGAAATTCACACTGACTGCTAGCTAT | |
| | | GCAGGCTCTGCAAACTATCTTAGTAGTAAGGTGTCTGCCACAGTGACCGTCCAG | |
| | | AAGGGCGATACTAGCATCAAACCAAGCGGAACCTCCATCGTGAAAGGAAACTC | |
| | | ATATAGCTTCACCTTGGTGGACGGCAGTGGGAAGGGCCTCGCAAATCAAAAGGT | |
| | | CGCCATCAAAATCAGCGGTAAGAGCTACTCCAGGACCACCAACTCTAACGGTGT | |
| | | GGCCAGTATCGCCATCAACCTGGCCGCTGGCAAGAAGTACTCTATCGTGTGTTCC | |
| | | TATGCCGGCAGCAGCAACTACAAGGCTTCCTCTAGCACCGTTAGTCTGAGTGTC | |
| | | ACCAACCCATCCACTAATAGCAAGACCTTCTCCATAGCCAAAATTGAAGCTGCT | |
| | | GCCACTAACCTCAAAGCCTATGTGAACAAGAACAAAGCAGTGCCAACAACCGTG | |
| | | AGCGTCGGAGGCACTAACTTGAAGATCAGTGAGTTTTCATACTTGATGTCTAAG | |
| | | GCCATTGTGAATCTGAATAGCAATAATACCAACGCAATCACCCTGCCTTCAGGT | |
| | | ATCTACAATGGGGCAAGCGCATCTAACTCACTGAACGCAACTGTCTATAAAGCA | |
| | | CAGTACGTCGACCTGTCAAAGAGAGTTTACAATTATATAGATAAAAACAAGGTG | |
| | | CCAGCCGCTTACGGAACCGTTTATAACGCTAACGGTGCCTCCCTGGGGAACGCT | |
| | | GGCTTTAATCTGTATACCTTTGCCTTTGCCAAGATCCTGGACTTCCACAAAACAA | |
| | | ACAAGTACCTGCCGAACTACTGTTCCTTCGACAGTTCCGTCTTTAAGGCCTCTAA | |
| | | TGGCTCAAGTAGCTCCAATAGTTCAAGTTCCACTAACTCTAGTAGTTCTACCAAT | |
| | | TCAAGCTCCGGCTCCAGCAACTCTAGTGGCTCTGGGTCCTCTACACCGGCCGTCA | |
| | | CCGTGAAGGCTACCTCACTGAAAGCCGCCTCAACCAGCGTTATCCGGGGCGACG | |
| | | ACTACTCTGTTACTCTGACTGACTCCAGCGGGAATGCCCTTGCTAATCAGAAAAT | |
| | | TACCTTCGCACTGTCCAGCAGCTCATATACACGCACCACAAACAGCAAGGGCGT | |
| | | GGCTTCACTCACACTCAATCTCGCTGGAGGGAAGTATTCAATCACAACCAGCTA | |
| | | CGCTGGAACATCCGCTTACAAGGCTTCCAAGCTTACCAACACCGTGACTATCAG | |
| | | CAATTCTTCTTCCAGATTCTTTTTGAACGACATTGAGACAGCAGCTGAGAATGTT | |
| | | AAGACTTATGTCACAAAAAACAAGGCACTTCCAAATACTGTCACCGTGGCTGGA | |
| | | ACCCAGCTGACACTGTCACAGTTCTCTTACGTCATGGCAAAAGCCATTCACAAC | |
| | | ATAAACGCCAGTAATAGCAACTATATATCCCTGAAAAGCGTGGCTAGCTCTAAC | |
| | | TCTACTGGAGACTACCTTGACACCACCGTGTACCGGGCCCAGTACATGAATCTC | |
| | | ACAAACCGAGTGATTTCTTTTGTGGAAAGCGATAAGATTACACCTACCTTTGCAA | |
| | | CCGTTTATAACAGCAATGGCAAGAGCGTGGGCAAGGCTGAGTTCAAGCTGTACA | |
| | | CCTTCGCCTTTGCCAAAATTCTGGCCTTTTACAAGACCAATAACTACCTGCCTAC | |
| | | CTATTGCACATTCCAGTCTAGCGCCATTGGGGTGGTGCCTGACGTGGCCACCAAC | |
| | | GTGACTATCAACTCCAAGATTAACGCTAACATGAATCAGTTCAAGGTGGGGCTG | |
| | | AATGAGAAGAACACCGTGTCAAACTTGTCCGCCTACCTGGTGGGCACAGGCCAA | |
| | | TCCACCATAACAACCAATATCAAGAACGTTGCTGCACAGCTCACCAAAGGCCTG | |
| | | AATTCCACCGCAACCAAGGCCCTCGCTATATACAACTTTGTGAGAGACGACATC | |
| | | TCCTACTCATACTACAGTGACTCTAGGAAGGGTGCAGACGGTACCCTGTCCAGT | |
| | | GGAAGTGGTAACTGTGTTGACCAGGCCAGTCTCGTCGTGGCTCTGTGCCGCGCC | |
| | | GCCGGGATCCCAGCTCGATATAGTCACGCACAGGGATGTACATTTTCAAGCGGG | |
| | | CTGGTTACAGGGCATGTGTGGGCTCAAATTCTGGTCGATGGCGTCTGGTACTCAG | |
| | | CCGACGCAACATCCGTGCGGAATTCACTGGGGAACATTGTGAATTGGAACACCA | |
| | | ACTCCTACCATAGCATGAAACAGTATGCTGCTGTCCCTTTTGGTGGCGGCGGTTC | |
| | | TGGGGGAGGCGGCAGCGGAGGTGGCGGCTCTATCGAAAACGCTGATAAGGCCATCA | |
| | | AGGATTTCCAAGATAATAAGGCCCCGCATGACAAGTCCGCTGCCTACGAGGCCAACTC | |
| | | CAAACTGCCGAAGGACCTCCGGGACAAGAATAACCGCTTTGTGGAGAAAGTGTCCATT | |
| | | GAAAAGGCCATCGTCCGCCACGACGAGAGGGTCAAGAGCGCCAACGATGCTATTTCC | |
| | | AAGCTTAACGAAAAGGACAGCATTGAGAACCGCCGGCTGGCACAGCGAGAAGTGAAT | |
| | | AAAGCACCTATGGATGTGAAGGAGCACCTGCAGAAACAACTGGACtaatgatagaccagc | |
| | | ctcaagaacacccgaatggagtctctaagctacataataccaacttacactttacaaaat | |
| | | gtgtcccccaaaatgtagccattcgtatctgctcctaataaaaagaaagtttcttcacat | |
| | | tct | |
| M2.2 | ppa2_ | MRFPSIFTAVLFAASSALAMPLSGGGSGGSGSMPLNTTTEDETAQIPAEAVIGYL | SEQ ID |
| | mru1499_sbi, | DLEGDFDVAVLPFSNSTNNGLLFINTTIASIAAKEEGVSRRLLRRLLRRLLAAIDI | NO.: 8 |
| | amino acid | DEASSSSDLSDSSISNDYLVANSGDDSVASSSASSSIAADDSDLSNNASSSNVNFENE | |
| | | VLSTNNNEDTESEIVKDSKNQLSSSSLQASTKTKTTLKGSGSSVYRGNPYYVTLTDS | |
| | | NGKVLASQKVTFNILGKNYTRTTDSKGVASININLAKGKYNIACLYAGTENYASSKL | |
| | | SVALTVNLMSTKINTGGSTVKKGNAYSVTLTDGNGKALSSQKVTLNILGKNYTRTT | |
| | | DSKGVASIAINLAAGKKFTLTASYAGSANYLSSKVSATVTVQKGDTSIKPSGTSIVKG | |
| | | NSYSFTLVDGSGKGLANQKVAIKISGKSYSRTTNSNGVASIAINLAAGKKYSIVCSYA | |
| | | GSSNYKASSSTVSLSVTNPSTNSKTFSIAKIEAAATNLKAYVNKNKAVPTTVSVGGT | |
| | | NLKISEFSYLMSKAIVNLNSNNTNAITLPSGIYNGASASNSLNATVYKAQYVDLSKR | |
| | | VYNYIDKNKVPAAYGTVYNANGASLGNAGFNLYTFAFAKILDFHKINKYLPNYCSF | |
| | | DSSVFKASNGSSSSNSSSSTNSSSSTNSSSGSSNSSGSGSSTPAVTVKATSLKAASTSVI | |
| | | RGDDYSVTLTDSSGNALANQKITFALSSSSYTRTTNSKGVASLTLNLAGGKYSITTSY | |
| | | AGTSAYKASKLINTVTISNSSSRFFLNDIETAAENVKTYVTKNKALPNTVTVAGTQL | |
| | | TLSQFSYVMAKAIHNINASNSNYISLKSVASSNSTGDYLDTTVYRAQYMNLTNRVIS | |
| | | FVESDKITPTFATVYNSNGKSVGKAEFKLYTFAFAKILAFYKTNNYLPTYCTFQSSAI | |
| | | GVVPDVATNVTINSKINANMNQFKVGLNEKNTVSNLSAYLVGTGQSTITTNIKNVA | |
| | | AQLTKGLNSTATKALAIYNFVRDDISYSYYSDSRKGADGTLSSGSGNCVDQASLVV | |
| | | ALCRAAGIPARYSHAQGCTFSSGLVTGHVWAQILVDGVWYSADATSVRNSLGNIVN | |
| | | WNTNSYHSMKQYAAVPFGGGGSGGGGSGGGGSIENADKAIKDFQDNKAPHDKSAAY | |
| | | EANSKLPKDLRDKNNRFVEKVSIEKAIVRHDERVKSANDAISKLNEKDSIENRRLAQREV | |
| | | NKAPMDVKEHLQKQLD* | |
| M2.3 | ppa2_cCXCL10, | cttgttctttttgcagaagctcagaataaacgctcaactttggccaccATGCGGTTCCCT | SEQ ID |
| | nucleotide | TCTATTTTCACCGCAGTCTTGTTCGCTGCTAGTAGCGCTTTGGCTATGCCACTGAGCGGA | NO.: 9 |
| | | GGCGGATCTGGGGGGTCAGGCTCTATGCCTCTGAACACTACCACAGAGGACGAGA | |
| | | CTGCACAGATACCTGCAGAGGCAGTCATCGGGTACCTCGACCTCGAGGGTG | |
| | | ACTTTGATGTGGCCGTTTTGCCTTTCTCTAATTCCACTAACAACGGCCTGCT | |
| | | GTTCATCAACACCACCATAGCCAGTATCGCCGCAAAAGAAGAGGGAGTCAG | |
| | | CCGGAGGTTGTTGCGGAGGTTGCTGAGAAGGTTGCTGGCCGCAGTGCCTTT | |
| | | GTCCCGGAATACACGGTGCTCTTGCATTGAGATTTCCAACGGTAGTGTGAACCCT | |
| | | CGGTCCCTTGAGAAACTCGAGGTGATCCCTGCCTCACAGTCTTGTCCTAGAGTGG | |
| | | AGATTATCGCTACCATGAAAAAGAATGGAGAGAAGCGATGTCTTAACCCTGAAT | |
| | | CCAAGACTATCAAGAATCTGCTGAAGGCTATCAATAAGCAGCGGACCAAGCGCT | |
| | | CACCACGGACACGGAAAGAGGCCtaatgatagaccagcctcaagaacacccgaatggagt | |
| | | ctctaagctacataataccaacttacactttacaaaatgttgtcccccaaaatgtagcca | |
| | | ttcgtatctgctcctaataaaaagaaagtttcttcacattct | |
| M2.3 | ppa2_cCXCL10, | MRFPSIFTAVLFAASSALAMPLSGGGSGGSGSMPLNTTTEDETAQIPAEAVIGYL | SEQ ID |
| | amino acid | DLEGDEDVAVLPFSNSTNNGLLFINTTIASIAAKEEGVSRRLLRRLLRRLLAAVP | NO.: 10 |
| | | LSRNTRCSCIEISNGSVNPRSLEKLEVIPASQSCPRVEIIATMKKNGEKRCLNPESKTIK | |
| | | NLLKAINKQRTKRSPRTRKEA | |
| M2.3 | ppa2_cVIP, | cttgttctttttgcagaagctcagaataaacgctcaactttggccaccATGCGGTTCCCT | SEQ ID |
| | nucleotide | TCTATTTTCACCGCAGTCTTGTTCGCTGCTAGTAGCGCTTTGGCTATGCCACTGAGCGGA | NO.: 11 |
| | | GGCGGATCTGGGGGGTCAGGCTCTATGCCTCTGAACACTACCACAGAGGACGAGA | |
| | | CTGCACAGATACCTGCAGAGGCAGTCATCGGGTACCTCGACCTCGAGGGTG | |
| | | ACTTTGATGTGGCCGTTTTGCCTTTCTCTAATTCCACTAACAACGGCCTGCT | |
| | | GTTCATCAACACCACCATAGCCAGTATCGCCGCAAAAGAAGAGGGAGTCAG | |
| | | CCGGAGGTTGTTGCGGAGGTTGCTGAGAAGGTTGCTGGCCGCACATAGTGA | |
| | | TGCCGTCTTTACCGACAACTACACTAGGTTGCGAAAGCAGATGGCCGTGAAGAA | |
| | | GTACCTGAACAGTATTTTGAACtaatgatagaccagcctcaagaacacccgaatggagtc | |
| | | tctaagctacataataccaacttacactttacaaaatgttgtcccccaaaatgtagccat | |
| | | tcgtatctgctcctaataaaaagaaagtttcttcacattct | |
| M2.3 | ppa2_cVIP, | MRFPSIFTAVLFAASSALAMPLSGGGSGGSGSMPLNTTTEDETAQIPAEAVIGYLDL | SEQ ID |
| | amino acid | EGDFDVAVLPFSNSTNNGLLFINTTIASIAAKEEGVSRRLLRRLLRRLLAAHSDAVFT | NO.: 12 |
| | | DNYTRLRKQMAVKKYLNSILN* | |
| M2.5 | ppa2_cAPRIL, | cttgttctttttgcagaagctcagaataaacgctcaactttggccaccATGCGGTTCCCT | SEQ ID |
| | nucleotide | TCTATTTTCACCGCAGTCTTGTTCGCTGCTAGTAGCGCTTTGGCTATGCCACTGAGCGGA | NO.: 13 |
| | | GGCGGATCTGGGGGGTCAGGCTCTATGCCTCTGAACACTACCACAGAGGACGAGA | |
| | | CTGCACAGATACCTGCAGAGGCAGTCATCGGGTACCTCGACCTCGAGGGTG | |
| | | ACTTTGATGTGGCCGTTTTGCCTTTCTCTAATTCCACTAACAACGGCCTGCT | |
| | | GTTCATCAACACCACCATAGCCAGTATCGCCGCAAAAGAAGAGGGAGTCAG | |
| | | CCGGAGGTTGTTGCGGAGGTTGCTGAGAAGGTTGCTGGCCGCAGCAGTGCT | |
| | | GACTCGGAAGCACAAGAAGAAACGAAGCGTGCTTCACCTCGTGCCGATCAACAT | |
| | | TACAAGCAAGGAAGATAGTGACGTCACCGAAGTGATGTGGCAGCCAGCACTCC | |
| | | AGCGGGGCCGGGGACTTGAGGCTCAGGGCTACGTGGTGAGGGTTTGGGACGCA | |
| | | GGGGTCTATCTGCTGTATAGCCAGGTGCTGTTTCACGATGAAACTTTCACCATGG | |
| | | GCCAGATGGTGTCCCGGGAGGGCCAGGGGAGGCAGGAAACACTGTTCCGGTGC | |
| | | ATTCAGAGCATGCCATCTAACCCTGACTGGGCCTACAATAGTTGCTACTCCGCCG | |
| | | GGGTGTTTCACCTTCACCAGGGGGATATACTGAGCGTTGTTATCCCACGGGCCA | |
| | | GAGCAAAGCTGTCTCTGAGCCCGCACGGCACCTTTTTGGGGCTCGTGAAGCTCtaa | |
| | | tgatagaccagcctcaagaacacccgaatggagtctctaagctacataataccaacttac | |
| | | actttacaaaatgtgtcccccaaaatgtagccattcgtatctgctcctaataaaaagaaa | |
| | | gtttcttcacattct | |
| M2.5 | ppa2_cAPRIL, | MRFPSIFTAVLFAASSALAMPLSGGGSGGSGSMPLNTTTEDETAQIPAEAVIGYL | SEQ ID |
| | amino acid | DLEGDFDVAVLPFSNSTNNGLLFINTTIASIAAKEEGVSRRLLRRLLRRLLAAAV | NO.: 14 |
| | | LTRKHKKKRSVLHLVPINITSKEDSDVTEVMWQPALQRGRGLEAQGYVVRVWDAG | |
| | | VYLLYSQVLFHDETFTMGQMVSREGQGRQETLFRCIQSMPSNPDWAYNSCYSAGV | |
| | | FHLHQGDILSVVIPRARAKLSLSPHGTFLGLVKL* | |
| C1.1 | mru_1726, | MFVFLVLLPLVSSAAENTDNALSTDTHSNDNVLSTDSRSNENALTNENTLLTDTHS | SEQ ID |
| | amino acid | NENALTTDPLTKENTHSYKDSEKSLSSDAFNKTIYVNKTGSDEGDGSEANPYATLKK | NO.: 15 |
| | | SISQLDDSDNAVIYIGPGNYTGENNSALEINLDHKDHDGSLSIIGDSNGGTVFDGENL | |
| | | NPIIISISEDSIVTLINITFTHGKNNMGSAIRSSGNLTIDNCIFTENYATNLAALYVDKH | |
| | | SPLTVMNSKFLENRAKQCADIYFSQNSEIILLNNLFEGSTAEYSYAYSPSVSLQTGKSL | |
| | | VKGNTFKNLTGAYYKGALYIAYNNGINIANITDNTFINCNYTGTDGAILFFQNAYLK | |
| | | NNKFIDCHSSTAFLYSNTEFNAYLSFEDAEIDGTTFFLKANVTDDMGNKVKNAKVIF | |
| | | YLNGENVGSASSDNNGVAMISIKKLLENGEYVISGTQSYSEINPFGVNVKNATARVN | |
| | | YDHSSLEVWVSTDGDDGSGNGSEDNPFKTLRKALDYGTASAVNLTVHVKNGIYNG | |
| | | DDNRDLSYSTLGKITIVGESYSNVVIDGENITKSIFAFSSTLDVTLINLTLINCPSTLIN | |
| | | AYTLSMMDNIVINSGTIRAQTGNNGVTIDNLRVINGTDQAITGYNLRLTNSRFENCDG | |
| | | LTHTGLIWLSTNNNKVTYLENNTFFNNTIAGSAGGGAAYYIQSDLISINNTEDSNWIT | |
| | | ESRGENVAYAGGRHIISINDKFINNEVPKYVAQYRSIGNEECEIIVENITFINNKASGN | |
| | | GAGLATTGAIVKGGKFINNSASGNGGAIYLLNHDNTSSYCQMSLEDVIFENNSATCG | |
| | | KDIFIEGSSGNNIFTYLNNLTIVANDLNVTSLSDNLTVSVFHPSGAIIGGGEISFYLDGE | |
| | | YIGKSTLVNQNASLEYVGFKNNTIYEFTSIYEYASLNDTYIDGIVSTKIPYALENIELY | |
| | | VSDGSGDDENGNGSISNPFKSISKALSEGYQKSTNITVHILEGTYTGSLNSNLRIPTTV | |
| | | NILLIGEGAAKTIISDSSSDYFITALKGKCELRISQMTLNRAARDTQSAIYIEEESNVAI | |
| | | DNVTFIGGQGNYGGAINTAGNLSIRNSYFHDNGYADRTLRANAYYGGAICNDGTLII | |
| | | DNTIFESDHAGRLSEIANQGTLYMNNSKVIDSINAYSINMDLVAIGAYGGQKGEITIE | |
| | | NSQFIVSSKTINELNDRIYDPTRALTCLGIGSCQHAILINSTFIGEGAVFSPYVFGGINS | |
| | | NNLASGGCTMIPGDLEVYNSTFRNVQAVNIIYSRTDNVRYHSQRVFEGCLFDNVEYI | |
| | | IAVLNTGNFSVEMHDCVILSDDLAKIGFGSEKTMKMDISNNWWSSNDGSYDNATLG | |
| | | TTNYISNCVVKLKEISSETVHPESYLILTLNSSNRTGLLQDAILAFKAYDGENISDYDG | |
| | | ALYPRNFEMSAINATGGGGSGGGGSGGGGSIENADKAIKDFQDNKAPHDKSAAYEANS | |
| | | KLPKDLRDKNNRFVEKVSIEKAIVRHDERVKSANDAISKLNEKDSIENRRLAQREVNKAP | |
| | | MDVKEHLQKQLD | |
| C1.1 | mru_1726, | cttgttctttttgcagaagctcagaataaacgctcaactttggccaccATGTTCGTCTTC | SEQ ID |
| | nucleotide | TTGGTGCTTCTGCCTCTCGTGTCTAGTGCTGCCGAGAACACAGACAACGCTCTGTCTACG | NO.: 16 |
| | | GACACCCATTCTAACGATAACGTGCTTTCTACAGACTCTCGGTCTAACGAGAACGCCTTG | |
| | | ACCAACGAGAACACCCTCCTCACCGACACCCACTCTAACGAGAACGCCCTGACGACCG | |
| | | ACCCTCTGACCAAGGAGAACACCCACTCCTACAAGGACTCAGAAAAAAGTCTCT | |
| | | CTTCTGACGCCTTCAACAAGACCATCTACGTGAACAAGACTGGTTCTGACGAGG | |
| | | GCGACGGGTCCGAAGCGAACCCTTACGCGACCTTGAAAAAGAGTATTTCTCAGC | |
| | | TCGACGATTCTGACAACGCCGTCATCTACATCGGGCCTGGCAACTACACCGGGG | |
| | | AGAACAATTCAGCCTTGGAGATCAACCTGGACCACAAGGACCACGACGGGTCCC | |
| | | TGAGCATCATCGGGGACTCAAACGGCGGGACGGTGTTCGACGGCGAGAACCTCA | |
| | | ACCCTATCATCATCTCAATCTCCGAGGACTCTATCGTGACCTTGATCAACATCAC | |
| | | GTTCACCCACGGCAAGAACAACATGGGCAGTGCCATCCGGTCTTCTGGCAACCT | |
| | | TACGATTGACAACTGCATCTTCACCGAGAACTATGCCACGAACTTGGCGGCCCT | |
| | | CTACGTGGACAAGCACTCCCCTCTGACGGTCATGAACTCTAAATTCCTCGAGAA | |
| | | CCGCGCGAAGCAGTGCGCCGACATCTACTTCAGTCAGAACTCTGAGATCATCCT | |
| | | CCTCAACAACCTTTTCGAGGGCTCTACAGCGGAGTACTCTTACGCCTACTCTCCT | |
| | | TCTGTCTCTCTTCAGACCGGGAAGTCTCTGGTGAAGGGGAATACCTTCAAGAAC | |
| | | CTGACCGGGGCGTACTACAAGGGCGCCCTGTACATAGCGTACAACAACGGCATC | |
| | | AATATCGCCAACATCACGGACAACACCTTCATCAACTGCAACTACACCGGCACC | |
| | | GACGGAGCCATCTTGTTCTTCCAGAACGCCTACCTGAAGAACAACAAGTTCATC | |
| | | GACTGCCACTCATCTACCGCATTCCTGTACTCAAACACGGAGTTCAACGCCTACC | |
| | | TGTCTTTCGAGGATGCCGAGATCGATGGCACCACCTTTTTTCTGAAGGCCAACGT | |
| | | GACCGACGACATGGGTAACAAGGTCAAGAACGCCAAGGTCATCTTCTACCTGAA | |
| | | CGGGGAGAACGTTGGCAGTGCCTCTTCTGACAACAACGGCGTCGCCATGATCTC | |
| | | TATCAAGAAACTGCTGGAGAACGGAGAGTATGTCATCTCTGGAACTCAGTCCTA | |
| | | CTCAGAGATCAACCCTTTCGGCGTGAACGTGAAGAACGCCACCGCCCGAGTCAA | |
| | | TTACGACCATAGCAGTTTGGAGGTCTGGGTGAGCACCGACGGCGACGACGGTTC | |
| | | TGGCAACGGCTCTGAGGACAACCCTTTCAAGACCCTGCGGAAGGCCCTTGACTA | |
| | | CGGGACGGCCTCTGCCGTGAACCTCACCGTGCACGTGAAGAACGGCATCTACAA | |
| | | TGGCGATGACAACAGAGACCTGTCTTACTCCACGCTGGGAAAGATTACCATAGT | |
| | | TGGCGAGAGTTACAGCAACGTTGTGATCGACGGTGAGAACATCACCAAGTCTAT | |
| | | ATTCGCATTCTCTTCCACCCTGGATGTGACCTTGATCAACCTCACCCTCATCAAC | |
| | | TGTCCTAGCACCCTTATCAACGCCTACACCCTGTCTATGATGGACAATATCGTCA | |
| | | TAAACTCTGGGACCATCCGCGCCCAGACCGGCAACAATGGGGTGACTATCGATA | |
| | | ACCTGCGGGTGATTAACGGCACGGACCAGGCGATAACCGGTTATAACCTCCGCC | |
| | | TGACCAACTCTCGCTTCGAGAACTGCGACGGCCTTACACACACCGGGCTGATCT | |
| | | GGCTGTCTACCAACAACAACAAAGTGACTTACCTGGAGAATAACACCTTCTTCA | |
| | | ACAACACGATCGCGGGCAGTGCAGGCGGGGGCGCCGCGTACTACATCCAGTCCG | |
| | | ACCTTATCAGTATTAACAATACGTTCGACTCTAACTGGATCACCGAGAGTCGAG | |
| | | GCGAGAACGTGGCGTACGCGGGCGGACGGCACATCATAAGCATCAACGATAAG | |
| | | TTCATAAACAACGAGGTGCCTAAATACGTCGCACAGTACCGATCAATCGGGAAC | |
| | | GAGGAGTGTGAGATCATCGTGGAAAACATCACCTTCATCAACAACAAGGCCTCC | |
| | | GGGAACGGCGCCGGGCTGGCCACCACCGGGGCCATCGTCAAGGGGGGGAAGTT | |
| | | CATTAACAACTCTGCCTCTGGGAATGGTGGGGCCATCTACCTGCTGAACCACGA | |
| | | CAACACGTCTTCGTACTGCCAGATGTCTCTGGAAGACGTGATCTTCGAGAACAA | |
| | | CTCTGCCACCTGCGGCAAGGACATCTTCATCGAGGGCTCATCTGGGAACAACAT | |
| | | CTTCACCTACCTGAACAACCTGACGATCGTGGCTAACGACCTGAACGTGACGTC | |
| | | ACTCTCAGACAACCTCACCGTCTCTGTGTTCCACCCTAGCGGCGCCATCATCGGG | |
| | | GGCGGGGAGATATCTTTCTACCTGGACGGCGAGTACATAGGAAAGTCTACTCTC | |
| | | GTCAACCAGAACGCATCCCTCGAGTACGTGGGGTTCAAGAACAACACTATCTAC | |
| | | GAATTCACGTCTATCTACGAGTACGCGTCTCTCAACGACACGTATATCGACGGC | |
| | | ATCGTCTCTACGAAGATCCCTTACGCTCTCGAGAACATTGAGCTGTACGTGTCAG | |
| | | ACGGCAGCGGCGACGACGAGAACGGCAACGGGAGTATCTCTAACCCTTTCAAGT | |
| | | CCATCTCTAAGGCCCTGTCTGAGGGTTATCAGAAGTCCACCAACATCACAGTGC | |
| | | ACATCTTGGAAGGTACATACACAGGCTCTCTGAACTCAAACCTCCGCATCCCTAC | |
| | | CACTGTCAACATCCTGCTGATCGGCGAGGGCGCCGCCAAGACAATCATCTCTGA | |
| | | CTCTTCAAGCGACTACTTCATCACAGCCCTGAAGGGGAAGTGCGAGCTGCGGAT | |
| | | CTCTCAGATGACCCTGAACCGCGCCGCCCGGGACACTCAGTCTGCTATCTACATC | |
| | | GAGGAGGAGAGTAACGTGGCAATCGACAACGTCACCTTCATAGGTGGCCAGGG | |
| | | CAACTACGGGGGAGCCATCAACACCGCTGGGAACCTCAGCATACGCAACAGCTA | |
| | | CTTCCACGACAATGGCTACGCCGACCGGACTCTCCGGGCCAATGCTTACTACGG | |
| | | AGGGGCAATCTGCAACGACGGCACCCTGATCATCGACAACACGATCTTCGAGAG | |
| | | CGACCACGCAGGTAGACTGTCTGAGATCGCTAACCAAGGCACTCTGTACATGAA | |
| | | CAACAGCAAGGTGATTGACTCTATCAACGCCTATTCTATCAACATGGACCTTGTG | |
| | | GCCATCGGGGCGTACGGGGGCCAAAAGGGCGAGATCACCATCGAGAATAGTCA | |
| | | ATTCATCGTCAGCTCTAAGACCATCAACGAGCTGAACGATAGGATTTACGACCC | |
| | | TACCCGGGCACTCACGTGCCTGGGAATCGGCTCTTGCCAGCACGCGATTCTCATC | |
| | | AACTCTACCTTCATTGGAGAGGGGGCCGTCTTCTCTCCTTACGTCTTCGGAGGGA | |
| | | TAAATTCAAACAACCTGGCCTCTGGGGGTTGCACCATGATTCCTGGGGACCTCG | |
| | | AGGTGTACAACTCTACGTTTAGGAACGTTCAGGCCGTGAACATAATCTACTCTCG | |
| | | CACTGACAACGTGAGGTACCACTCTCAGCGAGTCTTTGAGGGTTGCCTGTTCGAC | |
| | | AACGTTGAGTACATTATCGCCGTTCTGAACACAGGCAATTTCTCCGTTGAGATGC | |
| | | ACGACTGCGTGATCCTCAGTGACGATCTGGCCAAGATCGGCTTCGGGAGCGAGA | |
| | | AGACCATGAAGATGGACATCTCTAACAACTGGTGGTCTTCCAACGACGGCTCTT | |
| | | ACGACAACGCTACCCTCGGGACCACCAACTATATCTCCAACTGCGTGGTGAAGC | |
| | | TTAAGGAGATCTCTAGTGAGACCGTGCACCCTGAGTCTTACCTGATACTGACGCT | |
| | | CAACTCTTCTAATAGAACCGGACTTCTGCAGGACGCGATCCTGGCCTTTAAGGCC | |
| | | TACGACGGCGAGAACATCTCTGACTACGACGGCGCCCTGTACCCTCGGAATTTC | |
| | | GAGATGTCTGCCATCAACGCTACAGGCGGCGGCGGCTCTGGTGGGGGGGGCTCT | |
| | | GGCGGCGGAGGGTCTATCGAAAACGCCGACAAGGCAATCAAGGACTTCCAGGA | |
| | | CAACAAGGCCCCTCACGACAAATCTGCCGCCTACGAGGCCAACTCTAAGCTGCC | |
| | | TAAGGACCTGCGCGACAAGAACAATCGGTTTGTGGAAAAGGTGTCTATCGAGAA | |
| | | GGCCATCGTGAGGCACGACGAGCGCGTGAAGTCAGCCAACGACGCGATTTCTAA | |
| | | GCTGAACGAGAAGGACAGCATCGAGAACCGGCGCCTGGCCCAGCGCGAGGTGA | |
| | | ACAAGGCGCCTATGGACGTCAAGGAGCACTTGCAGAAGCAGCTCGACtaatgatagac | |
| | | cagcctcaagaacacccgaatggagtctctaagctacataataccaacttacactttaca | |
| | | aaatgtgtcccccaaaatgtagccattcgtatctgctcctaataaaaagaaagtttcttc | |
| | | acattct | |
| C1.2 | mru 1726, | MFVFLVLLPLVSSAALDDLEGTIINKAINPFEGVENSGYYIEAIVDNQKVNLTVHDS | SEQ ID |
| | amino acid | LSIGNAYILAENISINYNETQINVTVLEENGKRADGGNVSLKLHDKTYISEIINGTAIF | NO.: 17 |
| | | DIDVLPKGDYLLNYSLNRPKVYHSISNSSNLTVIPFKINAYANASNIKVGEDAIVIAYL | |
| | | DKDAGGNVTLGEEIQKVNDGTATFIISNLAKGDYTYQLSYSGDEKYDNETFHVSFSV | |
| | | NLKDASISVENDTLDLFVGSNETIVATISPIGLAVNYSCSNESVAMVDENGVVSAVG | |
| | | AGMAVITLTVGDDVTYSKNSSSVTVIVSKIPTIIEIVNDTISLEVTDSLDSIASLNPEEG | |
| | | GNLNYAISDDLIAKIDNGQITALSEGSAIITVSFDGNDKYTKAQNKSIKIIVNLKEASV | |
| | | SAFSNIDLLVGENDTLSPKTSPEGLDVRYESNDTSVVLVDNGQAIAVGEGNATITLT | |
| | | VGGDGVYAENTTTVKVSVSRIATMIEIEKDTIELKVNEESPIGAILEPDVGNLTYSISD | |
| | | ESIAKVENGKIIALAEGNASLSISFAGDERYIGTNASVEIRVNKINTILTETDITTTYKE | |
| | | EGYLIATLKDSQNNPISGAVLTVDLDGIKNYTTDSNGQIKIATNNLIPDTYTARISFAG | |
| | | NENYSSSNGNASVTVKRIGTKLNFNDMNTTAFDSNIEGRIGEYFYFQLVDCDGNPLA | |
| | | NKKVIIGFNGVKYNRQTNETGWAKIQINLKYANSYTFAIAFLGDDNYSGSFNFAVIR | |
| | | VSQQTPKLTASSKTYKSSAKTKTLTATLKSSSGKAIAGKKISFRLNGKVYTANTNSL | |
| | | GVATVKVSLNKKGTYSFTAQFSGDSTYKKVTKTAKLTIKGGGGSGGGGSGGGGSIE | |
| | | NADKAIKDFQDNKAPHDKSAAYEANSKLPKDLRDKNNRFVEKVSIEKAIVRHDERVKSAN | |
| | | DAISKLNEKDSIENRRLAQREVNKAPMDVKEHLQKQLD | |
| C1.2 | mru_1726, | cttgttctttttgcagaagctcagaataaacgctcaactttggccaccATGTTTGTCTTC | SEQ ID |
| | nucleotide | TTGGTCCTGCTCCCTTTGGTTAGCAGTGCTGCCCTGGATGACCTCGAAGGAACAATTATC | NO.: 18 |
| | | AATAAGGCCATCAATCCTTTTGAGGGCGTCGAGAATTCCGGATACTATATCGAGGCCATT | |
| | | GTGGATAATCAGAAGGTGAACTTGACAGTGCATGATAGCCTGTCAATCGGCAATGCCT | |
| | | ACATCCTGGCAGAGAATATCTCAATCAATTACAACGAGACTCAGATCAACGTGA | |
| | | CCGTGCTGGAAGAAAACGGCAAGAGGGCGGACGGGGGTAATGTGAGCCTGAAG | |
| | | CTCCATGACAAGACCTACATCAGTGAGATTATTAACGGGACCGCCATATTCGAC | |
| | | ATTGACGTGCTGCCTAAGGGGGACTACTTGCTCAACTATTCTTTGAACCGACCTA | |
| | | AAGTGTACCACAGTATATCCAATTCCTCAAACCTTACCGTTATACCTTTCAAAAT | |
| | | CAATGCATACGCCAACGCCAGCAACATTAAAGTGGGCGAGGATGCCATTGTGAT | |
| | | TGCTTATCTGGACAAGGACGCAGGAGGGAACGTTACACTCGGCGAGGAAATCCA | |
| | | GAAAGTTAATGATGGTACGGCAACATTTATAATTAGCAATTTGGCTAAAGGCGA | |
| | | CTACACCTACCAGCTGAGTTATTCCGGCGACGAGAAGTACGACAATGAGACCTT | |
| | | TCATGTCTCATTCTCAGTCAATCTGAAAGACGCTTCAATCAGCGTGGAGAATGAC | |
| | | ACATTGGACCTCTTTGTGGGATCTAACGAGACTATCGTCGCTACTATCTCTCCTA | |
| | | TCGGCCTTGCAGTGAACTACAGCTGCTCCAATGAATCCGTGGCGATGGTTGACG | |
| | | AAAATGGCGTGGTGAGCGCAGTGGGTGCGGGAATGGCAGTGATTACCCTGACA | |
| | | GTGGGAGACGATGTGACCTACTCCAAGAACAGCAGCTCTGTCACCGTGATAGTC | |
| | | AGCAAGATCCCTACCATTATCGAAATTGTTAATGACACCATTTCTCTTGAGGTCA | |
| | | CCGATTCACTGGACAGCATTGCATCTCTCAATCCTGAGGAGGGAGGTAACCTTA | |
| | | ATTATGCTATCTCCGACGATCTGATTGCAAAAATTGACAATGGGCAGATAACGG | |
| | | CCCTGTCTGAGGGGAGTGCAATCATCACCGTCAGCTTTGACGGGAATGACAAAT | |
| | | ATACCAAGGCCCAAAACAAGTCCATTAAAATTATCGTGAACCTCAAAGAGGCCT | |
| | | CAGTTAGTGCTTTCTCAAACATCGACCTGCTCGTGGGAGAGAACGATACCCTGTC | |
| | | CCCTAAGACGAGCCCTGAGGGCCTGGATGTCCGGTACGAGAGTAATGATACTAG | |
| | | TGTTGTGTTGGTCGACAACGGCCAAGCAATCGCCGTGGGCGAAGGCAACGCCAC | |
| | | GATTACACTCACGGTGGGAGGTGATGGGGTCTACGCCGAAAACACGACGACTGT | |
| | | TAAGGTGAGCGTTAGTCGGATAGCTACGATGATCGAGATCGAAAAGGATACCAT | |
| | | CGAACTGAAAGTGAACGAAGAATCCCCTATCGGTGCGATCCTGGAACCTGATGT | |
| | | GGGGAACCTTACCTACAGTATCTCTGATGAATCTATTGCCAAGGTGGAGAATGG | |
| | | CAAGATCATCGCGCTTGCAGAGGGCAATGCCTCCCTTTCCATCTCATTCGCCGGC | |
| | | GACGAACGGTATATTGGGACAAATGCTTCCGTGGAGATACGAGTGAATAAGATC | |
| | | AACACAATCCTGACCGAAACTGACATTACTACAACATATAAAGAGGAAGGGTAC | |
| | | CTGATTGCCACGCTGAAGGACTCACAAAATAACCCTATATCCGGTGCCGTGCTC | |
| | | ACAGTGGACCTCGACGGTATCAAGAACTATACCACGGATAGCAACGGCCAGATT | |
| | | AAGATCGCCACTAACAACCTGATTCCTGACACTTACACCGCCAGGATTTCCTTTG | |
| | | CCGGGAATGAAAATTATTCTTCCAGCAACGGAAACGCAAGCGTCACAGTGAAGC | |
| | | GCATAGGCACCAAACTGAACTTCAACGATATGAACACAACTGCCTTTGACAGCA | |
| | | ACATCGAGGGCAGGATCGGGGAGTATTTCTACTTCCAGCTGGTGGACTGTGATG | |
| | | GAAACCCTCTTGCCAACAAGAAGGTTATAATAGGATTCAACGGAGTGAAGTACA | |
| | | ACCGGCAGACAAATGAGACTGGCTGGGCGAAGATTCAGATCAATTTGAAATATG | |
| | | CCAACAGTTACACATTCGCTATCGCTTTTCTGGGCGATGATAACTACAGTGGCAG | |
| | | TTTTAACTTCGCGGTCATCAGGGTCTCTCAGCAGACTCCTAAACTGACCGCCAGC | |
| | | TCTAAAACATATAAAAGTAGCGCTAAGACCAAGACACTGACAGCGACCCTGAA | |
| | | GTCCTCAAGCGGTAAGGCAATTGCTGGCAAGAAAATCTCCTTCAGACTCAATGG | |
| | | GAAAGTCTATACCGCCAACACTAATTCTCTGGGGGTCGCCACTGTCAAAGTGTC | |
| | | CCTTAATAAAAAGGGTACCTACTCCTTCACTGCGCAGTTCTCTGGAGACTCTACC | |
| | | TACAAAAAGGTGACTAAAACCGCTAAGCTGACCATTAAGGGCGGCGGAGGATC | |
| | | CGGGGGAGGGGGTAGTGGAGGGGGTGGCTCCATTGAGAACGCCGATAAGGCCA | |
| | | TCAAGGATTTCCAGGATAACAAAGCCCCTCACGATAAGTCTGCCGCTTATGAGG | |
| | | CTAACTCTAAGCTGCCTAAAGACCTCAGAGACAAGAATAACAGATTTGTTGAGA | |
| | | AAGTGTCAATCGAGAAGGCTATAGTCCGCCACGATGAAAGAGTTAAGAGCGCTA | |
| | | ACGACGCAATCAGCAAACTTAACGAAAAAGACAGCATCGAGAACCGCCGGCTC | |
| | | GCCCAGCGCGAAGTGAACAAGGCTCCTATGGATGTCAAGGAGCACCTCCAAAA | |
| | | GCAGCTGGATtaatgatagaccagcctcaagaacacccgaatggagtctctaagctacat | |
| | | aataccaacttacactttacaaaatgttgtcccccaaaatgtagccattcgtatctgctc | |
| | | ctaataaaaagaaagtttcttcacattct | |
| C1.3 | WP_ | MFVFLVLLPLVSSAAFDNSNDTIGNNIALDGSDEMLNADESNGNILASASYNKTIYV | SEQ ID |
| | 042693350.1, | NYTGDDSGAGSQNSPYASLNKGISSVNASDNAVIYLSAGRFTGENNTDLSINLAHKN | NO.: 19 |
| | amino acid | YNGSLTIIGASNGQTILDGNGETCIFKSISADSIVTLINITFTHGKAEFGSAIRTSGNLT | |
| | | IDNCTFDSNYATTLAAIYAEDMEDVKITNSVFKNNYGFEGYADLYCTGTESITIDIINS | |
| | | TFINSTTENLYSLPSFSIQNTNANIIGNKFENMTGLYESGTFELRYGYGKRKVIDNIFV | |
| | | NCNYIGNRKGGIIYISNTYLKNNQFINCTSTNALICSVTEFNAYLKFKNLTVNGTEFK | |
| | | LICEVTDDLNNSVSTHGKVHFFIDGKGIGSDNANNGIASITVTKLLDNGDYTLSGTY | |
| | | YYSENPFEVNVKNATIHVDFNHDPLELWVSNDGNDTTGNGSKNNPFKTLRHALNY | |
| | | GFENTIDLTIHMKDGIYTGNDNKDLSYSNIGKLTIFGESYRNTIIDAEYNNTIFTFGKY | |
| | | LNVTLTNMTLRNTSGNVINAYILKICDSFIEHVDFLNGGYSDTSKIIFNNLTYTNSGGI | |
| | | YIYNAEIYNSKFKNCNNTKNNCLLQIISLENNIIHIENTTIINNTVNGQHGSSIVYISGN | |
| | | SILINNNYINNTVISQNAMYIFQSYANKLTSANETFIGNNVSSYIAYYDENGNNAEIIF | |
| | | ENITFKNNYAKIDGSGLVIKTGRIKGAKFINNTALNNGGAIIILPHYGNSPFPKCILED | |
| | | VFFENNNANNGKDIFIEKANNGYKNGQLDNITITFNNLITHNLQDIVTANISHPSGAVI | |
| | | GGGMIKFYLNGSYMGVAEVVNGVAKLNYLGFTKDGNYTLSGSYNYETNNTIYNNA | |
| | | TVNVMLSPLKENITLYVSDSRGDDENGNGSYENPYKTIENALNKGCKQSKVIFIKVL | |
| | | EGNYTDKFNNNITLFDSLNITIIGEGIDKTIITGNNTKNNWFISVLHAGNGFLKLVNMT | |
| | | ISEINYNYKNMNSQKSAVVIEDGANVMIDSVKFTKNRGENGGAINNKGKLNIVNSIF | |
| | | YNNGDSSYGGSIYNTGITIIDNSSFIANHAKFCADIYNEGILNIYNSTIQDSMRTNGWT | |
| | | GTPLVIGGLGNISIINSKIFRTGKTPLELINPGDTYADNPGFTISIGTTGSIILINTTID | |
| | | GHDAKYTGPSIYSTNNAAISWHVSSNIKIYNTSFLNLDSILNNQRGNITINSSFIKNVSN | |
| | | LIKSTSLYNLTVINSYFADGTISTEKYSNSNICLNNNWWGSNSKPTYKVANVDTNPET | |
| | | WLILTLNQTNQSVLSKNIILAFKVSDGENITDYTGQLYPRQFTMSSINGTGGGGSGG | |
| | | GGSGGGGSIENADKAIKDFQDNKAPHDKSAAYEANSKLPKDLRDKNNRFVEKV | |
| | | SIEKAIVRHDERVKSANDAISKLNEKDSIENRRLAQREVNKAPMDVKEHLQKQL | |
| | | D | |
| C1.3 | WP_ | cttgttctttttgcagaagctcagaataaacgctcaactttggccaccATGTTCGTGTTC | SEQ ID |
| | 042693350.1, | CTCGTGCTGCTTCCTTTGGTCTCCTCTGCCGCGTTCGACAACTCTAACGACACCATAGGG | NO.: 20 |
| | nucleotide | AACAACATCGCACTTGACGGCAGTGACGAGATGCTCAATGCCGACGAGAGTAACGGGAAC | |
| | | ATTCTGGCTTCCGCCTCTTACAACAAGACCATCTACGTGAACTACACCGGAGACGAC | |
| | | TCTGGGGCTGGCTCTCAGAACTCTCCTTACGCCAGCCTGAACAAGGGCATCTCTA | |
| | | GTGTGAACGCTTCAGACAACGCCGTGATCTACCTGTCAGCGGGCCGGTTCACCG | |
| | | GCGAGAATAACACCGACCTCTCTATCAACCTGGCCCATAAGAACTATAACGGGT | |
| | | CTCTTACGATCATCGGCGCCTCTAACGGCCAGACGATACTCGACGGGAACGGGG | |
| | | AGACCTGCATCTTCAAGTCTATCTCTGCCGACTCTATCGTGACCCTTATCAACAT | |
| | | CACATTCACGCACGGCAAGGCCGAGTTCGGCTCTGCCATCCGGACATCTGGGAA | |
| | | CCTGACGATAGACAACTGCACATTCGACTCTAACTACGCCACCACCCTGGCCGC | |
| | | CATCTACGCGGAGGACATGGAGGACGTTAAGATCACCAACTCAGTCTTCAAAAA | |
| | | CAACTACGGCTTCGAGGGGTACGCCGACCTGTACTGCACCGGCACCGAGTCTAT | |
| | | CACGATCGACATTATCAACTCAACTTTTATCAACTCTACGACCGAGAACCTGTAC | |
| | | TCTCTCCCTTCTTTCTCTATCCAAAACACAAACGCCAACATTATCGGAAACAAGT | |
| | | TCGAGAACATGACCGGTCTCTACGAGAGTGGGACCTTCGAGCTGCGGTACGGGT | |
| | | ACGGAAAAAGGAAAGTGATCGACAACATCTTCGTGAACTGCAACTACATCGGCA | |
| | | ACCGAAAAGGCGGCATCATCTACATCTCTAACACCTACCTGAAGAATAATCAAT | |
| | | TCATCAACTGCACCAGCACGAACGCCTTGATCTGCTCTGTCACCGAGTTCAACGC | |
| | | GTACCTGAAGTTCAAGAACTTGACCGTGAACGGGACCGAGTTCAAGCTGATCTG | |
| | | TGAGGTTACCGATGACCTGAACAACTCTGTGTCCACCCATGGCAAGGTGCACTT | |
| | | CTTCATCGACGGAAAGGGCATCGGGAGCGACAACGCCAACAACGGCATCGCCTC | |
| | | TATCACCGTGACCAAGTTGCTGGACAACGGCGACTACACACTCTCCGGCACCTA | |
| | | CTATTACTCCGAGAACCCTTTCGAGGTCAACGTGAAGAACGCCACGATCCACGT | |
| | | GGACTTCAACCACGACCCTCTGGAGCTTTGGGTGTCTAACGACGGTAACGACAC | |
| | | CACGGGCAACGGCTCTAAAAATAACCCTTTCAAGACACTTCGGCACGCACTGAA | |
| | | CTACGGCTTTGAGAACACCATCGACCTCACCATCCACATGAAGGACGGGATCTA | |
| | | CACAGGCAATGACAATAAGGACCTGTCCTACAGCAACATCGGAAAGCTGACGAT | |
| | | CTTCGGGGAGTCTTACAGGAACACCATTATCGACGCCGAGTACAACAACACTAT | |
| | | CTTCACCTTCGGCAAGTACCTCAACGTGACCCTCACGAACATGACCCTGCGCAA | |
| | | CACAAGTGGCAACGTCATTAACGCCTACATTCTGAAGATCTGCGACTCATTCATT | |
| | | GAACACGTCGACTTCCTCAACGGTGGCTACTCTGACACCTCCAAGATCATCTTCA | |
| | | ACAACCTGACCTACACCAATTCTGGAGGAATTTACATCTATAACGCCGAGATCT | |
| | | ACAACTCTAAGTTCAAGAACTGCAACAATACGAAGAACAATTGCCTGCTGCAGA | |
| | | TCATCAGCCTCGAGAACAACATCATCCACATCGAGAACACTACGATCATCAACA | |
| | | ACACGGTGAACGGGCAGCACGGCTCTTCTATCGTCTACATCTCTGGCAACTCTAT | |
| | | CCTTATCAACAACAACTACATCAATAACACGGTGATAAGTCAGAACGCCATGTA | |
| | | CATCTTCCAGAGCTACGCGAACAAGCTGACGTCTGCCAACGAGACGTTCATCGG | |
| | | GAACAATGTCTCATCCTACATCGCATATTACGACTTCAACGGAAACAACGCAGA | |
| | | GATCATCTTTGAGAACATCACCTTCAAAAACAACTATGCCAAGATCGACGGGTC | |
| | | TGGGCTCGTCATCAAGACCGGGCGCATCAAGGGGGCTAAGTTCATCAATAACAC | |
| | | TGCTCTGAACAATGGCGGCGCCATCATCATACTGCCTCACTACGGTAACTCTCCT | |
| | | TTCCCTAAGTGCATCTTGGAGGACGTGTTCTTCGAGAACAACAACGCGAACAAC | |
| | | GGAAAGGACATATTCATAGAGAAGGCGAACAACGGCTACAAGAACGGCCAGTT | |
| | | GGACAACATCACCATCACCTTCAATAACCTCATAACCCACAACCTGCAGGACAT | |
| | | CGTCACCGCCAACATCTCCCACCCTAGTGGCGCAGTTATCGGGGGTGGCATGAT | |
| | | AAAGTTCTACTTGAACGGGAGTTACATGGGCGTGGCCGAGGTGGTGAACGGCGT | |
| | | GGCCAAGCTGAACTACCTCGGATTCACCAAGGACGGCAACTACACCCTGTCCGG | |
| | | CTCCTACAACTACGAAACCAACAACACGATCTACAACAACGCCACCGTTAACGT | |
| | | CATGCTCAGCCCTCTGAAGGAGAACATCACCCTGTACGTGTCTGACTCACGGGG | |
| | | AGACGACGAGAACGGGAACGGGAGTTACGAGAACCCTTACAAGACTATCGAGA | |
| | | ACGCCCTGAATAAGGGCTGCAAGCAGTCTAAGGTGATCTTCATCAAGGTTCTTG | |
| | | AGGGCAACTACACTGACAAGTTTAACAACAACATCACCCTGTTCGACTCTCTGA | |
| | | ATATCACTATCATCGGGGAAGGCATAGACAAGACTATCATCACCGGCAACAACA | |
| | | CAAAGAACAACTGGTTCATCAGCGTTCTGCACGCGGGCAACGGCTTCCTCAAGC | |
| | | TGGTGAACATGACAATCTCTGAGATCAACTACAACTACAAGAACATGAACTCTC | |
| | | AGAAGAGCGCCGTGGTCATCGAGGATGGGGCCAACGTGATGATCGACTCTGTCA | |
| | | AGTTCACGAAGAACCGCGGCTTCAACGGGGGCGCCATCAACAATAAGGGAAAG | |
| | | CTGAACATTGTCAACTCCATCTTCTACAACAACGGGGACTCTTCATACGGAGGCT | |
| | | CTATTTACAACACGGGGATCACTATAATTGACAACTCTTCTTTCATCGCTAACCA | |
| | | CGCGAAGTTCTGCGCCGACATCTACAACGAGGGCATACTCAACATCTACAATAG | |
| | | TACCATCCAGGATTCTATGAGAACCAACGGGTGGACCGGCACCCCTCTCGTGAT | |
| | | CGGGGGCCTGGGGAACATATCTATCATCAACTCTAAGATCTTCAGAACCGGCAA | |
| | | GACCCCTCTCGAGCTCATCAACCCTGGTGATACTTACGCCGACAACCCTGGCTTC | |
| | | ACTATTTCTATCGGGACAACCGGCTCTATCATTCTGATCAACACGACCATCGATG | |
| | | GGCACGACGCCAAGTATACCGGGCCTTCTATTTATTCTACGAACAACGCCGCCA | |
| | | TCTCTTGGCACGTGTCTTCTAACATAAAGATTTACAACACCTCTTTCCTTAACCTG | |
| | | GACTCCATCTTGAACAACCAGCGAGGTAACATCACCATCAACAGCAGTTTCATT | |
| | | AAGAACGTCTCAAACCTGATAAAGAGTACCTCACTTTACAACCTGACGGTCATC | |
| | | AACTCTTACTTCGCGGACGGGACGATATCTACTGAGAAGTACTCTAACTCTAAC | |
| | | ATCTGCCTCAACAACAACTGGTGGGGGAGTAACTCTAAGCCTACCTACAAGGTG | |
| | | GCCAACGTGGACACCAACCCTGAGACCTGGCTGATCCTCACCCTCAACCAGACC | |
| | | AATCAGTCTGTCCTCTCTAAGAACATCATCCTGGCTTTTAAGGTGTCAGACGGCG | |
| | | AGAACATCACCGACTACACCGGCCAGCTGTACCCTCGGCAGTTCACAATGAGCT | |
| | | CAATCAATGGTACCGGTGGCGGGGGTTCTGGGGGGGGTGGCTCTGGCGGGGGTG | |
| | | GCTCTATCGAGAACGCAGACAAAGCGATCAAGGACTTTCAGGACAACAAAGCC | |
| | | CCTCACGACAAGTCAGCCGCCTACGAGGCGAACTCTAAGCTGCCTAAGGATCTG | |
| | | CGGGACAAGAACAACCGCTTCGTGGAGAAAGTGAGCATCGAGAAGGCCATCGT | |
| | | GCGCCACGACGAACGCGTGAAGTCTGCAAACGACGCCATCAGCAAGTTGAACG | |
| | | AGAAGGATTCTATCGAGAACCGGCGCCTGGCGCAGCGCGAAGTCAACAAGGCC | |
| | | CCTATGGACGTCAAGGAACACCTGCAGAAGCAGCTGGACtaatgatagaccagcctcaag | |
| | | aacacccgaatggagtctctaagctacataataccaacttacactttacaaaatgttgtc | |
| | | ccccaaaatgtagccattcgtatctgctcctaataaaaagaaagtttcttcacattct | |
| C1.4 | WP_ | MFVFLVLLPLVSSAALKSANGNITNHITNLINISKNATIYYVEATVDNQTVNLTQNIT | SEQ ID |
| | 042693350.1, | HNVTIIAKDIIFDYGTTTINVNVLFDNLPADSETITLNLNNKKYTAKTKNGTATFNIDII | NO.: 21 |
| | amino acid | PHGTYTLKYIINATKLHGEVLNSSILTVNRINANINATSHSVIVGNDVKVEINLPKDLT | |
| | | GTINVVLNNETYPVEIINNKAIVIIPNLAQGEYIAKITYSGDNKYLPTNTTVNIKVLGIN | |
| | | ITAPDVEKYYKGSEKLQIFIKDSEGNAIIGQNIQIKLNGKNYTATTNNEGIASIELDLNI | |
| | | GKYSATIIFNDKKINASITIKSTIHAPNMIRGYNSGLDYQTTLLNVDGTPLANTHITLKI | |
| | | GDKKYSLTTDANGVAKLNKKIAIGTYNILIINPTNLEKQTKTLKIISRINNNKNLAGD | |
| | | YLSGINYKIRIIDDNGKTAGTGKIVKITINKKHTTYLPIKMDTQHGGGGSGGGGSGGG | |
| | | GSIENADKAIKDFQDNKAPHDKSAAYEANSKLPKDLRDKNNRFVEKVSTEKAIVRHDERV | |
| | | KSANDAISKLNEKDSIENRRLAQREVNKAPMDVKEHLQKQLD | |
| C1.4 | WP_ | cttgttctttttgcagaagctcagaataaacgctcaactttggccaccATGTTCGTCTTC | SEQ ID |
| | 042693350.1, | CTGGTGTTGCTCCCTCTTGTCTCCTCCGCGGCCCTGAAGTCCGCCAACGGGAACATAACC | NO.: 22 |
| | nucleotide | AACCACATCACCAACCTGATCAACATTTCAAAGAACGCCACTATCTACTACGTCGAGGCA | |
| | | ACGGTTGACAACCAGACCGTGAACCTTACTCAAAACATTACCCACAACGTCACCATC | |
| | | ATCGCCAAGGACATTATCTTCGACTACGGAACCACGACCATCAACGTCAATGTG | |
| | | TTGTTCGATAACCTGCCTGCCGACAGCGAAACCATCACGCTCAACCTGAACAAT | |
| | | AAGAAGTATACAGCCAAGACTAAGAACGGCACCGCAACATTCAATATAGACAT | |
| | | AATTCCTCATGGCACTTACACCCTTAAGTACATCATCAACGCTACCAAACTCCAC | |
| | | GGTGAGGTGCTGAACTCAAGTATTCTGACGGTGAACAGGATAAACGCAAACATA | |
| | | AACGCGACCAGTCACTCCGTGATCGTTGGCAACGATGTGAAGGTCGAGATCAAC | |
| | | CTGCCTAAGGACCTGACTGGCACAATTAACGTGGTGCTCAACAACGAGACATAC | |
| | | CCTGTTGAGATCATTAACAACAAGGCCATCGTTATAATCCCTAACTTGGCCCAGG | |
| | | GTGAGTACATCGCCAAGATCACCTACAGTGGAGACAATAAGTACCTTCCTACAA | |
| | | ACACCACCGTGAACATCAAGGTGCTCGGGATCAACATCACCGCACCTGACGTCG | |
| | | AGAAATACTACAAGGGTTCTGAGAAGCTGCAGATCTTCATCAAGGACAGCGAGG | |
| | | GCAACGCGATCATCGGTCAGAACATACAGATCAAGCTGAACGGCAAGAACTAC | |
| | | ACGGCCACCACCAACAACGAGGGAATCGCGTCAATAGAGTTGGACCTGAATATT | |
| | | GGCAAGTACAGCGCCACGATTATCTTCAACGACAAGAAGATCAATGCCAGCATC | |
| | | ACCATCAAGTCAACCATCCACGCGCCTAACATGATACGGGGCTACAACTCTGGA | |
| | | CTGGATTACCAGACTACCCTGCTCAACGTGGACGGGACCCCTCTCGCCAACACC | |
| | | CACATAACACTGAAGATCGGGGACAAGAAGTACAGTCTTACTACGGACGCTAAC | |
| | | GGGGTGGCCAAACTGAACAAAAAAATCGCCATTGGCACGTACAACATCCTGATC | |
| | | ATCAATCCTACCAACCTGGAGAAACAGACGAAGACGCTCAAGATCATATCACGA | |
| | | ATCAACAACAACAAGAACCTGGCGGGCGACTATTTGAGTGGGATCAACTACAAG | |
| | | ATCCGAATCATCGACGACAACGGGAAGACAGCCGGGACCGGCAAGATCGTGAA | |
| | | GATCACCATCAATAAAAAGCACACCACTTATCTTCCTATCAAAATGGACACACA | |
| | | GCACGGCGGGGGTGGCTCCGGAGGGGGCGGGTCTGGCGGAGGTGGCAGCATTG | |
| | | AAAACGCCGACAAGGCTATCAAGGATTTCCAGGACAACAAGGCACCTCACGAC | |
| | | AAGTCCGCCGCATACGAGGCTAACTCTAAGCTCCCTAAGGACCTCCGCGACAAG | |
| | | AACAATCGCTTTGTGGAGAAGGTGTCAATCGAGAAGGCTATCGTGCGCCACGAC | |
| | | GAGAGGGTCAAATCTGCTAACGATGCCATCAGCAAGCTGAACGAGAAGGACAG | |
| | | TATCGAGAATCGGCGGCTGGCCCAGAGAGAAGTCAACAAGGCCCCTATGGACGT | |
| | | TAAGGAGCATTTGCAAAAGCAGCTTGACtaatgatagaccagcctcaagaacacccgaat | |
| | | ggagtctctaagctacataataccaacttacactttacaaaatgttgtcccccaaaatgt | |
| | | agccattcgtatctgctcctaataaaaagaaagtttcttcacattct | |
| C2.1 | mru_1923, | MFVFLVLLPLVSSAAAEDLESDIGSQSNPNSQVQLAPQMGHLHRMINKAASGEPGG | SEQ ID |
| | MtrE = | GGSGGGGSGGGGSIENADKAIKDFQDNKAPHDKSAAYEANSKLPKDLRDKNNRFVEKV | NO.: 23 |
| | WP_ | SIEKAIVRHDERVKSANDAISKLNEKDSIENRRLAQREVNKAPMDVKEHLQKQLD | |
| | 012956721.1 | | |
| | tetrahydrom- | | |
| | ethanopterin | | |
| | S- | | |
| | methyl- | | |
| | transferase | | |
| | subunit E | | |
| | [Methano- | | |
| | brevibacter | | |
| | ruminantium], | | |
| | fragment 1, | | |
| | amino acid | | |
| C2.1 | mru_1923, | cttgttctttttgcagaagctcagaataaacgctcaactttggccaccGCCGAGGATCTC | SEQ ID |
| | MtrE = | GAGTCAGACATCGGAAGCCAGTCAAACCCTAACTCTCAGGTCCAGCTGGCCCCTCAGATG | NO.: 24 |
| | WP_ | GGGCATCTTCACCGCATGATCAACAAAGCCGCCAGTGGCGAGCCTGGCGGCGGCGGGAGT | |
| | 012956721.1 | GGTGGCGGGGGGTCTGGTGGAGGGGGCAGCATAGAGAACGCCGACAAGGCTATC | |
| | tetrahydrom- | AAGGACTTCCAGGACAACAAGGCTCCTCACGACAAGTCCGCAGCATACGAGGCC | |
| | ethanopterin | AACTCAAAGCTGCCTAAGGATCTTCGGGACAAGAATAACCGGTTCGTGGAGAAA | |
| | S- | GTGTCCATTGAAAAGGCGATCGTCCGCCACGACGAGCGAGTGAAGTCCGCCAAT | |
| | methyl- | GACGCCATCTCTAAGCTGAACGAGAAGGACAGTATCGAGAACAGACGGCTGGC | |
| | transferase | GCAAAGGGAAGTGAACAAGGCGCCTATGGACGTTAAGGAGCACCTCCAGAAGC | |
| | subunit E | AGTTGGACtaatgatagaccagcctcaagaacacccgaatggagtctctaagctacataa | |
| | [Methano- | taccaacttacactttacaaaatgttgtcccccaaaatgtagccattcgtatctgctcct | |
| | brevibacter | aataaaaagaaagtttcttcacattct | |
| | ruminantium], | | |
| | fragment 1, | | |
| | nucleotide | | |
| C2.2 | mru_1923, | MFVFLVLLPLVSSAASHMGRIVGQSQFEQPLFMDVLTQSLGPGGGGSGGGGSGGG | SEQ ID |
| | MtrE = | GSIENADKAIKDFQDNKAPHDKSAAYEANSKLPKDLRDKNNREVEKVSTEKAIVRHDERV | NO.: 25 |
| | WP_ | KSANDAISKLNEKDSIENRRLAQREVNKAPMDVKEHLQKQLD | |
| | 012956721.1 | | |
| | tetrahydrom- | | |
| | ethanopterin | | |
| | S- | | |
| | methyl- | | |
| | transferase | | |
| | subunit E | | |
| | [Methano- | | |
| | brevibacter | | |
| | ruminantium], | | |
| | fragment 2, | | |
| | amino acid | | |
| C2.2 | mru_1923, | cttgttctttttgcagaagctcagaataaacgctcaactttggccaccATGTTTGTCTTC | SEQ ID |
| | MtrE = | CTGGTGCTGCTCCCTTTGGTGAGTTCAGCCGCCTCCCACATGGGGCGCATCGTGGGGCAG | NO.: 26 |
| | WP_ | TCACAATTCGAGCAGCCTCTGTTCATGGACGTGCTCACCCAGTCCCTTGGCCCTGGGGGG | |
| | 012956721.1 | GGGGCAGCGGGGGCGGCGGATCTGGTGGCGGCGGCAGCATCGAGAATGCAGACAA | |
| | tetrahydrom- | GGCCATAAAGGACTTCCAGGACAACAAAGCCCCTCACGACAAGTCTGCCGCATA | |
| | ethanopterin | CGAGGCGAACTCTAAGCTGCCTAAGGACCTCAGAGACAAGAACAACCGGTTCGT | |
| | S- | GGAGAAGGTCAGCATCGAGAAGGCCATCGTGCGGCACGACGAGCGAGTGAAGA | |
| | methyl- | GTGCCAACGATGCCATTTCTAAGCTGAACGAGAAGGACTCTATCGAAAACAGGC | |
| | transferase | GCCTGGCGCAGCGGGAGGTTAACAAGGCTCCTATGGACGTCAAGGAGCACCTCC | |
| | subunit E | AGAAGCAGCTGGACtaatgatagaccagcctcaagaacacccgaatggagtctctaagct | |
| | [Methano- | acataataccaacttacactttacaaaatgttgtcccccaaaatgtagccattcgtatct | |
| | brevibacter | gctcctaataaaaagaaagtttcttcacattct | |
| | ruminantium], | | |
| | fragment 2, | | |
| | nucleotide | | |
| C2.3 | mru_1923, | MFVFLVLLPLVSSAASSTGDVHYGAESEYQKFEFGGGTPVAIQGDIVINAPMGAKN | SEQ ID |
| | MtrE = | SMDVVNFCAKGGGGSGGGGSGGGGSIENADKAIKDFQDNKAPHDKSAAYEANSKLPK | NO.: 27 |
| | WP_ | DLRDKNNRFVEKVSIEKAIVRHDERVKSANDAISKLNEKDSIENRRLAQREVNKAPMDVK | |
| | 012956721.1 | EHLQKQLD | |
| | tetrahydrom- | | |
| | ethanopterin | | |
| | S- | | |
| | methyl- | | |
| | transferase | | |
| | subunit E | | |
| | [Methano- | | |
| | brevibacter | | |
| | ruminantium], | | |
| | fragment 3, | | |
| | amino acid | | |
| C2.3 | mru_1923, | cttgttctttttgcagaagctcagaataaacgctcaactttggccaccATGTTCGTCTTC | SEQ ID |
| | MtrE = | CTGGTGTTGCTGCCTCTGGTCTCTTCTGCCGCCAGTAGCACGGGCGATGTGCACTACGGT | NO.: 28 |
| | WP_ | GCGGAGTCTGAGTACCAAAAGTTCGAGTTCGGCGGGGGCACCCCTGTTGCTATTCAGGGG | |
| | 012956721.1 | GACATCGTGACCAACGCACCTATGGGGGCGAAGAACAGTATGGACGTGGTCAATTTT | |
| | tetrahydrom- | TGCGCCAAGGGCGGCGGCGGGTCAGGGGGGGGGGGGAGCGGTGGCGGAGGATC | |
| | ethanopterin | TATAGAGAACGCCGACAAGGCCATCAAGGACTTCCAGGACAACAAGGCCCCTC | |
| | S- | ACGACAAGTCCGCAGCCTACGAGGCCAACTCTAAGCTCCCTAAAGACCTTCGQG | |
| | methyl- | ACAAAAACAACAGGTTCGTTGAGAAGGTGTCTATCGAGAAGGCCATCGTGAGAC | |
| | transferase | ACGACGAGCGCGTGAAGTCTGCCAACGACGCGATCTCCAAGCTCAACGAGAAG | |
| | subunit E | GACTCAATCGAGAACCGGCGACTGGCCCAGCGGGAAGTGAACAAGGCTCCTAT | |
| | [Methano- | GGACGTCAAGGAGCACCTGCAGAAGCAGCTCGACtaatgatagaccagcctcaagaacac | |
| | brevibacter | ccgaatggagtctctaagctacataataccaacttacactttacaaaatgttgtccccca | |
| | ruminantium], | aaatgtagccattcgtatctgctcctaataaaaagaaagtttcttcacattct | |
| | fragment 3, | | |
| | nucleotide | | |
| C2.4 | WP_ | MFVFLVLLPLVSSAASSTGDVHYGAESEYQKFEFGGGTPVAIQGDIVTKAPIGAKNS | SEQ ID |
| | 004032919.1 | MDVVNFCAKFGGGGGSGGGGSGGGGSIENADKAIKDFQDNKAPHDKSAAYEANSKLP | NO.: 29 |
| | MULTISPECIES: | KDLRDKNNRFVEKVSIEKAIVRHDERVKSANDAISKLNEKDSIENRRLAQREVNKAPMDV | |
| | tetrahydrom- | KEHLQKQLD | |
| | tehanopterin | | |
| | S- | | |
| | methyl- | | |
| | transferase | | |
| | subunit | | |
| | E | | |
| | [Methano- | | |
| | brevibacter]. | | |
| | fragment 1, | | |
| | amino acid | | |
| C2.4 | WP_ | cttgttctttttgcagaagctcagaataaacgctcaactttggccaccATGTTCGTGTTC | SEQ ID |
| | 004032919.1 | CTTGTTCTGCTGCCTCTGGTGTCTAGCGCCGCCAGCTCTACGGGAGACGTGCATTACGGT | NO.: 30 |
| | MULTISPECIES: | GCTGAGTCCGAATACCAGAAGTTTGAGTTCGGAGGCGGCACCCCTGTCGCCATCCAGGGG | |
| | tetrahydrom- | GACATCGTGACCAAGGCACCTATCGGCGCTAAAAACAGTATGGACGTCGTGAACTTC | |
| | ethanopterin | TGCGCCAAGTTCGGAGGGGGCGGGGGCTCCGGGGGGGGTGGCAGTGGCGGCGG | |
| | S- | GGGTAGTATAGAGAATGCCGACAAGGCCATCAAGGACTTCCAGGACAACAAGG | |
| | methyl | CGCCTCACGACAAGTCAGCGGCCTACGAGGCAAACTCCAAGCTGCCTAAAGATC | |
| | transferase | TCCGGGATAAGAACAACAGATTCGTTGAGAAGGTGTCAATAGAGAAGGCAATC | |
| | subunit | GTGAGGCACGACGAGCGAGTCAAGTCTGCCAACGACGCGATTAGCAAACTTAAT | |
| | E | GAGAAGGACTCAATCGAAAACCGGCGCCTGGCCCAGCGCGAGGTGAACAAGGC | |
| | [Methano- | CCCTATGGACGTCAAGGAGCACTTGCAAAAGCAGCTCGACtaatgatagaccagcctcaa | |
| | brevibacter]. | gaacacccgaatggagtctctaagctacataataccaacttacactttacaaaatgtgtc | |
| | fragment 1, | ccccaaaatgtagccattcgtatctgctcctaataaaaagaaagtttcttcacattct | |
| | nucleotide | | |
| C2.5 | WP_ | MFVFLVLLPLVSSAATSHMGRIVGQSQFEQPLFMDVLTQSLGPIAGHGGGGSGGGG | SEQ ID |
| | 081738309.1 | SGGGGSIENADKAIKDFQDNKAPHDKSAAYEANSKLPKDLRDKNNRFVEKVSIEKAIVRH | NO.: 31 |
| | tetrahydrom- | DERVKSANDAISKLNEKDSIENRRLAQREVNKAPMDVKEHLQKQLD | |
| | ethanopterin | | |
| | S- | | |
| | methyl- | | |
| | transferase | | |
| | subunit E | | |
| | [Methano- | | |
| | brevibacter | | |
| | oralis] | | |
| | fragment 1, | | |
| | amino acid | | |
| C2.5 | WP_ | cttgttctttttgcagaagctcagaataaacgctcaactttggccaccATGTTCGTTTTT | SEQ ID |
| | 081738309.1 | CTGGTGTTGCTTCCTCTGGTTAGCTCCGCCGCCACGTCTCACATGGGGCGCATTGTGGGC | NO.: 32 |
| | tetrahydrom- | CAATCTCAGTTCGAGCAGCCTCTGTTCATGGACGTGTTGACCCAGAGTCTCGGCCCTATC | |
| | ethanopterin | GCCGGACATGGCGGCGGGGGCAGCGGTGGGGGCGGGTCAGGGGGTGGCGGATCTATCG | |
| | S- | AGAACGCAGACAAGGCCATCAAGGACTTCCAGGACAATAAGGCGCCTCACGAC | |
| | methyl- | AAGTCAGCCGCTTACGAGGCAAACAGTAAGCTCCCTAAGGACCTGCGGGACAA | |
| | transferase | AAACAACCGCTTCGTCGAGAAGGTGTCCATCGAGAAGGCCATAGTCAGACACGA | |
| | subunit E | TGAGCGGGTCAAGTCAGCTAACGACGCGATCTCCAAGCTGAACGAGAAGGACA | |
| | [Methano- | GTATCGAGAACCGGAGGCTGGCCCAGCGAGAAGTGAACAAGGCCCCTATGGAC | |
| | brevibacter | GTGAAAGAGCACCTTCAGAAGCAGCTCGATtaatgatagaccagcctcaagaacacccga | |
| | oralis], | atggagtctctaagctacataataccaacttacactttacaaaatgttgtcccccaaaat | |
| | fragment 1, | gtagccattcgtatctgctcctaataaaaagaaagtttcttcacattc | |
| | nucleotide | | |
| C2.6 | WP_ | MFVFLVLLPLVSSAASSTGDVHYGAESEYQKFEFGGGTPVAIQGDIVTKAPIGAKNS | SEQ ID |
| | 081738309.1 | MDVVNFCAKFGGGGGSGGGGSGGGGSIENADKAIKDFQDNKAPHDKSAAYEANSKLP | NO.: 33 |
| | tetrahydrom- | KDLRDKNNREVEKVSIEKAIVRHDERVKSANDAISKLNEKDSIENRRLAQREVNKAPMDV | |
| | ethanopterin | KEHLQKQLD | |
| | S- | | |
| | methyl- | | |
| | transferase | | |
| | subunit E | | |
| | [Methano- | | |
| | brevibacter | | |
| | oralis], | | |
| | fragment 2, | | |
| | amino acid | | |
| C2.6 | WP_ | cttgttctttttgcagaagctcagaataaacgctcaactttggccaccATGTTCGTGTTC | SEQ ID |
| | 081738309.1 | TTGGTGCTTCTGCCTCTTGTGAGCTCCGCCGCTAGTTCAACCGGCGACGTCCATTACGGG | NO.: 34 |
| | tetrahydrom- | GCCGAAAGTGAGTACCAGAAGTTCGAGTTCGGAGGCGGGACGCCTGTGGCGATCCAGGGA | |
| | ethanopterin | GACATCGTTACCAAGGCACCTATAGGTGCCAAGAACTCTATGGATGTGGTCAATTTT | |
| | S- | TGCGCGAAGTTCGGCGGTGGGGGAGGGAGCGGCGGGGGGGGCTCCGGCGGCGG | |
| | methyl- | CGGTTCAATCGAGAATGCTGACAAGGCAATAAAGGACTTCCAGGACAACAAAG | |
| | transferase | CCCCTCACGACAAGTCTGCCGCCTACGAGGCAAACTCAAAGCTGCCTAAGGACC | |
| | subunit E | TGCGGGACAAAAACAACCGATTQGTTGAGAAGGTGTCTATCGAGAAGGCCATTG | |
| | [Methano- | TGAGACACGACGAGCGGGTCAAGTCCGCCAACGATGCCATCAGCAAGCTGAAC | |
| | brevibacter | GAGAAGGACAGTATCGAAAACAGGCGCCTCGCCCAACGCGAGGTGAACAAAGC | |
| | oralis], | GCCTATGGACGTCAAGGAGCACCTCCAGAAGCAGCTGGACtaatgatagaccagcctcaa | |
| | fragment 2, | gaacacccgaatggagtctctaagctacataataccaacttacactttacaaaatgttgt | |
| | nucleotide | cccccaaaatgtagccattcgtatctgctcctaataaaaagaaagtttcttcacattct | |
| C3.1 | WP_ | MFVFLVLLPLVSSAAQHLNVVVSGSMEPVMYRGDIVVLQKANLFGIHEFDPHDVQ | SEQ ID |
| | 012956916.1 | VGDIVVYNAAWYDSPVIHRVINTAEINGTTCFEIKGDNNNKSDPYWVTPEQITDRVI | NO.: 35 |
| | signal | TINGQPLVIPKIGYITLWVKGLGGGGSGGGGSGGGGSIENADKAIKDFQDNKAPHDKS | |
| | peptidase I | AAYEANSKIPKDLRDKNNRFVEKVSIEKAIVRHDERVKSANDAISKLNEKDSIENRRLAQ | |
| | [Methano- | REVNKAPMDVKEHLQKQLD | |
| | brevibacter | | |
| | ruminantium], | | |
| | amino acid | | |
| C3.1 | WP_ | cttgttctttttgcagaagctcagaataaacgctcaactttggccaccATGTTCGTGTTC | SEQ ID |
| | 012956916.1 | CTGGTCCTTCTCCCTCTCGTTTCCTCTGCCGCACAGCACCTGAACGTTGTCGTGAGCGGC | NO.: 36 |
| | signal | TCTATGGAACCTGTGATGTACCGCGGCGACATCGTGGTGCTCCAGAAGGCCAACCTTTTT | |
| | peptidase I | GGTATCCACGAGTTCGACCCTCACGACGTCCAGGTGGGAGACATCGTCGTGTACAACGC | |
| | [Methano- | CGCCTGGTACGACTCTCCTGTGATCCACAGGGTCATTAACACCGCGGAGATAAA | |
| | brevibacter | CGGCACAACTTGCTTCGAGATCAAGGGGGACAACAATAATAAGTCAGATCCTTA | |
| | ruminantium], | TTGGGTGACCCCTGAGCAAATCACCGACCGGGTGATCACGATCAACGGGCAGCC | |
| | nucleotide | TCTGGTCATCCCTAAGATCGGGTACATCACCCTGTGGGTGAAGGGCCTCGGGGG | |
| | | CGGCGGTTCTGGCGGGGGCGGGTCAGGAGGGGGGGGCAGTATCGAGAACGCCG | |
| | | ACAAGGCTATCAAGGACTTCCAGGACAACAAGGCCCCTCACGATAAGTCTGCCG | |
| | | CTTACGAGGCCAACTCCAAGTTGCCTAAAGACCTGCGGGACAAGAACAACCGGT | |
| | | TCGTGGAGAAGGTCTCTATTGAGAAAGCCATCGTGCGGCACGACGAGCGCGTCA | |
| | | AGAGCGCGAACGACGCGATCTCTAAGCTGAACGAGAAGGACAGTATAGAGAAC | |
| | | CGAAGACTGGCACAGCGCGAGGTGAACAAGGCCCCTATGGACGTGAAGGAGCA | |
| | | TCTGCAGAAGCAGCTGGACtaatgatagaccagcctcaagaacacccgaatggagtctct | |
| | | aagctacataataccaacttacactttacaaaatgttgtcccccaaaatgtagccattcg | |
| | | tatctgctcctaataaaaagaaagtttcttcacattct | |
| C3.2 | WP_ | MFVFLVLLPLVSSAALNVVVSGSMEPVFYRGDIVAVEKADFLGIHEFDPSDVRVGD | SEQ ID |
| | 011954372.1 | IVVYDATWYNEPVIHRVINITQINGTTYYMIKGDHNSHPDPYYATADQINERVLTWD | NO.: 37 |
| | MULTISPECIES: | GHPIVIPYIGNISLWLRGLGGGGSGGGGSGGGGSIENADKAIKDFQDNKAPHDKSAAYE | |
| | signal | ANSKLPKDLRDKNNRFVEKVSIEKAIVRHDERVKSANDAISKLNEKDSIENRRLAQREVN | |
| | peptidase I | KAPMDVKEHLQKQLD | |
| | [Methano- | | |
| | brevibacter], | | |
| | amino acid | | |
| C3.2 | WP_ | cttgttctttttgcagaagctcagaataaacgctcaactttggccaccATGTTCGTGTTC | SEQ ID |
| | 011954372.1 | CTCGTTTTGCTGCCTCTGGTTTCTTCTGCCGCCCTGAACGTGGTCGTGTCAGGATCTATG | NO.: 38 |
| | MULTISPECIES: | GAGCCTGTGTTCTACCGCGGCGACATTGTGGCCGTGGAGAAAGCTGACTTCCTGGGCATC | |
| | signal | CATGAGTTCGATCCTAGCGATGTCAGGGTGGGGGACATCGTGGTCTATGACGCCACGT | |
| | peptidase I | GGTACAACGAGCCTGTGATACACAGAGTCATCAACATAACCCAGATCAACGGAA | |
| | [Methano- | CAACTTACTACATGATCAAGGGCGACCACAATTCACACCCTGACCCTTACTACG | |
| | brevibacter], | CGACCGCCGACCAGATCAACGAGCGGGTCTTGACCTGGGACGGTCACCCTATCG | |
| | nucleotide | TGATCCCTTACATCGGGAACATTTCTCTTTGGCTTCGAGGGCTGGGGGGTGGTGG | |
| | | CTCAGGCGGGGGCGGCAGTGGGGGCGGAGGCTCCATCGAGAACGCTGACAAGG | |
| | | CCATTAAGGACTTTCAGGACAACAAGGCCCCTCACGACAAGAGCGCAGCGTACG | |
| | | AGGCAAACTCCAAACTCCCTAAGGACCTCAGAGACAAGAATAACCGCTTCGTCG | |
| | | AGAAAGTTAGTATCGAAAAGGCAATAGTGCGGCACGACGAACGAGTCAAGAGT | |
| | | GCCAACGATGCCATCAGCAAGCTGAACGAGAAGGACTCCATCGAGAACAGGCG | |
| | | CCTGGCGCAGCGGGAGGTGAACAAGGCCCCTATGGACGTGAAGGAGCACCTGC | |
| | | AAAAGCAGCTCGACtaatgatagaccagcctcaagaacacccgaatggagtctctaagct | |
| | | acataataccaacttacactttacaaaatgttgtcccccaaaatgtagccattcgtatct | |
| | | gctcctaataaaaagaaagtttcttcacattct | |
| C3.3 | WP_ | MFVFLVLLPLVSSAALNVVVSGSMEPAFYRGDIVVIEKSDFLGIHEFNPTDVKVGD | SEQ ID |
| | 042694712.1 | VVVYDAAWYDQPVIHRVINITQINGSTYYVIKGDNNDSPDPYYVSPNQINERVVTFG | NO.: 39 |
| | signal | DNLCVIPYVGYLSLWLRGLGGGGSGGGGSGGGGSIENADKAIKDFQDNKAPHDKSAA | |
| | peptidase I | YEANSKIPKDLRDKNNRFVEKVSIEKAIVRHDERVKSANDAISKLNEKDSIENRRLAQR | |
| | [Methano- | EVNKAPMDVKEHLQKQLD | |
| | brevibacter | | |
| | oralis], | | |
| | amino acid | | |
| C3.3 | WP_ | cttgttctttttgcagaagctcagaataaacgctcaactttggccaccATGTTCGTCTTC | SEQ ID |
| | 042694712.1 | CTCGTGCTTCTCCCTCTGGTCAGCTCAGCTGCATTGAACGTCGTGGTCTCAGGGAGCATG | NO.: 40 |
| | signal | GAACCTGCGTTCTACCGCGGAGACATTGTTGTGATCGAGAAGTCCGATTTTTTGGGCATC | |
| | peptidase I | CACGAGTTCAACCCTACCGACGTGAAGGTCGGTGACGTGGTGGTGTACGACGCAGCGT | |
| | [Methano- | GGTATGACCAGCCTGTGATCCACCGGGTGATAAACATCACACAGATCAACGGCA | |
| | brevibacter | GTACCTACTACGTTATCAAGGGGGACAATAACGACTCTCCTGACCCTTACTACGT | |
| | oralis], | TAGCCCTAACCAAATCAACGAGAGAGTGGTCACGITCGGCGACAACCTGTGCGT | |
| | nucleotide | GATCCCTTACGTGGGGTACCTGTCCCTCTGGCTGAGGGGACTTGGCGGCGGTGG | |
| | | TTCTGGAGGCGGCGGGAGTGGGGGCGGGGGCTCTATCGAGAACGCCGATAAGG | |
| | | CCATAAAGGACTTCCAGGACAATAAAGCCCCTCACGATAAGTCTGCGGCTTACG | |
| | | AGGCCAACTCTAAACTCCCTAAGGACCTGCGGGACAAGAACAACCGATTCGTGG | |
| | | AGAAGGTCAGTATCGAGAAGGCCATTGTCCGCCACGACGAGAGGGTGAAGTCC | |
| | | GCCAATGACGCCATATCTAAGCTGAACGAGAAGGACTCAATCGAAAACCGACG | |
| | | GCTGGCCCAGCGCGAGGTGAACAAGGCCCCTATGGACGTTAAAGAGCATCTGCA | |
| | | GAAGCAGCTTGACtaatgatagaccagcctcaagaacacccgaatggagtctctaagcta | |
| | | cataataccaacttacactttacaaaatgttgtcccccaaaatgtagccattcgtatctg | |
| | | ctcctaataaaaagaaagtttcttcacattct | |
| C4.1 | mru_2047 | MFVFLVLLPLVSSAARGNNFESIQEYIDNEGSSFNSIFNNLVLYKDINDKANLKNEL | SEQ ID |
| | homology | LYILYLLGHGCSYESANFTGVQASGIFRDRWYSFDEYVLTFFNESRNRTIGILESTMY | NO.: 41 |
| | region, −500 | IQSQQLDLVNEITERLESKGYNVIPIYCPAGNAEQLNIMVKYWTSACSNISGFLENPQ | |
| | and +500 | DFDIYVDGIISMVAYGVGGENFTNATKFFEDANVPIFRAVHSEYITNEQWELSPVGLS | |
| | aa, amino | TTKSDKWWHVTIAESQGIFDATYVGGVDSYISNRTGAIILTFVPVHENIELLTDRVDA | |
| | acid | WVDLKYTPNEDKNISIVYYNYPPGKQNIGASYLDAITSVYNMLYTLKDEGYYLTDL | |
| | | PNNVSELEDMMIACGINVANWAPGEVEKLANRSGVALLPVDEYLEWFDSLDDIVK | |
| | | VQITEGPVAYIGQMVRRAVLINYTDEVETMVNDWYNQIKALLPENQTVAATNILDK | |
| | | LVNSLKLYANASSDGDENASLYYDEFLRYYDEFKSLNVSGLNGWGEAPGNIMLVN | |
| | | RNGTDYFVIPGLTFGNVFIGPEPQRGWEADIENLYHCTAVAPTHQYLAAYYYMQTR | |
| | | QSNAMVFVGRHATHEWLPGKEVLLSYNDYGSIVVGKVPQVYFYITDGLAEAIQAK | |
| | | RRGFAVLISHLDSPKSYTHLYGNLTVLATLLEEYDNNHIIIESDSDKDNQAITYQVIK | |
| | | DNQTITYQVINQELEDNLTRAIKDLVIANNYYLTIGFTAEELNNTDMFSLSSTLNAFL | |
| | | KNTQNTLYPLGLHAIGQKWTDEDLANTVAIIVSHDFEYGGKKTNLFDQLSLYYYGE | |
| | | KYSNLTPLKRDYILNRSVDVCKALIYWDTETVSDTIGIGSPEFIESLNIAKKYIDLYNQ | |
| | | CISLELEEMVSALNGGYVPVNIGGESVTVPQVLPTGANMYQDQSSELPTQKAWDYA | |
| | | KTLSLLTLADLNDTTEKIIMGIWCVETARDDGALVSTVLYLLGMEPVWHNSSSAGF | |
| | | DEEGIPTGKKVEDLPNVIALENLTRPDGWAKKRIDVTVITSGLFRDLYSSQARLMDN | |
| | | AYRMALACSYYTIVNNKTIMDSEYGPQVYDALRSIMRSISFKGGGGGSGGGGSGGG | |
| | | GSIENADKAIKDFQDNKAPHDKSAAYEANSKLPKDLRDKNNRFVEKVSIEKAIVRHDERV | |
| | | KSANDAISKLNEKDSIENRRLAQREVNKAPMDVKEHLQKQLD | |
| C4.1 | mru_2047 | cttgttctttttgcagaagctcagaataaacgctcaactttggccaccATGTTCGTCTTC | SEQ ID |
| | homology | CTGGTGCTCTTGCCTCTGGTGTCTAGTGCCGCGCGGGGCAACAACTTCGAGTCTATCCAG | NO.: 42 |
| | region, −500 | GAGTACATCGACAACGAGGGCAGTTCCTTCAACTCTATCTTCAACAACCTGGTGCTTTAT | |
| | and +500 | AAGGACATCAACGACAAGGCCAACCTGAAGAATGAGTTGCTGTACATCCTCTACCTCC | |
| | aa, | TGGGACACGGTTGCTCATACGAGAGTGCCAACTTCACCGGGGTCCAGGCCTCTG | |
| | nucleotide | GGATCTTCCGGGACCGCTGGTACTCTTTCGACGAATACGTCCTCACCTTCTTCAA | |
| | | TGAGTCTCGGAACCGGACGATCGGGATCCTGGAGTCCACGATGTACATCCAGTC | |
| | | TCAGCAGCTGGACCTCGTGAACGAGATCACAGAGCGACTGGAGTCTAAGGGCTA | |
| | | CAACGTTATCCCTATCTACTGTCCTGCAGGGAACGCGGAGCAGCTGAACATCAT | |
| | | GGTGAAGTACTGGACCTCTGCCTGCTCAAATATATCTGGATTCCTCGAGAATCCT | |
| | | CAAGACTTCGACATATACGTGGACGGCATCATATCTATGGTGGCGTACGGCGTG | |
| | | GGGGGGGAGAACTTTACCAACGCGACTAAGTTCTTCGAAGACGCAAACGTTCCT | |
| | | ATCTTCAGGGCCGTGCATAGTGAGTACATAACGAACGAGCAGTGGGAACTCTCT | |
| | | CCTGTCGGCCTGTCCACAACCAAGTCTGACAAGTGGTGGCACGTGACCATCGCC | |
| | | GAGTCTCAAGGCATCTTCGACGCCACCTACGTCGGCGGCGTCGACTCTTACATTT | |
| | | CTAACCGAACCGGCGCTATCATCTTGACCTTTGTCCCTGTGCACGAGAACATTGA | |
| | | GCTGCTGACCGACCGCGTCGACGCATGGGTGGACCTGAAGTACACACCTAACGA | |
| | | GGACAAGAACATCTCTATCGTGTACTACAACTACCCTCCTGGGAAGCAGAACAT | |
| | | AGGGGCCTCATACCTTGACGCCATCACCTCTGTTTATAACATGCTGTACACCCTG | |
| | | AAGGATGAGGGATACTACCTGACCGACCTGCCTAACAACGTGTCTGAGCTGGAG | |
| | | GACATGATGATCGCCTGCGGCATCAATGTGGCCAACTGGGCCCCTGGGGAGGTT | |
| | | GAAAAATTGGCCAATCGGTCTGGCGTGGCGCTCCTGCCTGTGGACGAGTACCTG | |
| | | GAGTGGTTCGACTCACTGGACGATATCGTGAAGGTGCAGATCACTGAGGGCCCT | |
| | | GTTGCCTACATCGGGCAGATGGTCCGGAGGGCGGTGCTCATCAACTACACCGAC | |
| | | GAGGTCGAGACCATGGTCAACGATTGGTACAACCAGATCAAGGCTCTGCTGCCT | |
| | | GAGAACCAGACCGTCGCCGCCACCAACATCCTGGACAAACTCGTTAACTCCCTT | |
| | | AAGCTGTACGCCAACGCATCCTCTGACGGCGACGAGAACGCCAGCCTCTACTAC | |
| | | GACGAGTTCCTGCGGTACTACGACGAGTTCAAGTCTCTGAACGTGTCTGGACTG | |
| | | AACGGGTGGGGCGAGGCACCTGGGAACATCATGCTGGTCAACAGAAACGGCAC | |
| | | GGACTACTTCGTGATCCCTGGCCTGACATTCGGGAACGTGTTCATCGGACCTGAA | |
| | | CCTCAGAGGGGCTGGGAGGCCGACATCGAGAACTTGTACCACTGCACCGCGGTG | |
| | | GCGCCTACCCACCAGTACCTGGCCGCCTACTATTACATGCAGACCCGCCAGTCTA | |
| | | ACGCCATGGTGTTCGTGGGGCGGCACGCCACCCACGAGTGGCTGCCTGGCAAGG | |
| | | AGGTCCTCCTGTCTTACAACGACTACGGCTCTATTGTTGTGGGCAAGGTGCCTCA | |
| | | GGTCTACTTTTACATCACCGACGGCCTCGCGGAGGCTATCCAGGCCAAGCGCCG | |
| | | CGGCTTCGCCGTGCTCATCTCACACCTGGACTCTCCTAAGTCTTACACGCACCTG | |
| | | TACGGCAACCTGACGGTGCTTGCCACTCTTCTCGAGGAGTACGACAACAATCAT | |
| | | ATCATAATTGAGTCTGATTCTGACAAAGACAACCAGGCCATCACATACCAAGTT | |
| | | ATCAAGGACAACCAGACAATCACGTACCAGGTCATTAACCAGGAGTTGGAGGA | |
| | | CAACCTGACGCGCGCCATCAAGGACCTGGTCATCGCCAACAACTACTACCTGAC | |
| | | TATCGGCTTCACCGCCGAGGAGCTGAACAACACCGACATGTTCTCACTTTCTTCA | |
| | | ACTCTGAACGCTTTCCTGAAGAATACCCAGAACACCCTCTACCCTCTCGGCCTGC | |
| | | ACGCCATCGGGCAGAAGTGGACCGATGAGGACCTCGCCAACACCGTGGCGATA | |
| | | ATCGTCAGCCACGACTTCGAGTACGGGGGCAAGAAGACGAACCTTTTCGACCAA | |
| | | CTGTCTTTGTACTATTACGGGGAGAAGTACTCAAACTTGACCCCTCTGAAGCGGG | |
| | | ACTACATACTCAACCGCTCTGTCGACGTGTGCAAGGCCCTTATCTACTGGGACAC | |
| | | GGAGACTGTGTCCGACACGATTGGCATCGGTAGCCCTGAGTTCATCGAGTCTCT | |
| | | GAACATCGCCAAGAAGTATATTGACCTGTACAACCAGTGCATCAGCCTCGAGCT | |
| | | GGAAGAGATGGTCTCCGCACTCAACGGGGGTTATGTGCCTGTGAACATCGGGGG | |
| | | CGAGAGTGTGACTGTCCCTCAGGTGCTGCCTACCGGCGCCAACATGTACCAGGA | |
| | | CCAGTCTTCTGAGTTGCCTACGCAGAAGGCTTGGGACTACGCCAAAACCCTCAG | |
| | | CCTGCTGACACTGGCGGACCTGAATGATACCACCGAGAAAATCATCATGGGCAT | |
| | | CTGGTGCGTGGAGACCGCGCGCGACGACGGAGCTCTCGTGAGTACCGTCTTGTA | |
| | | CCTGCTCGGCATGGAGCCTGTCTGGCACAACTCCTCTTCTGCCGGCTTCGACGAA | |
| | | GAGGGTATCCCTACCGGGAAGAAGGTGGAGGACCTCCCTAACGTGATAGCCCTT | |
| | | GAGAACCTGACGCGCCCTGACGGTTGGGCAAAGAAGAGAATCGACGTGACGGT | |
| | | GATCACCTCTGGTCTGTTCAGGGACTTGTACTCCTCTCAGGCACGGCTGATGGAT | |
| | | AACGCCTACAGAATGGCCCTCGCCTGCAGTTATTACACCATCGTCAACAACAAG | |
| | | ACCATCATGGACTCTGAGTACGGCCCTCAGGTGTACGACGCTCTTCGCAGTATCA | |
| | | TGCGCTCTATCAGCTTTAAGGGGGGCGGAGGGGGCTCAGGGGGTGGGGGCAGC | |
| | | GGGGGTGGGGGATCTATCGAGAACGCCGACAAGGCCATCAAGGACTTCCAGGA | |
| | | CAACAAGGCCCCTCACGACAAGTCTGCCGCGTACGAGGCCAACAGTAAGCTGCC | |
| | | TAAGGACCTTCGGGACAAAAACAACCGATTCGTGGAGAAGGTCAGCATTGAGA | |
| | | AGGCCATCGTGCGCCACGACGAGCGGGTGAAGTCTGCCAACGACGCGATCTCTA | |
| | | AGCTGAACGAGAAGGACAGCATCGAGAACCGGCGACTCGCTCAGAGAGAAGTG | |
| | | AACAAGGCCCCTATGGATGTGAAGGAGCACCTCCAGAAGCAGCTCGACtaatgataga | |
| | | ccagcctcaagaacacccgaatggagtctctaagctacataataccaacttacactttac | |
| | | aaaatgtgtcccccaaaatgtagccattcgtatctgctcctaataaaaagaaagtttctt | |
| | | cacattct | |
| C4.2 | WP_ | MFVFLVLLPLVSSAARGQNYDIYYEYITNGDGKLVNAEFNKAVLYKNYNNKENQI | SEQ ID |
| | 011954718.1 | NEILWALNITGYECKYSDPRFSKTYEYGIFREQYMTLEEYKKKYFDSSRPYTVGLLE | NO.: 43 |
| | that | SNMYVSNGQLQPYYALIKSLEAKGCNVIPVVAAGGSENQLKVMVKYFTNAPSYEA | |
| | aligns to | YLNNPLKYTNNVNAIISMPAYGIGGNLFDNTTKYFETAGVPVFRAVHSDYVSNEEW | |
| | C4.1 (use | ELSATGLPGNRSDKWWHVAIGEAQGIIEATFVGGVTHEISSKTGAQLSGFKAHEKNI | |
| | EMBOSS | DLFTKRIVSWINLQYTVNSDKKISLVYFNYPPGKQNIGSSYLDSITSVYNLLYELKSQ | |
| | Needle), up | GYNVGKLPTTVKELEDMMIKSGINVATWAPGELEKLSNQPNIVLLPVAEYENWENS | |
| | to 1,482 | LEPISKVQVIEGPVAYIGQLARNAIAINYTSPMKDIISDWYNGVKSLLPENYTESGVM | |
| | amino acids | LLDKIVAALNKYLQSGNDSDYQEYLSLKSQWKALNIPGLNGWGKAPGNIMTVTKN | |
| | in total, | GVAYFVIPGLKFGNIFIAPEPQRGWEAKSDLLYHSSAVAPTHQYLAAYYYMQKEYS | |
| | amino acid | SAMVFIGRHATHEWLPGKEVLLSTTDYGSIVVGDVPQIYFYISDGLGEGLEAKRRGF | |
| | | AVMITHLTSPLAYTSLYGNLTAIANLINKYENTTDKTQKDTIASNIKLLIEKNNYIQS | |
| | | MGLTQEEFEKLNLNEVVKAADKFLFEVQNTLYPLGLHAIGQNWTVTDISRSVVAAL | |
| | | SQEFTYDGITTTIFDEVAKYLFSKKYSELNALERDKVLNTSEQIVAALIFSNSTTVAN | |
| | | VLGSDNPSLIAAMNYARYYISLIYASINNELTSFINGLNGKYIPVVSDGDVININSLPT | |
| | | GGNFFHDQSQELPTEEAYNYAKTLTLLTLSSLNEKTQKIAMGIWCVETARDNGALIS | |
| | | VVLYLLGMQPVYTSSPSAGGKTEDGDSVGTKTKIMPKFVGLKDLVRPEGWAKKRI | |
| | | DIVVITSGNFRDLYSTQVSLLDNAFRVALARSYLTIINNKTLMESKYGKDMKEALDK | |
| | | VMEGIGYYGGGGGSGGGGSGGGGSIENADKAIKDFQDNKAPHDKSAAYEANSKLPKD | |
| | | LRDKNNRFVEKVSIEKAIVRHDERVKSANDAISKLNEKDSIENRRLAQREVNKAPMDVKE | |
| | | HLQKQLD | |
| C4.2 | WP_ | cttgttctttttgcagaagctcagaataaacgctcaactttggccaccATGTTCGTGTTC | SEQ ID |
| | 011954718.1 | CTGGTCCTGCTGCCTCTGGTGTCTTCTGCCGCTAGAGGACAGAACTACGACATCTACTAC | NO.: 44 |
| | that | GAGTACATCACCAACGGCGACGGCAAGCTTGTCAATGCCGAGTTCAACAAGGCCGTGCTT | |
| | aligns to | TACAAGAACTATAACAACAAGGAGAACCAGATCAACGAGATCCTGTGGGCTCTGAA | |
| | C4.1 (use | CATTACCGGCTACGAGTGCAAGTACTCAGACCCTCGGTTCTCTAAGACCTACGA | |
| | EMBOSS | GTACGGCATCTTCCGCGAGCAGTACATGACCCTCGAGGAGTACAAGAAGAAGTA | |
| | Needle), up | CTTCGACTCTTCTCGGCCTTACACTGTCGGGCTTCTCGAATCTAATATGTACGTGT | |
| | to 1,482 | CTAACGGGCAGCTGCAGCCTTACTACGCCCTCATCAAGAGCCTCGAGGCCAAGG | |
| | amino acids | GCTGCAACGTTATCCCTGTGGTGGCGGCAGGAGGCTCTGAGAACCAGCTCAAGG | |
| | in total, | TCATGGTGAAGTACTTTACGAACGCGCCTTCTTACGAGGCCTACCTCAACAATCC | |
| | nucleotide | TCTGAAGTACACCAATAATGTGAACGCCATCATCTCTATGCCTGCCTACGGCATA | |
| | | GGTGGCAACCTTTTCGACAACACCACGAAGTACTTCGAGACCGCCGGGGTGCCT | |
| | | GTGTTCCGGGCCGTGCACTCCGACTATGTGTCTAACGAGGAGTGGGAGCTCTCT | |
| | | GCCACCGGGCTGCCTGGCAACCGGTCTGACAAGTGGTGGCACGTCGCCATCGGC | |
| | | GAGGCCCAGGGCATCATCGAAGCGACCTTCGTGGGAGGTGTGACCCACGAGATC | |
| | | TCTTCCAAGACGGGCGCGCAATTGAGCGGTTTCAAGGCCCATGAGAAGAACATA | |
| | | GACCTGTTCACGAAACGGATCGTCTCTTGGATCAATCTGCAGTACACCGTGAAC | |
| | | AGCGACAAGAAGATCTCTCTGGTGTACTTCAACTACCCTCCTGGGAAGCAGAAC | |
| | | ATTGGCTCTAGCTACCTCGACTCTATCACCTCCGTCTACAACCTGCTTTACGAGC | |
| | | TTAAGTCTCAGGGTTACAACGTGGGGAAGCTGCCTACAACCGTGAAGGAGTTGG | |
| | | AGGACATGATGATTAAATCCGGGATAAACGTTGCCACATGGGCCCCTGGGGAAC | |
| | | TCGAGAAGCTGAGTAACCAGCCTAACATCGTTCTCCTGCCTGTGGCCGAGTACG | |
| | | AGAACTGGTTCAACTCCTTGGAGCCTATCTCAAAGGTCCAGGTGATCGAGGGAC | |
| | | CTGTGGCCTACATCGGGCAGCTGGCCCGGAACGCCATCGCTATCAACTACACCT | |
| | | CTCCTATGAAGGACATCATCTCTGACTGGTACAACGGAGTGAAGTCTCTTCTGCC | |
| | | TGAGAACTACACCGAGTCCGGGGTGATGCTCCTGGACAAGATCGTCGCTGCTCT | |
| | | CAACAAGTACCTGCAGTCTGGGAACGACTCCGATTACCAAGAGTACCTGTCTCT | |
| | | GAAGTCTCAGTGGAAGGCCCTCAACATTCCTGGGCTGAACGGGTGGGGCAAGGC | |
| | | CCCTGGCAACATAATGACTGTCACCAAGAACGGCGTGGCCTACTTTGTGATCCCT | |
| | | GGCTTGAAGTTTGGGAACATCTTCATCGCCCCTGAGCCTCAGAGAGGCTGGGAG | |
| | | GCTAAGTCTGATTTGCTGTACCACTCTTCTGCGGTGGCTCCTACTCACCAGTACC | |
| | | TTGCGGCATATTACTACATGCAAAAGGAGTACAGTAGTGCCATGGTCTTCATCG | |
| | | GCCGCCACGCCACGCACGAGTGGCTGCCTGGGAAGGAGGTGCTCCTGAGTACCA | |
| | | CCGACTATGGCTCTATCGTGGTCGGGGATGTCCCTCAGATCTACTTCTACATCTC | |
| | | TGACGGGCTGGGGGAAGGTCTTGAGGCCAAGCGACGCGGGTTCGCCGTGATGAT | |
| | | CACGCACCTGACTTCTCCTCTGGCCTATACATCACTGTACGGCAACCTGACCGCC | |
| | | ATCGCCAACCTCATTAACAAGTACGAGAACACGACAGACAAGACGCAGAAGGA | |
| | | TACCATCGCGTCTAACATCAAGCTGCTGATCGAAAAAAACAACTACATCCAGTC | |
| | | TATGGGGCTGACTCAGGAGGAGTTTGAGAAGCTGAACCTTAACGAGGTTGTGAA | |
| | | GGCCGCCGACAAGTTCCTCTTCGAAGTCCAGAACACGCTGTACCCTCTGGGCTTG | |
| | | CACGCGATAGGCCAGAACTGGACCGTCACCGACATCAGCCGCAGCGTTGTGGCC | |
| | | GCGCTCTCTCAGGAGTTCACCTACGACGGGATCACCACCACTATTTTCGACGAA | |
| | | GTTGCGAAATACCTGTTCTCTAAGAAGTACTCAGAGCTGAACGCCCTGGAGCGA | |
| | | GACAAAGTCCTTAACACCTCTGAGCAGATAGTGGCGGCCCTCATTTTCTCAAAC | |
| | | AGCACCACGGTGGCCAACGTGCTCGGCTCTGACAACCCTTCTCTGATCGCCGCC | |
| | | ATGAACTACGCCCGCTACTACATCTCTCTGATCTACGCATCTATCAACAACGAGC | |
| | | TGACCTCTTTCATTAATGGGCTGAACGGCAAGTACATCCCTGTGGTCAGTGACGG | |
| | | AGACGTTATCAATATCAACTCACTGCCTACGGGCGGGAACTTCTTCCACGACCA | |
| | | GAGCCAGGAGCTCCCTACCGAGGAGGCCTATAACTACGCGAAGACCCTCACACT | |
| | | GCTGACCCTCTCTTCCTTGAACGAGAAAACCCAGAAGATCGCGATGGGAATCTG | |
| | | GTGCGTCGAGACAGCACGGGACAACGGCGCCCTCATCTCTGTGGTGCTCTACCT | |
| | | GTTGGGAATGCAGCCTGTGTACACGTCATCTCCTTCTGCCGGCGGGAAGACGGA | |
| | | GGACGGCGACTCCGTGGGCACGAAGACCAAGATAATGCCTAAGTTCGTCGGGCT | |
| | | GAAGGACCTGGTCCGGCCTGAGGGCTGGGCCAAGAAGCGCATCGACATCGTGGT | |
| | | GATCACCTCTGGCAACTTCCGCGACCTGTATAGTACCCAGGTCAGTCTCCTCGAC | |
| | | AACGCCTTCCGGGTGGCCCTGGCCCGGTCATACCTGACCATCATCAACAACAAG | |
| | | ACCCTGATGGAGAGCAAGTACGGGAAAGACATGAAGGAGGCGCTGGACAAGGT | |
| | | TATGGAGGGCATCGGTTACTACGGCGGCGGTGGAGGCAGTGGTGGCGGGGGCTC | |
| | | TGGCGGCGGGGGCTCTATCGAGAACGCTGACAAGGCCATCAAGGACTTCCAGGA | |
| | | CAACAAGGCCCCTCACGACAAATCTGCCGCATACGAGGCGAACTCTAAGCTCCC | |
| | | TAAGGACTTGAGAGACAAGAACAACCGCTTCGTGGAGAAGGTGTCAATCGAGA | |
| | | AGGCCATCGTGAGGCACGACGAGAGGGTCAAGAGTGCAAACGATGCAATATCT | |
| | | AAGCTGAATGAGAAGGACTCTATCGAGAACAGGCGACTCGCCCAGCGCGAGGT | |
| | | CAACAAGGCACCTATGGACGTGAAGGAGCACCTGCAGAAGCAATTGGACtaatgata | |
| | | gaccagcctcaagaacacccgaatggagtctctaagctacataataccaacttacacttt | |
| | | acaaaatgttgtcccccaaaatgtagccattcgtatctgctcctaataaaaagaaagttt | |
| | | cttcacattct | |
| C5.1 | mru_1383 | MFVFLVLLPLVSSAAYTGTGFSHDIPFSKYSSQSNSDILNKYNNTDCHSEIKGICTYV | SEQ ID |
| | staphy- | ADGDTIDVEGVGRVRFVGVNTPERGVTAYICSKRFVQKFCLNKEVSLDVDDSKRND | NO.: 45 |
| | lococcal | RYGRTLAVVIVDGKNLNEMLLKEGLAEIMYIPPSEFYPYDWSSDSTTSSSYTSGSSSS | |
| | nuclease | NSGGSYSSSSFTSGSTVSAPYVGSANSHKFHYSTCKWGKKISDKNRVTFNSRSDAIS | |
| | domain- | QGYAPCKACQPGGGGSGGGGSGGGGSIENADKAIKDFQDNKAPHDKSAAYEANSKLP | |
| | containing | KDLRDKNNRFVEKVSIEKAIVRHDERVKSANDAISKLNEKDSIENRRLAQREVNKAPMDV | |
| | protein = | KEHLQKQLD | |
| | WP_ | | |
| | 012956182.1 | | |
| | thermo- | | |
| | nuclease | | |
| | family | | |
| | protein | | |
| | [Methano- | | |
| | brevibacter | | |
| | ruminantium], | | |
| | amino acid | | |
| C5.1 | mru_1383 | cttgttctttttgcagaagctcagaataaacgctcaactttggccaccATGTTCGTCTTC | SEQ ID |
| | staphy- | CTTGTGCTGCTGCCTTTGGTCAGTTCCGCCGCTTACACCGGGACCGGCTTCTCCCACGAC | NO.: 46 |
| | lococcal | ATCCCTTTCTCCAAGTACAGTAGCCAGTCTAACTCTGATATCCTGAACAAGTACAACAAC | |
| | nuclease | ACAGACTGCCACAGCGAAATCAAAGGGATCTGCACCTACGTCGCCGACGGAGACACCA | |
| | domain- | TAGATGTTGAGGGGGTGGGACGCGTCCGGTTTGTGGGAGTGAACACCCCTGAGA | |
| | containing | GAGGTGTCACCGCGTACATCTGCAGTAAGCGGTTCGTGCAGAAGTTCTGCCTTA | |
| | protein = | ACAAGGAGGTGTCACTCGACGTTGACGACTCCAAGCGAAACGACCGCTACGGCA | |
| | WP_ | GGACGCTGGCGGTTGTGATCGTGGACGGTAAGAATCTTAACGAGATGTTGCTCA | |
| | 012956182.1 | AGGAGGGTCTGGCAGAGATCATGTACATCCCTCCTTCGGAGTTCTACCCTTATGA | |
| | thermo- | CTGGAGCTCCGACTCTACCACGTCTAGTAGCTACACTTCTGGCTCTTCTTCTTCA | |
| | nuclease | AACTCTGGCGGCTCATATAGTTCATCTTCATTCACGAGCGGCAGTACCGTGTCCG | |
| | family | CCCCTTACGTGGGGTCTGCAAACTCACACAAGTTCCACTACAGCACTTGCAAGT | |
| | protein | GGGGGAAGAAGATCTCTGACAAGAACCGGGTGACATTCAACTCAAGATCAGAC | |
| | [Methano- | GCAATCAGTCAGGGATACGCGCCTTGTAAGGCCTGCCAACCTGGGGGAGGCGGC | |
| | brevibacter | TCTGGCGGGGGCGGTAGCGGCGGGGGGGGGAGTATAGAAAACGCCGACAAAGC | |
| | ruminantium], | TATCAAGGATTTCCAGGACAACAAGGCCCCTCATGACAAGTCTGCCGCCTACGA | |
| | nucleotide | GGCCAATAGCAAGCTGCCTAAGGACCTCCGAGACAAAAATAACAGGTTTGTCGA | |
| | | GAAGGTCTCTATCGAAAAGGCCATTGTGCGGCACGACGAGCGCGTGAAGTCCGC | |
| | | GAACGACGCTATATCTAAATTGAACGAGAAGGACTCCATTGAGAACCGGCGCCT | |
| | | GGCCCAGCGAGAGGTTAACAAGGCCCCTATGGACGTGAAAGAGCACCTCCAGA | |
| | | AGCAGCTGGATtaatgatagaccagcctcaagaacacccgaatggagtctctaagctaca | |
| | | taataccaacttacactttacaaaatgttgtcccccaaaatgtagccattcgtatctgc | |
| | | tcctaataaaaagaaagtttcttcacattct | |
| C5.2 | WP_ | MFVFLVLLPLVSSAAYTGTGFSHDIPLSKYSDSSYKNILDKYNDTECEAEVSGICTR | SEQ ID |
| | 063720174.1 | VVDGDTIYVDGVGKVRFVGVNTPENGVEGGDVSKYFVQKLCMNQEVGLDIDDSK | NO.: 47 |
| | thermo- | QQDKYGRTLAVVIIDDKNLNEMLLKEGLAEIMYIPPSEFDPYSWSNGSTDINEHAHS | |
| | nuclease | KSTDTSSDSSGKYVASMNSDKFHKPSCRWAEKIYEQNKISFNSRESAINNGYQPCKV | |
| | family | CNPGGGGSGGGGSGGGGSIENADKAIKDFQDNKAPHDKSAAYEANSKLPKDLRDKNNR | |
| | protein | FVEKVSIEKAIVRHDERVKSANDAISKLNEKDSIENRRLAQREVNKAPMDVKEHLQKQLD | |
| | [Methano- | | |
| | brevibacter | | |
| | oralis], | | |
| | amino acid | | |
| C5.2 | WP_ | cttgttctttttgcagaagctcagaataaacgctcaactttggccaccATGTTCGTGTTC | SEQ ID |
| | 063720174.1 | CTTGTTCTGCTCCCTTTGGTGAGTTCTGCCGCCTACACGGGCACCGGCTTTAGCCATGAC | NO.: 48 |
| | thermo- | ATCCCTCTGAGTAAGTACTCCGACTCTTCCTACAAGAACATTCTTGATAAGTACAACGAC | |
| | nuclease | ACAGAGTGTGAGGCAGAGGTGTCCGGGATCTGCACCAGAGTGGTGGACGGTGACACTA | |
| | family | TCTATGTGGACGGCGTCGGTAAGGTGCGCTTCGTCGGCGTGAACACACCTGAGA | |
| | protein | ACGGCGTCGAGGGTGGCGACGTTAGTAAGTATTTTGTCCAGAAGCTGTGCATGA | |
| | [Methano- | ACCAGGAGGTGGGCCTTGATATCGACGACTCTAAGCAGCAAGACAAGTACGGG | |
| | brevibacter | AGGACCCTGGCCGTGGTGATCATCGACGACAAGAACCTCAACGAGATGCTGCTC | |
| | oralis], | AAGGAGGGGCTCGCCGAGATCATGTACATCCCTCCTTCTGAGTTCGACCCTTACT | |
| | nucleotide | CCTGGAGCAACGGGTCTACGGACATAAACGAGCACGCTCACTCTAAGTCAACCG | |
| | | ATACCTCCAGCGACTCATCTGGTAAATACGTCGCCAGTATGAATTCAGACAAGT | |
| | | TCCACAAACCTTCTTGCAGGTGGGCCGAGAAGATCTACGAACAGAACAAGATAA | |
| | | GTTTCAACTCTCGGGAGTCAGCCATCAACAATGGATACCAGCCTTGCAAGGTTT | |
| | | GCAACCCTGGCGGAGGGGGATCCGGCGGCGGCGGATCAGGCGGGGGGGGGAGC | |
| | | ATCGAGAACGCGGACAAGGCAATTAAGGACTTCCAGGACAATAAGGCCCCTCA | |
| | | CGACAAAAGTGCCGCTTACGAAGCAAACAGCAAGCTGCCTAAGGACCTGCGGG | |
| | | ACAAGAACAACCGATTCGTGGAGAAGGTGAGCATTGAGAAGGCGATCGTTCGG | |
| | | CACGATGAGCGCGTGAAGTCTGCCAACGACGCTATATCTAAATTGAACGAAAAG | |
| | | GACTCAATCGAGAATAGACGATTGGCGCAGCGCGAGGTCAACAAGGCCCCTATG | |
| | | GATGTCAAGGAGCACCTGCAGAAACAGCTGGACtaatgatagaccagcctcaagaacacc | |
| | | cgaatggagtctctaagctacataataccaacttacactttacaaaatgttgtcccccaa | |
| | | aatgtagecattcgtatctgctcctaataaaaagaaagtttcttcacattct | |
| C6.1 | mru_1923, | MFVFLVLLPLVSSAAAEDLESDIGSQSNPNSQVQLAPQMGHLHRMINKAASGEPVA | SEQ ID |
| | MtrE = | YGCWCGISGAIAALAMGMGIIPIVAIAMGSTVAALVHAIYTVTSHMGRIVGQSQFEQ | NO.: 49 |
| | WP_ | PLFMDVLTQSLGPIAAHGFIASFGIVGIAYLMTLPLDGLGHPFPLPLLAVLWGITIGAI | |
| | 012956721.1 | GSSTGDVHYGAESEYQKFDYGGGTPVAIQGDIVTKAPLGAKNSIDVGNFCAKYGGP | |
| | tetrahydrom- | LTGFCFGLIVFVSFWITVVFGALGGQIVGIVIVILLIAANYLLEKSTRAKFGPYEEGGG | |
| | ethanopterin | GSGGGGSGGGGSIENADKAIKDFQDNKAPHDKSAAYEANSKLPKDLRDKNNRFVEKVSI | |
| | S- | EKAIVRHDERVKSANDAISKLNEKDSIENRRLAQREVNKAPMDVKEHLQKQLD | |
| | methyl- | | |
| | transferase | | |
| | subunit E | | |
| | [Methano- | | |
| | brevibacter | | |
| | ruminantium], | | |
| | amino acid | | |
| C6.1 | mru_1923, | cttgttctttttgcagaagctcagaataaacgctcaactttggccaccATGTTCGTTTTC | SEQ ID |
| | MtrE = | CCTGGTGCTCTTGCCTTGGTCTCTTCAGCAGCCGCCGAGGACCTGGAAAGCGACATCGGC | NO.: 50 |
| | WP_ | ATCCCAGAGTACCCTAACTCACAGGTGCAGCTGGCCCCTCAGATGGGCCACCTGCACCGG | |
| | 012956721.1 | ATGATCAACAAGGCCGCCTCTGGGGAGCCTGTCGCGTACGGCTGCTGGTGCGGGATC | |
| | tetrahydrom- | TCTGGAGCCATCGCGGCTCTGGCCATGGGCATGGGCATCATCCCTATCGTGGCC | |
| | ethanopterin | ATCGCCATGGGCTCTACCGTGGCCGCCCTGGTGCACGCCATATACACGGTCACCT | |
| | S- | CTCACATGGGCAGGATCGTGGGCCAGAGCCAGTTCGAGCAGCCTCTGTTCATGG | |
| | methyl- | ACGTGCTTACGCAGAGTCTCGGCCCTATCGCCGCCCATGGGTTCATTGCGTCTTT | |
| | transferase | CGGCATCGTCGGTATCGCCTACCTTATGACCCTGCCTCTGGACGGCCTCGGGCAC | |
| | subunit E | CCTTTCCCTCTTCCTCTGCTGGCCGTGCTCTGGGGAATTACCATAGGCGCTATAG | |
| | [Methano- | GGTCCTCTACCGGCGACGTGCACTACGGGGCTGAGTCTGAGTACCAGAAGTTCG | |
| | brevibacter | ACTACGGCGGAGGTACGCCTGTGGCCATCCAGGGTGACATCGTTACCAAAGCAC | |
| | ruminantium], | CTCTTGGCGCCAAGAACAGTATCGACGTGGGCAACTTCTGCGCCAAGTACGGCG | |
| | nucleotide | GGCCTCTCACTGGGTTTTGCTTCGGGCTCATCGTCTTCGTGTCTTTCTGGATCACA | |
| | | GTGGTCTTCGGTGCACTGGGGGGCCAGATCGTCGGGATTGTCATCGTTATCCTGC | |
| | | TCATCGCGGCCAACTACCTGTTGGAAAAGTCTACCCGAGCGAAGTTCGGCCCTT | |
| | | ATGAGGAGGGAGGCGGGGGATCTGGCGGGGGGGGGTCAGGCGGTGGGGGGTCG | |
| | | ATCGAGAACGCCGACAAGGCGATCAAGGACTTCCAAGACAACAAGGCCCCTCA | |
| | | CGACAAGTCTGCCGCGTACGAGGCTAACAGCAAGCTCCCTAAGGACCTGCGCGA | |
| | | TAAGAACAACAGATTTGTGGAGAAGGTGTCTATCGAGAAGGCCATCGTGCGGCA | |
| | | CGACGAGCGCGTGAAGTCTGCGAACGACGCCATCTCCAAGCTGAACGAGAAGG | |
| | | ACTCTATCGAGAATCGGCGGTTGGCCCAGCGCGAGGTGAACAAGGCACCTATGG | |
| | | ACGTCAAGGAGCACCTGCAGAAACAGCTCGATtaatgatagaccagcctcaagaacaccc | |
| | | gaatggagtctctaagctacataataccaacttacactttacaaaatgttgtcccccaaa | |
| | | atgtagccattcgtatctgctcctaataaaaagaaagtttcttcacattct | |
| C6.2 | WP_ | MFVFLVLLPLVSSAAAEDLESDIGSQSNPNSQVQLAPQMGHLHRMINKAASGEPVA | SEQ ID |
| | 004032919.1 | YGVWCGVAGSIAYVLIMLGFVPIISIAMGSCVAAFVHAIYTVTSHMGRIVGQSQFEQ | NO.: 51 |
| | MULTISPECIES: | PLFMDVLTQSLGPIVGHGFITSFCIVGISYLMUIPLNGTTLHVFPLPLLAVLWGIALGAI | |
| | tetrahydrom- | GSSTGDVHYGAESEYQKFEFGGGTPVAIQGDIVINAPMGAKNSMDVVNFCAKFGG | |
| | ethanopterin | PLTGFCFGLVVFFSFWNTVVFGIYGGLVVGFIIVILLIIMNDRLEVFARNRYGPYEED | |
| | S- | GGGGSGGGGSGGGGSIENADKAIKDFQDNKAPHDKSAAYEANSKLPKDLRDKNNRFVE | |
| | methyl- | KVSIEKAIVRHDERVKSANDAISKLNEKDSIENRRLAQREVNKAPMDVKEHLQKQLD | |
| | transferase | | |
| | subunit E | | |
| | [Methano- | | |
| | brevibacter], | | |
| | amino acid | | |
| C6.2 | WP_ | cttgttctttttgcagaagctcagaataaacgctcaactttggccaccATGTTCGTGTTC | SEQ ID |
| | 004032919.1 | CTGGTTCTGCTGCCTCTGGTTTCATCTGCGGCCGCGGAGGACCTCGAGTCTGACATCGGA | NO.: 52 |
| | MULTISPECIES: | AGCCAGTCAAACCCTAACTCTCAAGTGCAGCTTGCGCCTCAGATGGGCCACCTCCACCGG | |
| | tetrahydrom- | ATGATCAATAAGGCAGCCTCTGGCGAGCCTGTGGCTTACGGGGTTTGGTGCGGGGTG | |
| | ethanopterin | GCAGGATCCATCGCTTACGTCCTGATCATGTTGGGTTTCGTGCCTATCATCTCTA | |
| | S- | TCGCCATGGGGAGTTGCGTCGCCGCCTTCGTCCACGCGATCTACACTGTGACCAG | |
| | methyl- | CCACATGGGCAGAATCGTCGGCCAGTCTCAGTTCGAACAGCCTCTGTTCATGGA | |
| | transferase | CGTCTTGACCCAGTCACTCGGGCCTATCGTGGGCCACGGGTTCATCACCTCTTTC | |
| | subunit E | TGCATCGTGGGGATATCCTACCTGATGATCATCCCTCTGAACGGGACGACCCTGC | |
| | [Methano- | ACGTGTTCCCTCTGCCTCTGCTCGCTGTGCTGTGGGGTATCGCGTTGGGCGCCAT | |
| | brevibacter], | CGGGTCTTCTACAGGAGACGTGCACTACGGCGCCGAGTCTGAGTACCAGAAGTT | |
| | nucleotide | TGAGTTCGGCGGCGGGACGCCTGTGGCCATTCAGGGGGACATCGTGACCAACGC | |
| | | CCCTATGGGCGCCAAGAACTCTATGGACGTGGTGAACTTCTGCGCCAAGTTCGG | |
| | | CGGCCCTCTCACCGGCTTCTGCTTCGGTCTGGTCGTGTTCTTCTCTTTTTGGAACA | |
| | | CCGTGGTCTTCGGGATTTACGGCGGCCTCGTCGTGGGATTCATCATCGTGATCCT | |
| | | GCTTATCATAATGAACGACCGCCTTGAAGTTTTCGCCAGGAACCGCTACGGCCCT | |
| | | TATGAGGAGGACGGAGGGGGGGGCTCTGGGGGGGGGGGGTCTGGCGGCGGTGG | |
| | | GTCTATCGAGAACGCCGACAAGGCGATCAAGGACTTCCAGGATAACAAGGCCCC | |
| | | TCACGACAAGTCTGCCGCCTACGAGGCCAACTCTAAGCTGCCTAAGGATCTGCG | |
| | | GGACAAGAACAACCGGTTCGTCGAGAAAGTGTCCATCGAGAAGGCCATAGTCC | |
| | | GCCATGACGAGCGCGTGAAGAGTGCCAACGACGCCATCAGCAAGCTCAACGAG | |
| | | AAAGACAGTATTGAGAACCGGCGACTCGCCCAGCGGGAGGTCAATAAGGCACC | |
| | | TATGGACGTGAAGGAGCACCTGCAGAAGCAGCTCGACtaatgatagaccagcctcaagaa | |
| | | cacccgaatggagtctctaagctacataataccaacttacactttacaaaatgttgtccc | |
| | | ccaaaatgtagccattcgtatctgctcctaataaaaagaaagtttcttcacattct | |
| C6.3 | WP_ | MFVFLVLLPLVSSAAAEDLESDIGSQSNPNSQVQLAPQMGHLHRMINKAASGEPVA | SEQ ID |
| | 081738309.1 | YGVWCGVAGAIAYILIHFGIFPIVGIAIGASVAAFVHSIYTVTSHMGRIVGQSQFEQPL | NO.: 53 |
| | tetrahydrom- | FMDVLTQSLGPIAGHGFITSFCIVGISYLMTIPLNGTALHVFPLPLLAMLWGIALGAIG | |
| | ethanopterin | SSTGDVHYGAESEYQKFEFGGGTPVAIQGDIVTKAPIGAKNSMDVVNFCAKFGGPL | |
| | S- | TGFCFGLVVFFSFWNTVVFGIYGGIIVGLIIVVLLGVMNDRLEVFAREKYGPYEEGG | |
| | methyl- | GGSGGGGSGGGGSIENADKAIKDFQDNKAPHDKSAAYEANSKLPKDLRDKNNRFVEKV | |
| | transferase | SIEKAIVRHDERVKSANDAISKLNEKDSIENRRLAQREVNKAPMDVKEHLQKQLD | |
| | subunit E | | |
| | [Methano- | | |
| | brevibacter | | |
| | oralis], | | |
| | amino acid | | |
| C6.3 | WP_ | cttgttctttttgcagaagctcagaataaacgctcaactttggccaccATGTTCGTGTTC | SEQ ID |
| | 081738309.1 | CTCGTGCTCCTGCCTCTGGTGTCTTCCGCAGCGGCCGAGGACTTGGAGAGCGACATCGGC | NO.: 54 |
| | tetrahydrom- | AGTCAGTCAAACCCTAACTCTCAAGTGCAGCTGGCCCCTCAGATGGGCCACCTCCACCGC | |
| | ethanopterin | ATGATCAATAAGGCCGCTTCTGGCGAGCCTGTTGCCTACGGCGTCTGGTGCGGGGTC | |
| | S- | GCCGGCGCCATCGCTTACATCCTGATACACTTCGGCATTTTTCCTATCGTGGGCA | |
| | methyl- | TCGCCATCGGCGCAAGCGTGGCCGCCTTCGTTCACTCTATCTACACCGTTACGAG | |
| | transferase | TCACATGGGTAGGATCGTCGGACAATCACAGTTCGAGCAGCCTCTGTTCATGGA | |
| | subunit E | CGTGCTTACTCAGTCTCTGGGCCCTATCGCCGGGCACGGCTTCATCACCTCTTTC | |
| | [Methano- | TGCATCGTGGGCATCAGCTACCTCATGACCATCCCTCTGAATGGCACAGCCCTGC | |
| | brevibacter | ACGTCTTCCCTCTGCCTCTCTTGGCGATGCTCTGGGGGATTGCCCTTGGGGCCAT | |
| | oralis], | CGGGTCCTCTACCGGAGACGTCCACTACGGGGCCGAGTCCGAGTACCAGAAGTT | |
| | nucleotide | CGAGTTCGGTGGTGGGACACCTGTGGCAATCCAGGGCGACATCGTGACCAAGGC | |
| | | CCCTATCGGAGCGAAGAACTCAATGGATGTGGTGAACTTCTGTGCTAAGTTCGG | |
| | | CGGCCCTCTGACGGGGTTCTGCTTCGGGCTCGTCGTTTTTTTCAGTTTTTGGAACA | |
| | | CCGTGGTCTTCGGCATCTACGGGGGGATCATAGTCGGTCTTATCATAGTGGTGCT | |
| | | GCTGGGCGTTATGAATGACCGGCTTGAAGTGTTCGCTCGAGAGAAGTATGGCCC | |
| | | TTACGAGGAGGGGGGTGGGGGAAGTGGAGGCGGAGGTTCCGGCGGGGGAGGTA | |
| | | GCATAGAGAACGCCGATAAGGCCATAAAAGACTTCCAGGACAACAAGGCGCCT | |
| | | CATGACAAAAGCGCTGCGTACGAGGCAAACTCAAAATTGCCTAAGGATCTTCGC | |
| | | GACAAGAACAACAGGTTCGTCGAGAAGGTGTCAATCGAAAAGGCCATTGTGCG | |
| | | GCACGACGAGCGCGTCAAGAGTGCGAACGACGCCATTTCCAAGTTGAACGAGA | |
| | | AGGACTCTATTGAGAACCGACGGCTGGCACAGAGAGAGGTGAACAAGGCCCCT | |
| | | ATGGACGTGAAGGAACATTTGCAGAAGCAGCTGGACtaatgatagaccagcctcaagaac | |
| | | acccgaatggagtctctaagctacataataccaacttacactttacaaaatgttgtcccc | |
| | | caaaatgtagccattcgtatctgctcctaataaaaagaaagtttcttcacattct | |
| C7.1 | WP_ | MFVFLVLLPLVSSAAATTVFLTSDNIMGTNDDADMLNSIKTYIEEISNGKINVIVDS | SEQ ID |
| | 011953874.1 | QSPGPGEGTRAIEADSNVSVVFAAVDPGNFLVLSKYSTATTDKQIIFVNTGDYDLDT | NO.: 55 |
| | before | AESLRRAWDDNYSKTIFAGINNPGTFLNDGGISYIQPLKEYHDAGSDGIINQNNDDV | |
| | TM and | NKYIAQEIVNNINNYNNTKHYDNNLVITHKLAPSNMAHGSQSLLESNDNEMNGTYN | |
| | short C-term | SYSAPQLLYLTSSYLNGNGLENPGDYKAPDSPLKYSILTKDSYSIYDYIKMGGIVKN | |
| | loop, amino | YMDENGQAPNYINYEGAYISYYDLQYNFAKITANHTDGSHMDFDREYHFDKVNDSI | |
| | acid | GGGGSGGGGSGGGGSIENADKAIKDFQDNKAPHDKSAAYEANSKLPKDLRDKNNRFVE | |
| | | KVSIEKAIVRHDERVKSANDAISKLNEKDSIENRRLAQREVNKAPMDVKEHLQKQLD | |
| C7.1 | WP_011953 | cttgttctttttgcagaagctcagaataaacgctcaactttggccaccATGTTTGTGTTC | SEQ ID |
| | 874.1 | CTGGTGTTGCTCCCTCTGGTGTCTAGCGCGGCCGCCACGACCGTGTTCCTTACCTCCGAC | NO.: 56 |
| | before | AACATCATGGGCACGAACGACGACGCCGACATGCTGAATAGCATCAAGACCTACATCGAG | |
| | TM and | GAAATTTCAAACGGGAAGATCAACGTCATTGTGGACTCCCAGTCACCTGGACCTGGG | |
| | short C-term | GAGGGAACCCGCGCCATCGAAGCCGACAGCAACGTGAGTGTCGTTTTCGCCGCC | |
| | loop, | GTGGACCCTGGCAACTTCCTGGTGCTGTCTAAGTACTCTACCGCCACCACCGACA | |
| | nucleotide | AGCAGATCATCTTCGTGAACACGGGCGACTATGACCTCGACACTGCCGAGTCTC | |
| | | TGCGGCGGGCCTGGGACGACAACTACAGCAAAACCATCTTCGCAGGGATAAAC | |
| | | AACCCTGGCACATTCCTGAACGACGGGGGCATATCTTACATCCAGCCTCTGAAG | |
| | | GAGTACCACGACGCCGGCTCTGACGGCATTATCAACCAGAACAACGACGACGTG | |
| | | AACAAGTACATCGCCCAGGAGATCGTCAACAATATCAACAACTACAACAACACC | |
| | | AAGCACTACGACAACAACCTTGTCATCACCCACAAGCTGGCTCCTAGCAACATG | |
| | | GCCCACGGCTCTCAGTCCCTCCTGGAGTCTAACGACAACGAGATGAATGGGACC | |
| | | TACAACTCTTACTCTGCGCCTCAGTTGTTGTACCTTACTTCGAGTTACCTGAACG | |
| | | GGAACGGGCTGGAGAACCCTGGAGACTATAAGGCGCCTGACAGTCCTCTGAAGT | |
| | | ACAGTATCCTCACGAAGGACTCTTACTCTATCTACGATTACATCAAGATGGGCG | |
| | | GCATCGTCAAGAACTACATGGATGAGAACGGCCAAGCCCCTAACTACATCAACT | |
| | | ACGAGGGGGCCTACATCTCTTATTACGACCTCCAGTACAACTTCGCGAAGATCA | |
| | | CCGCAAACCACACAGACGGGTCTCACATGGACTTCGACCGCGAGTACCATTTCG | |
| | | ACAAGGTCAACGACTCTATCGGCGGTGGTGGATCAGGCGGGGGCGGTTCCGGGG | |
| | | GCGGCGGCTCTATCGAGAACGCGGACAAGGCCATCAAAGACTTCCAGGACAAC | |
| | | AAGGCCCCTCACGATAAGTCTGCCGCCTACGAGGCTAACTCTAAGCTCCCTAAG | |
| | | GACCTCCGCGACAAGAACAACCGATTCGTCGAGAAAGTGTCTATCGAGAAGGCC | |
| | | ATAGTGCGGCACGATGAGCGGGTGAAGTCTGCAAATGACGCTATCTCAAAGCTC | |
| | | AACGAGAAGGACTCAATCGAGAATAGGAGACTGGCGCAGCGCGAGGTGAACAA | |
| | | GGCCCCTATGGACGTTAAGGAGCACCTGCAGAAGCAGCTGGACtaatgatagaccagcct | |
| | | caagaacacccgaatggagtctctaagctacataataccaacttacactttacaaaatgt | |
| | | tgtcccccaaaatgtagccattcgtatctgctcctaataaaaagaaagtttcttcacatt | |
| | | ct | |
| C7.2 | WP_ | MFVFLVLLPLVSSAAAQTVFITSDNIIDHDTDVRMLNSIKEHIEELSNGELQVIVDNQ | SEQ ID |
| | 042694202.1 | APGPGEGYRSIQVTSDICVNIAASDAGNYLQLANYTVYNDKHIIFVNSGSYDLDNSS | NO.: 57 |
| | before | NYLRRAWDDNYSNQYFAGVHDPGTLLKNSGVTYIQPLKEYPDAGDGDNIDKYDEE | |
| | TM and | MNKYIAQEVVNQVKNYNYESKILSDSLIVYHHIEPSIMASASKELVKSNDTEMNGTY | |
| | short C-term | GGYSAPQLLYQTSSYLNGNGLDNPKNFTAPESPMKFSLLTKGSYTINDYIKMGGIVK | |
| | loop, amino | TYMDENGQAPDYINYEGAYISYYDLVYNFAKITQNHTNTQHMGFDSEYEFEKTNDS | |
| | acid | GGGGSGGGGSGGGGSIENADKAIKDFQDNKAPHDKSAAYEANSKLPKDLRDKNNRFVE | |
| | | KVSIEKAIVRHDERVKSANDAISKLNEKDSIENRRLAQREVNKAPMDVKEHLQKQLD | |
| C7.2 | WP_ | cttgttctttttgcagaagctcagaataaacgctcaactttggccaccATGTTCGTGTTT | SEQ ID |
| | 042694202.1 | CTCGTGCTTCTGCCTCTCGTGTCAAGTGCCGCCGCCCAGACAGTGTTCATCACCTCCGAC | NO.: 58 |
| | before | AACATAATCGACCACGATACCGACGTTAGGATGTTGAACAGTATCAAGGAACACATCGAG | |
| | TM and | GAGCTGTCAAACGGGGAGCTGCAGGTTATCGTGGATAACCAGGCCCCTGGGCCTGG | |
| | short C-term | GGAGGGTTACCGAAGTATCCAAGTCACTTCCGACATCTGCGTCAATATCGCTGCT | |
| | loop, | AGCGACGCTGGCAACTATCTGCAACTCGCCAACTATACTGTCTACAACGACAAG | |
| | nucleotide | CATATAATCTTCGTCAACTCAGGCAGCTACGACCTGGACAACTCTTCCAATTACC | |
| | | TGAGACGAGCATGGGATGACAACTACTCTAACCAGTACTTTGCAGGAGTGCACG | |
| | | ACCCTGGCACATTGCTGAAGAACTCAGGTGTCACCTACATCCAGCCTCTGAAAG | |
| | | AGTACCCTGACGCCGGGGACGGAGACAACATCGACAAGTACGACGAGGAGATG | |
| | | AACAAGTACATCGCTCAGGAAGTTGTGAACCAGGTGAAGAACTACAACTACGA | |
| | | GTCTAAGATCCTGTCTGACAGTCTGATCGTGTACCACCACATTGAGCCTAGCATA | |
| | | ATGGCCTCTGCATCTAAGGAGTTGGTCAAGAGTAACGACACGGAGATGAATGGA | |
| | | ACCTACGGGGGCTACTCAGCCCCTCAGCTCCTGTACCAGACTAGCTCTTACCTTA | |
| | | ATGGCAACGGCCTTGACAACCCTAAGAACTTCACCGCCCCTGAGTCACCTATGA | |
| | | AGTTCTCTCTCCTGACGAAAGGAAGCTACACCATCAATGACTACATCAAGATGG | |
| | | GCGGGATAGTGAAAACGTATATGGACGAGAACGGCCAGGCGCCTGACTACATC | |
| | | AACTATGAGGGGGCCTACATCAGTTACTATGATCTGGTGTACAACTTCGCCAAG | |
| | | ATCACCCAGAACCACACCAACACCCAGCACATGGGCTTCGACTCTGAGTACGAG | |
| | | TTCGAGAAAACAAACGACTCTGGGGGTGGTGGCTCTGGTGGGGGCGGAAGCGG | |
| | | GGGCGGCGGCTCTATCGAGAACGCGGACAAGGCCATTAAGGACTTCCAAGATA | |
| | | ACAAGGCCCCTCACGACAAGTCCGCAGCCTACGAGGCGAATTCTAAGCTGCCTA | |
| | | AGGACTTGCGCGACAAGAACAACCGCTTCGTGGAGAAGGTGTCCATAGAAAAG | |
| | | GCCATCGTGCGGCATGACGAGCGGGTGAAGTCTGCGAACGATGCAATTTCCAAG | |
| | | CTTAACGAGAAGGACTCTATTGAGAACCGGCGCCTCGCCCAGAGGGAGGTTAAC | |
| | | AAGGCGCCTATGGACGTCAAGGAACACCTCCAGAAACAGCTTGACtaatgatagaccag | |
| | | cctcaagaacacccgaatggagtctctaagctacataataccaacttacactttacaaaa | |
| | | tgttgtcccccaaaatgtagccattcgtatctgctcctaataaaaagaaagtttcttcac | |
| | | attct | |
| C7.3 | ABQ87089.1 | MFVFLVLLPLVSSAANDLSTLGENTTVPNIIIVGDAPEVPDVPDIPDIPDFPVDPDNP | SEQ ID |
| | adhesin- | DIDDQNDSDTVNLTIFNIDEYFVDGTLGVEHSNTKFVLTQNEDNLGLLKIEANNVTIL | NO.: 59 |
| | like protein | GNNFTLQNVAFLINGKDVTLANFTLVNDFDFKDADGAAILTLANNTHIRDCVINYN | |
| | [Methano- | VPRDSEGYGISAVGRRIAPISGLEVINCIINFEGHNYKANTYNYALKVSNCPNALIAN | |
| | brevibacter | NSIYTQLPLRDVNFGAVGADLNSNYVASVGIEYSNNLTFIGNIVASIVNKRPGSPFPT | |
| | smithii | LDGIIIADSNDCLVKNNTLYMEDFLTFPGLNNYLYGIDVWRVNSLTLDSNNIAILTTG | |
| | ATCC | GMLSAGTAYPIQITGPSKKINITNNDLYSISNGPNIGIYSQNYYGDTQLYIAHNKINVT | |
| | 35061], | GLAGNDSWALVAGIEVQDSNDTIINNTIEVHSVAEVKDNDNMYGISYSQSTKGNHT | |
| | amino acid | FVIKNNTVTSDAKYAISLISAENSVIVDNLLISTRKDAKASYDAFNTKGKFYNTSYYN | |
| | | NRVVNAFDYYAEIYNHVDGGSEFNYTTPTNVNNLTNKVDGSKIKPWFPDFPNRNPL | |
| | | LPKPGDGSSVIVTPDDGDDKNPNVPDRPDGDAGYVDVPDLSGDDGGSKNPSNGTSS | |
| | | TDKNSNKLGVSLLDALINFLNSNTDTGDSRSNSYGGTVRTNSSTSSSDSPSLDGNPSP | |
| | | ASSTKSSSGSNAKSAGSSAGDDSKSVKAYEIDKNIMKSNPNTGGGGSGGGGSGGGG | |
| | | SIENADKAIKDFQDNKAPHDKSAAYEANSKLPKDLRDKNNRFVEKVSIEKAIVRHDERVK | |
| | | SANDAISKLNEKDSIENRRLAQREVNKAPMDVKEHLQKQLD | |
| C7.3 | ABQ87089.1 | cttgttctttttgcagaagctcagaataaacgetcaactttggccaccATGTTCGTGTTC | SEQ ID |
| | adhesin- | CTGGTGCTGTTGCCTCTCGTTTCCAGTGCGGCCAACGACCTCAGCACGCTGGGTGAAAAC | NO.: 60 |
| | like protein | ACGACCGTCCCTAACATCATCATCGTCGGAGACGCCCCTGAGGTGCCTGATGTGCCTGAC | |
| | [Methano- | ATCCCTGACATCCCTGATTTCCCTGTGGATCCTGATAATCCTGACATCGACGACCAGAA | |
| | brevibacter | CGACAGTGACACAGTTAACCTGACGATCTTTAATATAGACGAGTACTTTGTTGAC | |
| | smithii | GGCACTCTGGGAGTGGAGCACAGTAATACAAAGTTCGTGCTTACGCAGAACTTC | |
| | ATCC | GACAACCTGGGCCTGTTGAAAATAGAGGCGAACAACGTCACTATACTGGGAAAC | |
| | 35061], | AACTTCACCCTGCAGAACGTGGCGTTCCTCATTAACGGCAAGGATGTTACACTTG | |
| | nucleotide | CCAACTTCACCCTTGTGAACGACTTCGACTTCAAGGACGCCGACGGAGCCGCAA | |
| | | TCCTGACGCTCGCCAACAACACACACATACGCGATTGCGTGATCAATTACAACG | |
| | | TGCCTAGGGATAGCGAGGGGTACGGCATCTCCGCTGTGGGCAGGCGAATCGCCC | |
| | | CTATCAGTGGTCTGGAGGTCATAAACTGCATCATTAACTTCGAGGGTCACAACT | |
| | | ATAAGGCAAACACTTACAACTACGCTTTGAAAGTGTCAAACTGTCCTAACGCCC | |
| | | TCATCGCTAACAACTCTATCTATACCCAGCTCCCTTTGAGAGACGTGAACTTCGG | |
| | | TGCCGTGGGCGCCGATTTGAACTCCAACTACGTGGCCTCTGTCGGGATCGAGTAT | |
| | | TCAAATAACCTCACCTTCATCGGTAACATTGTGGCCTCAATCGTGAACAAGCGG | |
| | | CCTGGATCCCCTTTCCCTACACTGGACGGAATCATCATCGCAGACAGCAACGAC | |
| | | TGCCTGGTGAAGAACAACACTCTCTACATGGAGGACTTTCTCACCTTTCCTGGGC | |
| | | TGAACAACTACCTGTATGGCATCGACGTCTGGCGGGTGAATTCCCTTACACTGG | |
| | | ACAGTAACAACATCGCGATTCTGACCACCGGGGGAATGTTGTCCGCCGGCACCG | |
| | | CTTACCCTATCCAAATTACAGGCCCTTCAAAAAAGATAAACATTACCAACAACG | |
| | | ACCTTTACTCTATCAGCAACGGGCCTAACATCGGAATATACAGCCAGAATTACT | |
| | | ACGGAGACACTCAACTGTACATCGCTCACAATAAAATCAACGTCACCGGCCTTG | |
| | | CCGGCAACGACTCTTGGGCTCTGGTGGCCGGTATCGAGGTCCAGGACTCAAACG | |
| | | ACACCATTATTAACAACACTATTGAGGTTCACTCCGTCGCAGAGGTCAAGGACA | |
| | | ACGACAACATGTACGGTATCTCATACTCTCAGTCAACTAAAGGTAACCATACGT | |
| | | TCGTCATAAAGAACAACACAGTTACGTCTGACGCCAAGTACGCTATCAGCCTTA | |
| | | TATCCGCCGAGAACTCTGTGATTGTCGACAACCTGCTCATCAGTACAAGAAAAG | |
| | | ACGCCAAGGCATCCTATGACGCCTTCAACACCAAGGGGAAGTTCTACAATACCA | |
| | | GTTATTACAATAACCGAGTCGTGAATGCCTTCGACTACTACGCCGAGATCTACA | |
| | | ATCACGTCGACGGAGGGAGCGAGTTTAACTATACTACCCCTACCAACGTTAACA | |
| | | ACCTGACCAATAAAGTGGACGGCTCCAAGATAAAGCCTTGGTTCCCTGATTTCC | |
| | | CTAACAGGAACCCTCTTCTTCCTAAGCCTGGGGATGGGAGCAGCGTGATAGTCA | |
| | | CCCCTGACGATGGCGATGACAAGAACCCTAACGTGCCTGACCGGCCTGACGGCG | |
| | | ACGCAGGGTACGTCGACGTTCCTGACCTGAGTGGGGACGACGGGGGCTCAAAG | |
| | | AACCCTTCAAATGGCACCTCCTCCACCGACAAGAATTCCAACAAGCTGGGCGTG | |
| | | AGTCTTTTGGACGCTCTGATAAACTTTCTTAACTCAAACACCGATACAGGCGACA | |
| | | GCCGCAGCAATAGTTACGGCGGAACTGTTAGAACCAATAGTTCTACCTCTTCTA | |
| | | GCGACTCCCCTTCACTTGACGGAAATCCTAGCCCTGCGTCTTCTACGAAGTCCTC | |
| | | TAGCGGCAGCAACGCCAAGTCTGCCGGGTCATCAGCCGGCGACGACTCTAAGTC | |
| | | AGTGAAGGCCTACGAGATCGACAAGAACATTATGAAAAGTAACCCTAACACTG | |
| | | GTGGAGGGGGTTCAGGCGGGGGCGGTAGTGGTGGGGGGGGTAGTATCGAAAAC | |
| | | GCTGACAAGGCAATCAAGGACTTCCAGGACAATAAGGCACCTCACGACAAATCT | |
| | | GCTGCCTACGAGGCAAACTCCAAGTTGCCTAAGGATTTGCGCGACAAAAACAAC | |
| | | CGCTTCGTTGAAAAGGTGAGTATCGAGAAGGCGATAGTTCGACACGACGAGCGG | |
| | | GTTAAGAGTGCGAATGATGCAATCTCAAAGCTCAACGAAAAGGACAGCATTGA | |
| | | AAACCGAAGGTTGGCACAGAGAGAGGTGAATAAGGCTCCTATGGACGTGAAGG | |
| | | AGCATTTGCAAAAGCAGTTGGATtaatgatagaccagcctcaagaacacccgaatggagt | |
| | | ctctaagctacataataccaacttacactttacaaaatgttgtcccccaaaatgtagcca | |
| | | ttcgtatctgctcctaataaaaagaaagtttcttcacattct | |
| C7.4 | WP_ | MFVFLVLLPLVSSAASNNLIESLSTNNNGSEAIYIPNSDLNLLSGNGNDVETPDIPDLI | SEQ ID |
| | 052331828.1 | VNDTFYVTSDDIEDYFPNRQLESKYYNKTLIFTGNFENVGKLIIDVNNVTLKGVGSY | NO.: 61 |
| | hypothetical | LKNTVFDLRADNITLDSFNMDLDSEFEDNDGAAIEFIANNIVLSNLRINYIVPRNVEA | |
| | protein | YAIYGIGQPYRSIKNEKMENSIINFEGHNDYVNKYNYAVKLVDCVDSVMENNSLVT | |
| | [Methano- | SLPLRNVVFGAMGASLDSDFVLSVGVENCHNFTFIGNSIIANVNNRPAFTYPTLDCFL | |
| | brevibacter | ISKCDNSSILNNSIYMTDFLTFPGVENYLYGIDVYNLNNLTIAYNNISIITTGGKLAAG | |
| | oralis], | TAYPIQITGPISAVNITYNDIYTFSNGPNIGIYSQNYYGNTSLSITHNRINVTGLAGSHE | |
| | amino acid, | WALVAGIETQDSNSTIQNNTIEVHSVAPVDIGDNIYGISYRQKTSGNHTYNIQNNTVF | |
| | | SDGFYSVYLLSSVNSNVINNLLVSYNDKVKAGLNGFNYNEFSSHIGINFYNNKVINA | |
| | | FDYFANKYNNIDGGEGFNYTNPTNINSISNNIDGSSVIAIPSNNHYNYNPLIPGNSNNQ | |
| | | GNPNNQGSNQQNNQGNSNGNSSQGGSGNGVNDNNSGEGNSSGNKLSLRDLLANFF | |
| | | NSNSDKGKVNRSNYNSNANHEITSNNTDQTPSTEGEDALMSDVKSVESTSESPSASD | |
| | | SASKKAYELDDLVKENKLFIPSGGGGSGGGGSGGGGSIENADKAIKDFQDNKAPHDK | |
| | | SAAYEANSKLPKDLRDKNNRFVEKVSIEKAIVRHDERVKSANDAISKLNEKDSIENRRLA | |
| | | QREVNKAPMDVKEHLQKQLD | |
| C7.4 | WP_ | cttgttctttttgcagaagctcagaataaacgctcaactttggccaccATGTTCGTGTTT | SEQ ID |
| | 052331828.1 | CTGGTTCTTCTGCCTCTCGTGTCTTCTGCCGCTAGCAACAACTTGATTGAGTCCCTGTCA | NO.: 62 |
| | hypothetical | ACAAACAACAATGGTAGCGAGGCCATTTATATCCCTAACAGCGATCTTAACCTGTTGTCT | |
| | protein | GGCAATGGGAACGATGTCGAAACACCTGACATCCCTGACCTCATTGTCAACGACACCTT | |
| | [Methano- | CTACGTGACGTCTGACGATATCGAGGATTACTTCCCTAACAGGCAGCTCGAGTCT | |
| | brevibacter | AAATACTATAACAAAACCTTGATCTTTACAGGTAACTTTGAGAATGTCGGGAAG | |
| | oralis], | CTTATTATTGACGTTAATAACGTCACTCTGAAGGGGGTTGGGTCCTATCTGAAGA | |
| | nucleotide | ACACAGTCTTTGACCTGCGAGCCGACAATATTACACTGGACAGCTTCAACATGG | |
| | | ATCTGGATTCTGAATTCGAGGACAACGATGGAGCTGCCATCGAATTTATCGCAA | |
| | | ATAATATTGTGCTGAGCAACCTTAGAATTAACTACATTGTTCCTCGCAACGTCGA | |
| | | AGCTTACGCCATATACGGAATAGGTCAACCTTACAGATCCATTAAAAACTTCAA | |
| | | GATGTTCAACTCAATCATTAACTTTGAGGGTCATAATGACTATGTGAACAAGTAC | |
| | | AACTATGCAGTGAAGTTGGTGGACTGTGTGGATTCAGTTATGGAGAATAACAGC | |
| | | CTGGTCACCAGCCTCCCTCTCAGAAACGTGGTGTTTGGTGCCATGGGCGCTTCAC | |
| | | TGGATAGCGACTTCGTCCTTAGCGTGGGCGTCGAGAACTGCCACAACTTCACCTT | |
| | | CATAGGGAATTCTATCATAGCAAACGTGAATAACAGGCCTGCGTTTACATATCC | |
| | | TACCCTGGATTGTTTCTTGATCTCAAAGTGCGATAATAGCAGTATCCTCAACAAT | |
| | | AGCATTTATATGACGGACTTCCTGACCTTCCCTGGGGTCGAAAATTATTTGTATG | |
| | | GCATCGACGTGTACAATCTCAACAACCTTACTATCGCTTACAATAACATTTCAAT | |
| | | TATTACAACAGGCGGCAAACTGGCCGCCGGCACCGCATACCCTATACAGATCAC | |
| | | GGGGCCTATCAGCGCTGTGAATATTACATACAACGATATATATACCTTCAGTAAT | |
| | | GGACCTAACATCGGCATTTATTCCCAGAATTACTACGGGAACACCAGCCTCAGC | |
| | | ATCACTCATAATAGAATCAACGTCACCGGGCTGGCCGGAAGTCATGAGTGGGCC | |
| | | CTGGTCGCCGGCATCGAGACACAGGATAGTAATAGCACCATCCAGAATAACACT | |
| | | ATCGAGGTTCACAGCGTGGCTCCTGTGGACATAGGTGATAACATCTACGGTATC | |
| | | TCATACCGCCAGAAAACCTCTGGTAATCACACCTACAACATCCAAAACAACACC | |
| | | GTGTTTTCAGACGGCTTCTACTCCGTGTATCTGCTCAGTAGTGTGAACAGCAACG | |
| | | TGATTAATAACCTGCTCGTCAGCTATAACGACAAGGTGAAAGCAGGACTGAACG | |
| | | GGTTTAATTACAACGAATTCTCTTCCCACATCGGAATTAACTTTTACAACAACAA | |
| | | AGTGATCAACGCATTCGACTACTTTGCGAACAAGTACAATAACATCGACGGCGG | |
| | | CGAGGGCTTTAACTACACTAATCCTACTAACATCAATTCAATCAGTAACAATATT | |
| | | GATGGGTCATCCGTTATCGCCATACCTTCTAATAATCACTATAATTACAATCCTC | |
| | | TTATCCCTGGCAACTCTAACAATCAGGGCAATCCTAACAACCAAGGAAGTAATC | |
| | | AACAGAATAATCAGGGAAACAGCAATGGAAATAGCTCTCAGGGAGGATCCGGA | |
| | | AACGGGGTGAATGACAATAACTCCGGGGAGGGGAACTCCTCTGGCAATAAGCT | |
| | | GTCCTTGCGCGACCTCCTGGCTAACTTCTTTAACAGTAATTCCGATAAGGGAAAA | |
| | | GTGAACCGGAGCAACTATAATTCAAATGCGAACCATGAAATAACTTCCAACAAC | |
| | | ACTGACCAGACGCCTAGTACGGAAGGCGAGGATGCCCTGATGAGCGATGTGAA | |
| | | GTCCGTTGAGTCCACCTCCGAGTCTCCTAGCGCCTCTGACAGCGCCTCCAAGAAA | |
| | | GCATATGAGCTGGATGATCTGGTCAAAGAGAATAAGCTCTTCATCCCTTCCGGA | |
| | | GGCGGTGGTAGTGGGGGCGGAGGCAGTGGGGGGGGCGGCTCCATCGAGAATGC | |
| | | AGACAAAGCGATAAAGGACTTTCAGGATAATAAAGCTCCTCACGACAAAAGTG | |
| | | CGGCTTACGAAGCCAATTCCAAGCTGCCTAAGGATTTGCGAGATAAGAACAATA | |
| | | GGTTCGTGGAAAAAGTGTCAATCGAAAAGGCCATCGTTCGGCATGACGAACGGG | |
| | | TGAAGAGTGCCAACGACGCTATTTCCAAACTGAACGAAAAGGACTCTATTGAAA | |
| | | ACAGGCGCCTCGCACAGCGGGAGGTCAATAAGGCCCCTATGGACGTGAAGGAG | |
| | | CACCTGCAGAAGCAGCTTGACtaatgatagaccagcctcaagaacacccgaatggagtct | |
| | | ctaagctacataataccaacttacactttacaaaatgttgtcccccaaaatgtagccatt | |
| | | cgtatctgctcctaataaaaagaaagtttcttcacattct | |
| C8.1 | mru0493 = | MFVFLVLLPLVSSAAIDVDTNDNLDDGSSSNSDLISSSSLDSSSSDDVSSGSSEVSSS | SEQ ID |
| | ADC46344.1, | DESLDGNNLSDGNVSSSDESVGADNLSDGNVSSSDESVGADNLSDDESSSDALSEEL | NO.: 63 |
| | adhesin- | PKTETVIKADPINYNYASVKGLTINLTDSAGLALSNKTLTVKVSALNKTSNLTTNSK | |
| | like protein | GQAIFKLSASVGSYDVFISFTGDESYAPSNASSKITIKKSSTKIKLSNIHGYLTISNYVS | |
| | [Methano- | VTLLDSAGKPIKSKSVTIQVNKAKYNVKTDSKGIAKVKVANKIGTYSVNAKFSGDK | |
| | brevibacter | NYYASSNSSKLTITKMKVYIKAPSVKYYMTNSSAPYLTINLTNVKGSPLAKKKVSVK | |
| | ruminantium], | IGKKTYTLKTNSQGIAKFKFTKKVSSYNCKINFKATSNFYGASVNSKMTIQKMPTSL | |
| | amino acid | KAPSVSINSTNYGKVLISLKDGKGKALKNTTVTVNVTELKKVFTLKTNASGVATFSF | |
| | | NGEKTFNLKIKYAGNKNYAASSVSSKINVKQIKVKLSDVIGASRVLIDYVNRTKDLP | |
| | | SNVQYNNYNFTVTQLTYLASKAVKNINNKNYGDIVLISVPKSYKSSGEIYDTVYKK | |
| | | DFVKIANSVVGSSYNYKNKEYVSYSIYKVPFKVYSISFAKVLNFYGNNKKLPNYSLF | |
| | | TLADFAKVKDNGGYNFYLTTDNIAGKKSDLNMLKSLAKTLKSMGYNAVIVGIGPDI | |
| | | HNVAYRYGCTGNNSVLLACFGGVDVGCIEEWAGDLGDLNGHSFVNSYQGAHVLG | |
| | | LWFTKPYGASVSLNKKVGIAWDADYGFPLNTPAKYMKSHNISYIETGTVANACKLL | |
| | | SEGKMGGPQLISGGGGSGGGGSGGGGSIENADKAIKDFQDNKAPHDKSAAYEANSKLP | |
| | | KDLRDKNNRFVEKVSIEKAIVRHDERVKSANDAISKLNEKDSIENRRLAQREVNKAPMDV | |
| | | KEHLQKQLD | |
| C8.1 | mru0493 = | cttgttctttttgcagaagctcagaataaacgctcaactttggccaccATGTTCGTCTTC | SEQ ID |
| | ADC46344.1, | CTGGTCCTCCTCCCTCTGGTGTCTTCTGCAGCTATCGACGTCGATACAAATGACAACCTT | NO.: 64 |
| | adhesin- | GACGACGGGAGCTCATCTAACTCCGACTTGATTAGCTCCAGTTCTCTGGACTCTTCTTCA | |
| | like protein | AGTGATGACGTGTCCTCCGGGTCCAGTGAAGTTAGCTCTAGTGACGAGTCACTGGACG | |
| | [Methano- | GTAACAATCTTAGCGACGGTAATGTGTCCAGCAGCGACGAGTCCGTGGGAGCAG | |
| | brevibacter | ACAACTTGTCTGATGGCAATGTCTCTTCATCAGATGAGAGCGTGGGAGCAGATA | |
| | ruminantium], | ATCTCTCTGATGACGAGTCCTCCTCTGACGCCCTGTCTGAGGAGCTGCCTAAAAC | |
| | nucleotide | TGAGACCGTTATAAAGGCTGATCCTATAAATTACAATTACGCATCTGTTAAAGGT | |
| | | CTTACAATCAATCTGACTGATTCCGCTGGCCTGGCCCTCAGCAACAAAACTCTGA | |
| | | CCGTGAAAGTCTCCGCACTGAATAAGACCAGCAACCTGACTACAAACAGTAAGG | |
| | | GTCAGGCCATCTTCAAACTGAGCGCAAGTGTCGGATCATACGATGTGTTCATCTC | |
| | | TTTCACGGGGGACGAAAGCTACGCTCCTTCAAACGCATCCTCAAAGATAACCAT | |
| | | TAAGAAGAGCTCCACCAAGATCAAGCTTTCAAACATCCATGGGTATCTTACCAT | |
| | | CTCAAACTATGTTTCTGTGACCCTTTTGGACAGCGCCGGAAAGCCTATTAAATCC | |
| | | AAGAGTGTGACGATCCAGGTGAATAAGGCCAAGTATAACGTGAAGACCGACTCT | |
| | | AAGGGCATCGCGAAAGTCAAGGTCGCAAATAAGATAGGGACATATAGCGTCAA | |
| | | CGCCAAGTTCAGCGGCGATAAAAATTACTACGCATCCAGCAACTCTTCAAAGCT | |
| | | GACGATAACGAAAATGAAGGTGTACATTAAAGCCCCTTCCGTCAAATACTACAT | |
| | | GACCAACAGTTCTGCCCCTTATCTGACTATCAACCTCACTAATGTTAAGGGCTCC | |
| | | CCTCTCGCCAAGAAGAAGGTCTCTGTCAAGATTGGGAAGAAAACGTATACCCTG | |
| | | AAGACAAACAGCCAAGGAATTGCTAAATTTAAATTTACAAAGAAAGTGTCCTCT | |
| | | TACAACTGTAAGATCAACTTTAAGGCCACCAGCAACTTCTATGGAGCATCAGTC | |
| | | AATTCAAAAATGACGATCCAAAAGATGCCTACATCACTGAAGGCCCCTTCCGTG | |
| | | AGCATTAACAGTACCAACTATGGCAAGGTTCTGATAAGTCTGAAGGATGGGAAG | |
| | | GGCAAGGCCCTCAAGAACACGACTGTGACCGTGAATGTCACCGAGCTGAAAAA | |
| | | GGTGTTTACACTCAAGACCAACGCTAGCGGCGTCGCGACTTTTAGTTTTAACGGT | |
| | | GAAAAAACTTTTAACCTGAAGATTAAGTACGCCGGCAACAAGAATTACGCTGCT | |
| | | TCTTCCGTTAGCAGTAAAATCAATGTGAAGCAGATAAAGGTGAAGCTGAGCGAC | |
| | | GTGATCGGCGCGTCCCGAGTGTTGATTGACTACGTGAATAGGACTAAAGACCTG | |
| | | CCTAGTAATGTTCAGTATAATAATTACAATTTTACAGTGACACAGTTGACCTATC | |
| | | TGGCTAGCAAAGCGGTCAAGAATATCAACAACAAGAACTACGGGGATATCGTTC | |
| | | TGATTAGTGTGCCTAAATCCTATAAAAGCTCTGGCGAGATTTATGATACCGTCTA | |
| | | CAAGAAGGACTTTGTGAAGATTGCTAACTCCGTGGTTGGCTCCTCATACAATTAT | |
| | | AAAAACAAAGAATACGTGTCCTATAGTATTTACAAAGTGCCTTTCAAAGTGTAC | |
| | | TCCATCTCATTCGCTAAGGTCCTGAATTTTTACGGAAACAATAAGAAGCTCCCTA | |
| | | ATTACAGTTTGTTCACACTCGCTGATTTCGCCAAAGTTAAAGATAACGGAGGCTA | |
| | | TAATTTCTACCTTACCACAGACAATATCGCGGGTAAGAAGTCCGATCTGAACAT | |
| | | GCTCAAATCTCTGGCCAAGACCCTGAAGAGTATGGGATACAACGCTGTGATTGT | |
| | | TGGAATTGGCCCTGACATCCACAACGTGGCTTATAGATATGGGTGCACAGGAAA | |
| | | TAATTCCGTTCTCCTCGCCTGCTTCGGAGGGGTGGATGTCGGCTGCATCGAGGAA | |
| | | TGGGCCGGGGACCTTGGCGACCTGAACGGCCATAGCTTCGTCAATTCATACCAG | |
| | | GGTGCGCACGTGCTGGGCCTTTGGTTCACTAAACCTTACGGTGCTAGTGTGTCCC | |
| | | TCAATAAAAAGGTGGGGATCGCCTGGGATGCCGACTATGGATTTCCTTTGAACA | |
| | | CCCCTGCCAAGTATATGAAAAGTCATAACATCAGCTACATCGAGACTGGCACCG | |
| | | TGGCCAACGCCTGTAAACTCTTGAGCGAAGGCAAGATGGGAGGACCTCAACTGA | |
| | | TATCCGGGGGGGGAGGGAGCGGTGGCGGGGGTAGCGGTGGGGGGGGCAGCATC | |
| | | GAGAACGCCGACAAGGCCATCAAGGATTTCCAGGACAACAAAGCACCTCACGA | |
| | | TAAGAGCGCAGCCTACGAGGCGAACAGCAAACTCCCTAAGGATTTGAGAGATA | |
| | | AGAACAACAGGTTTGTGGAGAAAGTGAGTATTGAAAAAGCAATCGTGCGCCAC | |
| | | GATGAACGGGTGAAAAGCGCCAACGACGCTATCAGCAAGCTGAATGAAAAAGA | |
| | | CTCCATTGAGAACCGGCGCCTGGCGCAGCGGGAAGTCAACAAAGCCCCTATGGA | |
| | | CGTGAAGGAGCACCTCCAGAAACAGCTGGACtaatgatagaccagcctcaagaacacccg | |
| | | aatggagtctctaagctacataataccaacttacactttacaaaatgttgtcccccaaaa | |
| | | tgtagccattcgtatctgctcctaataaaaagaaagtttcttcacattct | |
| C8.2 | mru0824 = | MFVFLVLLPLVSSAAQSLDNINYANDGFDSDEMINCDLHKDSSQKSLKSNALSNKK | SEQ ID |
| | ADC46675.1, | TTNTVKLTDMKKAESNDVKQTGAAKASNTKSTSKSTTKTNATKSNTTKSTATKTTA | NO.: 65 |
| | adhesin- | NSSSTKKATQNTTTINTQTLAKSSSSYMAYVEKNAKLQEPITISKKKYKSPEYLYLVS | |
| | like protein | KAVSNISKTKVEIKDKLITNYSNTDCKSVNGTINKTEYVQVAKKTVSFIEKNHRAPN | |
| | with trans- | WIASSKGNIPRNQLILVESKCLDQYNKSGKLPSSIKLNDLDLNKMKQKIDSSKKVNST | |
| | glutaminase | STKKTNTSSTKINSTSAKKTNTTSTKKINPTATSTNNNKSLVESTLDSIKSILNNIENK | |
| | domain | LNPTNKVLSTTGTKKNTVTVNSSKVNVQISSSSTVNVKISAKDNTNSGKNTNSGSAK | |
| | [Methano- | KTNTTSTKKTNTTSTKKIDTNSTKKTNTTSTKNNTSSAKKTNTTSTKNNTSSAKKTN | |
| | brevibacter | TTSTKNNTSSAKKVNTSSSKTNTSAKNNTSTTAKSSSNSKYLSTSVLNDKYLGESLK | |
| | ruminantium | KYLAVGKNCQVINKAIKTLANTLTSKLKSDYKKGEKIFNWVRDNIGYEKYRNTKK | |
| | M1], | GALKTLQTRGGNCVDHAHLIVALSRAAGLPARYVNANNCKFSSGYVSGHVWAQV | |
| | amino acid | LVGNTWVVADATSNRNKFGVVKNWNVNSYKLVGKYSSISFGGGGSGGGGSGGGG | |
| | | SIENADKAIKDFQDNKAPHDKSAAYEANSKLPKDLRDKNNRFVEKVSIEKAIVRHDERV | |
| | | KSANDAISKLNEKDSIENRRLAQREVNKAPMDVKEHLQKQLD | |
| C8.2 | mru0824 = | cttgttctttttgcagaagctcagaataaacgctcaactttggccaccATGTTTGTCTTT | SEQ ID |
| | ADC46675.1, | CTCGTGTTGCTCCCTCTGGTCTCCTCTGCAGCCCAGAGCCTGGATAACATTAACTACGCC | NO.: 66 |
| | adhesin- | AACGACGGCTTCGATTCAGATGAAATGATTAATTGCGATTTGCACAAGGATAGTTCCCAG | |
| | like protein | AAGTCCCTTAAGAGCAACGCTCTGTCCAACAAGAAAACGACAAATACCGTGAAACTC | |
| | with trans- | ACGGATATGAAGAAAGCAGAGTCAAACGACGTGAAGCAGACCGGGGCGGCCAA | |
| | glutaminase | GGCCAGCAATACAAAAAGCACATCCAAGTCTACAACGAAAACCAACGCAACCA | |
| | domain | AAAGCAACACAACGAAAAGTACTGCCACCAAAACGACCGCTAACTCTTCCTCCA | |
| | [Methano- | CAAAAAAAGCTACCCAAAACACTACCACTATCAATACTCAGACTCTCGCCAAGT | |
| | brevibacter | CCAGTTCCTCCTATATGGCCTATGTGGAGAAAAACGCGAAGCTCCAGGAACCTA | |
| | ruminantium | TTACCATTAGCAAGAAGAAGTACAAATCTCCTGAATACCTGTATCTGGTGAGCA | |
| | M1], | AAGCGGTGAGTAACATCAGCAAAACCAAGGTTGAGATAAAGGACAAGCTGATA | |
| | nucleotide | ACAAACTACAGTAATACAGACTGTAAGTCAGTTAATGGCACCATCAACAAAACC | |
| | | GAGTACGTGCAGGTCGCGAAGAAGACCGTCTCTTTCATAGAGAAGAACCATCGC | |
| | | GCCCCTAACTGGATCGCAAGCAGCAAAGGAAACATCCCTCGCAACCAATTGATC | |
| | | CTCGTGTTCAGTAAGTGTCTTGACCAGTACAATAAAAGCGGAAAGCTTCCTAGC | |
| | | TCTATCAAGCTGAACGATCTGGACCTCAATAAGATGAAGCAGAAAATAGACAGC | |
| | | AGTAAAAAGGTGAACTCCACTTCTACTAAGAAAACAAATACCTCTTCAACTAAG | |
| | | ACAAACAGCACAAGCGCTAAGAAGACTAATACTACCAGTACCAAGAAGACAAA | |
| | | CCCTACGGCCACATCTACCAATAATAACAAATCACTGGTGGAAAGTACGTTGGA | |
| | | CTCTATAAAGTCCATTCTTAATAATATTGAGAACAAGCTGAACCCTACTAATAAA | |
| | | GTTCTCTCAACAACAGGGACCAAGAAGAACACTGTGACTGTGAATTCATCCAAA | |
| | | GTGAATGTTCAGATCTCCTCTTCCTCAACAGTTAACGTGAAAATCTCAGCCAAAG | |
| | | ACAACACCAATTCTGGAAAAAACACCAACTCCGGATCCGCCAAAAAGACCAAT | |
| | | ACAACCAGCACCAAGAAGACGAACACGACAAGTACCAAGAAGATCGATACAAA | |
| | | CTCCACTAAAAAGACGAACACTACTAGCACTAAGAATAACACCTCTTCAGCAAA | |
| | | AAAAACCAATACCACCAGTACGAAGAACAACACCTCCAGTGCAAAGAAGACAA | |
| | | ATACCACGAGTACAAAGAATAATACATCCAGCGCCAAGAAAGTGAACACTTCCT | |
| | | CCAGTAAAACCAATACCAGTGCCAAGAACAACACCAGCACCACCGCAAAAAGC | |
| | | TCTAGCAACAGCAAATATCTGAGTACCTCCGTTCTGAATGACAAATACCTCGGG | |
| | | GAGTCTCTGAAGAAATACCTTGCTGTCGGGAAGAACTGCCAGGTCACCAACAAG | |
| | | GCTATCAAGACGCTGGCCAATACACTGACGAGCAAGTTGAAGTCAGACTATAAG | |
| | | AAGGGTGAAAAGATTTTCAATTGGGTCCGAGATAACATTGGCTACGAGAAGTAC | |
| | | AGGAACACTAAAAAGGGCGCACTTAAGACTCTGCAAACAAGGGGCGGTAATTG | |
| | | CGTGGACCACGCCCACCTTATTGTCGCCCTGTCCAGGGCTGCGGGCCTGCCTGCT | |
| | | CGGTACGTGAATGCCAATAATTGTAAATTTTCATCCGGCTATGTGTCTGGTCACG | |
| | | TGTGGGCCCAGGTGCTCGTCGGCAATACTTGGGTGGTTGCCGACGCAACCAGCA | |
| | | ACCGGAACAAATTTGGGGTCGTGAAAAACTGGAATGTGAACTCATATAAGCTCG | |
| | | TCGGAAAGTACTCCTCAATCTCATTCGGTGGCGGTGGCAGTGGGGGAGGCGGCA | |
| | | GCGGAGGAGGGGGGAGCATCGAAAATGCAGACAAGGCTATCAAGGACTTCCAG | |
| | | GACAACAAAGCCCCTCATGATAAATCTGCGGCCTATGAGGCTAATAGCAAACTG | |
| | | CCTAAGGACCTGCGGGATAAGAACAACAGATTTGTGGAAAAGGTGAGCATCGA | |
| | | GAAGGCTATCGTGCGGCACGACGAAAGAGTCAAATCTGCTAACGATGCTATTAG | |
| | | CAAGTTGAATGAAAAAGATTCTATCGAGAACCGAAGACTGGCACAGCGCGAGG | |
| | | TCAACAAGGCCCCTATGGACGTTAAGGAGCATCTGCAGAAACAACTGGATtaatgat | |
| | | agaccagcctcaagaacacccgaatggagtctctaagctacataataccaacttacactt | |
| | | tacaaaatgtgtcccccaaaatgtagccattcgtatctgctcctaataaaaagaaagttt | |
| | | cttcacattct | |
| C8.3 | mru_1604 | MFVFLVLLPLVSSAASNASDNLDDLTISDSNSLDLVSTSNSDILSSDSGVSSDDSSND | SEQ ID |
| | ADC47454.1, | ASGDVLGSDVSSNESNNQSQSTLDSNNQSQSGLDSDNSTLLDSQSNNQSNSESSDSS | NO.: 67 |
| | adhesin- | DSSETVIKNATSISVSSKTVVRGNSLNITLKDNASTLLSNKTVTFTFNGKTYNKTTNA | |
| | like protein | KGIASLTLTATPKKYLVKIAFVGDELYEASSKSVNVTLSKTPTSISNSGKSIVRGKLY | |
| | with trans- | KLTLKDAKGKALSGKKISISFNGKKYTKTTNSNGQVNLTINVNVGKTYKMTYKFAG | |
| | glutaminase | DSNYLSSSGSVSIKVKMGTSIIGSGSSIVKGKSYTVTLKNANGAVLSNQKIAFTLSGK | |
| | domain | TYNRTTNAKGQASLKIGLSSGKTYNLTYKYAGNSYYGGSSGKVSLFVKTPTTMKNS | |
| | [Methano- | GKTIVSGETYKVTLKDADGKSLANKKVSITFNNKTYAKTTNSNGQASLTIKGTFGRS | |
| | brevibacter | YPLSYKFAGDSKYGPSSGSLCLRVKKATSLKGSASSIVQGKSYTVTLKDSNSTPLAN | |
| | ruminantium | QTIVFTLDTKKYNRTTNAKGQASLKIGLAAGKTYNLAYKYSGTSYYNGSSGSVKLK | |
| | M1], | VKFPTSLTNSGKSVMNGTGYNIVLKDSKSNLVSNKTISIGENGKTYDEITDANGTVT | |
| | amino acid | LLIDANVPKTYKMTYKFAGDSDYGASSGTVNLTVKFKNAFTISQUISASSSLKSYVLK | |
| | | NKKVPATVSVNGVSLNLTSFTYLMAKATISINSNKTSGSILLVPVDSNYTNNGSRINA | |
| | | NLYKANYIDLAKKVISSAEANKLVPNSVSTNIGLVSHDLYSFGLAKALVFFNSDHYL | |
| | | PNYLILSSDDVGEKHSTVIPSNARGNASQFKAGLNEAETLTAAQIAKYLVASGHDAT | |
| | | NSEIKALAAKLVSGKTSLWDKANAIFTFARDNITYSYYADSKKGAAGTLSSKSGNC | |
| | | CDHSNLIVSLCRAANITARFSHAQGCTFSSGLVAGHVWAQIYIDGVWYTADATSRR | |
| | | NSLGNIVNWNTNHYNTLKQYDHLSFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIE | |
| | | NRRLAQREVNKAPMDVKEHLQKQLD | |
| C8.3 | mru1604 = | cttgttctttttgcagaagctcagaataaacgctcaactttggccaccATGTTCGTTTTC | SEQ ID |
| | ADC47454.1, | CTGGTCCTGCTCCCTCTGGTCTCCTCCGCAGCGTCAAACGCCTCTGACAATCTTGACGAC | NO.: 68 |
| | adhesin- | CTCACTATCTCTGACTCAAACTCTCTGGACCTCGTGTCTACCTCTAACTCTGACATCCTT | |
| | like protein | TCTTCTGACTCTGGCGTGAGTTCCGATGACAGCTCTAACGACGCCTCTGGCGACGTGCTG | |
| | with trans- | GGGAGTGACGTTTCTAGTAACGAGAGCAACAACCAGTCTCAGTCTACGCTGGAT | |
| | glutaminase | TCGAACAACCAGTCTCAGAGCGGGCTTGACAGTGACAACTCTACACTGCTCGAC | |
| | domain | TCCCAGTCTAACAACCAGTCTAACTCTGAGTCTTCTGACTCTTCAGACTCTTCTG | |
| | [Methano- | AGACCGTGATCAAGAACGCCACGTCCATCTCTGTCAGTTCAAAGACCGTGGTGC | |
| | brevibacter | GGGGGAACTCTTTGAACATCACGCTTAAGGACAACGCCTCTACCCTGCTCTCTAA | |
| | ruminantium | CAAGACTGTTACCTTCACGTTTAACGGCAAGACCTACAACAAGACCACGAACGC | |
| | M1], | CAAGGGGATCGCCTCTCTCACTCTGACGGCCACTCCTAAGAAGTACCTGGTTAA | |
| | nucleotide | GATCGCCTTCGTCGGGGACGAGCTCTACGAGGCATCTTCTAAGTCTGTGAACGTC | |
| | | ACGCTCTCTAAGACCCCTACCTCTATTAGCAACTCTGGGAAATCTATCGTCCGCG | |
| | | GAAAGTTGTATAAGCTGACGCTGAAAGACGCCAAGGGGAAGGCCCTGTCTGGTA | |
| | | AAAAGATCTCTATCAGCTTCAACGGCAAGAAGTACACAAAGACCACAAACTCTA | |
| | | ATGGCCAGGTGAACCTGACAATCAATGTCAATGTGGGCAAGACCTACAAGATGA | |
| | | CCTACAAGTTCGCCGGCGACTCCAATTATCTGTCTTCATCAGGCTCAGTGTCTAT | |
| | | CAAGGTCAAGATGGGCACCTCCATCATCGGCTCTGGCTCGTCAATCGTGAAGGG | |
| | | CAAGTCTTACACCGTGACTCTCAAGAACGCGAATGGCGCCGTGCTGTCTAACCA | |
| | | GAAAATCGCATTCACCCTCAGTGGCAAGACGTACAATAGGACAACCAACGCTAA | |
| | | GGGCCAAGCGTCTCTCAAGATCGGCCTGAGTAGCGGCAAGACCTACAACCTCAC | |
| | | ATACAAGTACGCCGGGAACTCTTACTACGGCGGCTCTTCTGGGAAAGTCTCTTTG | |
| | | TTCGTGAAGACGCCTACGACCATGAAGAACAGCGGCAAGACGATCGTGTCAGG | |
| | | GGAGACGTACAAGGTGACCCTCAAGGACGCTGACGGCAAGTCTCTGGCGAACA | |
| | | AGAAGGTCTCCATCACTTTCAACAATAAGACATACGCCAAGACCACCAACTCTA | |
| | | ACGGGCAGGCAAGTCTTACCATTAAAGGGACCTTCGGCCGCTCTTACCCTCTGTC | |
| | | CTACAAGTTCGCCGGCGACTCTAAGTACGGTCCTAGCTCTGGATCTCTGTGCCTG | |
| | | CGCGTGAAGAAGGCGACCTCTCTGAAGGGGTCTGCCTCTAGTATCGTCCAGGGG | |
| | | AAGTCTTACACCGTCACCTTGAAGGACTCCAACTCTACCCCTCTGGCTAACCAGA | |
| | | CCATCGTGTTCACACTGGACACGAAGAAGTACAACCGGACCACCAACGCCAAGG | |
| | | GCCAAGCCTCTCTGAAGATCGGCCTGGCCGCCGGTAAGACGTACAACTTGGCCT | |
| | | ACAAGTACTCTGGCACCTCTTATTACAACGGCAGCTCTGGGAGCGTGAAGCTCA | |
| | | AGGTGAAGTTCCCTACCTCTCTGACCAACAGCGGAAAGTCTGTGATGAACGGGA | |
| | | CCGGGTACAACATCGTGCTGAAGGACTCCAAGTCTAACCTGGTGAGTAACAAGA | |
| | | CCATATCTATAGGTTTCAACGGGAAGACCTACGACGAGATCACAGATGCCAACG | |
| | | GCACGGTCACCCTTCTCATCGACGCTAACGTCCCTAAGACCTACAAGATGACCT | |
| | | ACAAGTTCGCCGGTGACTCTGACTACGGCGCCTCTAGCGGAACTGTGAACCTGA | |
| | | CGGTCAAATTTAAGAACGCCTTCACCATCTCACAGATCATCTCTGCAAGCTCTAG | |
| | | TCTCAAGTCTTACGTGCTCAAGAACAAAAAGGTGCCTGCGACTGTGTCTGTCAA | |
| | | CGGCGTGAGTCTCAATCTGACCAGTTTCACCTACCTGATGGCGAAGGCCACCAT | |
| | | CTCGATTAACTCAAACAAGACCAGCGGTTCTATCCTGCTGGTGCCTGTCGACTCT | |
| | | AACTACACCAACAACGGCTCAAGAATCAATGCCAACCTGTACAAGGCCAACTAC | |
| | | ATAGACCTGGCCAAGAAAGTGATCTCTTCTGCCGAGGCCAACAAGCTGGTGCCT | |
| | | AACTCTGTTTCCACCAACATAGGGCTGGTTTCCCACGACCTGTACTCTTTCGGAC | |
| | | TCGCCAAGGCCTTGGTGTTCTTCAACAGCGACCACTACCTGCCTAACTATCTTAT | |
| | | CCTGTCTAGCGATGACGTGGGGGAGAAGCACAGTACCGTGATCCCTTCTAACGC | |
| | | CCGAGGCAACGCCTCTCAGTTCAAGGCCGGTCTGAACGAGGCAGAGACCCTTAC | |
| | | GGCTGCCCAGATAGCCAAGTATCTGGTGGCGTCTGGCCACGATGCAACGAACTC | |
| | | TGAGATCAAGGCGCTCGCGGCCAAGTTGGTGTCTGGCAAGACCTCTCTGTGGGA | |
| | | CAAGGCCAACGCCATCTTCACCTTCGCTAGAGACAACATCACGTACAGTTACTA | |
| | | CGCTGACTCTAAGAAGGGAGCCGCCGGGACCCTCTCTTCTAAGTCAGGCAACTG | |
| | | CTGCGACCACTCTAACTTGATAGTGTCCCTGTGCCGCGCCGCCAACATCACTGCG | |
| | | CGGTTTAGTCACGCACAGGGATGCACCTTCTCATCTGGCTTGGTCGCCGGCCACG | |
| | | TGTGGGCTCAGATTTACATTGACGGCGTCTGGTACACCGCCGACGCAACATCCC | |
| | | GCCGAAACTCACTTGGAAACATCGTTAACTGGAACACCAACCACTACAACACCC | |
| | | TCAAGCAGTACGACCACCTGTCTTTCGGCGGGGGGGGGTCAGGGGGAGGGGGG | |
| | | TCAGGCGGCGGTGGGTCTATCGAGAACGCCGACAAGGCCATCAAGGACTTCCAG | |
| | | GACAACAAAGCCCCTCACGACAAGAGTGCCGCGTACGAGGCGAACTCCAAGCT | |
| | | GCCTAAGGACCTCCGGGACAAGAACAACCGCTTCGTGGAGAAGGTCTCTATCGA | |
| | | GAAGGCGATCGTCCGGCACGACGAGCGGGTGAAGTCCGCGAACGACGCCATCT | |
| | | CTAAACTCAACGAGAAGGACAGCATCGAGAACCGCAGGCTGGCCCAGCGGGAA | |
| | | GTGAACAAGGCCCCTATGGATGTGAAGGAACATCTGCAGAAGCAGCTGGACtaatg | |
| | | atagaccagcctcaagaacacccgaatggagtctctaagctacataataccaacttacac | |
| | | tttacaaaatgttgtcccccaaaatgtagccattcgtatctgctcctaataaaaagaaag | |
| | | tttcttcacattct | |
| C8.4 | mru1499 = | MFVFLVLLPLVSSAAIDIDEASSSSDLSDSSISNDYLVANSGDDSVASSSASSSIAAD | SEQ ID |
| | ADC47349.1, | DSDLSNNASSSNVNFENEVLSTNNNEDTESEIVKDSKNQLSSSSLQASTKTKTTLKGS | NO.: 69 |
| | adhesin- | GSSVYRGNPYYVTLTDSNGKVLASQKVTFNILGKNYTRTTDSKGVASININLAKGK | |
| | like protein | YNIACLYAGTENYASSKLSVALTVNLMSTKINTGGSTVKKGNAYSVTLTDGNGKAL | |
| | with trans- | SSQKVTLNILGKNYTRTTDSKGVASIAINLAAGKKFTLTASYAGSANYLSSKVSATV | |
| | glutaminase | TVQKGDTSIKPSGTSIVKGNSYSFTLVDGSGKGLANQKVAIKISGKSYSRTTNSNGV | |
| | domain | ASIAINLAAGKKYSIVCSYAGSSNYKASSSTVSLSVTNPSTNSKTFSIAKIEAAATNLK | |
| | [Methano- | AYVNKNKAVPTTVSVGGTNLKISEFSYLMSKAIVNLNSNNTNAITLPSGIYNGASAS | |
| | brevibacter | NSLNATVYKAQYVDLSKRVYNYIDKNKVPAAYGTVYNANGASLGNAGENLYTFAF | |
| | ruminantium | AKILDFHKTNKYLPNYCSFDSSVFKASNGSSSSNSSSSTNSSSSTNSSSGSSNSSGSGSS | |
| | M1], | TPAVTVKATSLKAASTSVIRGDDYSVTLTDSSGNALANQKITFALSSSSYTRTTNSKG | |
| | amino acid | VASLTLNLAGGKYSITTSYAGTSAYKASKLTNTVTISNSSSRFFLNDIETAAENVKTY | |
| | | VTKNKALPNTVTVAGTQLTLSQFSYVMAKAIHNINASNSNYISLKSVASSNSTGDYL | |
| | | DTTVYRAQYMNLINRVISFVESDKITPTFATVYNSNGKSVGKAEFKLYTFAFAKILA | |
| | | FYKTNNYLPTYCTFQSSAIGVVPDVATNVTINSKINANMNQFKVGLNEKNTVSNLSA | |
| | | YLVGTGQSTITTNIKNVAAQLTKGLNSTATKALAIYNFVRDDISYSYYSDSRKGADG | |
| | | TLSSGSGNCVDQASLVVALCRAAGIPARYSHAQGCTFSSGLVTGHVWAQILVDGV | |
| | | WYSADATSVRNSLGNIVNWNTNSYHSMKQYAAVPFGGGGSGGGGSGGGGSIENAD | |
| | | KAIKDFQDNKAPHDKSAAYEANSKLPKDLRDKNNRFVEKVSIEKAIVRHDERVKSANDAI | |
| | | SKLNEKDSIENRRLAQREVNKAPMDVKEHLQKQLD | |
| C8.4 | mru1499 = | cttgttctttttgcagaagctcagaataaacgctcaactttggccaccATGTTCGTGTTC | SEQ ID |
| | ADC47349.1, | CTTGTCCTTCTGCCTCTCGTTTCTAGCGCCGCGATCGACATCGATGAGGCCTCTTCTTCC | NO.: 70 |
| | adhesin- | TCTGACCTCAGCGACTCCTCTATCTCTAACGACTACCTGGTGGCTAACAGCGGGGACGAC | |
| | like protein | TCAGTTGCCAGCTCTTCCGCCTCTAGCTCTATCGCGGCAGACGACTCTGACCTGTCTAA | |
| | with trans- | CAACGCCTCTTCTTCTAACGTCAACTTCGAGAACGAGGTCCTGTCCACCAACAAT | |
| | glutaminase | AACGAGGATACCGAGTCTGAGATCGTCAAGGACTCTAAGAATCAGCTGTCCTCT | |
| | domain | TCTAGCCTCCAGGCGTCTACAAAGACCAAGACCACCCTCAAGGGCTCCGGTTCT | |
| | [Methano- | TCTGTGTACCGGGGGAACCCTTACTACGTTACTCTCACTGACTCTAACGGCAAGG | |
| | brevibacter | TGCTGGCCAGTCAGAAGGTGACGTTCAATATACTGGGCAAGAACTACACCAGGA | |
| | ruminantium | CCACCGACAGTAAAGGCGTGGCAAGTATCAACATAAATCTGGCCAAGGGCAAG | |
| | M1], | TACAACATCGCTTGCCTCTACGCCGGAACGGAGAACTACGCATCTTCTAAGCTGT | |
| | nucleotide | CTGTTGCCCTGACTGTGAACCTCATGTCTACGAAAATTAACACGGGCGGGTCTAC | |
| | | CGTGAAGAAGGGGAACGCCTACTCCGTGACCCTGACTGACGGAAACGGGAAGG | |
| | | CTCTGTCTTCTCAGAAAGTCACCCTTAACATCCTTGGGAAGAACTACACCAGAAC | |
| | | TACGGACTCTAAGGGGGTTGCTTCTATCGCGATAAACCTGGCCGCCGGTAAGAA | |
| | | GTTCACCCTCACCGCCTCTTATGCCGGCTCTGCCAACTACCTCAGCTCTAAGGTG | |
| | | TCCGCGACCGTGACCGTGCAGAAGGGCGACACATCTATTAAGCCTAGTGGGACC | |
| | | AGCATCGTGAAAGGGAACTCCTATTCTTTCACACTGGTGGACGGCTCTGGGAAG | |
| | | GGCCTGGCGAATCAAAAGGTCGCGATCAAGATCTCTGGCAAATCCTATAGCCGG | |
| | | ACCACCAACTCTAACGGGGTGGCCTCAATTGCCATCAATTTGGCGGCCGGCAAG | |
| | | AAGTACTCTATCGTCTGCTCTTACGCGGGTTCTAGTAACTATAAGGCTTCTTCTTC | |
| | | AACCGTGTCTCTGTCTGTGACCAACCCTTCAACCAACAGCAAAACCTTCTCTATC | |
| | | GCCAAGATCGAGGCCGCCGCCACCAACCTGAAGGCTTACGTGAACAAGAACAA | |
| | | GGCCGTGCCTACCACAGTGTCTGTCGGCGGCACCAACCTGAAGATCTCTGAATT | |
| | | CTCTTACTTGATGTCTAAGGCCATCGTCAACCTGAACTCTAACAACACAAACGCG | |
| | | ATAACGCTCCCTTCTGGCATCTACAACGGGGCCTCAGCCTCTAACTCTCTCAACG | |
| | | CCACCGTGTACAAGGCCCAGTACGTGGACTTGAGCAAGCGAGTCTACAACTACA | |
| | | TCGACAAGAACAAGGTCCCTGCGGCCTACGGCACCGTCTACAACGCCAACGGCG | |
| | | CTTCTCTTGGCAACGCCGGCTTCAACCTGTACACCTTCGCGTTCGCCAAGATCTT | |
| | | GGACTTCCACAAGACCAACAAGTACCTCCCTAACTACTGCTCTTTCGACTCTTCT | |
| | | GTCTTCAAGGCCTCTAACGGCTCTAGCTCTAGTAACTCATCTTCTTCTACCAACT | |
| | | CATCTTCATCTACGAACTCATCTTCCGGCTCTTCTAACAGTAGTGGGTCTGGGAG | |
| | | TTCTACGCCTGCCGTTACCGTGAAGGCGACCTCTTTGAAGGCCGCCAGTACCTCC | |
| | | GTGATCCGGGGCGATGACTACTCAGTGACGCTCACGGACTCTTCTGGGAACGCA | |
| | | CTGGCGAACCAGAAGATTACGTTTGCCCTCTCATCTAGTAGTTACACCCGGACCA | |
| | | CCAACTCTAAGGGCGTTGCCAGCCTCACGCTGAACCTGGCCGGTGGCAAGTATA | |
| | | GCATCACCACTTCATACGCCGGAACATCTGCCTACAAGGCCTCTAAGCTGACAA | |
| | | ACACCGTCACGATCTCTAATTCTTCATCTCGCTTCTTCCTGAACGACATTGAGAC | |
| | | CGCCGCCGAGAACGTGAAGACGTACGTCACCAAGAACAAGGCTCTCCCTAACAC | |
| | | GGTGACGGTCGCCGGCACCCAGCTGACCCTGTCTCAGTTCTCCTACGTGATGGCA | |
| | | AAGGCGATCCATAACATCAACGCTAGTAACTCTAACTACATATCTCTGAAGTCT | |
| | | GTGGCCAGTTCAAACAGTACCGGGGACTACCTCGACACGACCGTGTACAGGGCC | |
| | | CAGTACATGAACCTGACCAACCGCGTCATCTCTTTCGTGGAGTCCGATAAGATC | |
| | | ACCCCTACCTTTGCCACCGTCTACAACTCAAACGGAAAGTCCGTCGGGAAGGCC | |
| | | GAGTTCAAGTTGTACACATTCGCTTTCGCCAAGATCCTGGCCTTCTACAAAACCA | |
| | | ATAACTACCTGCCTACGTACTGCACGTTTCAGAGCTCTGCCATCGGCGTGGTGCC | |
| | | TGACGTGGCCACGAACGTGACCATCAACAGTAAGATCAACGCCAACATGAACCA | |
| | | GTTCAAAGTCGGTCTGAACGAGAAGAACACTGTGTCTAACCTGTCTGCATACCT | |
| | | GGTGGGTACCGGCCAGTCTACTATCACAACTAACATCAAGAACGTCGCCGCCCA | |
| | | GCTGACCAAGGGACTCAACTCTACAGCCACTAAGGCCCTGGCTATCTACAACTT | |
| | | CGTGCGAGACGACATCTCTTACTCCTACTACTCTGACAGCCGCAAGGGAGCCGA | |
| | | CGGCACCCTGTCTTCCGGGTCCGGGAACTGCGTCGATCAGGCATCTTTGGTGGTG | |
| | | GCCCTGTGCCGGGCAGCCGGGATCCCTGCACGCTACTCACACGCGCAGGGGTGT | |
| | | ACCTTCTCTAGCGGCCTTGTCACGGGCCACGTGTGGGCCCAAATCCTGGTGGAC | |
| | | GGAGTCTGGTACTCTGCAGACGCCACCTCAGTGCGGAACTCTTTGGGCAACATA | |
| | | GTGAACTGGAATACCAACTCTTACCACTCAATGAAGCAGTACGCCGCGGTGCCT | |
| | | TTCGGTGGGGGGGGTCTGGAGGGGGCGGTTCTGGCGGCGGGGGCTCTATCGAG | |
| | | AACGCGGACAAGGCGATCAAGGACTTCCAGGACAACAAGGCGCCTCACGACAA | |
| | | GAGTGCCGCATACGAAGCCAACAGCAAGCTCCCTAAGGACCTTCGCGACAAGA | |
| | | ATAATCGGTTCGTCGAGAAGGTGTCTATCGAGAAGGCCATCGTGCGCCACGACG | |
| | | AGAGAGTTAAATCTGCCAACGACGCCATCTCTAAGCTGAACGAGAAGGACAGTA | |
| | | TCGAGAACCGCCGGCTTGCCCAGCGCGAGGTGAACAAGGCCCCTATGGACGTGA | |
| | | AGGAGCACCTGCAGAAGCAGCTCGACtaatgatagaccagcctcaagaacacccgaatgg | |
| | | agtctctaagctacataataccaacttacactttacaaaatgtgtcccccaaaatgtagc | |
| | | cattcgtatctgctcctaataaaaagaaagtttcttcacattct | |
| PPa2del | swapped ssp | MRFPSIFTAVLFAASSALAMPLSGGGSGGSGSMPLNTTTEDETAQIPAEAVIGYL | SEQ ID |
| SIP- | for PPa_v2 | DLEGDEDVAVLPFSNSTNNGLLFINTTIASIAAKEEGVSAAIDVDTNDNLDDGSSS | NO.: 71 |
| C8.1 | without SIP | NSDLISSSSLDSSSSDDVSSGSSEVSSSDESLDGNNLSDGNVSSSDESVGADNLSDGN | |
| | sites, | VSSSDESVGADNLSDDESSSDALSEELPKTETVIKADPINYNYASVKGLTINLTDSAG | |
| | mru0493 = | LALSNKTLTVKVSALNKTSNLTTNSKGQAIFKLSASVGSYDVFISFTGDESYAPSNAS | |
| | ADC46344.1, | SKITIKKSSTKIKLSNIHGYLTISNYVSVTLLDSAGKPIKSKSVTIQVNKAKYNVKTDS | |
| | adhesin- | KGIAKVKVANKIGTYSVNAKFSGDKNYYASSNSSKLTITKMKVYIKAPSVKYYMTN | |
| | like protein | SSAPYLTINLTNVKGSPLAKKKVSVKIGKKTYTLKTNSQGIAKFKFTKKVSSYNCKIN | |
| | [Methano- | FKATSNFYGASVNSKMTIQKMPTSLKAPSVSINSTNYGKVLISLKDGKGKALKNTTV | |
| | brevibacter | TVNVTELKKVFTLKTNASGVATFSFNGEKTFNLKIKYAGNKNYAASSVSSKINVKQI | |
| | ruminantium], | KVKLSDVIGASRVLIDYVNRTKDLPSNVQYNNYNFTVTQLTYLASKAVKNINNKNY | |
| | amino acid | GDIVLISVPKSYKSSGEIYDTVYKKDFVKIANSVVGSSYNYKNKEYVSYSIYKVPFKV | |
| | | YSISFAKVLNFYGNNKKLPNYSLFTLADFAKVKDNGGYNFYLTTDNIAGKKSDLNM | |
| | | LKSLAKTLKSMGYNAVIVGIGPDIHNVAYRYGCTGNNSVLLACFGGVDVGCIEEWA | |
| | | GDLGDLNGHSFVNSYQGAHVLGLWFTKPYGASVSLNKKVGIAWDADYGFPLNTPA | |
| | | KYMKSHNISYIETGTVANACKLLSEGKMGGPQLISGGGGSGGGGGGGGSIENADKA | |
| | | IKDFQDNKAPHDKSAAYEANSKLPKDLRDKNNRFVEKVSIEKAIVRHDERVKSANDAIS | |
| | | KLNEKDSIENRRLAQREVNKAPMDVKEHLQKQLD | |
| PPa2del | swapped ssp | cttgttctttttgcagaagctcagaataaacgctcaactttggccaccATGCGCTTCCCT | SEQ ID |
| SIP- | for PPa_v2 | TCTATTTTCACCGCCGTGCTCTTCGCCGCCTCTTCAGCACTCGCAATGCCTCTGTCTGGG | NO.: 72 |
| C8.1 | without SIP | GGCGGGTCTGGCGGCAGCGGCTCTATGCCTCTGAACACCACTACCGAGGACGAGACGGCG | |
| | sites,CAGA | TACCTGCGGAGGCCGTGATCGGGTACCTCGATCTGGAGGGCGACTTCGACGTGG | |
| | mru0493 = | CCGTGCTCCCTTTCAGTAACTCAACCAACAACGGGCTCCTGTTCATCAACACCAC | |
| | ADC46344.1, | CATCGCTAGTATCGCCGCAAAGGAGGAGGGCGTTTCAGCCGCCATCGACGTCGA | |
| | adhesin- | CACCAACGACAACCTGGATGACGGCTCTTCTTCTAATTCTGACCTGATCAGCTCC | |
| | like protein | TCTTCTTTGGACAGCTCTAGCAGTGACGACGTGTCCAGCGGGTCAAGTGAAGTG | |
| | [Methano- | TCATCCTCTGACGAGTCTCTGGACGGCAATAACTTGAGCGACGGAAACGTGTCT | |
| | ebrvibacter | TCATCGGACGAGAGCGTTGGGGCCGACAACCTGTCTGATGGCAACGTCTCTTCT | |
| | ruminantium], | AGTGACGAGTCTGTGGGCGCCGACAACCTTTCTGACGACGAGTCATCTTCTGAC | |
| | nucleotide | GCCCTTTCTGAGGAACTGCCTAAGACCGAGACTGTGATCAAGGCCGACCCTATA | |
| | | AACTACAACTACGCGTCCGTGAAGGGTCTGACCATAAACTTGACCGACTCTGCG | |
| | | GGCCTGGCACTGTCTAACAAGACCCTGACCGTGAAGGTGTCTGCCCTTAATAAG | |
| | | ACCTCTAACCTGACAACGAACTCCAAGGGGCAGGCCATCTTCAAGCTTTCTGCCT | |
| | | CCGTTGGGTCTTACGACGTCTTCATCTCTTTCACCGGAGACGAGAGCTACGCCCC | |
| | | TAGTAACGCATCTTCTAAGATCACCATCAAGAAGTCTAGTACTAAGATCAAACT | |
| | | TTCTAACATCCATGGGTACCTGACCATCAGTAACTATGTGTCTGTGACTCTGTTG | |
| | | GACTCTGCCGGAAAGCCTATCAAGTCCAAATCCGTGACCATCCAAGTGAACAAG | |
| | | GCGAAGTACAACGTCAAGACGGACTCTAAGGGGATCGCCAAGGTGAAGGTGGC | |
| | | CAACAAGATCGGGACCTACTCTGTGAACGCTAAGTTCAGCGGGGACAAGAACTA | |
| | | CTACGCGTCTTCTAACTCTAGTAAGCTCACCATCACGAAGATGAAAGTGTACATC | |
| | | AAAGCCCCTTCTGTTAAGTACTACATGACAAACTCTTCTGCCCCTTACCTGACGA | |
| | | TCAACCTGACGAACGTGAAAGGATCTCCTCTGGCCAAGAAGAAGGTTAGCGTGA | |
| | | AGATAGGGAAGAAGACCTACACGCTGAAGACCAACTCTCAGGGAATCGCCAAG | |
| | | TTCAAGTTTACGAAGAAGGTGTCTTCTTACAACTGCAAGATCAACTTCAAGGCC | |
| | | ACCTCTAATTTCTACGGCGCCTCCGTGAACTCTAAGATGACCATCCAGAAGATGC | |
| | | CTACAAGTCTGAAGGCCCCTTCTGTGTCTATCAACTCAACAAACTACGGGAAGG | |
| | | TCCTGATCTCACTGAAGGACGGCAAGGGGAAGGCCCTCAAGAACACGACAGTC | |
| | | ACGGTCAACGTGACCGAGTTGAAGAAGGTCTTCACCCTGAAGACAAACGCCTCT | |
| | | GGTGTGGCTACCTTCTCTTTTAACGGCGAAAAGACCTTCAACCTCAAGATTAAGT | |
| | | ATGCGGGAAACAAAAACTACGCGGCCTCTTCTGTCTCTTCTAAGATCAACGTTA | |
| | | AGCAGATTAAGGTCAAACTCTCTGACGTCATCGGCGCCTCTCGGGTGCTCATCG | |
| | | ACTACGTCAACCGGACCAAGGATCTGCCTTCAAACGTGCAGTACAACAACTACA | |
| | | ACTTCACCGTGACGCAGCTCACCTATCTGGCTTCTAAGGCCGTGAAGAACATCA | |
| | | ACAACAAGAACTACGGCGACATCGTCCTCATATCTGTCCCTAAGAGTTACAAGT | |
| | | CATCTGGCGAGATCTACGACACGGTGTACAAGAAGGACTTCGTGAAGATCGCTA | |
| | | ACTCCGTGGTGGGGTCTTCTTACAACTACAAGAACAAGGAGTATGTTTCTTACTC | |
| | | AATCTACAAGGTGCCTTTCAAGGTCTACTCTATTTCTTTCGCCAAGGTGCTGAAT | |
| | | TTTTACGGCAACAACAAAAAGCTGCCTAACTACTCCCTCTTCACCCTCGCCGACT | |
| | | TCGCGAAAGTGAAGGATAACGGCGGTTACAACTTCTACCTGACCACCGACAATA | |
| | | TCGCCGGAAAGAAGTCAGACCTGAACATGCTGAAGTCCCTCGCCAAGACCCTGA | |
| | | AGTCTATGGGTTACAATGCCGTCATTGTGGGCATCGGGCCTGACATCCACAACG | |
| | | TCGCCTACAGATACGGGTGCACTGGGAACAACTCTGTCCTCCTCGCGTGCTTCGG | |
| | | CGGAGTCGACGTGGGCTGCATCGAGGAGTGGGCCGGCGACCTGGGCGACCTGA | |
| | | ACGGGCACTCTTTCGTGAACTCTTATCAGGGCGCGCACGTCCTGGGGCTCTGGTT | |
| | | CACTAAGCCTTACGGCGCCTCCGTCAGTCTGAACAAGAAGGTTGGCATCGCCTG | |
| | | GGACGCGGACTACGGCTTCCCTCTCAACACGCCTGCAAAGTACATGAAGAGTCA | |
| | | CAACATCAGCTACATCGAGACCGGTACCGTGGCAAACGCATGCAAGCTGCTCAG | |
| | | CGAGGGGAAGATGGGCGGCCCTCAGCTTATCAGCGGCGGCGGCGGCTCTGGCG | |
| | | GGGGGGGCAGCGGTGGTGGGGGTTCTATAGAGAATGCGGACAAGGCCATCAAG | |
| | | GATTTCCAGGACAACAAGGCTCCTCACGACAAGTCAGCCGCTTACGAGGCCAAC | |
| | | TCTAAGTTGCCTAAAGACCTGCGCGACAAGAACAACCGCTTCGTGGAGAAGGTG | |
| | | TCTATTGAGAAAGCCATCGTGCGGCACGACGAGCGCGTGAAGTCTGCCAACGAC | |
| | | GCCATCTCCAAGCTGAACGAGAAGGACAGTATCGAGAACAGGCGGTTGGCCCA | |
| | | GCGAGAGGTCAATAAGGCTCCTATGGACGTCAAGGAGCACCTTCAGAAGCAGCT | |
| | | GGACtaatgatagaccagcctcaagaacaccegaatggagtctctaagctacataatacc | |
| | | aacttacactttacaaaatgtgtcccccaaaatgtagccattcgtatctgetcctaataa | |
| | | aaagaaagtttcttcacattct | |
| PPa2del | swapped ssp | MRFPSIFTAVLFAASSALAMPLSGGGSGGSGSMPLNTTTEDETAQIPAEAVIGYL | SEQ ID |
| SIP- | for PPa_v2 | DLEGDFDVAVLPFSNSTNNGLLFINTTIASIAAKEEGVSAAQSLDNINYANDGEDS | NO.: 73 |
| C8.2 | without SIP | DEMINCDLHKDSSQKSLKSNALSNKKTTNTVKLTDMKKAESNDVKQTGAAKASNT | |
| | sites, | KSTSKSTTKTNATKSNTTKSTATKTTANSSSTKKATQNTTTINTQTLAKSSSSYMAY | |
| | mru0824 = | VEKNAKLQEPITISKKKYKSPEYLYLVSKAVSNISKTKVEIKDKLITNYSNTDCKSVN | |
| | ADC46675.1, | GTINKTEYVQVAKKTVSFIEKNHRAPNWIASSKGNIPRNQLILVESKCLDQYNKSGK | |
| | adhesin- | LPSSIKLNDLDLNKMKQKIDSSKKVNSTSTKKTNTSSTKINSTSAKKTNTTSTKKTNP | |
| | like protein | TATSTNNNKSLVESTLDSIKSILNNIENKLNPTNKVLSTTGTKKNTVTVNSSKVNVQI | |
| | with trans- | SSSSTVNVKISAKDNTNSGKNTNSGSAKKTNTTSTKKTNTTSTKKIDTNSTKKTNTTS | |
| | glutaminase | TKNNTSSAKKTNTTSTKNNTSSAKKTNTTSTKNNTSSAKKVNTSSSKTNTSAKNNTS | |
| | domain | TTAKSSSNSKYLSTSVLNDKYLGESLKKYLAVGKNCQVTNKAIKTLANTLTSKLKS | |
| | [Methano- | DYKKGEKIFNWVRDNIGYEKYRNTKKGALKTLQTRGGNCVDHAHLIVALSRAAGL | |
| | brevibacter | PARYVNANNCKFSSGYVSGHVWAQVLVGNTWVVADATSNRNKFGVVKNWNVNS | |
| | ruminantium | YKLVGKYSSISFGGGGSGGGGSGGGGSIENADKAIKDFQDNKAPHDKSAAYEANSKLP | |
| | M1], | KDLRDKNNRFVEKVSIEKAIVRHDERVKSANDAISKLNEKDSIENRRLAQREVNKAPMDV | |
| | amino acid | KEHLQKQLD | |
| PPa2del | swapped ssp | cttgttctttttgcagaagctcagaataaacgctcaactttggccaccATGAGGTTCCCT | SEQ ID |
| SIP- | for PPa_v2 | AGCATTTTTACCGCGGTTCTCTTTGCCGCTTCTAGCGCTCTCGCAATGCCTCTCTCCGGG | NO.: 74 |
| C8.2 | without SIP | GGGGGAAGCGGAGGGTCAGGCTCTATGCCTTTGAACACCACCACAGAAGACGAGACTGCG | |
| | sites, | CAAATACCTGCGGAGGCCGTGATCGGCTACCTGGATCTGGAAGGGGATTTCGACGTG | |
| | mru0824 = | GCAGTGCTGCCTTTTAGCAACTCTACCAACAATGGACTTCTGTTCATTAACACAA | |
| | ADC46675.1, | CAATCGCCTCCATCGCAGCTAAGGAGGAAGGGGTGTCAGCTGCCCAGAGTCTGG | |
| | adhesin- | ATAACATCAATTATGCCAACGACGGATTCGACTCTGACGAAATGATCAACTGCG | |
| | like protein | ACCTCCACAAGGACTCCAGTCAGAAAAGCCTCAAGTCTAACGCACTGTCCAACA | |
| | with trans- | AGAAGACGACCAATACCGTCAAGCTGACAGATATGAAAAAAGCTGAGTCAAAC | |
| | glutaminase | GACGTGAAGCAAACAGGCGCAGCTAAGGCCAGCAATACTAAAAGTACTTCTAA | |
| | domain | GAGTACTACAAAGACCAACGCTACTAAATCTAATACCACAAAGTCAACTGCCAC | |
| | [Methano- | CAAGACTACCGCAAATTCTTCAAGCACAAAGAAAGCCACGCAAAATACCACTAC | |
| | brevibacter | AATCAACACCCAGACGCTGGCCAAGTCCTCATCCAGCTACATGGCATATGTGGA | |
| | ruminantium | GAAGAATGCCAAGTTGCAAGAGCCTATCACCATTTCTAAGAAAAAATATAAGTC | |
| | M1], | TCCTGAGTATCTGTACCTGGTGTCTAAAGCCGTGAGCAACATCAGCAAAACAAA | |
| | nucleotide | GGTGGAGATCAAGGACAAATTGATTACCAATTACTCCAACACGGATTGCAAAAG | |
| | | CGTGAACGGCACAATCAATAAAACAGAATACGTGCAGGTCGCCAAAAAAACCG | |
| | | TGAGTTTCATAGAGAAGAATCATCGGGCTCCTAATTGGATCGCCTCAAGCAAGG | |
| | | GGAACATACCTAGAAATCAGCTTATTCTGGTCTTTAGCAAATGTCTCGATCAGTA | |
| | | CAACAAATCAGGTAAACTCCCTTCAAGTATAAAACTGAACGATTTGGACCTGAA | |
| | | CAAGATGAAGCAGAAGATCGACTCCTCCAAGAAGGTTAATTCCACTAGCACAAA | |
| | | GAAGACCAACACCTCCAGCACTAAAACAAACTCCACCTCAGCAAAGAAGACTA | |
| | | ACACGACATCCACAAAAAAGACTAATCCTACCGCCACGAGCACAAATAATAATA | |
| | | AGTCCCTCGTCGAGAGCACTCTGGATAGTATCAAGAGCATTTTGAATAATATTG | |
| | | AGAACAAGCTTAATCCTACGAACAAAGTCCTTTCCACAACCGGAACCAAGAAAA | |
| | | ACACTGTTACCGTTAACAGTTCTAAAGTGAATGTTCAGATTAGCAGCTCTAGCAC | |
| | | CGTGAATGTCAAGATCAGTGCCAAGGATAACACCAACAGCGGGAAGAACACCA | |
| | | ACTCCGGCAGCGCCAAAAAGACCAACACTACCAGCACTAAGAAAACCAATACC | |
| | | ACCTCAACAAAAAAGATTGATACAAATTCCACGAAAAAAACCAACACCACGAG | |
| | | TACAAAAAATAACACGTCCAGCGCCAAGAAGACTAATACGACCTCAACCAAAA | |
| | | ACAATACTTCCAGTGCCAAGAAGACGAACACAACTTCCACTAAGAACAACACAA | |
| | | GTTCCGCGAAAAAAGTGAACACTTCTTCCAGTAAAACCAACACGTCCGCAAAGA | |
| | | ACAATACATCTACGACGGCTAAGTCATCCAGCAATTCCAAGTACCTCAGCACCT | |
| | | CCGTGCTCAACGACAAGTACCTGGGCGAGAGTTTGAAGAAATATCTGGCCGTTG | |
| | | GAAAGAATTGTCAGGTGACCAATAAGGCTATTAAAACACTTGCCAACACCCTGA | |
| | | CTTCCAAACTCAAGAGCGATTACAAAAAGGGGGAAAAGATCTTTAACTGGGTCC | |
| | | GGGACAACATAGGCTACGAAAAGTATAGAAACACCAAAAAGGGAGCCCTGAAG | |
| | | ACCCTTCAGACACGGGGGGGCAATTGTGTCGATCATGCACACTTGATAGTGGCG | |
| | | CTGAGTCGAGCCGCTGGTCTGCCTGCCAGGTATGTCAATGCCAACAACTGCAAA | |
| | | TTTTCAAGTGGCTACGTGTCTGGGCATGTGTGGGCTCAGGTCCTGGTCGGTAACA | |
| | | CTTGGGTTGTGGCCGACGCAACCAGCAACCGCAATAAGTTCGGAGTGGTCAAAA | |
| | | ACTGGAATGTGAACTCTTACAAGCTGGTGGGCAAGTATTCAAGCATCAGTTTCG | |
| | | GTGGTGGCGGTTCTGGGGGAGGCGGCTCTGGTGGCGGCGGAAGCATCGAAAAC | |
| | | GCTGACAAAGCGATCAAGGACTTCCAGGATAACAAGGCGCCTCACGATAAGTCT | |
| | | GCTGCATACGAGGCTAATTCTAAACTGCCTAAGGACCTTCGCGACAAGAATAAC | |
| | | AGATTCGTTGAAAAGGTGTCAATCGAGAAGGCCATTGTGAGGCACGACGAACGC | |
| | | GTTAAGAGCGCCAATGATGCGATCAGTAAACTGAACGAAAAAGATTCCATTGAG | |
| | | AACCGACGCCTGGCTCAGCGGGAGGTGAACAAAGCACCTATGGACGTCAAGGA | |
| | | GCACCTCCAGAAGCAGCTGGACtaatgatagaccagcctcaagaacaccegaatggagtc | |
| | | tctaagctacataataccaacttacactttacaaaatgttgtcccccaaaatgtagccat | |
| | | tcgtatctgcetcctaataaaaagaaagtttcttcacattct | |
| PPa2del | swapped ssp | MRFPSIFTAVLFAASSALAMPLSGGGSGGSGSMPLNTTTEDETAQIPAEAVIGYL | SEQ ID |
| SIP- | for PPa_v2 | DLEGDEDVAVLPFSNSTNNGLLFINTTIASIAAKEEGVSAASNASDNLDDLTISDSN | NO.: 75 |
| C8.3 | without SIP | SLDLVSTSNSDILSSDSGVSSDDSSNDASGDVLGSDVSSNESNNQSQSTLDSNNQSQS | |
| | sites, | GLDSDNSTLLDSQSNNQSNSESSDSSDSSETVIKNATSISVSSKTVVRGNSLNITLKDN | |
| | mru1604 = | ASTLLSNKTVTFTFNGKTYNKTTNAKGIASLTLTATPKKYLVKIAFVGDELYEASSK | |
| | ADC47454.1, | SVNVTLSKTPTSISNSGKSIVRGKLYKLTLKDAKGKALSGKKISISFNGKKYTKTTNS | |
| | adhesin- | NGQVNLTINVNVGKTYKMTYKFAGDSNYLSSSGSVSIKVKMGTSIIGSGSSIVKGKS | |
| | like protein | YTVTLKNANGAVLSNQKIAFTLSGKTYNRTTNAKGQASLKIGLSSGKTYNLTYKYA | |
| | with trans- | GNSYYGGSSGKVSLFVKTPTTMKNSGKTIVSGETYKVTLKDADGKSLANKKVSITF | |
| | glutaminase | NNKTYAKTTNSNGQASLTIKGTFGRSYPLSYKFAGDSKYGPSSGSLCLRVKKATSLK | |
| | domain | GSASSIVQGKSYTVTLKDSNSTPLANQTIVFTLDTKKYNRTTNAKGQASLKIGLAAG | |
| | [Methano- | KTYNLAYKYSGTSYYNGSSGSVKLKVKFPTSLTNSGKSVMNGTGYNIVLKDSKSNL | |
| | brevibacter | VSNKTISIGFNGKTYDEITDANGTVTLLIDANVPKTYKMTYKFAGDSDYGASSGTVN | |
| | ruminantium | LTVKFKNAFTISQIISASSSLKSYVLKNKKVPATVSVNGVSLNLTSFTYLMAKATISIN | |
| | M1], | SNKTSGSILLVPVDSNYTNNGSRINANLYKANYIDLAKKVISSAEANKLVPNSVSTNI | |
| | amino acid | GLVSHDLYSFGLAKALVFFNSDHYLPNYLILSSDDVGEKHSTVIPSNARGNASQFKA | |
| | | GLNEAETLTAAQIAKYLVASGHDATNSEIKALAAKLVSGKTSLWDKANAIFTFARD | |
| | | NITYSYYADSKKGAAGTLSSKSGNCCDHSNLIVSLCRAANITARFSHAQGCTFSSGL | |
| | | VAGHVWAQIYIDGVWYTADATSRRNSLGNIVNWNTNHYNTLKQYDHLSFGGGGS | |
| | | GGGGSGGGGSIENADKAIKDFQDNKAPHDKSAAYEANSKLPKDLRDKNNRFVEKVSIEK | |
| | | AIVRHDERVKSANDAISKLNEKDSIENRRLAQREVNKAPMDVKEHLQKQLD | |
| PPa2del | swapped ssp | cttgttctttttgcagaagctcagaataaacgctcaactttggccaccATGCGCTTCCCT | SEQ ID |
| SIP- | for PPa_v2 | AGTATCTTCACCGCCGTGCTGTTCGCTGCCTCTTCTGCCTTGGCCATGCCTCTGTCTGGC | NO.: 76 |
| C8.3 | without SIP | GGAGGCTCTGGGGGCTCAGGTTCTATGCCTCTCAACACGACGACGGAGGACGAGACCGCC | |
| | sites, | CAGATACCTGCCGAGGCCGTGATCGGGTACCTCGACCTCGAAGGTGACTTTGACGTCG | |
| | mru1604 = | CGGTGCTCCCTTTCTCTAACTCTACAAACAACGGCTTGCTGTTCATCAACACCAC | |
| | ADC47454.1, | GATCGCGTCAATCGCGGCCAAGGAGGAGGGCGTCTCTGCCGCCTCCAATGCCTC | |
| | adhesin- | CGATAACCTGGACGATTTGACCATTAGCGACAGTAACTCCCTGGATCTTGTGAG | |
| | like protein | CACCTCTAACTCTGACATACTGTCTTCTGACTCTGGCGTCTCTTCTGACGACTCTA | |
| | with trans- | GTAACGACGCCAGTGGAGACGTCCTGGGCAGTGATGTGTCATCTAACGAGTCCA | |
| | glutaminase | ACAACCAGTCCCAGTCTACCCTCGACTCTAACAACCAGTCTCAGTCTGGTCTCGA | |
| | domain | CTCTGACAATTCTACCCTCCTTGACTCACAGTCTAACAACCAGAGCAACTCTGAG | |
| | [Methano- | TCTTCTGACTCTAGCGACTCTTCAGAGACTGTCATCAAGAATGCAACCTCTATCT | |
| | brevibacter | CTGTGAGCTCTAAGACCGTCGTGCGGGGAAACTCCCTGAACATCACCCTGAAGG | |
| | ruminantium | ACAACGCTTCAACCCTGCTTTCCAACAAAACCGTCACCTTCACGTTCAATGGAAA | |
| | M1], | GACTTACAACAAGACCACCAACGCGAAGGGGATCGCCTCTCTGACCCTGACTGC | |
| | nucleotide | CACCCCTAAGAAGTACCTTGTGAAGATCGCGTTCGTCGGGGACGAGCTGTACGA | |
| | | GGCGTCTAGCAAGTCTGTGAACGTCACCCTTTCTAAGACGCCTACAAGCATATCT | |
| | | AACTCTGGCAAAAGCATCGTGCGAGGGAAGCTGTACAAGCTCACGCTCAAGGAC | |
| | | GCCAAGGGGAAAGCCCTGTCTGGAAAGAAGATCTCTATCTCTTTCAACGGCAAG | |
| | | AAGTACACCAAGACCACCAACAGTAACGGTCAAGTTAACCTGACGATCAACGTG | |
| | | AACGTGGGCAAGACATATAAGATGACCTACAAGTTCGCCGGTGACTCTAACTAC | |
| | | CTGTCATCTAGTGGCAGCGTGTCTATCAAGGTCAAAATGGGGACCTCTATTATCG | |
| | | GGTCTGGGTCTTCTATCGTTAAGGGTAAGTCATACACCGTCACACTGAAGAACG | |
| | | CCAACGGGGCCGTGCTGTCTAACCAGAAGATCGCATTCACACTCTCCGGCAAGA | |
| | | CGTACAACAGAACGACCAACGCCAAGGGCCAGGCCTCCCTGAAGATCGGGCTGT | |
| | | CTTCTGGGAAGACCTACAACCTGACGTACAAGTACGCCGGGAACTCTTACTACG | |
| | | GAGGGAGTTCTGGCAAGGTGTCTCTGTTCGTCAAGACCCCTACCACAATGAAGA | |
| | | ACTCTGGGAAGACCATCGTCTCTGGCGAGACCTACAAGGTGACCCTCAAGGACG | |
| | | CCGACGGCAAGAGCCTTGCAAACAAGAAGGTGTCAATCACCTTCAACAATAAGA | |
| | | CCTACGCCAAGACGACCAACAGTAACGGGCAGGCCTCTCTGACGATTAAGGGAA | |
| | | CCTTCGGACGCTCTTATCCTCTGTCTTACAAGTTTGCGGGCGACAGCAAGTACGG | |
| | | GCCTTCCAGTGGCAGCCTGTGCCTGCGCGTCAAGAAGGCGACGAGCCTCAAGGG | |
| | | GTCTGCCTCCTCTATCGTGCAGGGCAAGTCTTACACCGTGACTCTGAAGGACTCA | |
| | | AACAGTACGCCTCTCGCCAACCAGACCATAGTGTTCACTCTTGACACCAAGAAG | |
| | | TACAACAGGACCACCAACGCCAAGGGCCAGGCCTCTTTGAAGATCGGGCTGGCC | |
| | | GCCGGGAAGACCTACAACCTGGCCTACAAGTACTCTGGGACCTCTTACTACAAC | |
| | | GGCTCAAGTGGGTCAGTGAAGCTGAAGGTTAAGTTCCCTACCTCTCTCACCAACT | |
| | | CCGGGAAGTCTGTGATGAACGGCACCGGCTACAACATCGTCCTCAAGGACTCTA | |
| | | AGAGCAACCTGGTTTCTAATAAGACCATCTCCATCGGCTTCAACGGCAAGACGT | |
| | | ACGACGAGATCACCGATGCGAACGGCACCGTTACCCTCCTCATCGACGCTAACG | |
| | | TCCCTAAGACGTACAAGATGACTTACAAGTTCGCGGGCGACTCCGACTACGGGG | |
| | | CGTCTAGCGGGACAGTGAACCTCACTGTGAAATTCAAGAACGCCTTCACGATCA | |
| | | GTCAGATCATTAGTGCCAGCTCTTCTCTGAAGTCTTACGTGCTCAAGAACAAGAA | |
| | | GGTGCCTGCCACGGTGTCAGTCAATGGCGTGTCTCTGAACCTGACCAGTTTCACC | |
| | | TACCTGATGGCGAAGGCAACCATCTCCATTAACTCTAACAAGACCTCTGGCTCTA | |
| | | TACTGCTCGTGCCTGTCGACTCAAACTACACCAACAACGGCTCTCGGATCAACG | |
| | | CCAATCTGTACAAGGCCAATTACATCGACCTGGCCAAGAAGGTCATCTCTTCTGC | |
| | | TGAGGCCAACAAATTGGTGCCTAACAGCGTGTCCACAAACATCGGGCTGGTGTC | |
| | | TCACGACTTGTACTCCTTCGGACTCGCCAAAGCGTTGGTCTTCTTCAACAGTGAC | |
| | | CACTACTTGCCTAACTACCTGATCCTTTCTTCTGACGACGTCGGGGAGAAACACT | |
| | | CTACGGTGATCCCTTCTAACGCTCGCGGCAACGCATCTCAGTTCAAGGCGGGCCT | |
| | | CAACGAGGCCGAGACTCTCACAGCCGCACAAATCGCCAAATATCTGGTGGCGTC | |
| | | TGGGCACGACGCCACGAACTCTGAGATCAAGGCCTTGGCCGCCAAGCTGGTGTC | |
| | | AGGCAAGACCTCTCTGTGGGACAAGGCAAACGCCATTTTCACCTTTGCTCGCGA | |
| | | CAACATCACCTACAGTTATTACGCCGACAGTAAGAAGGGCGCCGCTGGCACCTT | |
| | | GAGCTCAAAATCTGGCAACTGCTGCGACCACTCTAACCTTATCGTGTCTCTGTGC | |
| | | CGGGCCGCCAACATAACCGCCCGGTTCTCTCACGCACAGGGGTGCACATTCTCTT | |
| | | CTGGGCTGGTGGCCGGCCACGTCTGGGCACAGATCTACATCGACGGCGTGTGGT | |
| | | ACACTGCCGACGCCACGTCTAGGCGCAACTCCCTTGGTAACATCGTGAATTGGA | |
| | | ACACCAACCACTATAACACTCTGAAGCAGTACGACCACCTGTCTTTCGGCGGCG | |
| | | GCGGTTCTGGCGGAGGTGGTTCTGGCGGCGGGGGCTCTATCGAAAACGCCGACA | |
| | | AGGCCATCAAAGACTTCCAGGATAACAAGGCACCTCACGACAAGTCAGCGGCCT | |
| | | ACGAGGCTAACTCTAAGCTGCCTAAGGACCTCCGGGACAAGAACAACCGCTTCG | |
| | | TTGAGAAGGTGTCTATCGAGAAGGCTATCGTTAGACACGACGAGCGGGTGAAGT | |
| | | CTGCTAACGACGCGATCTCAAAGCTGAACGAGAAGGACTCTATCGAAAACCGGC | |
| | | GGCTGGCCCAGCGAGAGGTGAACAAGGCCCCTATGGACGTGAAGGAGCATCTC | |
| | | CAGAAGCAGCTGGACtaatgatagaccagcctcaagaacacccgaatggagtctctaagc | |
| | | tacataataccaacttacactttacaaaatgttgtcccccaaaatgtagccattcgtatc | |
| | | tgctcctaataaaaagaaagtttcttcacattct | |
| PPa2del | swapped ssp | MRFPSIFTAVLFAASSALAMPLSGGGSGGSGSMPLNTTTEDETAQIPAEAVIGYL | SEQ ID |
| SIP- | for PPa_v2 | DLEGDEDVAVLPFSNSTNNGLLFINTTIASIAAKEEGVSAAIDIDEASSSSDLSDSSI | NO.: 77 |
| C8.4 | without SIP | SNDYLVANSGDDSVASSSASSSIAADDSDLSNNASSSNVNFENEVLSINNNEDTESEI | |
| | sites, | VKDSKNQLSSSSLQASTKTKTTLKGSGSSVYRGNPYYVTLTDSNGKVLASQKVTFNI | |
| | mru1499 = | LGKNYTRTTDSKGVASININLAKGKYNIACLYAGTENYASSKLSVALTVNLMSTKIN | |
| | ADC47349.1, | TGGSTVKKGNAYSVTLTDGNGKALSSQKVTLNILGKNYTRTTDSKGVASIAINLAA | |
| | adhesin- | GKKFTLTASYAGSANYLSSKVSATVTVQKGDTSIKPSGTSIVKGNSYSFTLVDGSGK | |
| | like protein | GLANQKVAIKISGKSYSRTTNSNGVASIAINLAAGKKYSIVCSYAGSSNYKASSSTVS | |
| | with trans- | LSVTNPSTNSKTFSIAKIEAAATNLKAYVNKNKAVPTTVSVGGTNLKISEFSYLMSK | |
| | glutaminase | AIVNLNSNNTNAITLPSGIYNGASASNSLNATVYKAQYVDLSKRVYNYIDKNKVPA | |
| | domain | AYGTVYNANGASLGNAGFNLYTFAFAKILDFHKTNKYLPNYCSFDSSVFKASNGSS | |
| | [Methano- | SSNSSSSTNSSSSTNSSSGSSNSSGSGSSTPAVTVKATSLKAASTSVIRGDDYSVTLTD | |
| | brevibacter | SSGNALANQKITFALSSSSYTRTTNSKGVASLTLNLAGGKYSITTSYAGTSAYKASKL | |
| | ruminantium | TNTVTISNSSSRFFLNDIETAAENVKTYVTKNKALPNTVTVAGTQLTLSQFSYVMAK | |
| | M1, | AIHNINASNSNYISLKSVASSNSTGDYLDTTVYRAQYMNLTNRVISFVESDKITPTFA | |
| | amino acid | TVYNSNGKSVGKAEFKLYTFAFAKILAFYKTNNYLPTYCTFQSSAIGVVPDVATNVT | |
| | | INSKINANMNQFKVGLNEKNTVSNLSAYLVGTGQSTITTNIKNVAAQLTKGLNSTAT | |
| | | KALAIYNFVRDDISYSYYSDSRKGADGTLSSGSGNCVDQASLVVALCRAAGIPARYS | |
| | | HAQGCTFSSGLVTGHVWAQILVDGVWYSADATSVRNSLGNIVNWNTNSYHSMKQ | |
| | | YAAVPFGGGGSGGGGSGGGGSIENADKAIKDFQDNKAPHDKSAAYEANSKLPKDLRDK | |
| | | NNREVEKVSIEKAIVRHDERVKSANDAISKLNEKDSIENRRLAQREVNKAPMDVKEHLQK | |
| | | QLD | |
| PPa2del | swapped ssp | cttgttctttttgcagaagctcagaataaacgctcaactttggccaccATGCGCTTCCCT | SEQ ID |
| SIP- | for PPa_v2 | TCAATCTTCACAGCCGTCCTGTTCGCGGCCTCTTCTGCCCTCGCTATGCCTTTGTCTGGC | NO.: 78 |
| C8.4 | without SIP | GGCGGCTCTGGCGGGTCCGGCTCAATGCCTCTCAACACGACCACAGAAGACGAGACGGCG | |
| | sites, | CAGATCCCTGCCGAGGCGGTTATCGGATATCTGGACCTGGAGGGAGATTTCGACGTGGC | |
| | mru1499 = | GGTGCTGCCTTTCAGCAACAGCACTAACAACGGGCTCCTGTTCATCAACACCAC | |
| | ADC47349.1, | CATCGCCTCTATTGCAGCGAAGGAGGAGGGCGTGTCTGCCGCCATAGACATCGA | |
| | adhesin- | CGAGGCCTCCTCCTCTTCTGATCTCTCTGACTCTTCTATCTCTAACGACTACTTGG | |
| | like protein | TGGCCAACTCTGGCGACGACTCCGTCGCGTCTTCTTCTGCCAGTTCTAGTATCGC | |
| | with trans- | CGCAGACGACTCAGACCTCAGTAACAACGCTTCTTCTTCTAACGTCAACTTCGAG | |
| | glutaminase | AACGAGGTCCTCTCTACAAACAACAATGAGGACACCGAGAGTGAGATCGTCAA | |
| | domain | GGACAGTAAGAACCAGCTGAGCTCTTCCAGCCTGCAAGCTAGCACGAAGACCAA | |
| | [Methano- | AACTACCCTTAAGGGCTCCGGGTCTTCTGTGTACCGGGGTAACCCTTACTACGTG | |
| | brevibacter | ACTCTGACTGACTCTAACGGCAAGGTGCTCGCCAGCCAGAAGGTCACCTTCAAC | |
| | ruminantium | ATCTTGGGGAAGAACTACACTAGAACGACCGACTCTAAGGGCGTGGCCAGTATC | |
| | M1, | AACATCAACCTTGCCAAGGGCAAGTACAACATTGCCTGCCTCTACGCGGGGACG | |
| | nucleotide | GAGAACTACGCCTCCTCTAAACTCTCAGTGGCTCTCACCGTGAACCTGATGAGC | |
| | | ACTAAGATCAATACGGGGGGTTCTACCGTCAAGAAGGGCAACGCCTACTCTGTG | |
| | | ACCCTGACAGACGGCAACGGCAAGGCATTGTCTAGCCAGAAGGTCACCCTGAAC | |
| | | ATTCTGGGGAAGAACTACACCCGGACCACCGACAGTAAGGGCGTGGCGTCAATC | |
| | | GCCATAAACCTGGCGGCCGGTAAGAAGTTCACTCTGACCGCCTCCTACGCCGGG | |
| | | TCTGCCAACTACCTGTCTTCCAAGGTGTCAGCCACCGTTACGGTGCAGAAGGGC | |
| | | GACACATCTATCAAGCCTTCTGGCACCTCAATCGTCAAGGGGAATTCATACTCTT | |
| | | TCACGCTTGTGGACGGCTCTGGGAAGGGCCTGGCCAACCAAAAGGTCGCCATCA | |
| | | AGATCAGCGGGAAGTCTTACTCCCGGACCACGAATTCAAACGGGGTGGCTTCCA | |
| | | TCGCCATCAACCTGGCCGCCGGGAAGAAGTACTCAATCGTGTGCAGTTACGCGG | |
| | | GCTCTTCTAACTACAAGGCCTCTTCAAGCACCGTGTCTCTGTCTGTGACGAACCC | |
| | | TAGTACCAACTCTAAGACATTTAGTATCGCGAAGATCGAGGCCGCAGCGACCAA | |
| | | CCTGAAGGCTTACGTGAACAAGAATAAGGCAGTGCCTACCACAGTGTCTGTGGG | |
| | | GGGCACCAACCTGAAGATATCTGAGTTCTCTTACCTGATGAGTAAGGCCATCGT | |
| | | CAACCTGAACTCTAACAACACCAACGCGATCACGCTGCCTTCTGGAATCTACAA | |
| | | CGGGGCCTCTGCCTCTAACTCTCTCAATGCCACCGTGTACAAGGCCCAGTACGTC | |
| | | GACCTGTCTAAGAGGGTCTATAACTACATCGACAAGAACAAGGTGCCTGCCGCC | |
| | | TACGGCACAGTGTACAACGCCAACGGGGCCTCTCTGGGCAACGCAGGCTTCAAC | |
| | | CTGTACACGTTCGCCTTCGCCAAGATCCTGGACTTCCACAAGACTAATAAATATT | |
| | | TGCCTAATTACTGCTCTTTCGATTCTTCAGTGTTCAAGGCCTCTAACGGCTCTTCA | |
| | | TCTTCAAACTCTTCTAGCAGTACCAACTCTTCTTCTTCTACCAACTCTAGTTCTGG | |
| | | GAGCTCCAACTCTTCTGGATCTGGCTCCTCTACCCCTGCCGTGACCGTGAAGGCC | |
| | | ACCAGTCTTAAGGCAGCTTCTACCTCTGTCATCCGGGGCGACGACTACTCAGTGA | |
| | | CCCTGACCGACAGCAGCGGGAACGCCCTGGCCAACCAGAAGATTACGTTTGCTC | |
| | | TGTCTTCTTCTTCCTACACCAGGACAACGAACTCTAAGGGGGTTGCCTCTCTGAC | |
| | | CCTCAACCTTGCGGGCGGCAAGTATTCCATCACCACGTCTTACGCCGGGACCTCA | |
| | | GCGTACAAGGCCAGTAAACTGACCAACACCGTCACCATCAGCAACTCCTCTTCT | |
| | | CGATTCTTCCTCAACGACATCGAGACCGCAGCCGAGAACGTGAAAACGTACGTC | |
| | | ACCAAGAACAAGGCGTTGCCTAACACCGTGACTGTCGCGGGGACCCAGCTGACG | |
| | | CTCTCTCAGTTCTCTTATGTGATGGCCAAGGCGATCCACAACATCAACGCGTCTA | |
| | | ACTCTAACTACATCTCCCTGAAGTCTGTGGCTTCATCTAACTCCACCGGAGACTA | |
| | | CCTGGACACCACTGTTTACCGCGCCCAGTACATGAACCTGACCAACCGAGTGAT | |
| | | CAGCTTCGTTGAGTCTGACAAGATCACCCCTACCTTCGCAACGGTCTACAACTCT | |
| | | AACGGAAAGTCTGTCGGCAAGGCCGAGTTCAAACTCTATACGTTCGCCTTCGCC | |
| | | AAGATTCTCGCGTTCTACAAGACGAACAACTACCTTCCTACCTACTGCACATTCC | |
| | | AGAGTTCTGCCATCGGCGTCGTGCCTGACGTCGCCACCAACGTGACCATCAACT | |
| | | CTAAGATCAACGCCAACATGAACCAGTTCAAGGTGGGTCTCAACGAAAAGAAC | |
| | | ACCGTCTCTAATCTTTCTGCCTACCTGGTCGGGACCGGCCAGTCTACCATCACAA | |
| | | CCAATATTAAGAATGTGGCCGCCCAGCTCACGAAGGGCCTCAACAGTACCGCCA | |
| | | CCAAGGCCCTCGCCATATACAACTTCGTCCGCGACGACATCTCTTACAGTTACTA | |
| | | CTCTGACTCTCGGAAGGGTGCCGACGGTACCCTTTCTTCTGGGTCTGGCAACTGT | |
| | | GTGGACCAGGCCTCACTGGTTGTGGCCCTGTGCCGGGCCGCGGGAATACCTGCC | |
| | | CGCTACTCTCACGCCCAGGGCTGCACCTTCTCTTCTGGGTTGGTCACCGGCCACG | |
| | | TGTGGGCCCAGATCCTGGTGGACGGCGTTTGGTACTCTGCAGACGCTACGAGCG | |
| | | TGCGGAACAGCTTGGGGAACATCGTGAACTGGAACACTAACTCTTACCACTCTA | |
| | | TGAAACAGTACGCCGCCGTGCCTTTTGGTGGAGGGGGTTCCGGAGGCGGCGGGT | |
| | | CTGGGGGCGGTGGCAGCATCGAGAACGCTGACAAGGCCATCAAGGACTTCCAG | |
| | | GACAACAAGGCACCTCACGATAAGTCTGCAGCCTACGAGGCCAACTCTAAACTC | |
| | | CCTAAGGACCTCCGCGACAAAAACAACCGCTTCGTGGAGAAGGTCAGTATCGAG | |
| | | AAGGCAATCGTTCGCCACGACGAGCGGGTGAAGTCAGCCAACGACGCGATATCT | |
| | | AAGCTGAACGAGAAGGACTCTATCGAAAACCGGCGCCTGGCTCAGAGAGAGGT | |
| | | GAACAAGGCCCCTATGGATGTGAAGGAGCATCTGCAGAAGCAGCTGGACtaatgata | |
| | | gaccagcctcaagaacaccegaatggagtctctaagctacataataccaacttacacttt | |
| | | acaaaatgttgtcccccaaaatgtagccattcgtatctgctcctaataaaaagaaagttt | |
| | | cttcacattct | |
| C9.1 | M1.WP_ | MFVFLVLLPLVSSAACGAFQTTAATVPGSSDPMWFAMDYVKENSTNDTVIISWWD | SEQ ID |
| | 012955194.1, | FGYLFQVASDHPTSFDGGSQTGDRAYWVGKSLTTSDYAQSKGILQMLATTGSNAS | NO.: 79 |
| | Methano- | MLLSEYTGSNVTAVHALDETLGKSRSEAQKILTSKYNLINDQAKAVVKQSHPSNPN | |
| | brevibacter | NVSFVLSSDMIGKAGWWSYFGSWNFDTLNSTNYQYYMANDYVPIKQNTQGNITIL | |
| | ruminantium | NESGIIYQAVVNRGKNGTNETTAQMETIWDNNRSKIDLNGTEYNPLKASNLICIENS | |
| | 83816, | YLTVNKTLNKDGNYTLYLLGSGDDYTAILMDNNLKDSVFTRLFLLGGIGQDTFELS | |
| | amino acid | NMQDGVSVWTLRDGSSNSDDAGSQGGGGSGGGGSGGGGSIENADKAIKDFQDNKAP | |
| | | HDKSAAYEANSKLPKDLRDKNNRFVEKVSIEKAIVRHDERVKSANDAISKLNEKDSIEN | |
| | | RRLAQREVNKAPMDVKEHLQKQLD | |
| C9.1 | M1.WP_ | cttgttctttttgcagaagetcagaataaacgctcaactttggccaccATGTTTGTGTTC | SEQ ID |
| | 012955194.1, | TTGGTCCTGCTCCCTCTGGTTTCAAGCGCTGCCTGTGGGGCATTCCAGACCACCGCAGCC | NO.: 80 |
| | Methano- | ACTGTGCCTGGATCCTCTGACCCTATGTGGTTCGCCATGGACTACGTCAAGGAAAACTCT | |
| | brevibacter | ACTAATGATACAGTGATCATTTCATGGTGGGACTTCGGGTATCTGTTTCAGGTGGCGTCA | |
| | ruminantium | GATCATCCTACCTCATTTGATGGCGGGTCTCAGACAGGCGATAGGGCTTACTGG | |
| | 83816, | GTGGGGAAGAGCCTGACGACATCAGACTACGCACAGTCAAAGGGGATCTTGCA | |
| | nucleotide | GATGCTGGCTACTACCGGCTCCAACGCTTCCATGCTGCTGAGCGAGTATACCGG | |
| | | AAGTAATGTGACGGCTGTGCACGCCCTTGATGAAACCCTCGGCAAGAGTCGCAG | |
| | | TGAGGCACAAAAGATTCTCACTAGCAAGTACAACCTTACCAATGACCAGGCCAA | |
| | | AGCCGTGGTCAAACAGTCTCACCCTTCCAATCCTAACAACGTCAGCTTCGTGCTG | |
| | | TCCAGCGACATGATTGGCAAAGCTGGCTGGTGGAGTTACTTCGGATCTTGGAAT | |
| | | TTCGACACCCTGAATTCCACCAATTACCAGTACTACATGGCCAACGACTATGTGC | |
| | | CTATAAAGCAAAACACCCAGGGTAATATCACGATCCTCAACGAGAGCGGAATCA | |
| | | TTTACCAGGCCGTCGTTAATAGGGGGAAAAACGGCACAAACGAAACGACCGCC | |
| | | CAAATGGAAACCATCIGGGATAACAACCGGAGCAAGATTGACCTCAATGGGAC | |
| | | AGAGTATAACCCTTTGAAAGCCAGCAACCTGATTTGCATCGAAAACAGTTACCT | |
| | | CACAGTGAATAAAACACTCAACAAGGATGGTAATTATACTCTGTATCTGTTGGG | |
| | | CTCTGGTGATGACTACACAGCAATCCTGATGGACAATAACCTCAAGGACTCTGT | |
| | | GTTCACTCGCCTGTTTCTTCTCGGCGGGATAGGGCAAGACACCTTTGAGTTGTCC | |
| | | AACATGCAGGATGGCGTTTCCGTGTGGACTCTGAGGGACGGCAGTTCTAACAGC | |
| | | GACGACGCCGGTAGCCAGGGTGGCGGCGGTTCCGGCGGAGGAGGAAGTGGAGG | |
| | | GGGAGGATCCATTGAGAATGCGGATAAAGCCATCAAGGATTTCCAAGACAACA | |
| | | AGGCACCTCACGACAAGTCTGCCGCATATGAAGCGAACTCCAAACTTCCTAAGG | |
| | | ACCTGAGAGACAAAAATAATCGGTTTGTCGAGAAGGTTTCCATCGAGAAGGCTA | |
| | | TAGTGAGACATGATGAACGAGTTAAAAGCGCCAACGATGCTATCAGTAAGCTGA | |
| | | ACGAGAAGGATAGCATCGAGAATAGACGCCTTGCGCAGCGGGAGGTCAACAAA | |
| | | GCCCCTATGGATGTGAAGGAGCACCTGCAGAAACAGCTGGACtaatgatagaccagcct | |
| | | caagaacacccgaatggagtctctaagctacataataccaacttacactttacaaaatgt | |
| | | tgtcccccaaaatgtagccattcgtatctgctcctaataaaaagaaagtttcttcacatt | |
| | | c | |
| C9.2 | M2.WP_ | MFVFLVLLPLVSSAAYQTANQVVPGTSDAMWDSMVWINENTADNTVVASWWDF | SEQ ID |
| | 011954062.1, | GYLFEIAADRQVIFDGGSQSGNSRAFWLGQAMTTDNMDLSAGIFRMLGTSGENATN | NO.: 81 |
| | Methano- | TLTDYTGSPGKATDILIDILPKNAQDAKNALINTYGLTTEQANTIIPLTHPDNPRPVIF | |
| | brevibacter | VASSDMLQKAGWWSYFGAWDFDKQNSTNFNYYVPSQQVTVEPGSTGRLALINESG | |
| | smithii | LEYDAVITRGTGNNTTTGHTEAVYSNNGSLLKINGSEFNPLNVSRIMVIEGNQLVKN | |
| | 2173, amino | ESIAGAPSDSNYTLFLMGENNVYTPIIMHNKLADSMFTRLYLLGGMGQNVFSMVHM | |
| | acid | ENGVSLWQVNYNNTAAGGTSTPAASGATDGNIGTTSPDRAGGGGSGGGGSGGGGSI | |
| | | ENADKAIKDFQDNKAPHDKSAAYEANSKLPKDLRDKNNRFVEKVSIEKAIVRHDERVKSA | |
| | | NDAISKLNEKDSIENRRLAQREVNKAPMDVKEHLQKQLD | |
| C9.2 | M2.WP_ | cttgttctttttgcagaagctcagaataaacgctcaactttggccaccATGTTTGTGTT | SEQ ID |
| | 011954062.1, | CCTGGTCCTTCTGCCTCTCGTCAGTAGCGCTGCCTATCAAACTGCCAATCAGGTGGTTCC | NO.: 82 |
| | Methano- | TGGCACCTCTGACGCTATGTGGGATTCTATGGTGTGGATTAACGAGAACACGGCCGACA | |
| | brevibacter | ACACGGTGGTGGCCTCATGGTGGGATTTCGGGTACCTGTTTGAGATTGCCGCCGACCGAC | |
| | smithii | AGGTTATTTTCGATGGGGGCTCCCAGAGCGGAAATTCCAGGGCTTTTTGGCTCGGG | |
| | 2173, | CAGGCTATGACCACGGACAATATGGACCTCTCAGCCGGCATTTTCCGGATGCTC | |
| | nucleotide | GGTACTTCTGGCGAAAACGCCACTAACACCCTGACAGATTATACCGGCTCACCT | |
| | | GGTAAGGCGACTGATATACTTATCGACATTCTGCCTAAAAATGCACAAGACGCC | |
| | | AAGAATGCCTTGATCAACACATATGGCCTGACCACAGAGCAGGCAAATACAATC | |
| | | ATTCCTTTGACACACCCTGATAATCCTAGGCCTGTCATCTTCGTCGCCAGCTCCG | |
| | | ACATGCTTCAAAAGGCAGGCTGGTGGTCATACTTCGGCGCTTGGGATTTTGACA | |
| | | AACAAAATAGTACGAATTTTAATTACTATGTGCCTTCTCAGCAGGTTACCGTGGA | |
| | | GCCTGGAAGCACGGGGCGCCTGGCACTGATAAATGAAAGTGGCCTCGAGTATGA | |
| | | TGCAGTCATCACTAGGGGTACCGGGAACAACACCACTACCGGCCATACTGAGGC | |
| | | AGTGTACTCCAACAACGGCAGCCTGCTGAAAATCAACGGGTCCGAATTTAACCC | |
| | | TCTGAACGTCTCCCGAATAATGGTGATTGAGGGTAACCAGCTGGTGAAGAACGA | |
| | | AAGCATCGCGGGAGCACCTAGCGACAGTAATTATACACTGTTCTTGATGGGTGA | |
| | | AAACAACGTGTACACACCTATCATCATGCATAACAAACTTGCGGACAGCATGTT | |
| | | CACCCGCCTGTACCTGTTGGGGGGATGGGACAGAACGTTTTCTCTATGGTCCAC | |
| | | ATGGAAAACGGAGTCTCACTGTGGCAGGTGAACTACAATAATACAGCCGCCGGT | |
| | | GGCACCTCCACACCTGCGGCTTCCGGAGCAACCGATGGCAATATTGGCACCACT | |
| | | TCACCTGATCGCGCTGGTGGGGGAGGCTCTGGGGGAGGAGGATCCGGAGGGGG | |
| | | CGGGTCCATAGAGAATGCTGATAAGGCTATTAAGGACTTTCAGGACAACAAAGC | |
| | | CCCTCATGACAAGTCTGCTGCATACGAAGCGAACAGCAAGCTCCCTAAGGATCT | |
| | | TAGAGACAAGAATAACCGGTTCGTGGAAAAAGTTAGTATCGAAAAGGCCATCGT | |
| | | GCGCCACGACGAGCGGGTCAAGAGCGCCAACGACGCTATCAGCAAACTCAATG | |
| | | AGAAGGACAGTATCGAGAACAGACGGCTCGCCCAGAGAGAGGTGAACAAGGCC | |
| | | CCTATGGATGTGAAAGAGCACCTGCAGAAACAGTTGGACtaatgatagaccagcctcaag | |
| | | aacaccegaatggagtctctaagctacataataccaacttacactttacaaaatgtgtcc | |
| | | cccaaaatgtagccattcgtatctgctcctaataaaaagaaagtttcttcacattct | |
| C9.3A | M3.WP_ | MFVFLVLLPLVSSAAYITSENVIPGTSDPMWNSMEWINEHTSNDTVITSWWDFGYL | SEQ ID |
| | 042693613.1, | FEIAADRQVTFDGGSQTGSRAFWLGQAMTTDNLELSAGIFRMLDTSGERAVDALIN | NO.: 83 |
| | Methano- | YTGDTGKTTKILIDILPMTKQNAQKTLIDKYDLSTDQAKEIVGYTHPSKPRPVIFVAS | |
| | brevibacter | SDMLQKAGWWSYFGAWDFKNQSSENYNYYVPTEQVKVESGSSGKLSLIVDGGMT | |
| | oralis | VNAVITRGTGNNTTSAYTEAVYTHNGSQIYVNKTVYNPLNISNLIVVEDGYLMKNE | |
| | 66851, | SVGNVKDANYTLFLMGEKNKYTPILISNKLANSMFTKLYLLGGAGQDIFTNVHTEE | |
| | amino acid | GVMLWQVNFNNTVAGKGGGGSGGGGSGGGGSIENADKAIKDFQDNKAPHDKSAAYE | |
| | | ANSKLPKDLRDKNNRFVEKVSIEKAIVRHDERVKSANDAISKLNEKDSIENRRLAQREV | |
| | | NKAPMDVKEHLQKQLD | |
| C9.3A | M3.WP_ | cttgttctttttgcagaagctcagaataaacgctcaactttggccaccATGTTCGTCTTT | SEQ ID |
| | 042693613.1, | CTGGTGCTGCTTCCTTTGGTGAGTAGCGCCGCTTATATCACTTCTGAGAACGTTATTCCT | NO.: 84 |
| | Methano- | GGTACATCTGACCCTATGTGGAATTCCATGGAGTGGATCAACGAACATACTAGTAACGAC | |
| | brevibacter | ACTGTTATCACCTCTTGGTGGGACTTTGGGTACCTCTTCGAGATCGCGGCAGACAGACA | |
| | oralis | AGTGACCTTCGATGGCGGGAGCCAAACAGGGAGCAGGGCATTCTGGTTGGGCCA | |
| | 66851, | GGCCATGACTACTGATAACCTCGAGCTCAGCGCAGGTATCTTTCGCATGCTCGAT | |
| | nucleotide | ACGAGTGGGGAACGGGCCGTGGACGCCCTGATAAACTATACGGGAGACACCGG | |
| | | CAAAACAACCAAGATCTTGATTGACATCCTGCCTATGACGAAACAGAACGCCCA | |
| | | GAAAACTCTGATTGATAAGTACGATCTGAGTACAGATCAGGCCAAAGAGATTGT | |
| | | CGGGTACACACATCCTAGCAAGCCTAGGCCTGTTATATTTGTCGCTTCTAGCGAC | |
| | | ATGCTGCAGAAGGCGGGTTGGTGGTCATACTTTGGAGCCTGGGATTTCAAAAAC | |
| | | CAGAGTTCAGAAAATTACAACTACTATGTGCCTACAGAACAAGTCAAGGTGGAG | |
| | | TCCGGATCATCTGGCAAGCTTTCCCTGATCGTTGATGGCGGCATGACCGTGAACG | |
| | | CTGTGATCACCCGCGGCACCGGCAACAATACCACCAGTGCCTACACTGAGGCTG | |
| | | TGTATACCCATAATGGGTCCCAGATCTATGTGAATAAGACCGTTTATAATCCTCT | |
| | | GAATATCTCAAATCTTATTGTCGTGGAGGACGGCTACCTGATGAAGAATGAGAG | |
| | | CGTTGGTAACGTGAAGGACGCTAATTACACCCTGTTCTTGATGGGCGAAAAGAA | |
| | | CAAATATACGCCTATACTGATCTCCAACAAGCTGGCCAACTCTATGTTCACCAAG | |
| | | CTGTACCTCCTCGGAGGAGCTGGCCAGGACATTTTCACAAATGTCCACACAGAG | |
| | | GAAGGAGTGATGCTCTGGCAGGTGAACTTCAACAACACGGTGGCAGGAAAAGG | |
| | | GGGCGGAGGCTCTGGTGGAGGGGGGAGCGGCGGTGGGGGATCAATTGAGAACG | |
| | | CCGACAAGGCGATTAAGGATTTTCAGGATAATAAAGCACCTCACGATAAATCCG | |
| | | CTGCTTACGAGGCCAACAGCAAGCTTCCTAAAGACCTCCGAGATAAAAACAATC | |
| | | GGTTTGTCGAAAAGGTCTCCATCGAAAAAGCCATCGTGCGGCACGACGAGAGAG | |
| | | TCAAGTCCGCGAACGACGCCATAAGCAAGCTGAATGAGAAAGACTCCATTGAA | |
| | | AATCGCAGACTGGCACAGAGGGAAGTGAACAAGGCCCCTATGGATGTGAAGGA | |
| | | GCACCTTCAGAAGCAATTGGACtaatgatagaccagcctcaagaacacccgaatggagtc | |
| | | tctaagctacataataccaacttacactttacaaaatgttgtcccccaaaatgtagccat | |
| | | tcgtatctgctcctaataaaaagaaagtttcttcacattct | |
| C9.3B | M3.WP_ | MFVFLVLLPLVSSAASLIDTIDTINRYDHYTDTTMELINNEDNDTIVIYSIEHEFIRFH | SEQ ID |
| | 042693055.1, | EQLKGTNEFLVPNKNRLFNPNDYNLSVMPIDKIVNENPHKDIYLILRIQKSNENFGEG | NO.: 85 |
| | Methano- | IKVNKSAYIGPSFVKLEKINGGGGSGGGGSGGGGSIENADKAIKDFQDNKAPHDKSAA | |
| | brevibacter | YEANSKLPKDLRDKNNRFVEKVSIEKAIVRHDERVKSANDAISKLNEKDSIENRRLAQRE | |
| | oralis | VNKAPMDVKEHLQKQLD | |
| | 66851, | | |
| | amino acid | | |
| C9.3B | M3.WP_ | cttgttctttttgcagaagctcagaataaacgctcaactttggccaccATGTTTGTGTTT | SEQ ID |
| | 204693055.1, | CTGGTGCTTCTGCCTCTTGTGTCAAGTGCGGCCTCTCTCATTGACACAATCGATACAATT | NO.: 86 |
| | Methano- | AACCGGTATGACCACTACACTGACACCACCATGGAGCTTATTAACAATTTCGATAATGAC | |
| | brevibacter | ACCATAGTGATCTACTCCATCGAACACGAATTTATCAGATTCCATGAGCAACTCAAGG | |
| | oralis | GCACGAATGAGTTCCTGGTTCCTAATAAGAACAGGTTGTTTAACCCTAACGATTA | |
| | 66851, | TAACCTGTCAGTTATGCCTATCGACAAAATCGTCAACGAGAACCCTCACAAAGA | |
| | nucleotide | CATCTATCTCATTCTGCGCATACAGAAATCTAACGAAAACTTCGGGGAGGGGAT | |
| | | AAAAGTCAATAAGTCTGCTTACATTGGCCCTAGCTTTGTCAAATTGGAGAAGAT | |
| | | CAACGGTGGGGGAGGCAGTGGAGGCGGGGGATCCGGTGGCGGAGGCAGCATTG | |
| | | AGAATGCTGACAAGGCCATCAAGGACTTCCAGGACAATAAAGCCCCTCATGACA | |
| | | AAAGCGCGGCCTACGAAGCAAACTCCAAGCTGCCTAAGGATCTGCGAGATAAG | |
| | | AACAATAGATTCGTCGAAAAGGTGAGCATCGAGAAAGCAATCGTGCGGCATGA | |
| | | TGAACGGGTGAAGAGCGCTAATGATGCCATCTCCAAACTCAATGAGAAGGATAG | |
| | | TATTGAGAACCGCCGCCTGGCACAGAGGGAGGTTAACAAGGCCCCTATGGACGT | |
| | | GAAGGAACACTTGCAGAAGCAGCTGGACtaatgatagaccagcctcaagaacacccgaat | |
| | | ggagtctctaagctacataataccaacttacactttacaaaatgttgtcccccaaaatgt | |
| | | agccattcgtatctgctcctaataaaaagaaagtttcttcacattct | |
| C9.4A | M4.WP_ | MFVFLVLLPLVSSAAYFMGYYTTYTKNDWRGFSGQLSSITNEGDYVVVMPDYITK | SEQ ID |
| | 042706503.1, | PLNYYYSNSTDGTIEVGASSAATLEQINAKRLNSTGNPAAYYIITWDISAANPSGDAL | NO.: 87 |
| | Methanomic | DWISKNAQFIGQNMGIYVFRSAGGGGSGGGGSGGGGSIENADKAIKDFQDNKAPHDK | |
| | robium | SAAYEANSKLPKDLRDKNNRFVEKVSIEKAIVRHDERVKSANDAISKLNEKDSIENRRL | |
| | mobile | AQREVNKAPMDVKEHLQKQLD | |
| | 2205, amino | | |
| | acid | | |
| C9.4A | M4.WP_ | cttgttctttttgcagaagctcagaataaacgctcaactttggccaccATGTTCGTGTTC | SEQ ID |
| | 042706503.1, | CTGGTGCTTCTGCCTTTGGTCAGCTCCGCCGCCTATTTCATGGGTTACTACACCACCTAC | NO.: 88 |
| | Methanomic | ACCAAAAACGATTGGCGCGGGTTCAGTGGGCAACTGTCATCCATCACAAACGAGGGAGAC | |
| | robium | TACGTGGTGGTTATGCCTGACTATATCACTAAGCCTCTTAACTATTATTATTCTAACAG | |
| | mobile | CACAGACGGCACGATTGAAGTGGGCGCTTCTAGCGCAGCCACTCTCGAGCAGAT | |
| | 2205, | CAATGCAAAAAGGCTCAACTCTACAGGGAATCCTGCCGCGTACTACATCATTAC | |
| | nucleotide | CTGGGACATTTCCGCCGCTAACCCTAGTGGGGATGCCCTCGATTGGATCAGTAA | |
| | | GAATGCACAGTTTATAGGACAAAATATGGGCATTTACGTGTTCCGGAGCGCCGG | |
| | | AGGGGGTGGTAGCGGCGGCGGAGGTTCCGGCGGCGGAGGCTCAATCGAGAATG | |
| | | CTGATAAGGCGATCAAGGACTTTCAGGACAATAAAGCTCCTCATGATAAGTCAG | |
| | | CAGCCTACGAGGCCAACTCCAAACTGCCTAAGGATTTGAGGGACAAGAACAATC | |
| | | GCTTTGTCGAAAAGGTGAGCATTGAGAAGGCCATAGTCAGACACGATGAACGG | |
| | | GTTAAATCCGCAAACGACGCGATAAGTAAACTGAATGAAAAGGATTCTATCGAG | |
| | | AACCGAAGACTCGCTCAGCGGGAAGTGAACAAAGCTCCTATGGACGTCAAGGA | |
| | | GCACCTGCAGAAGCAGCTGGACtaatgatagaccagcctcaagaacacccgaatggagtc | |
| | | tctaagctacataataccaacttacactttacaaaatgttgtcccccaaaatgtagccat | |
| | | tcgtatctgctcctaataaaaagaaagtttcttcacattct | |
| C9.4B | M4.WP_ | MFVFLVLLPLVSSAAHTSFTTGDIADPIVQVQNSEELREVFPYMDNTTKIAVSNYVR | SEQ ID |
| | 052358913.1, | WPFVWYYQGDYPKRISYYSDPGYNKDIGPENYDLIIVHDGEKVDSIEGFEKHTYKK | NO.: 89 |
| | Methanomic | CYWIDVNTLIQSYGKPIDIFNANDRALLVEDLKRVIHYYFTRDAGLGSINIDVFVKKD | |
| | robium | RAGGGGSGGGGSGGGGSIENADKAIKDFQDNKAPHDKSAAYEANSKLPKDLRDKNNRF | |
| | mobile | VEKVSIEKAIVRHDERVKSANDAISKLNEKDSIENRRLAQREVNKAPMDVKEHLQKQLD | |
| | 2205, amino | | |
| | acid | | |
| C9.4B | M4.WP_ | cttgttctttttgcagaagctcagaataaacgctcaactttggccaccATGTTTGTCTTC | SEQ ID |
| | 052358913.1, | CTGGTTCTGCTGCCTTTGGTGAGCAGCGCCGCTCACACCTCCTTTACCACAGGAGACATA | NO.: 90 |
| | Methanomic | GCCGACCCTATAGTGCAGGTCCAGAACTCCGAGGAGCTGAGAGAGGTGTTTCCTTACATG | |
| | robium | GATAACACTACCAAGATTGCAGTGTCCAATTACGTGCGGTGGCCTTTCGTGTGGTACT | |
| | mobile | ATCAAGGAGACTATCCTAAACGAATCTCTTACTATTCCGACCCTGGCTACAATAA | |
| | 2205, | GGATATCGGGCCTGAAAACTATGATCTCATTATCGTCCATGACGGGGAAAAAGT | |
| | nucleotide | GGACAGCATCGAGGGGTTTGAGAAACATACGTACAAGAAGTGCTATTGGATTGA | |
| | | CGTCAACACTCTCATCCAGAGTTACGGCAAACCTATTGACATCTTCAACGCAAA | |
| | | CGACAGGGCCCTGCTGGTGGAAGATCTTAAACGGGTGATTCACTACTACTTCAC | |
| | | AAGGGACGCAGGGCTCGGATCAATCAACATCGACGTTTTCGTCAAAAAGGACCG | |
| | | GGCTGGAGGCGGTGGCTCTGGCGGTGGAGGGTCAGGCGGTGGCGGCAGCATCG | |
| | | AGAATGCTGATAAGGCCATTAAGGACTTCCAGGACAATAAAGCCCCTCACGATA | |
| | | AGAGCGCTGCGTATGAAGCGAACTCTAAGCTCCCTAAAGATCTGCGCGACAAGA | |
| | | ACAACAGATTTGTGGAAAAGGTCTCCATCGAGAAAGCAATAGTGAGGCATGATG | |
| | | AGCGAGTGAAGAGTGCCAACGATGCCATTTCAAAGCTTAATGAAAAAGATAGTA | |
| | | TCGAGAATCGCCGCTTGGCTCAAAGAGAGGTTAATAAGGCCCCTATGGATGTTA | |
| | | AGGAACACCTTCAGAAGCAGCTGGACtaatgatagaccagcctcaagaacacccgaatgg | |
| | | agtctctaagctacataataccaacttacactttacaaaatgtgtcccccaaaatgtagc | |
| | | cattcgtatctgctcctaataaaaagaaagtttcttcacattct | |
| C9.4C | M4.WP_ | MFVFLVLLPLVSSAAFSATHDIALGNAMKQGGMTPDWEEALSWMGENTPDTGID | SEQ ID |
| | 042705279.1, | YYAIYTRDSYENPEEAYGIMTWWDYGHWITFISKRAPNSNPFQRGVAGPNGAAAYF | NO.: 91 |
| | Methanomic | IQQDEAASNAILDNLDTRYVITDAEMDTAKFWAMCTWYNSSAGTGQYQQTFAMIN | |
| | robium | PDNSISTGNLYSEKYYNTMISRLHNLDGSMVEPTSVYYVEYVDGSLYSIPIPIVTDAR | |
| | mobile | EMSYADAKAAVDAYNANARAGYGAGIYSLSLTAPSGEVPALEHYRLIHESPSSPFSA | |
| | 2205, amino | ATTLKYVKVFEYVKGAKVKGDGIIEIDLESDQGRKFTYSQRSTDGYFTVPYSTTGNN | |
| | acid | YGTKVLGDYRIRGTQQTFKVSEDAVMNGLSVNGGGGSGGGGSGGGGSIENADKAIK | |
| | | DFQDNKAPHDKSAAYEANSKLPKDLRDKNNRFVEKVSIEKAIVRHDERVKSANDAISKL | |
| | | NEKDSIENRRLAQREVNKAPMDVKEHLQKQLD | |
| C9.4C | M4.WP_ | cttgttctttttgcagaagctcagaataaacgctcaactttggccaccATGTTTGTGTTC | SEQ ID |
| | 042705279.1, | CTCGTGCTCTTGCCTCTGGTCTCTTCTGCAGCTTTTAGCGCAACACACGACATTGCCCTG | NO.: 92 |
| | Methanomic | GGCAATGCTATGAAACAGGGAGGCATGACACCTGACTGGGAGGAGGCCCTCTCCTGGATG | |
| | robium | GGGGAGAACACTCCTGATACGGGAATCGATTACTACGCCATATACACCAGGGACTCC | |
| | mobile | TACGAAAACCCTGAAGAGGCCTATGGGATTATGACATGGTGGGACTACGGTCAC | |
| | 2205, | TGGATCACTTTCATCAGCAAACGGGCACCTAACTCTAACCCTTTTCAAAGAGGC | |
| | nucleotide | GTCGCGGGACCTAATGGCGCTGCCGCTTACTTTATCCAGCAAGATGAAGCTGCC | |
| | | AGCAACGCCATCCTGGACAACCTTGACACTAGATATGTGATTACTGATGCGGAG | |
| | | ATGGACACGGCCAAGTTCTGGGCCATGTGCACCTGGTACAATAGCAGTGCCGGA | |
| | | ACAGGCCAGTATCAGCAGACCTTTGCCATGATAAACCCTGATAATAGCATCAGT | |
| | | ACGGGGAATTTGTATTCCGAGAAATATTACAACACGATGATAAGCCGCCTGCAT | |
| | | AACCTGGATGGTAGCATGGTCGAGCCTACAAGTGTTTACTATGTGGAGTACGTG | |
| | | GATGGCTCCCTGTACAGTATCCCTATCCCTATCGTCACAGATGCTAGGGAGATGT | |
| | | CATATGCGGATGCCAAAGCCGCTGTGGACGCTTACAACGCCAACGCGAGGGCCG | |
| | | GATATGGAGCCGGTATCTATAGCCTGTCTCTGACCGCACCTTCCGGCGAAGTTCC | |
| | | TGCACTTGAACATTATCGGCTCATTCATGAGAGCCCTTCCTCACCTTTCTCAGCG | |
| | | GCAACCACCCTGAAATATGTTAAAGTGTTCGAGTACGTTAAGGGCGCCAAGGTG | |
| | | AAGGGGGATGGGATCATTGAGATTGACCTTGAAAGTGACCAGGGTCGAAAGTTT | |
| | | ACCTATTCTCAGAGATCCACTGACGGCTACTTCACCGTGCCTTACTCAACCACAG | |
| | | GGAACAATTACGGCACTAAGGTGTTGGGCGACTACAGGATACGAGGGACCCAA | |
| | | CAGACCTTCAAGGTGTCTGAAGATGCCGTCATGAACGGCCTGTCCGTCAATGGA | |
| | | GGGGGCGGAAGTGGGGGAGGCGGGTCTGGAGGCGGTGGTAGTATTGAAAACGC | |
| | | TGACAAGGCCATTAAGGACTTCCAGGATAATAAGGCTCCTCACGATAAATCCGC | |
| | | GGCATACGAAGCTAATTCAAAACTGCCTAAGGACCTCCGCGACAAGAATAACCG | |
| | | CTTCGTGGAGAAAGTGAGCATCGAGAAAGCTATTGTGCGGCACGACGAGAGAG | |
| | | TTAAATCAGCAAATGACGCAATCTCCAAGCTGAACGAGAAGGACAGCATCGAG | |
| | | AACCGGCGGCTTGCCCAGCGCGAAGTCAATAAGGCCCCTATGGACGTGAAGGA | |
| | | ACACCTGCAGAAGCAGCTCGATtaatgatagaccagcctcaagaacacccgaatggagtc | |
| | | tctaagctacataataccaacttacactttacaaaatgttgtcccccaaaatgtagccat | |
| | | tcgtatctgctcctaataaaaagaaagtttcttcacattct | |
| C9.5 | M5.WP_ | MFVFLVLLPLVSSAAADHLASSQAVGSINDDMYNTLTWIKANTSQDTVLASWWD | SEQ ID |
| | 011405971.1, | FGHLFTAVADRQVVFDGGSQNNMRAYWIGNALTSTDEAKSAGILRMLANSGEDAS | NO.: 93 |
| | Methano- | NTLDLYTNNTEKTVEILNAILPMDRTEANSALTGTYGLSQQEANSVLDLTHPAKVKP | |
| | asphera | VNLILSSDMLSKAAWWSYFGSWDFKNQNSTHYSYYPSQSNIENINGREFTLGMDNG | |
| | stadtmanae | VIGVSSPTNETNGSTMTFAYVDQSKLNKSLNMSTLEDKQRMSKELSDGTGNTLLKP | |
| | 2317, amino | HKLIVVENNQLTEKIVNNNSNMSIMAIHQNDGSYFTVLFDSHLEESLFTKLYLKSGL | |
| | acid | NVTRFNMTHSEPGISVWDVSEYANTTANSTTNSSTNGTNVQTNTSTGGGGSGGGGS | |
| | | GGGGSIENADKAIKDFQDNKAPHDKSAAYEANSKLPKDLRDKNNRFVEKVSIEKAIVRHD | |
| | | ERVKSANDAISKLNEKDSIENRRLAQREVNKAPMDVKEHLQKQLD | |
| C9.5 | M5.WP_ | cttgttctttttgcagaagctcagaataaacgctcaactttggccaccATGTTTGTGTTC | SEQ ID |
| | 011405971.1, | CTGGTGTTGCTTCCTCTGGTGAGTAGCGCCGCGGCTGATCACCTGGCCTCCTCTCAGGCG | NO.: 94 |
| | Methano- | GTCGGAAGTACAAATGACGACATGTACAATACCCTTACTTGGATCAAGGCAAATACTAGT | |
| | asphera | CAGGACACGGTCCTCGCCAGTTGGTGGGACTTTGGTCACCTCTTCACTGCAGTCGCCG | |
| | stadtmanae | ACAGACAGGTGGTTTTTGATGGCGGTTCACAGAACAACATGCGCGCTTACTGGA | |
| | 2317, | TCGGCAACGCACTGACGTCTACAGATGAGGCTAAGAGCGCTGGTATCCTTAGAA | |
| | nucleotide | TGTTGGCAAATTCCGGCGAGGACGCCTCAAATACATTGGATCTGTACACAAACA | |
| | | ATACCGAAAAGACGGTCGAAATTCTGAACGCCATCTTGCCTATGGATCGCACAG | |
| | | AGGCCAATTCTGCCCTGACCGGTACATACGGACTCTCCCAGCAAGAAGCAAATA | |
| | | GCGTTCTGGATCTCACCCATCCTGCTAAGGTTAAACCTGTGAACCTCATTCTGTC | |
| | | CTCTGATATGCTGTCAAAAGCAGCTTGGTGGAGCTATTTCGGTAGCTGGGACTTC | |
| | | AAGAACCAGAACAGCACTCATTATTCTTACTATCCTTCACAGTCAAACATAGAG | |
| | | AACATCAACGGCAGGGAGTTCACCCTTGGGATGGACAATGGCGTGATTGGCGTG | |
| | | TCCAGTCCTACCAATGAAACCAACGGAAGCACCATGACCTTTGCCTACGTTGAC | |
| | | CAGTCCAAGTTGAATAAGTCCCTGAACATGTCCACCCTCGAGGACAAGCAGCGG | |
| | | ATGTCAAAAGAGTTGAGTGACGGGACAGGAAACACGCTGCTGAAGCCTCACAA | |
| | | ACTGATTGTCGTGGAGAATAACCAACTTACCGAAAAGATCGTGAACAACAACAG | |
| | | CAACATGAGCATCATGGCGATTCATCAGAATGATGGAAGCTATTTCACAGTGCT | |
| | | GTTCGACTCCCACCTGGAGGAGTCCCTGTTTACTAAGCTGTATCTCAAGTCCGGA | |
| | | CTCAATGTGACCCGGTTTAACATGACCCACTCTGAGCCTGGGATTAGCGTCTGGG | |
| | | ATGTGAGTGAGTACGCTAACACTACTGCCAATAGCACCACTAATTCCTCTACTAA | |
| | | TGGGACAAACGTGCAAACAAACACGTCAACCGGGGGCGGCGGGAGTGGGGGCG | |
| | | GCGGAAGCGGAGGCGGCGGGAGTATCGAAAACGCCGACAAAGCCATCAAGGAC | |
| | | TTCCAGGACAATAAGGCCCCTCACGATAAGTCTGCTGCATACGAAGCGAACTCC | |
| | | AAGCTGCCTAAGGATCTGCGGGATAAAAACAACAGATTCGTCGAGAAGGTCTCT | |
| | | ATCGAAAAAGCTATTGTGAGGCATGACGAGCGAGTTAAAAGCGCCAACGACGC | |
| | | CATATCTAAACTCAATGAAAAAGATAGCATAGAGAACCGCAGGCTTGCCCAGCG | |
| | | AGAGGTGAATAAAGCGCCTATGGACGTGAAAGAACACCTCCAGAAGCAACTGG | |
| | | ACtaatgatagaccagcctcaagaacacccgaatggagtctctaagctacataataccaa | |
| | | cttacactttacaaaatgttgtcccccaaaatgtagccattcgtatctgctcctaataaa | |
| | | aagaaagtttcttcacattct | |
| primer | T7- | gaattTAATACGACTCACTATAAGGcttgttctttttgcagaagc | SEQ ID |
| | AGG_fwd | | NO.: 95 |
| primer | 120pA_rev | TTTTTTTTTTTTTTTTTTTTTTTTTTTTTTTTTTTTTTTTTTTTTTTTTTTTTTTTTTTT | SEQ ID |
| | | TTTTTTTTTTTTTTTTTTTTTTTTTTTTTTTTTTTTTTTTTTTTTTTTTTTTTTTTTTTT | NO.: 96 |
| | | agaatgtgaagaaactttctttttattag | |
| C7.B.1 | MBQ2354108.1/ | MFVFLVLLPLVSSAAAKTVFITSDNIIDTDTDLQMLNSIKNYIEEISGGELQVIVDNQ | SEQ ID |
| | Methano- | APSPGEGWRAIEVTSDVSICLAACDAGNFIQLAGASTNSDKQIILVNTGDYDLDNHT | NO.: 97 |
| | brevibacter | NFLRRAWDDNYSDAYLAGLRDPGTFLKNSGVYYIQPVKEFPNNANEGYIDRYDDE | |
| | sp. | MNKQIAQEIVDIVNGHGNDTRIFSDELVSKNIVNPGVMAKSSQALINSGDNEMKGTY | |
| | adhesin | GNYTAAQLLYQTSSYLNGNGLDVPKTFSEPDNPMGISFLTRDSYSIYDYFRMGGIVR | |
| | variant, | EYMDQNGKAPDSIEYEGAHIGYYDLLYNFAKITQNHTDAKHMGFDSEYHFDKVND | |
| | amino acid | SILLHIFPFVLILLVLFVAYRFYKRLRRYGGGGSGGGGSGGGGSIENADKAIKDFQDNK | |
| | | APHDKSAAYEANSKLPKDLRDKNNRFVEKVSIEKAIVRHDERVKSANDAISKLNEKDSIE | |
| | | NRRLAQREVNKAPMDVKEHLQKQLD | |
| C7.B.2 | WP_ | MFVFLVLLPLVSSAATKTVFITTDNIVDHDFDVGLITAIKNYVQELSGGELQVVIDN | SEQ ID |
| | 292606916.1/ | QAPAAGEGYRSIEVTSDASIDLAASDAGNYIQLANYSAHSNKQIVFVNIGDYDLDNS | NO.: 98 |
| | Methano- | SNYLRRAWDDNYTNETIAGVHDPGTLLRNSGIFYVQPAKEFPDKYRNGILDNYDDE | |
| | brevibacter | MAKQIAQEIVGIINTHDNDTKVFSDDLVVKNKISPAGMANASKELLNSGDKEFNGTF | |
| | sp. adhesin | GAYTAPQLLYQTSSYLNGNGLDIPKTFKEPENPMGVSIFAKGSYSISDYFKMGGIVR | |
| | variant, | NYMDEHGQAPDSIEYEGAQISYYDLLYNFAKITQNHTDSAHMGFENEYQFEKVNSS | |
| | amino acid | FLLDVFPFILVLFILFLAYLVYKRIRRGGGGSGGGGSGGGGSIENADKAIKDFQDNKAP | |
| | | HDKSAAYEANSKLPKDLRDKNNRFVEKVSIEKAIVRHDERVKSANDAISKLNEKDSIENR | |
| | | RLAQREVNKAPMDVKEHLQKQLD | |
| C7.B.3 | WP_ | MFVFLVLLPLVSSAATKTVFITTDNIVDHDFDVGLITAIKNYVQELSGGELQVVIDN | SEQ ID |
| | 292880253.1/ | QAPAAGEGYRSIEVTSDASIDLAASDAGNYIQLANYSAHSNKQIVFVNIGDYDLDNS | NO.: 99 |
| | Methano- | SNYLRRAWDDNYTNETIAGVHDPGTLLRNSGIFYVQPAKEFPDKYKNGILDNYDDE | |
| | brevibacter | MAKQIAQEIVGIINTHDNDTKVFSEDLVVKNKISPAGMANASKELLNSGDKEFNGTF | |
| | sp. adhesin | GAYTAPQLLYQTSSYLNGNGLDIPKTFKEPENPMGVSIFAKGSYSISDYFKMGGIVR | |
| | variant, | NYMDEHGQAPDSIEYEGAQISYYDLLYNFAKITQNHTDSAHMGFENEYQFEKVNSS | |
| | amino acid | FLLDVFPFILVLFILFLAYLVYKRIRRGGGGSGGGGSGGGGSIENADKAIKDFQDNKAP | |
| | | HDKSAAYEANSKLPKDLRDKNNRFVEKVSIEKAIVRHDERVKSANDAISKLNEKDSIENR | |
| | | RLAQREVNKAPMDVKEHLQKQLD | |
| C7.B.4 | MBQ2226434.1/ | MFVFLVLLPLVSSAASKTVFITSDNIIDTDTDLQMLNSIKNYIEEISGGELQVIVDNQ | SEQ ID |
| | Methano- | APSPGEGWRAIEVTSDVSICLAACDAGNFIQLAGASTNSDKQIILVNTGDYDLDNQT | NO.: |
| | brevibacter | NFLRRACDDNYSDAYLAGLRDPGTFLKNSGVYGGGGSGGGGSGGGGSIENADKAIK | 100 |
| | sp. | DFQDNKAPHDKSAAYEANSKLPKDLRDKNNRFVEKVSIEKAIVRHDERVKSANDAISKLN | |
| | adhesin | EKDSIENRRLAQREVNKAPMDVKEHLQKQLD | |
| | variant, | | |
| | amino acid | | |
| C7.B.5 | MBQ2635481.1/ | MFVFLVLLPLVSSAATKTVFLTSDNVIDHDTDLKVLNSLKNYIEEISGGELQVIIDNQ | SEQ ID |
| | Methano- | APGPGEGWRAISVTSDVSIALAAADAGNFLQLATSTVDINKQIILINIGDYDLDNNSN | NO.: |
| | brevibacter | YLRRAWDDNYSNESLAGIRDPGTFLKNAGIYYLQPVKEFPNNAHDGILSNYDEEMN | 101 |
| | sp. | RNLAEQIVKLINAHENDTKVLSDSLIVINKLSPKGMANASKLLVNSDDKEMKGPYG | |
| | adhesin | SYSAPQLLYQTSAYLNGDGIDIPKEYSEPENPMGISFLVKDTYSIYDYMNMAGIVKN | |
| | variant, | YMDENGQAPDSIEYEGAHIGYYDLLYNFAKITQNHTDAKHMGFESEYHFDKVNDSI | |
| | amino acid | LLHIFPFVLIFLVLLIAYVFLKRIRRFGGGGSGGGGSGGGGSIENADKAIKDFQDNKAP | |
| | | HDKSAAYEANSKLPKDLRDKNNRFVEKVSIEKAIVRHDERVKSANDAISKLNEKDSIENRR | |
| | | LAQREVNKAPMDVKEHLQKQLD | |
| C7.B.6 | WP_ | MFVFLVLLPLVSSAAAKTVFITSDNIIDHDSDVKLLNSLKSYIEELSGGELQVIVDNQ | SEQ ID |
| | 296783736.1/ | SPAPGEGWRSIEVTSDVSVDLAASDAGNYLQLASSTVNSNKQIVFVNIGNYDLDNHT | NO.: |
| | Methano- | NFLRRAWDDNYSNETLAGMHDPGTFLKNAGIQYVQPAKEFPNNVNDGVISNYDEE | 102 |
| | brevibacter | MNKQIAQEIVDIINTHGNDEKTLSDGLVTHNIIKPSIMAKASQELIKSNDKEMKGTYG | |
| | sp. adhesin | NYSAPQLLYQTSSYLNGNGLDIPKSYDEPENPMGISFMAKDTYSVYDYFKMGGIVR | |
| | variant, | EYMDQNGRAPDSIEYDGAQISYYDLLYNFAKITQSHTDVKHMGFESEYHFDRVNDS | |
| | amino acid | ILLHIFPFILILFVLFLAYLFLKRIRRFGGGGSGGGGSGGGGSIENADKAIKDFQDNKAP | |
| | | HDKSAAYEANSKLPKDLRDKNNRFVEKVSIEKAIVRHDERVKSANDAISKLNEKDSIENR | |
| | | RLAQREVNKAPMDVKEHLQKQLD | |
| C7.B.7 | MDO5810390.1/ | MFVFLVLLPLVSSAAAKTVFITSDNIIDHDNDIKMLNSLKQYIEEISGGELQVIVDNQ | SEQ ID |
| | Methano- | APAPGEGWRAIEVTSDVSICLAASDAGNYLQLATATTNSDKQIVFVNTGSYDLDNH | NO.: |
| | brevibacter | SNYLRRAWDDNYSNESLAGLHDPGTFLKNAGIYYIQPSQDFPDNAKDGYLAKYDES | 103 |
| | sp. | MNKQLAQEIVDIIKTHENDTTILSDGLITHNIIKPSIMANASKELIKSGDKEMDGTYGN | |
| | adhesin | YSAPQLLYQTSSYLNGNGLDVPKAYDAPENPMGISFMAKDTYSIYDYFKMGGIVRD | |
| | variant, | YMDQNGRAPDSIEYEGAHISYYDLMYNFAKITQNHTNGKNMGFESEYHFDKVNDSI | |
| | amino acid | LLHIFPFVAILFILFLAYLLYKRLRRFGGGGSGGGGSGGGGSIENADKAIKDFQDNKAP | |
| | | HDKSAAYEANSKLPKDLRDKNNRFVEKVSIEKAIVRHDERVKSANDAISKINEKDSIENR | |
| | | RLAQREVNKAPMDVKEHLQKQLD | |
| C7.B.8 | MBR1610780.1/ | MFVFLVLLPLVSSAATKTVFITSDNIIDHDTDIEMLNSLKNYIEEISGGELQVIIDNQA | SEQ ID |
| | Methano- | PGPGEGWRAINVTSDVCINLAAADAGNYLQLAKATVYSDKQIVLINTGDYDLDNNT | NO.: |
| | brevibacter | NYLRRAWDDNYSNESLAGIRDPGTFLKNAGIYYLQPVKEFPDNAHDGYLDRYDEE | 104 |
| | sp. | MNRKLAEEIVEIVNKHGNDTKILSDSLIVTNKIPPTGMANASKMLVNSEDKEMKGPY | |
| | adhesin | GSYSAPQLLYQTSAYLNGDGIDIPKEYTEPDHPMGLSFLVRDSYSCYDYMHMAGLD | |
| | variant, | KNYMVGGGGSGGGGSGGGGSIENADKAIKDFQDNKAPHDKSAAYEANSKLPKDLRDK | |
| | amino acid | NNRFVEKVSIEKAIVRHDERVKSANDAISKLNEKDSIENRRLAQREVNKAPMDVKEHLQK | |
| | | QLD | |
| C7.B.9 | MBQ2227026.1/ | MFVFLVLLPLVSSAADNIIDTDTDLQMLNSIKNYIEEISGGELQVIVDNQAPSPGEG | SEQ ID |
| | Methano- | WRAIEVTSDVSICLAACDAGNFIQLAGASTNSDKQILVNTGDYDLDNHTNFLRRAW | NO.: |
| | brevibacter | DDNYSDAYLAGLRDPGTFLKNSGVYYIQPVKEFPNNANEGYIDRYDDEMNKQIAQE | 105 |
| | sp. | IVDIVNGHGNDTRIFSDELVSNNIVNPGVMAKSSQALINSGDNEMKGTYGNYTAAQ | |
| | adhesin | LLYQTSSYLNGNGLDVPKTFSEPDKPMGISFLTRDSYSIYDNFRMGGLVREYMDQN | |
| | variant, | GKAPDSIEYEGAHIGYYDLLYNFAKITQNHTDAKHMGFDSEYHFDKVNDSILLHIFP | |
| | amino acid | FVIILLVLFVAYWFYKRLRRYGGGGSGGGGSGGGGSIENADKAIKDFQDNKAPHDKS | |
| | | AAYEANSKLPKDLRDKNNRFVEKVSIEKAIVRHDERVKSANDAISKLNEKDSIENRRLAQ | |
| | | REVNKAPMDVKEHLQKQLD | |
| C7.B.10 | MBR1610530.1/ | MFVFLVLLPLVSSAAEEMNKQIAQEIVDIVNTHENDTKILSDGLITHNIIKPAVMAN | SEQ ID |
| | Methano- | ASKALIKSNDTEMKGTYGNYTGPQLLYQTSSYLNGNGLDIPKAYDEPEDPMGISFLA | NO.: |
| | brevibacter | KDTYSVYDYFKMGGIVREYMDANGRAPDSIEYQGAHIGYYDLLYNFAKITQNHTD | 106 |
| | sp. | TAHMGFESEYHFDRVNDSILLHIFPFILILFVLFIAYLFLKRIRRFGGGGSGGGGSGGG | |
| | adhesin | GSIENADKAIKDFQDNKAPHDKSAAYEANSKLPKDLRDKNNRFVEKVSIEKAIVRHDERV | |
| | variant, | KSANDAISKLNEKDSIENRRLAQREVNKAPMDVKEHLQKQLD | |
| | amino acid | | |
| C7.B.11 | ADC47454.1/ | MFVFLVLLPLVSSAASNASDNLDDLTISDSNSLDLVSTSNSDILSSDSGVSSDDSSND | SEQ ID |
| | Methano- | ASGDVLGSDVSSNESNNQSQSTLDSNNQSQSGLDSDNSTLLDSQSNNQSNSESSDSS | NO.: |
| | ebrvibacter | DSSETVIKNATSISVSSKTVVRGNSLNITLKDNASTLLSNKTVTFTFNGKTYNKTTNA | 107 |
| | ruminantium | KGIASLTLTATPKKYLVKIAFVGDELYEASSKSVNVTLSKTPTSISNSGKSIVRGKLY | |
| | M1. | KLTLKDAKGKALSGKKISISFNGKKYTKTTNSNGQVNLTINVNVGKTYKMTYKFAG | |
| | adhesin-like | DSNYLSSSGSVSIKVKMGTSIIGSGSSIVKGKSYTVTLKNANGAVLSNQKIAFTLSGK | |
| | protein with | TYNRTTNAKGQASLKIGLNSGKTYNLTYKYAGNSYYGGSSGKVSLFVKTPTTMKNS | |
| | transglutami | GKTIVSGETYKVTLKDADGKSLANKKVSITFNNKTYAKTTNSNGQASLTIKGTFGRS | |
| | nase domain | YPLSYKFAGDSKYGPSSGSLCLRVKKATSLKGSASSIVQGKSYTVTLKDSNSTPLAN | |
| | variant, | QTIVFTLDTKKYNRTTNAKGQASLKIGLAAGKTYNLAYKYSGTSYYNGSSGSVKLK | |
| | amino acid | VKFPTSLTNSGKSVMNGTGYNIVLKDSKSNLVSNKTISIGFNGKTYDEITDANGTVT | |
| | | LLIDANVPKTYKMTYKFAGDSDYGASSGTVNLTVKFKNAFTISQHISASSSLKSYVLK | |
| | | NKKVPATVSVNGVSLNLTSFTYLMAKATISINSNKTSGSILLVPVDSNYTNNGSRINA | |
| | | NLYKANYIDLAKKVISSAEANKLVPNSVSTNIGLVSHDLYSFGLAKALVFFNSDHYL | |
| | | PNYLILSSDDVGEKHSTVIPSNARGNASQFKAGLNEAETLTAAQIAKYLVASGHDAT | |
| | | NSEIKALAAKLVSGKTSLWDKANAIFTFARDNITYSYYADSKKGAAGTLSSKSGNC | |
| | | CDHSNLIVSLCRAANITARFSHAQGCTESSGLVAGHVWAQIYIDGVWYTADATSRR | |
| | | NSLGNIVNWNTNHYNTLKQYDHLSFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIEN | |
| | | RRLAQREVNKAPMDVKEHLQKQLD | |
| C7.B.12 | WP_ | MFVFLVLLPLVSSAASKTVFITSDNIIDQENDVKVLNSIKEYIEEISDGELQVIVDNEA | SEQ ID |
| | 292782159.1/ | PAPGEGYRAIQVTSDISICLSAADGGNYLQLAEASADSDKQIILINIGSYDLNNNSNYL | NO.: |
| | Methano- | RRAWDDNYSNETFMGIHDPGTFLKNAGIYYIQPVEEFPDNFNDDGYLAKYDEEMN | 108 |
| | brevibacter | KQIAQEIVDIANEHANDTTMLSDNLVVRNTISPAEMAEASKALLNSNDTEMNGEYG | |
| | sp. adhesin | NYTAPQLLYQTSSYLNGNGLDMPRSFDAPENPMGISFMVKDSYSIYDYMKMAGIVR | |
| | variant, | NYMDENGQAPDSIEYEGAHIGYYDLVYNFAKITQNHTDAEHMDFEQEYKFDKVND | |
| | amino acid | SILLKAFPFILILFILFLAYLGYQKIRRFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIEN | |
| | | RRLAQREVNKAPMDVKEHLQKQLD | |
| C7.B.13 | MBQ6813241.1/ | MFVFLVLLPLVSSAAMDIDEVGSSDDISDSGVVLESGADAGAYGSSNADVDSANQ | SEQ ID |
| | Methano- | LSSNDTAGNVNSESEVLSTDTNENSLESDIIDKDSKNVLSSSSLQAAAKKKTVIKGSG | NO.: |
| | brevibacter | TSITRGSSYYITLTDSNGNVLSNQKVTFSIKGKTYNKTTNSKGVASLKISLAKGKYDF | 109 |
| | sp. | VCAYAGTGAYYSSKLSVSLTVNAMATNIRTSGNSIKRGKSYSITLTDANSNLLANEK | |
| | Ig-like | VSFNISGHVYNRTTNSKGVASLTMNTAAKKYSLVCSYGGSSYYKASSVALTLTVLK | |
| | domain | GDTHIKISTDTVKKGNAVVMTLLDVRNKVLSAQKVSFTIKGKTYNLTTNSNGTAKLI | |
| | repeat | INLAAGKYPLVCSYDGSSNFMASKASENLTITDTVKTFSIADIETAATNLKAYVLKN | |
| | protein | KVLPSTVTVGGASLKISEFSYLMAKAVTNLNSNNKNKITLITGISNGDSATYVLNAK | |
| | variant, | VYKNQYVNVSKRVYSYIDSNKVPATYATVYSSSGANVGKAGFNLYTFAFAKILAFH | |
| | amino acid | KTENYLPNYCTFESSALKASTAAAGGSSSTSSANNQSKLKTTSLKAQSTSITRGDSYS | |
| | | VTLKDGSGNALPNQKITFTVSNKQYSDTTDSKGIAYLGADLLSGKYSITASFAGSSA | |
| | | YKSSKLSNTVTVKNSSTRFFLDDIENAAVNVKNYVSKNKALPTTVTVANTKLTIAQF | |
| | | SYIMAKAVHNINAGNKKYISLISISNCKSSGNYLDTTVYKAQYMNLTNRVISFTESN | |
| | | KVPPVYATVYGTNGKSVGNSEFNLYTFAFAKILAFHKTNNYLPNYCTFQSSAIGVKK | |
| | | PSSTTTTVVVNGPIKANSSQFKTGLNEKNTVGNLSAYLVDSGYSKITSSIQNLANQLT | |
| | | KNLNSTASKALAIYNYVRDEISYSYYANSRYGASGTLSVGSGNCVDQANLIVALCR | |
| | | ASGIEARYAHAKGCTFSSGLVTGHVWAQILVNGVWYSADATSVRNSLGNIVNWNT | |
| | | NSYYSLKQYTAIPFGGGGSGGGGSGGGGSIENADKAIKDFQDNKAPHDKSAAYEANSK | |
| | | LPKDLRDKNNRFVEKVSIEKAIVRHDERVKSANDAISKLNEKDSIENRRLAQREVNKAP | |
| | | MDVKEHLQKQLD | |
| C7.B.14 | WP_ | MFVFLVLLPLVSSAAMDIDDSSSIDDSNDLSGASVSSNSNENLASDSNSNNLESDNA | SEQ ID |
| | 292788318.1/ | GSSNVNSDYEILNTDNTVEDSDLEHNSNIANTKAMLGASSQSDSAVLQANAKVKTT | NO.: |
| | Methano- | LKGSSSSIYRGNYYTLTLTDSKGKVLSGQKLSYNINGKTYTLTTDSKGSTYLQINLKE | 110 |
| | brevibacter | GKYTMVCSYGGSSAYESSRLSVTLSVLKNPNAFTVKEIEDAATNVKNFVLKNKRLP | |
| | sp. | NTVKVGSKTLKISEFSYLSSQLISNLNSNKKGDIILLSGISDGKSSSASLKTTVYKAQY | |
| | pseudomurein- | LDLAKNVVSYIGSKKVAPNEILVKDASKRSVGNANFNLYTFAFAKILDFHKSKNYLP | |
| | binding | NYCTFESSAFNQAVSLKATILKGSSNSIYRGNYYTLTLTDGNGKALSGQKLRYAING | |
| | repeat- | KTYTLTTDSKGSTYLQINLKEGKYPMVCSYAGSKVYKSAKNSVTLTVLKNSNAFSV | |
| | containing | NEIETAATNVKNYVLKNKRLPNTVKVGSKTLKISEFTYLSSKAVSNLNSNNKKDIVL | |
| | protein | LNGISNGGSSTYSLKFTVYKAQYVDLAKRSASNIESKKVPANYLSVKDGSNKAGNA | |
| | variant, | NYNLYTFAFAKILDFHKSHNNLPNYCTFESSVYAPLKKSTSIKASSNSVNKGDSYSV | |
| | amino acid | TLVDNAGNALANQKITFNFSGKLYSQTTNSKGVASLKIGASQGTYSVVSSYAGSSAC | |
| | | EASKLSSTVTVKDTNRFSISEIEVAASNVKEYINIKNVRPGTVTVANKRLTISEFSYLM | |
| | | AKAVYNINAGNTNYITLPSGISGGNSEGDSMDATVYKAQYVDLSKRVVSFEESNKV | |
| | | SPVYAKVYSSSGSSLGNAGFDLYTYSFAKILDFHKSHKNLPNYCTFDSSVFKSSVVP | |
| | | AGISSDIPYNSNQFKAGLNEKNTESDLSKYLIGTGQSAITSSISNLANQLTKGLKSNEA | |
| | | KAQAIYNYVRDEIDYSYYANSKYGASGTLSAGSGNCVDQASLVVALCRASGIEARY | |
| | | AHAKGCTFSSGLVTGHVWAQILVNGVWYSADATSVRNKLGNIQNWNINSYSNLN | |
| | | RYAAVPFGGGGSGGGGSGGGGSIENADKAIKDFQDNKAPHDKSAAYEANSKLPKDLRD | |
| | | KNNRFVEKVSIEKAIVRHDERVKSANDAISKLNEKDSIENRRLAQREVNKAPMDVKEHLQ | |
| | | KQLD | |
| C7.B.15 | MBE6499844.1/ | MFVFLVLLPLVSSAATKTVFLTSDNIIDHEHDLQVLNSLKRHIEEISGGKLQVIVDNQ | SEQ ID |
| | Methano- | APAPGEGWRAIEVTSDVSICLAASDAGNYLQLATASTNSNKQIVFVNIGSYDLDNNT | NO.: |
| | brevibacter | NFLRRAWDDNYSPESLAGMNDPGTFLKNAGIYYVQPTKEFPENANDGVLSNYDEA | 111 |
| | thaueri | MNEKIAQEIVDIINKHGNDHKILSDGLVTHNNVKPSVMAKASKELIESNDFSMDGKY | |
| | adhesin | GRYTGPQLLYQTSSYLNGNGLDIPKDYGEPENPMTTSFLAKDTYSVYDFFKMGGIV | |
| | variant, | RAYMDDTGKAPDSIEYEGAHISYYDLLYNFAKITQTHTSAKHMGFESEYHFDKVND | |
| | amino acid | SILLHIFPFILIIFVLFIAYLFIKRIRRFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIEN | |
| | | RRLAQREVNKAPMDVKEHLQKQLD | |
| C7.B.16 | MBQ6630139.1/ | MFVFLVLLPLVSSAASKTVYITSDNIVDHDTDVKMLNSIKKYIEEISGGSLQVIVDN | SEQ ID |
| | Methano- | QAPAPGEGYRAIQVTSDISICLAAADAGNFYQVSENSADTDKQFVLINTGDYDLDNH | NO.: |
| | brevibacter | SNYLRRAWDDNYSNESLMGIRDPGTLLKNSGIYYIQPLKEFPNNGKNGYIDRYDEE | 112 |
| | sp. | MNKKIAQDIVDIVNGHANDTKILSDNLIVINKISPSAMANASKALINSNDTEMNGTY | |
| | adhesin | GNYTAAQLLYQTSSYLNGNGLDIPHSFNGPEKPMGISFLVKDTYSIYDYMNMAGIV | |
| | variant, | RNYMDANGRAPDSIEYEGAHISYYDLLYNFAKITQNHTDAKHMGFNQEYKFEKVN | |
| | amino acid | DSILLHIFPFVLILFVLFLAYLGYQKIRRFGGGGSGGGGSGGGGSIENADKAIKDFQDN | |
| | | KAPHDKSAAYEANSKIPKDLRDKNNRFVEKVSIEKAIVRHDERVKSANDAISKLNEKDSI | |
| | | ENRRLAQREVNKAPMDVKEHLQKQLD | |
| C7.B.17 | WP_ | MFVFLVLLPLVSSAASKTVFLTSDNIIDQDNDVKMLNSIKNYIEEISGGELQVIVDNQ | SEQ ID |
| | 298501801.1/ | APSPGEGYRAIQVTSDISICLAAADAGNYLQLGQTSANSNKQIVLINTGDYDLDNHS | NO.: |
| | Methano- | NYLRRAWDDNYSNETFMGMHDPGTLLKNSGIYYIEPIKEFPDNGKNGYIDRYDEDM | 113 |
| | brevibacter | NKQIAQEIVDIVNGHGNDTKTFSDNLVVTNTISPKVMADASKELLSSNDTEMNGTY | |
| | sp. adhesin | GNYTAGQLLYQTSSYLNGNGLDIPKSYDGPEHPMGISFLVKDTYSIYDYINMAGIVK | |
| | variant, | NYMDENGRAPDSIEYQGAHISYYDLQYNFAKITQNHTDAKHMGFDHEYKFDKVND | |
| | amino acid | SILLHIFPFILILFVLFLAYLGYKKIRRFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIEN | |
| | | RRLAQREVNKAPMDVKEHLQKQLD | |
| C7.B.18 | WP_ | MFVFLVLLPLVSSAATKTVFITTDNIVDHDFDVGLITAIKNYVQELSGGELQVVIDN | SEQ ID |
| | 296862675.1/ | QAPAAGEGYRSIEVTSDASIDLAASDAGNYIQLANYSAHSNKQIVFVNIGDYDLDNS | NO.: |
| | Methano- | SNYLRRAWDDNYTNETIAGVHDPGRLLRNSGIFYVQPAKEYPDKYKNGVLNYYDD | 114 |
| | brevibacter | AMAKQISQEIVDIINTHDNDTKVFSEDLVVKNKISPAGMANASKELLNSGDKEFNGT | |
| | sp. adhesin | FGAYTAPQLLYQTSSYLNGNGLDIPKTFKEPENPMGVSIFAKGSYSISDYFKMGGIVR | |
| | variant, | NYMDEHGQAPDSIEYEGAQISYYDLLYNFAKITQNHTDSAHMGFENEYQFEKVNSS | |
| | amino acid | FLLDAFPFILVLFILFLAYLVYKRIRRGGGGSGGGGSGGGGSIENADKAIKDFQDNKAP | |
| | | HDKSAAYEANSKLPKDLRDKNNRFVEKVSIEKAIVRHDERVKSANDAISKLNEKDSIENR | |
| | | RLAQREVNKAPMDVKEHLQKQLD | |
| C7.B.19 | MBR6927838.1/ | MFVFLVLLPLVSSAAAKTVFLTSDNIVDHDTDLQVLNSIKSYIEEISGGELQVIVDNE | SEQ ID |
| | Methano- | APAAGEGWRAIAVTSDVSICLAASDAGNYLQLGTASANSDKQYIFVNVGDYDLDN | NO.: |
| | brevibacter | HTNFLRRAWDDNYSNESLAGMHDPGTFLKNAGVYYIQPTKEFPQNTDDGIMDRYD | 115 |
| | sp. | EGMNRQIAQEIVDIVNSHGGDSKVLSDSLVTHNIVKPAVMAQASKALVESGDKEMQ | |
| | adhesin | GTYGNYTAAQLLYQTSSYLNGNGLDVPKSYDPPSDPLGISFFTKDTYSVYDYFNMA | |
| | variant, | GIVREYMDQNGKAPDSIEYEGAQISYYDLQYNFAKITQNHTDAEHMGFESEYHEDK | |
| | amino acid | VNDSILLHLFPFVVILFVLLIAYRFFKRIRRFGGGGSGGGGSGGGGSIENADKAIKDFQ | |
| | | DNKAPHDKSAAYEANSKLPKDLRDKNNRFVEKVSIEKAIVRHDERVKSANDAISKLNEKD | |
| | | SIENRRLAQREVNKAPMDVKEHLQKQLD | |
| C7.B.20 | WP_ | MFVFLVLLPLVSSAAAKTVFITSDNIIDHDSDLKMLNSLKSYIEEISGGSLQVIVDNQ | SEQ ID |
| | 294998163.1/ | APAAGEGWRSIEVTSDVSIDIAASDAGNYVQLASYTANSDKQIVFVNIGNYDLDNHT | NO.: |
| | Methano- | NFLRRAWDDNYSNENLAGLHNPGTFLKNAGIYYIQPAKEFPENAKSGILDTNDDEM | 116 |
| | brevibacter | YKKMAQEIVDIINTHENDEKVLSDGLISRNIVKPSVMANASKELINSNDKEMTGTYG | |
| | sp. adhesin | NYTAPQLLYMTSSYLNGNGLDVPKSFEEPENPMGISFMARDKYSVYDYFKMGGIVK | |
| | variant, | EYMDENGRAPDSIEYEGAHISYYDLMYNFAKITQNHTDAKHMGFDSEYHFDKVND | |
| | amino acid | SILLHIFPFILILFVLFIAYLLYKRIRRFGGGGSGGGGSGGGGSIENADKAIKDFQDNKA | |
| | | PHDKSAAYEANSKLPKDLRDKNNRFVEKVSIEKAIVRHDERVKSANDAISKLNEKDSIEN | |
| | | RRLAQREVNKAPMDVKEHLQKQLD | |
| C7.B.21 | WP_ | MFVFLVLLPLVSSAATKTVFITTDNIVDHDFDVGLINSIKNYVQELSGGELQVVIDN | SEQ ID |
| | 303372282.1/ | QAPRPGEGYRSIAVTSDVSIDLAASDAGNYLQLANYSANSDKQIVFVNIGDYDLDNS | NO.: |
| | Methano- | SNYLRRAWDDNYTNESFAGVHDPGTLLRNAGIFYVQPAKEYPDYYHDGILDKYDD | 117 |
| | brevibacter | EMAKKISQEIVDIINTHDNDTKVFSDELVVKNNIPPKGMADAAKEVVNSDDKELKG | |
| | sp. adhesin | PFGAYTGPQLLYQTSSYLNGNGLDVPKTYKDPESPMGISFLTKDSYSIYDYFKMAGI | |
| | variant, | VRNYMDEHGQAPDSIEYEGAHIGYYDLLYNFAKITQNHTDTAHMGFESEYHFDKV | |
| | amino acid | NDSFLLHIFPFILILFILFLAYLGYKKIRRFKGGGGSGGGGSGGGGSIENADKAIKDFQD | |
| | | NKAPHDKSAAYEANSKLPKDLRDKNNRFVEKVSIEKAIVRHDERVKSANDAISKLNEKD | |
| | | SIENRRLAQREVNKAPMDVKEHLQKQLD | |
| C7.B.22 | MBE6491313.1/ | MFVFLVLLPLVSSAATKTVFITTDNIVDHDFDVGLITAIKNYVQELSGGELQVVIDN | SEQ ID |
| | Methano- | QAPAAGEGYRSIEVTSDASIDLAASDAGNYIQLANYSAHSNKQIVFVNIGDYDLDNS | NO.: |
| | brevibacter | SNYLRRAWDDNYTNETIAGVHDPGTLLRNSGIFYVQPAKEFPDKYRNGILDNYDDE | 118 |
| | sp. | MAKQIAQEIVGIINTHDNDTKVFSDDLIVKNKISPAGMANASKELLNSGDKEFNGTF | |
| | adhesin | GAYTAPQLLYQTSSYLNGNGLDIPKTFKEPENPMGVSIFAKGSYSISDYFKMGGIVR. | |
| | variant, | NYMDEHGQAPDSIEYEGAQISYYDLLYNFAKITQNHTDSEHMGFEHEYKFEKVNSS | |
| | amino acid | FLLDVFPFILVLFVLFLVYLVYKRMRRGGGGSGGGGSGGGGSIENADKAIKDFQDNK | |
| | | APHDKSAAYEANSKLPKDLRDKNNRFVEKVSIEKAIVRHDERVKSANDAISKLNEKDSI | |
| | | ENRRLAQREVNKAPMDVKEHLQKQLD | |
| C7.B.23 | WP_ | MFVFLVLLPLVSSAAQSVNDSDDLDEKILKSCSLSKNATDKNISIKAKANSTKKTSN | SEQ ID |
| | 296855518.1/ | NKISTSKTKANTTTINRKTLAKTSTSFMNHVEKNGKFPKVKISNKNYSNTEYLYLMT | NO.: |
| | Methano- | KAVENNSNSKIEIKRNLVKRYNNTNSKSVKGTLNKTEYVKIASKTRKFIDKNKRAPN | 119 |
| | brevibacter | WVSSSKGNIPYNQLILSYSKCLDYFNKNNRLPNSIRLDDLDLDKINSKLNRNKTKNPS | |
| | sp. | NASVKIKKQTANKTVNKTTSNIVKTNTNKTRNTTTNTAPNKDDKSSKNSSLVERTL | |
| | trans- | NSINNILNNILYRLDPSKYQLDLTKSDEANLKLNTSKINVDINGKSTVNVKVSAKNA | |
| | glutaminase | TKATSKKSTASKAKTNKTKSSVSKAKTKTVNSNKASITESLMKYLSSSKNCQVKNK | |
| | domain- | SIQDLAKTLTSKLKSDYEKGKKLFTWVRDNIQYKKYRNTRRGAVKTLQTKKGNCV | |
| | containing | DQSHLLIALSRAAGIPARYVKGGNCKFSSGYGSGPIWTQMYINNKWVVADTTSHRN | |
| | protein | SLGKIKNWNTKNYKLYGQFSSINFGGGGSGGGGSGGGGSIENADKAIKDFQDNKAPH | |
| | variant, | DKSAAYEANSKLPKDLRDKNNRFVEKVSIEKAIVRHDERVKSANDAISKLNEKDSIENR | |
| | amino acid | RLAQREVNKAPMDVKEHLQKQLD | |
| C7.B.24 | WP_ | MFVFLVLLPLVSSAAQSISADNLDEKTIKSDSSSVKSSTATKVKTSTANKKTTSTDN | SEQ ID |
| | 292787867.1/ | EKTTSASKKASASATKENKTTVNKKTLAKTSTSFINYVEKNGKFPKVRISNKNYSNT | NO.: |
| | Methano- | EYLYLMTKAVENNSNPKIEINRNMVKKYNNSNSKSVKGSLNKTEYVKIAGKTRKFI | 120 |
| | brevibacter | EKNRRAPNWVSSSKGNIPYNQLILSYSKCLDFFNKNSRLPNTVRLDDLDLDKINDKL | |
| | sp. trans- | NKNKSESAVNASSKTSKQTSNKTVNKTTGNIAKTNANKTKNTTTKTASNKEDDSSK | |
| | glutaminase | NASLVERTLNNIHNILNNILDRLDPSKYQADVTKTDEANVKLNTSKINVDINGKSTV | |
| | domain- | NIKVSAKNTTKATAKKTATKSTVKKTASKTVSSNKNSINEVLRKCLASSKNCQVYK | |
| | containing | RQIQDLAKTLTSKLKSDYEKGKKLFTWVRDNIQYKKYRNTRRGAVKTLQTKKGNC | |
| | protein | VDQSHLLIALSRASGIPARYVKGGNCKFSNGYVSGHIWAQMYINKKWVVADTTSH | |
| | variant, | RNTLGNIRNWNTKNYQLCGQFSSINEGGGGSGGGGSGGGGSIENADKAIKDFQDNKA | |
| | amino acid | PHDKSAAYEANSKLPKDLRDKNNRFVEKVSIEKAIVRHDERVKSANDAISKLNEKDSIE | |
| | | NRRLAQREVNKAPMDVKEHLQKQLD | |
| C7.B.25 | MDO5819761.1/ | MASVSASDVNDINDGNDHISLSEDSASSDLANDDLALSSADADSIDDDLDLASSSNE | SEQ ID |
| | Methano- | AYEDFSANPSNGESSYGPQSNALSESGDSNSQTTTNTSIESSTSNVVKGENYSVTLKD | NO.: |
| | brevibacter | KDGNVLSGKNIIFTLNGTNFTRNTNSNGIASLTLNTRVGNYLIVVSFLGDDSYANSSF | 121 |
| | sp. | SQQLTVSKIPTAIENSTGLAVIGRAYSVVLKDKKGNPLSSKAVTLTFNGKTYKRTTN | |
| | trans- | SQGIVSITLDGKRGNSYNLTYKFAGDSSYMASSGSVSLKLKMSTKIVGTDARIVQGK | |
| | glutaminase | VFTVTLKDASNKLLANKKVTVLHNGKTYNRTSNSKGIVSLTLSQTPGKYYNISYRFA | |
| | domain- | GDSTYTGSSKKLSVFIKTPTKFVNSGSFVCKGNVYYVTLKDSNDNVLANKTVKVTY | |
| | containing | GTKNYTLTTNSKGKVGVKINSAKGKVYKFTYKENGNSLYGPSSGSLNLRTKLATSLI | |
| | protein | GSSATIIKGNPYKVTLKDSDGAVIANQNITFNFSGAKYVRTTNSKGIASLVINPSALK | |
| | variant, | TYNLSYSYAGNSLYNKSSGNVSLAVKLGTTIKNSGTTVANNSSYEVTLKDSNNNAL | |
| | amino acid | ASKVIIFTLDGKTYRNTTNSKGVASLFISEKNFTTVNLTYKFAGDSMYVASSGSVKV | |
| | | RVVSDKVFTFNQIVAASKALRKYVEKNSELPSTVTVNGLKVNITSFAYLMSKSIVNV | |
| | | NNGKKSSVEVVSVSSNYSNSGSGLINANLYKAGYLNLSNYLISYTKSNHKIPNHINT | |
| | | RIGDLSPNLYIFGLSKALDFYSNNSYLPNYLILNSEDVCGKSNASIKHGNASQNKKGL | |
| | | NEAQSLNATQLAVYLKSSGNDALNDAIRNLAKQLTSGKSTTLAKANAIFTYVRDNV | |
| | | AYEYYADTKYKATGTLSAKRGNCCDHANLVVALCRAANIPARYSHAQGCTFSSGL | |
| | | VTGHVWAQIYIEGVWYSADATSKRNGLGNFHIWITNYFNSLKQYVHLPFGGGGSGG | |
| | | GGSGGGGSIENADKAIKDFQDNKAPHDKSAAYEANSKLPKDLRDKNNRFVEKVSIEKAIV | |
| | | RHDERVKSANDAISKLNEKDSIENRRLAQREVNKAPMDVKEHLQKQLD | |
| C7.B.26 | MBQ6813109.1/ | MFVFLVLLPLVSSAAQSLDDLNYGDSDIGLNDSGNPELNRDSTFKSLQSTVIESSNT | SEQ ID |
| | Methano- | DKTEKINTNESLGAKAISSNCEKTDTYEDKSVKTSTAKASNTTQVKENKTSMKITTL | NO.: |
| | brevibacter | AKSASSYFAYVKKNGKLPNPVSISNKKYQSAQYLYLVSKAVSNISASTVEIKDSLVN | 122 |
| | sp. | NFSYINYKTANGTLSKKECEELANKTARFIEKNHRAPTWIGSDKGNIPCNQLILLFTK | |
| | trans- | CLDYYNNNSKLPSSVKLSDVDLDKISGKSNVSSSKASNSSKNAGNSEILLNSTLNAIS | |
| | glutaminase | SILTDLNNILNPSISTTTTITISSNSKTNTTTATIKTNTTVSTKSNTSAKTNSSAKTNTS | |
| | domain- | AKTNNSTKTNNSTKTNTNTKVNTTAKTNSSTKTNTNTKVNTTAKTNTNTATSSSSKN | |
| | containing | SLSSWVDKAITAVGKSLIKFIDSSILNDKYNGESLKKYLSASKNCQSTSSAIKSLAKK | |
| | protein | LTSSLTSDYDKGEKIFNWVRDNIEYQKYSNTKKGALNTLNSKSANCVDQAHLVVAL | |
| | variant, | ARAAGLPARYVNANNCKFTSGYVSGHVWAQILVGITWVVADTTSSRNSFGIVKNW | |
| | amino acid | NVKSYKLVGKYSSISEGGGGSGGGGSGGGGSIENADKAIKDFQDNKAPHDKSAAYEAN | |
| | | SKIPKDLRDKNNREVEKVSIEKAIVRHDERVKSANDAISKLNEKDSYENRRLAQREVNKA | |
| | | PMDVKEHLQKQLD | |

For SEQ ID Nos. 1-94, the below key indicates sequence features: Nucleotide sequences in lower case correspond to the untranslated region (UTR); nucleotide sequences in UPPER CASE correspond to the open reading frame. Amino acid sequences in bold correspond to the signal peptide, amino acid sequences with no mark correspond to the antigen, and amino acid sequences in italics correspond to a linker and the Sbi adjuvant fusion.

### EXEMPLIFICATION

### Example 1: Description of Methods Used in Examples Described Herein

IVT Template production: For experiments testing RNA vaccine candidates in cattle, constructs defined in this Example, which encode bovine-optimized versions of methanogen-targeted antigens or cytokines, were amplified from respective custom-synthesized plasmids with pUC19 backbones (GenScript). Amplification was carried out at an annealing temperature of 50° C. in a 20 μL reaction consisting of 0.25 μM each primer T7-AGG_fwd and 120pA_rev, 1× Herculase II buffer, 25 mM each dNTP, 15 ng plasmid (GeneScript), and 0.4 μL Herculase II enzyme (Agilent). After the PCR program completed, 20 U DpnI (NEB) was added to each PCR reaction and incubated at 37 C for 15 minutes to digest plasmid template. DpnI treated PCR product was purified using the DNA Clean & Concentrator-25 kit (Zymo Research) and eluted into 60 μL. Nuclease free water.

The sequences of primers used were as follows:

T7-AGG_fwd:
(SEQ ID NO.: 95)
gaattTAATA CGACTCACTA TAAGGcttgt tctttttgca gaagc
120pA_rev:
(SEQ ID NO.: 96)
TTTTTTTTTT TTTTTTTTTT TTTTTTTTTT TTTTTTTTTT
TTTTTTTTTT TTTTTTTTTT TTTTTTTTTT TTTTTTTTTT
TTTTTTTTTT TTTTTTTTTT TTTTTTTTTT TTTTTTTTTT
agaatgtgaa gaaactttct ttttattag

### In Vitro Transcription (IVT) of RNA Vaccine Candidates

For each vaccine candidate, RNA was synthesized in 22 μL IVT reactions consisting of 200 ng of the respective template, 20 mM MgCl2, 7.5 mM each NTP, 7.5 mM CleanCap AG (TriLink), 1× HiScribe Transcription Buffer, 2M Betaine, and 2 μL HiScribe polymerase mix (NEB) and incubated at 37° C. for 2 hours. For RNA conditions using chemically modified nucleotides, either UTP was substituted by 5-hydroxymethyluridine triphosphate (TriLink) or cytidine was substituted by N4-acetylCTP (Jena BioScience) at the indicated ratio in the IVT mixture.

All IVT products were cleaned up using Monarch 500 μg RNA Clean Up kit (NEB) and eluted into 83 μL nuclease-free water. Eluted products were then digested in 100 μL reactions consisting of 1× DNase I buffer, 10 U of DNase I (RNase-free) (New England Biolabs) and 100 U. of CIAP (Promega) at 37° C. for 5 minutes to degrade DNA template and remove residual 5′ triphosphates on RNA. Treated samples were cleaned up using Monarch 500 ug RNA Clean Up kit (New England Biolabs) and eluted into 100 μL 1 mM sodium citrate, pH 6.5.

One-Pot In vitro Transcription (IVT) of Multiplexed RNA Vaccine Candidates For multiplexed RNA Vaccine Candidates, RNA can be synthesized in separate reactions, as in the IVI method above, and then normalized and pooled in a final step, or in a single or “one-pot” reaction. In the latter case, multiplexed RNA was synthesized by mixing 20 mM MgCl2, 7.5 mM each NTP, 7.5 mM CleanCap AG (TriLink), 1× HiScribe Transcription Buffer, 2M Betaine, 2 uL HiScribe polymerase mix (NEB), and equimolar or predetermined molar ratio amounts of each respective DNA template, to a total of 200 ng DNA, in 22 μL IVT reactions and incubated at 37° C. for 2 hours. For RNA conditions using chemically modified nucleotides, either UTP was substituted by 5-hydroxymethyluridine triphosphate (TriLink) or cytidine was substituted by N4-acetylCTP (Jena BioScience) at the indicated ratio in the IVT mixture.

All IVT products were cleaned up using Monarch 500 μg RNA Clean Up kit (NEB) and eluted into 83 μL nuclease-free water. Eluted products were then digested in 100 μL reactions consisting of 1× DNase I buffer, 10 U of DNase I (RNase-free) (New England Biolabs) and 100 U of CIAP (Promega) at 37° C. for 5 minutes to degrade DNA template and remove residual 5′ triphosphates on RNA. Treated samples were cleaned up using Monarch 500 ug RNA Clean Up kit (New England Biolabs) and eluted into 100 μL 1 mM sodium citrate, pH 6.5.

### Formulation for In Vivo RNA Experiments

Formulations of RNA in lipid nanoparticles (RNA-LNPs) were prepared using a microfluidic mixer (Precision Nanosystems, Vancouver, BC). Briefly, GenVoy-ILM lipid mixture (Precision Nanosystems NWW0042) was diluted to 12.5 mM in anhydrous ethanol and combined with an aqueous solution of RNA (0.14 mg/mL) in PNI buffer (Precision Nanosystems NWW0043), using the manufacturer-recommended formulation parameters. Formulations were immediately diluted 30:1 in phosphate-buffered saline (Gibco 10010023), concentrated using Amicon centrifugation filters (MilliporeSigma UFC901008), and adjusted to the appropriate final volume with PBS. Formulations were stored at 4° C. for up to 2 days prior to the prime dose and up to 25 days prior to the boost dose.

### RNA Administration Study in Cattle

Animal experiments were carried out in accordance with the guidelines set forth by Texas A&M University (TAMU, College Station, MA, USA) and were approved by the TAMU Institutional Animal Care and Use (IACUC) committee. Cattle were housed at TAMU McGregor Research Facility, consisting of a breed of Angus crossbred cattle with no more than 0.25 Bos indicus composition of Bos indicus. Calves were weaned at approximately 205 days of age for a minimum of 4 weeks post weaning before being trained to the GreenFeed Pasture Systems (C-Lock Inc.) and a minimum of 8 weeks before receiving vaccination treatments.

During some RNA vaccine studies, such as M1 and M2, calves (n=4 or 5 per condition) received two RNA administrations at Day 0 (prime) and Day 21 (boost). RNA injections consisted of 2 mL RNA-LNP formulation (0.5 mg RNA dose per animal) delivered via neck intramuscular injection, unless noted otherwise. Collection of blood, saliva, and rumen fluid samples were taken from each animal on Days −1, 21, 34, and 90 and shipped on dry ice for analysis, unless noted otherwise.

During other RNA vaccine studies, such as M3, calves (n=10 per condition) received two RNA administrations at Day 0 (prime, 1°), Day 25 (boost, 2°) and Day 104* (boost, 3°). RNA injections consisted of 2 mL RNA-LNP formulation (0.55 mg RNA dose per animal) delivered via neck intramuscular injection, unless noted otherwise. Blood samples were collected on Days −1, 25, 42, 90, 109*, and 122*, and rumen fluid samples were collected on −1, 90, 109*, and 122*. Samples were shipped on dry ice for analysis, unless noted otherwise. (*Only conditions F1 and F3.)

Blood was collected via jugular venipuncture (8-10 mL) and stored on ice for >2 hrs to allow for clotting before centrifugation. Serum was collected and stored at −20° C. until thawed for assay.

Saliva was collected using a vacuum into a conical flask. >10 mL saliva was harvested per collection and stored at −20° C. until thawed for assay.

Rumen fluid was collected using an esophageal tube and pump to withdraw rumen fluid, and was either immediately snap frozen in liquid nitrogen or placed on ice until being centrifuged at −4° C. The solid fraction of the centrifuged sample (i.e the pellet) was withdrawn and placed in microtubes and snap frozen in liquid nitrogen prior to being stored at −80 C and shipped on dry ice to CosmosID Inc. (Germantown, MD) for sequencing. The liquid fraction was stored at −20° C. until thawed for assay. For Study M3, separate samples were processed for Volatile Fatty Acid (VFA) analysis For studies M1 and M2, animals were randomly distributed by body weight (BW) into treatment groups and penned together.

Enteric methane (CH4), oxygen (O2), and carbon dioxide (CO2) emissions per individual calf was measured via the GreenFeed Pasture System (C-Lock Inc.) on Days −8 to −1, representing a pre-vaccine baseline, and again on Days 22 to 29. Feed intake per individual calf was measured via GrowSafe Feed Intake Systems (GrowSafe Systems, Ltd.) and RFID ear tags for the duration of the acclimation and experiment time, Days −32 to 90.

For Study M3, animals were monitored for CH4 emissions and dry matter intake (DMI) for a pre-trial baseline period, and distributed into treatment groups based on initial CH4/DMI and BW and penned separately. Gas emissions per individual calf were measured continuously from D-22 to the end of the study and a pre-vaccine enteric methane baseline was determined after sorting into final pens and before injection (D-6 to D0). Feed intake per individual calf was measured via GrowSafe Feed Intake Systems (GrowSafe Systems, Ltd.) and RFID ear tags for the duration of the acclimation and experiment time.

### Sequencing and Bioinformatics Analysis

For DNA extraction, DNA from samples was isolated using the QIAGEN DNeasy PowerSoil Pro Kit, according to the manufacturer's protocol. Non-fractioned samples were allowed to thaw at 4° C. for a maximum of 16 hours, and optionally homogenized and/or centrifuged before extraction. Extracted DNA samples were quantified using Qubit 4 fluorometer and Qubit dsDNA HS Assay Kit (ThermoFisher Scientific).

For library preparation and sequencing, DNA libraries were prepared using the Nextera XT DNA Library Preparation Kit (Illumina) and IDT Unique Dual Indexes with total DNA input of 1 ng Genomic DNA was fragmented using a proportional amount of Ilumina Nextera XT fragmentation enzyme. Unique dual indexes were added to each sample followed by 12 cycles of PCR to construct libraries. DNA libraries were purified using AMpure magnetic beads (Beckman Coulter) and eluted in QIAGEN EB buffer. DNA libraries were quantified using Qubit 4 fluorometer and Qubit dsDNA HS Assay Kit. Libraries were then sequenced on an Illumina NextSeq 2000 platform 2×150 bp

For bioinformatics analysis, unassembled sequencing reads were directly analyzed by CosmosID-HUB Microbiome Platform (CosmosID Inc., Germantown, MD) described elsewhere (Ottensen et al., 2016, Ponnusamy et al., 2016, Hasan et al., 2014, Lax et al., 2014) for multi-kingdom microbiome analysis and profiling of antibiotic resistance and virulence genes and quantification of organisms' relative abundance. Briefly, the system utilizes curated genome databases and a high-performance data-mining algorithm that rapidly disambiguates hundreds of millions of metagenomic sequence reads into the discrete microorganisms engendering the particular sequences.

### ELISA

ELISA assays measured antigen-specific IgG and IgA response to RNA vaccine formulations. For coating ELISA plates, antigen protein was either reconstituted in H2O from a lyophilized state (e.g., OVA protein, 2000 ug/mL) or synthesized via an E. coli-based cell-free protein expression system (Liberum Biotech) and a plasmid template DNA comprising the sequence of the antigen protein (e.g., mru1499), as well as start/stop codons, an upstream T7 promoter and ribosome binding site, and a downstream T7 terminator. This reaction incubated for 8 hrs at 27° C. and then was diluted 1:10 in PBS. Nunc ELISA plates (ThermoFisher Scientific) were then coated with an antigen protein solution (50 μL/well) for an overnight incubation at 4° C.

Plates were washed three times with PBS-Tween 1% (PBST) and blocked (1 hr, room temperature) with 200 μL of SuperBlock (Thermo Fisher Scientific). A diluent was created from a 1:2 mixture of SuperBlock (Thermo Fisher Scientific) and H2O. Serial dilutions of serum samples (range 1:10-1.1010) in diluent were added (90-135 μL/well) to each well and incubated (2 hr, room temperature). The plates were washed three times in PBST. For IgG titers, plates were then incubated for 1 h at room temperature with 50 μL/well of HRP-conjugated sheep anti-cow IgG (ab112618, Abcam), 1:3,000 in diluent. For IgA titers, plates were then incubated for 1 h at room temperature with 50 μL/well of HRP-conjugated sheep anti-cow IgA H&L (ab112755, Abcam), 1:1,000 in diluent. The plates were washed three times in PBST and incubated for 10 min at room temperature with SigmaFast OPD Solution (Sigma Aldrich) at 100 μL/well. The reactions were stopped with addition of 50 μL/well of 2M HCl. The absorbance read at 450 nm on a GloMax Plate Reader (Promega).

Endpoint titers are defined here as the reciprocal of the highest analyte dilution that gives a reading above the cutoff. The cutoff is calculated by summing the average of the untreated (UTD) values plus the standard deviation of the untreated (UTD) values. The geometric mean titer (GMT) and geometric standard deviation (GSD) are taken across condition group.

Vaccine formulations used in Example 2 through 5

Ssp refers to Secpep1. HNpep refers to a PPa2 secretory signal. HNadj refers to an Sbi adjuvant. HNmod1 refers to N4-acetyl CTP. HNmod2 refers to 5-hydroxymethyluridine triphosphate.

Vaccine formulations used in Example 5.

Approach for antigen cluster generation.

One approach utilized to generate multi-component RNA vaccines in Examples 4 and 5 comprises using bioinformatic methods to identify groupings of two or more antigens (e.g., DNA or amino acid sequences) that are related to one another (e.g., share a defined level of sequence identity or structural identity, such as >80%) and are associated with two or more species of methanogen or methanogen associated protein in the rumen (e.g., antigens within a grouping have >80% identity to more than one methanogen genome, collectively).

In one example, such groupings were generated from a pool of antigens that were 50aa in length and represented all possible 50aa-frames present in a set of open reading frames (ORF) generated from metagenomic data of a rumen In some instances, this set of ORFs was limited to entries that shared a defined sequence identity (e.g., >80%) to the genome of a methanogen species or a methanogen associated protein. In some instances, “hypothetical proteins” and/or sequences for de novo peptide designs that shared structural similarity to methanogen-associated proteins (e.g., structure identity via AlphaFold or LLM prediction) were included. Alternatively, in some instances, pools of 50aa-frames were generated from protein or genome databases and limited to select methanogen species (e.g, methanogen species most abundant in rumen samples, Methanobrevibacter ruminantium, Methanobrevibacter smithii, Methanobrevibacter oralis, Methanomicrobium mobile, Methanosphaera stadtmanae). In some instance, this set of ORFs was limited to entries that were algorithmically predicted to be surface-associated proteins (e.g., >0.5 confidence index). In some instance, the grouping of antigens that were generated consisted of 2, 3, 4, or 5 antigens with >80% identity to each other and the grouping contained antigens that were associated with 2, 3, 4, or 5 different methanogen species. In some instances, the final antigen groupings were defined as the full protein sequences from which the 50aa-frames were derived from.

Approach for multi-component formulation composition.

Multi-component RNA vaccines targeted to multiple methanogen proteins and/or proteins associated with multiple species of Methanogen/Archaea, used in Examples 4 and 5 were generated by using algorithmic or rational methods to select combinations of antigens and antigen grouping to provide coverage across multiple species of methane-associated microbes present in the rumen. Vaccine formulations, as shown in Table 3 or Table 5, are described to consist of multiple antigens (e.g., 3, 8, or 5 antigens per formulation), are associated with multiple methane-associated species (e.g., 3 or 5 species per formulation), and are combined at specified proportions with respect to molarity or mass.

Specifically, in this example, F2 comprises three antigens C3.1 (SEQ ID NO: 35), C3.2 (SEQ ID NO: 37), and C3.3 (SEQ ID NO: 39), generated via an antigen cluster generation workflow described above. The antigens of F2 formulation have regions that share a define level of similarity (e.g., sequence identity) and provide coverage of at least three methane-associated species. Also, the antigens of the F2 formulation are associated with the name/functionality of “signal peptidase” (ex. BlastP highest match). Thereby, they are characterized as being membrane-bound, enzymes, and/or are critical for converting secretory proteins to their mature forms by cleaving signal peptides from the N-termini proteins during or after that protein's translocation through the membrane.

F3 comprises two grouping of two antigens each (C7.1 (SEQ ID NO: 55), C7.2 (SEQ ID NO.: 57), C7.3 (SEQ ID NO.: 59), C7.4 (SEQ ID NO.: 61)), and one grouping of 4 antigens (C8.1 (SEQ ID NO.: 63), C8.2 (SEQ ID NO.: 65), C8.3 (SEQ ID NO.: 67), C8.4 (SEQ ID NO.: 69)), in which the former two groupings were generated via an antigen cluster generation workflow and the later grouping was selected via rational design. The former two antigen groupings each respectively have regions that share a define level of similarity (e.g., sequence identity) and provide coverage of at least two methane-associated species. Also, the antigens of the F2 formulation are associated with the name/functionality of “adhesin”, “adhesin-like”, or “immunoglobin (Ig)-like” and/or have regions that share a defined level of similarity to adhesin/adhesin-like/lg-like domain-containing domains (ex. BlastP matched) Thereby, they are characterized as being cell-surface components that facilitate binding to other cells or surfaces. Overall, the F3 Formulation comprises eight antigens that cover at least five methane associated species.

F4 comprises a grouping of five antigens: C9.1 (SEQ ID NO.: 79), C9.2 (SEQ ID NO.: 81), C9.3A (SEQ ID NO.: 83), C9.4C (SEQ ID NO.: 91), and C9.5 (SEQ ID NO.: 93), generated via a rational design method The antigens of F4 formulation are associated with the name/functionality of “archaeal oligosaccharyltransferase” or “AgB” and/or have regions that share a defined level of similarity to oligosaccharyltransferase/AgB proteins (e.g., BlastP matched). Thereby, they are characterized as enzymes critical to N-glycosylation—the covalent attachment of oligosaccharides to target proteins. Overall, the F4 Formulation comprises five antigens that cover at least five methane associated species.

F1 is a non-methanogen antigen control, ovalbumin (OVA), derived from Gallus gallus and intended to generate an immune response that is not specific to methanogens.

TABLE 3
Compositions for various mRNA vaccine formulation.

| Formulation | Antigen | Species Association | Molarity | Mass |
|---|---|---|---|---|
| Name | Name | (per antigen) | (%) | (%) |
| F1 | Ova_Sbi | Gallus gallus | 100% | 100% |
| | (Control) | (Non-methanogen) | | |
| F2 | C3.1 | Methanobrevibacter | 33% | 33% |
| | | ruminantium | | |
| | C3.2 | Methanobrevibacter | 33% | 33% |
| | | smithii | | |
| | C3.3 | Methanobrevibacter | 33% | 33% |
| | | oralis | | |
| F3 | C7.1 | Methanobrevibacter | 7.5% | 5.9% |
| | | smithii | | |
| | C7.2 | Methanobrevibacter | 7.5% | 5.9% |
| | | oralis | | |
| | C7.3 | Methanobrevibacter | 7.5% | 11% |
| | | smithii | | |
| | C7.4 | Methanobrevibacter | 7.5% | 10% |
| | | oralis | | |
| | C8.1 | Methanobrevibacter | 17.5% | 16% |
| | | ruminantium | | |
| | C8.2 | Methanobrevibacter | 17.5% | 13% |
| | | ruminantium | | |
| | C8.3 | Methanobrevibacter | 17.5% | 19% |
| | | ruminantium | | |
| | C8.4 | Methanobrevibacter | 17.5% | 19% |
| | | ruminantium | | |
| F4 | C9.1 | Methanobrevibacter | 28.6% | 31% |
| | | ruminantium | | |
| | C9.2 | Methanobrevibacter | 17.9% | 17% |
| | | smithii | | |
| | C9.3A | Methanobrevibacter | 17.9% | 17% |
| | | oralis | | |
| | C9.4C | Methanomicrobium | 17.9% | 17% |
| | | mobile | | |
| | C9.5 | Methanosphaera | 17.9% | 17% |
| | | stadtmange | | |

TABLE 5
Additional exemplary compositions for various mRNA vaccine formulation.
Formulation name, Antigen name, Species association, Input ratio
by molarity (%), and Input ratio by mass (%) are specified per
row of table. Formulations comprise of combinations of antigens
and antigen groupings derived from the methods described above.

| Formulation | Antigen | Species | Protein Name | Molarity | |
|---|---|---|---|---|---|
| Name | Name | Association | Associations | (%) | Mass |
| F3+.A | C7.1 | Methanobrevibacter | Adhesin-like protein, | 2.40% | n/a |
| | | smithii | Transglutaminase | | |
| | C7.2 | Methanobrevibacter | domain-containing | 2.40% | |
| | | oralis | protein, Ig-like | | |
| | C7.3 | Methanobrevibacter | domain-containing | 2.40% | |
| | | smithii | protein, | | |
| | C7.4 | Methanobrevibacter | Right-handed | 2.40% | |
| | | oralis | parallel beta-helix | | |
| | C8.1 | Methanobrevibacter | repeat-containing | 5.70% | |
| | | ruminantium | protein | | |
| | C8.2 | Methanobrevibacter | | 5.70% | |
| | | ruminantium | | | |
| | C8.3 | Methanobrevibacter | | 5.70% | |
| | | ruminantium | | | |
| | C8.4 | Methanobrevibacter | | 5.70% | |
| | | ruminantium | | | |
| F3+.B | C7.B.1 | Methanobrevibacter | Adhesin | 2.60% | n/a |
| | | sp. | | | |
| | C7.B.2 | Methanobrevibacter | Adhesin | 2.60% | |
| | | sp. UBA188 | | | |
| | C7.B.3 | Methanobrevibacter | Adhesin | 2.60% | |
| | | sp. UBA188 | | | |
| | C7.B.4 | Methanobrevibacter | Adhesin, partial | 2.60% | |
| | | sp. | | | |
| | C7.B.5 | Methanobrevibacter | Adhesin | 2.60% | |
| | | sp. uncultured | | | |
| | C7.B.6 | Methanobrevibacter | Adhesin | 2.60% | |
| | | sp. | | | |
| | C7.B.7 | Methanobrevibacter | Adhesin | 2.60% | |
| | | sp. | | | |
| | C7.B.8 | Methanobrevibacter | Adhesin, partial | 2.60% | |
| | | sp. | | | |
| | C7.B.9 | Methanobrevibacter | Adhesin, partial | 2.60% | |
| | | sp. | | | |
| | C7.B.10 | Methanobrevibacter | Adhesin, partial | 2.60% | |
| | | sp. | | | |
| | C7.B.11 | Methanobrevibacter | Adhesin-like | 2.60% | |
| | | ruminantium M1 | protein with | | |
| | | | Transglutaminase | | |
| | | | domain adhesin | | |
| | C7.B.12 | Methanobrevibacter | Adhesin | 2.60% | |
| | | sp. | | | |
| | C7.B.13 | Methanobrevibacter | Ig-like domain | 2.60% | |
| | | sp. | repeat protein | | |
| | C7.B.14 | Methanobrevibacter | pseudomurein- | 2.60% | |
| | | sp. | binding repeat- | | |
| | | | containing protein | | |
| | C7.B.15 | Methanobrevibacter | Adhesin | 2.60% | |
| | | thaueri | | | |
| | C7.B.16 | Methanobrevibacter | Adhesin | 2.60% | |
| | | sp. uncultured | | | |
| | C7.B.17 | Methanobrevibacter | Adhesin | 2.60% | |
| | | sp. uncultured | | | |
| | C7.B.18 | Methanobrevibacter | Adhesin | 2.60% | |
| | | sp. | | | |
| | C7.B.19 | Methanobrevibacter | Adhesin | 2.60% | |
| | | sp. uncultured | | | |
| | C7.B.20 | Methanobrevibacter | Adhesin | 2.60% | |
| | | sp. | | | |
| | C7.B.21 | Methanobrevibacter | Adhesin | 2.60% | |
| | | sp. | | | |
| | C7.B.22 | Methanobrevibacter | Adhesin | 2.60% | |
| | | sp. uncultured | | | |
| | C7.B.23 | Methanobrevibacter | Transglutaminase | 2.60% | |
| | | sp. | domain-containing | | |
| | | | protein | | |
| | C7.B.24 | Methanobrevibacter | Transglutaminase | 2.60% | |
| | | sp. | domain-containing | | |
| | | | protein | | |
| | C7.B.25 | Methanobrevibacter | Transglutaminase | 2.60% | |
| | | sp. | domain-containing | | |
| | | | protein | | |
| | C7.B.26 | Methanobrevibacter | Transglutaminase | 2.60% | |
| | | sp. | domain-containing | | |
| | | | protein | | |

### Example 2: Generation of Antigen-Specific Antibodies with a Multi-Component Vaccine Formulation

This example describes antigen specific IgG titers generated from the vaccination of cattle with an exemplary vaccine against OVA and mru1499. The methods used in this Example are provided in Example 1 above.

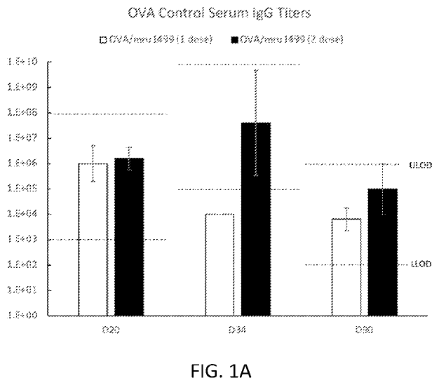

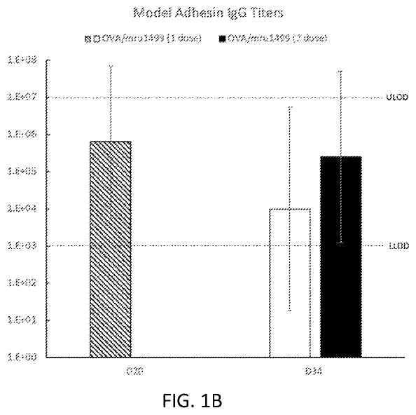

As shown in FIGS. 1A-1. antigen-specific IgG titers for OVA-IgG and mru499-IgG (adhesin) were detected by ELSA in the serum or saliva of animals receiving either one dose or two doses of the multi-component RNA vaccine. Further as shown in Table 4, antigen specific IgA titers were also detected from the serum or saliva of vaccinated animals.

TABLE 4
Antigen-specific IgA and IgG titers from serum or saliva, for various RNA vaccine formulation.
RNAExperimental group size, dosing conditions, and RNA vaccine formulations are specified
per row of table. ELISA setups are shown in the table below and recorded titers
measure IgA or IgG response to an exemplary multi-component RNA vaccine
formulation and exemplary RNA vaccine formulations targeting Methanogen proteins
and with various secretion signals. ELISAs all used Day 34 Serum or saliva samples.

| Experi- | | | ELISA | ELISA | Group | | |
|---|---|---|---|---|---|---|---|
| ment | Condition | Formulation | Coating | Ab2 | Size (n=) | GMT | GSD |
| M1 | 1 Dose | 0.5 mg of 50% OVA_HNadj | OVA | IgG | 5 | 3E+4 | 8 |
| | | (85% HNmod1) + 50% | | | | | |
| | | mru1499_HNadj (85% HNmod1) | | | | | |
| M1 | 2 Dose | 0.5 mg of 50% OVA_HNadj | OVA | IgG | 5 | 3E+7 | 66 |
| | | (85% HNmod1) + 50% | | | | | |
| | | mru1499_HNadj (85% HNmod1) | | | | | |
| M1 | 1 Dose | 0.5 mg of 50% OVA_HNadj | OVA | IgA | 3 | 50 | 2 |
| | | (85% HNmod1) + 50% | | | | | |
| | | mru1499_HNadj (85% HNmod1) | | | | | |
| M1 | 2 Dose | 0.5 mg of 50% OVA_HNadj | OVA | IgA | 2 | 160 | 3 |
| | | (85% HNmod1) + 50% | | | | | |
| | | mru1499_HNadj (85% HNmod1) | | | | | |
| M1 | 1 Dose** | 0.5 mg of 50% OVA_HNadj | OVA | IgG | 5 | 331 | 15 |
| | | (85% HNmod1) + 50% | | | | | |
| | | mru1499_HNadj (85% HNmod1) | | | | | |
| M1 | 2 Dose** | 0.5 mg of 50% OVA_HNadj | OVA | IgG | 5 | 3162 | 4 |
| | | (85% HNmod1) + 50% | | | | | |
| | | mru1499_HNadj (85% HNmod1) | | | | | |
| M1 | 1 Dose** | 0.5 mg of 50% OVA_HNadj | OVA | IgA | 5 | 833 | 36 |
| | | (85% HNmod1) + 50% | | | | | |
| | | mru1499_HNadj (85% HNmod1) | | | | | |
| M1 | 2 Dose** | 0.5 mg of 50% OVA_HNadj | OVA | IgA | 5 | 1821 | 15 |
| | | (85% HNmod1) + 50% | | | | | |
| | | mru1499_HNadj (85% HNmod1) | | | | | |
| M2.1 | F2 | 0.5 mg of mru1499_HNadj | mru1499 | IgG | 4 | 1E+6 | 1 |
| | | (100% HNmod1) | | | | | |
| M2.1 | F4 | 0.5 mg of | mru1499 | IgG | 4 | 3E+5 | 4 |
| | | HNpep_mru1499_HNadj (100% | | | | | |
| | | HNmod1) | | | | | |
| M2.1 | F2 | 0.5 mg of mru1499_HNadj | mru1499 | IgA | 4 | 57 | 6 |
| | | (100% HNmod1) | | | | | |
| M2.1 | F4 | 0.5 mg of | mru1499 | IgA | 4 | 320 | 2 |
| | | HNpep_mru1499_HNadj (100% | | | | | |
| | | HNmod1) | | | | | |

*Saliva Sample.

This data demonstrates that a single component or multi-component RNA vaccine formulations can generate an antigen-specific antibody response in cattle. This data supports the development of a single component or multiplexed vaccination approach (e.g., where two or more antigens are used), to vaccinate an animal, e.g., a ruminant.

### Example 3: In Vivo Effect on Methanogen Abundance and Methane Emissions in Cattle Vaccinated Against a Methanogen Protein

This Example describes the in vivo effect on methanogen abundance and methane emissions in cattle vaccinated with exemplary RNA vaccine formulations targeting Methanogen proteins with various secretion signals. The methods used in this Example are provided in Example 1 above.

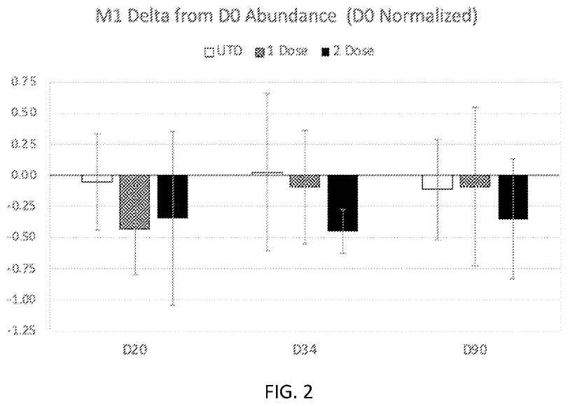

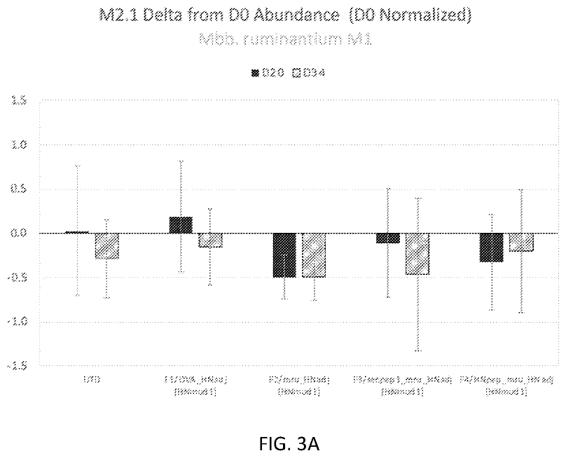

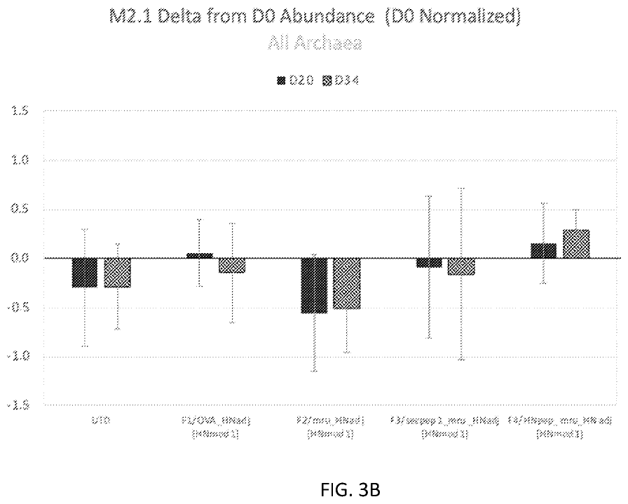

As shown in FIG. 2, calves vaccinated with an exemplary multi-component RNA vaccine showed a decrease in Methanogen species Methanobrevibacter (Mbb.) ruminantium M1 in rumen fluid at Days 20, 34 and 90 post-vaccination as compared to control animals which were either left untreated or not dosed with the vaccine formulation. Similarly, FIGS. 3A-3B show a decrease in Methanogen species Methanobrevibacter (Mbb) ruminantium M1 in rumen fluid of calves vaccinated with an exemplary RNA vaccine targeting Methanogen proteins with various secretion signals. The normalized relative abundance of Archaeal species was also generally decreased in rumen fluid of calves vaccinated with RNA vaccine targeting Methanogen proteins (FIG. 3B). Animals vaccinated with RNA vaccine construct having a HNpep secretory signal, the mru1499 antigen, and HNadjuvant had an increase in Archaeal abundance.

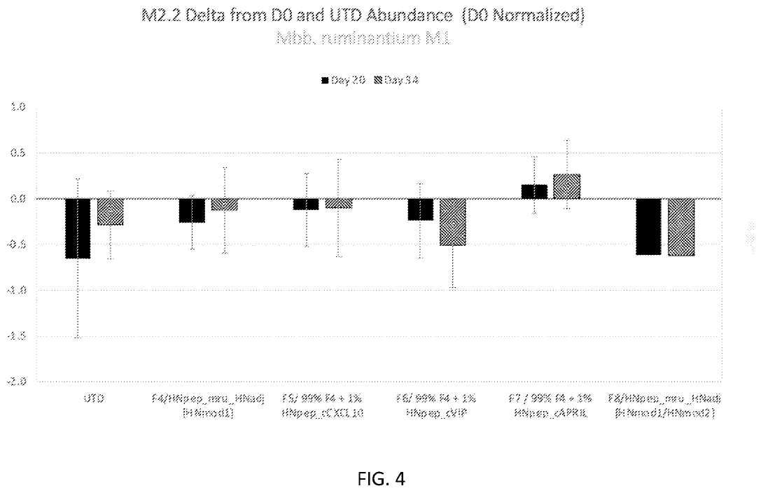

FIG. 4 shows the abundance of methanogen and archaeal species in rumen fluid in cattle vaccinated with exemplary RNA vaccine formulations targeting methanogens, and in some instances including cytokines The RNA used also included the Ac4C and/or 5hmU modifications, as indicated in the figure. Animals vaccinated with RNA vaccine formulations targeting methanogens (mru1499) and those additionally with cytokines CXCL10 or VIP showed lower methanogen and archaeal species compared to each groups' respective Day 0 levels (apparent as negative delta values). Additionally, these groups showed a decrease in total Archaeal abundance compared to the untreated group. The RNA vaccination formulation with both Ac4C and 5hmU modifications resulted in a decrease in abundance of Methanogen species Mbb. ruminantium M1 compared to the untreated group, however, an increase in total Archaeal abundance.

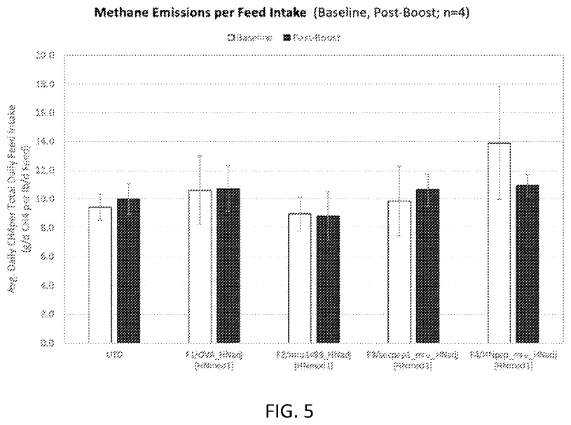

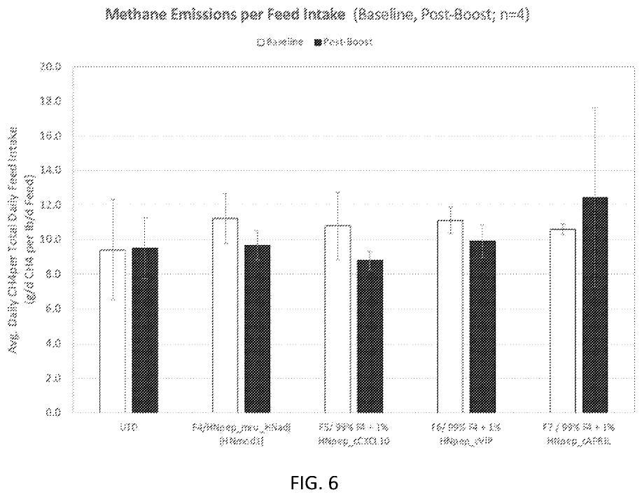

Additionally, methane emissions based on feed intake were also measured for calves vaccinated with an exemplary RNA vaccine formulation targeting Methanogen proteins with various secretion signals. The data in FIG. 5 shows that calves vaccinated with an exemplary construct targeting mru1499 and having a PPa2 secretory peptide and a Sbi adjuvant had lower methane emissions compared to before the vaccination (compare last two bars on right). FIG. 6 shows reduced methane emissions in animals vaccinated with exemplary RNA vaccine formulations targeting methanogens and/or treated with RNA expressing cytokine (CXCL 10, VIP).

Taken together, the data in this Example demonstrates that vaccination with RNA vaccine formulations targeting methanogens and/or treatment with RNA expressing cytokines expressed by cattle can reduce the abundance of methanogens and/or reduce methane emissions. This data further supports the development of a vaccination approach comprising methanogen antigens and/or ruminal-associated antigens for reducing methanogens in the digestive tract of an animal and/or reducing methane emissions.

### Example 4: In Vivo Effect on Methanogen Abundance and Methane Emissions of Cattle Vaccinated with a Multi-Component RNA Vaccine Against Multiple Methanogen Proteins

This Example describes the in vivo effect on methanogen abundance and methane emissions in cattle vaccinated with exemplary multi-component RNA vaccine formulations targeting multiple methanogen proteins. The methods used in this Example are provided in Example 1 above.

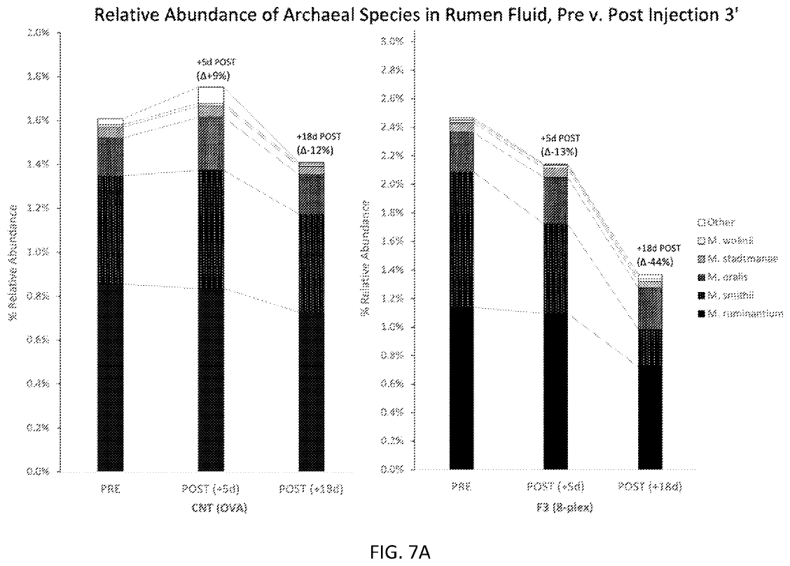

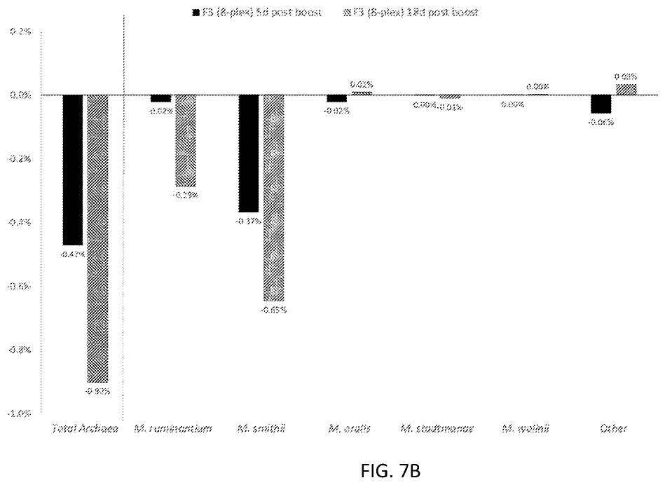

As shown in FIGS. 7A-7B, calves vaccinated with an exemplary multi-component RNA vaccine formulation targeting multiple Methanogen proteins showed a decrease in Total Archaea at +5d POST and +18d POST, relative to abundance at the PRE time point. From the PRE to +18d POST timepoint, calves vaccinated with the F3 multi-component RNA vaccine formulation achieved a 44% decrease in total archaeal abundance (−32%, normalized to the CNT(OVA) group). In addition, calves vaccinated with an exemplary multi-component RNA vaccine formulation targeting multiple methanogen proteins showed a decrease in each individual archaeal species identified at +5d POST, relative to abundance at the PRE time point and normalized to the CNT(OVA) group. Note, the CNT(OVA) negative control is a vaccine formulation targeted to non-methanogen protein, ovalbumin, to generate a non-methanogen specific immune response.

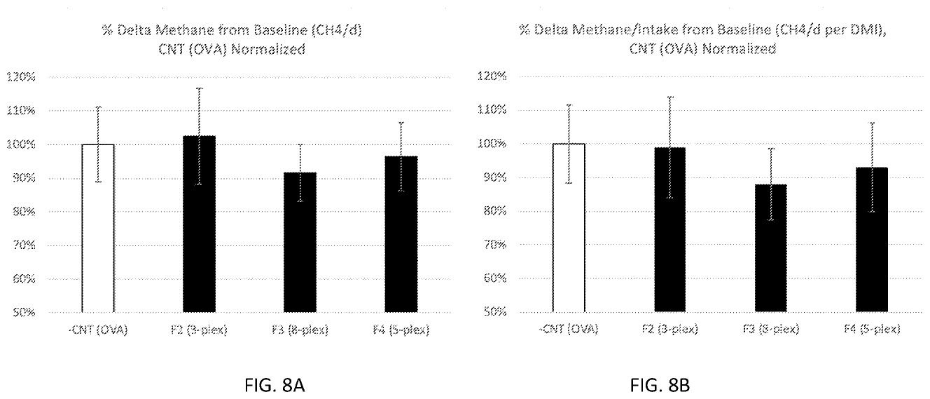

Additionally, methane emissions based on feed intake were also measured for calves vaccinated with exemplary multi-component RNA vaccine formulations targeting multiple methanogens. The data in FIGS. 8A-8B shows that calves vaccinated with F3 or F4 vaccine formulations (as described in Table 3) had lower methane emissions (CH4/day) post-injection relative to the control CNT-OVA (FIG. 8A, last two bars on right). Further, all tested exemplary multi-component RNA vaccine formulations targeting multiple methanogen proteins demonstrated lower methane emissions per feed intake (CH4/day per lb feed) post-injection relative to the control CNT-OVA (FIG. 8B).

Taken together, the data in this Example demonstrates that vaccination with multi-component RNA vaccine formulations targeting one or more methanogen antigens and/or one or more methanogen species can reduce the abundance of methanogens and reduce methane emissions. This data further supports the development of a vaccination approach comprising methanogen antigens or ruminal-associated antigens for reducing methanogens in the digestive tract of an animal and reducing methane emissions.

### Example 5: In Vivo Effect on Growth Efficiency of Cattle Vaccinated Against a Methanogen Protein

This Example describes the in vivo effect on growth efficiency, measured via average daily gain (ADG, lb/day) or the average amount of weight an animal gained per day during a defined feeding period, of cattle vaccinated with exemplary RNA vaccine formulations targeting Methanogen proteins with various secretion signals, and in some instances multi-component formulations, cytokines, and the Ac4C and/or 5hmU modifications. The methods used in this Example are provided in Example 1 above.

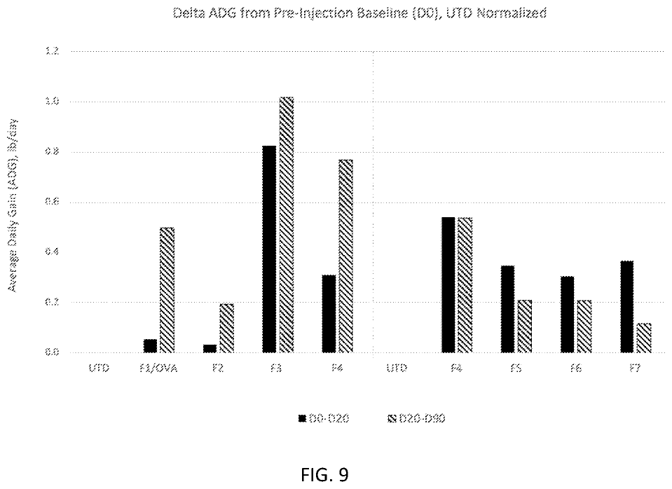

As shown in FIG. 9, calves vaccinated with an exemplary RNA vaccine targeting methanogen proteins and having either a secpep1 or HNpep secretory signal (F3, F4) had significant increases in average daily gain (ADG) during time period “DO-D20” (a 20-day period post prime injection) and “D20-D90” (70-day period post boost injection) as compared to control animals which were either left untreated or not dosed with the vaccine formulation. For the secpep1 formulation, average ADG normalized to untreated (UTD) increased over 0.8 lbs/day (+53% change from baseline ADG) for the DO-D20 period, and over 1.0 lbs/day (+65% change from baseline ADG) for the D20-D90 period. Additionally, the data showed that exemplary RNA vaccine formulations targeting methanogens and with cytokines and/or the 5hmU modification also increased ADG during time period DO-D20 and D20-D90 as compared to control animals which were either left untreated or not dosed with the vaccine formulation.

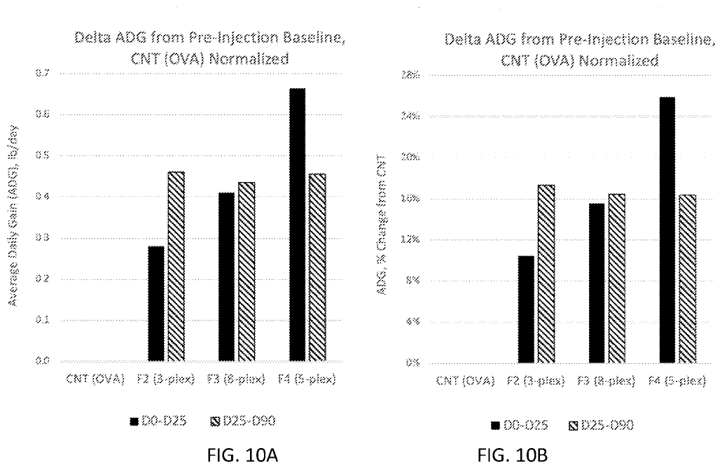

In addition, this Example describes the in vivo effect on growth efficiency, measured via average daily gain (ADG, lb/day), of cattle vaccinated with an exemplary multi-component RNA vaccine against multiple methanogen proteins. As shown in FIGS. 10A-10B, calves vaccinated with multi-component RNA vaccine against multiple Methanogen proteins had increased ADG during time period “D0-D25” (a 25-day period post prime injection) and “D25-D90” (65-day period post boost injection) as compared to animals in the CNT(OVA) group which were vaccinated with a formulation targeted to non-methanogen protein, ovalbumin, to generate a non-methanogen specific immune response.

The data in this Example demonstrates that vaccination with RNA vaccine formulations targeting methanogens can increase growth efficiency of cattle, by way of their average daily weight gained. Additionally, the data demonstrates that vaccination with RNA vaccine formulations targeting one or more methanogen antigens and/or one or more methanogen species can increase growth efficiency of cattle relative to a non-methanogen specific vaccine. Without wishing to be bound by any particular theory, in some embodiments, vaccination with polynucleotides comprising one or more ruminal-associated antigens increases the growth efficiency of an animal (e.g., a ruminant) by reducing one or more microorganisms (e.g., methanogens) in a digestive tract of an animal thus lowering methane emissions. Further without wishing to be bound by any particular theory, since methane produced in the digestive tract of an animal is energy lost (e.g., not absorbed by the body) from food consumed by an animal, reducing methane emissions allows for more energy from the food consumed by the animal to be used for growth, thus increasing growth efficiency of an animal.

This data further supports the development of a vaccination approach comprising ruminal-associated antigens (e.g., ruminal antigens and/or methanogen antigens) for increasing the growth efficiency of ruminant animals.

### NUMBERED EMBODIMENTS

Embodiment 1. An isolated polynucleotide encoding one or more ruminal-associated antigens, fragments thereof or variants thereof.

Embodiment 2. The isolated polynucleotide of embodiment 1, wherein the one or more ruminal-associated antigens comprise one or more ruminal antigens and/or one or more methanogen antigens.

Embodiment 3 The isolated polynucleotide of embodiment 1 or 2, wherein the one or more ruminal-associated antigens comprise one or more ruminal antigens.

Embodiment 4. The isolated polynucleotide of any one of the preceding embodiments, wherein the one or more ruminal antigens are derived from: a polypeptide that is involved in attachment to fermenting bacteria, or a fragment or variant thereof.

Embodiment 5 The isolated polynucleotide of any one of the preceding embodiments, wherein the polynucleotide comprises at least 2, at least 3, at least 4, at least 5, at least 6, at least 7, at least 8, at least 9, or at least 10 ruminal antigens.

Embodiment 6. The isolated polynucleotide of embodiment 1 or 2, wherein the one or more ruminal-associated antigens comprise one or more methanogen antigens.

Embodiment 7. The isolated polynucleotide of embodiment 6, wherein the polynucleotide comprises at least 2, at least 3, at least 4, at least 5, at least 6, at least 7, at least 8, at least 9, at least 10, at least 15, or at least 20 methanogen antigens.

Embodiment 8. The isolated polynucleotide of embodiment 6, wherein the polynucleotide comprises about 2 to about 20, about 2 to about 15, about 2 to about 10, about 2 to about 9, about 2 to about 8, about 2 to about 7, about 2 to about 6, about 2 to about 5, about 2 to about 5, about 2 to about 4, about 2 to about 3, about 3 to about 20, about 4 to about 20, about 5 to about 20, about 6 to about 20, about 7 to about 20, about 8 to about 20, about 9 to about 20, about 10 to about 20, or about 15 to about 20 methanogen antigens.

Embodiment 9. The isolated polynucleotide of any one of embodiments 6-8, wherein the one or more methanogen antigens are the same, e.g., having the same sequence.

Embodiment 10. The isolated polynucleotide of any one of embodiments 6-8, wherein the one or more methanogen antigens are different, e.g., having different sequences.

Embodiment 11. The isolated polynucleotide of any one of embodiments 1-2 or 6-10, wherein the one or more methanogen antigens is derived from a polypeptide found on the cell surface of a wild-type methanogen, or a fragment or variant thereof.

Embodiment 12. The isolated polynucleotide of any one of embodiments 1-2 or 6-11, wherein the one or more methanogen antigens is secreted.

Embodiment 13. The isolated polynucleotide of any one of embodiments 1-2 or 6-12, wherein the one or more methanogen antigens comprise a peptide that is involved in adhesion, attachment, or mobility, or a fragment or variant of a peptide that is involved in adhesion, attachment, mobility.

Embodiment 14. The isolated polynucleotide of embodiment 12 or 13, wherein the secreted methanogen antigen comprises a signal peptide.

Embodiment 15. The isolated polynucleotide of embodiment 14, wherein the signal peptide can be predicted using a prediction algorithm and optionally wherein the prediction score is at least 0.5.

Embodiment 16. The isolated polynucleotide of any one of embodiments 1-2 or 6-15, wherein the one or more methanogen antigens comprise one or more peptides having at least 80% sequence identity to a methanogen protein.

Embodiment 17. The isolated polynucleotide of any one of embodiments 1-2 or 6-16, wherein the polynucleotide comprises a plurality of methanogen antigens each having at least 80% sequence identity to each other.

Embodiment 18. The isolated polynucleotide of any one of embodiments 1-2 or 6-17, wherein the polynucleotide comprises a plurality of methanogen antigens wherein each methanogen antigen in the plurality is associated with a different methanogen species.

Embodiment 19. The isolated polynucleotide of any one of embodiments 1-2 or 6-18, wherein the polynucleotide comprises a plurality of methanogen antigens, and wherein the methanogen antigens in the plurality are associated with at least 2 different, at least 3 different, at least 4 different or at least 5 different methanogen species.

Embodiment 20. The isolated polynucleotide of any one of embodiments 1-2 or 6-19, wherein the polynucleotide comprises a plurality of methanogen antigens and wherein each methanogen antigen in the plurality is associated with the same methanogen species.

Embodiment 21. The isolated polynucleotide of any one of embodiments 18-20, wherein the methanogen species comprises: Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methancaldoococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, or Mathanosarcina acetivorans.

Embodiment 22. The isolated polynucleotide of any one of embodiments 18-21, wherein the methanogen species is or comprises Methanobrevibacter ruminantium, Methanobrevibacter smithii, or Methanobrevibacter oralis.

Embodiment 23. The isolated polynucleotide of any one of embodiments 18-21, wherein the methanogen species is or comprises Methanobrevibacter ruminantium Methanobrevibacter smithii Methanobrevibacter oralis Methanomicrobium mobile or Methanosphaera stadtmanae.

Embodiment 24. The isolated polynucleotide of any one of embodiments 1-2 or 6-23, wherein the one or more methanogen antigens comprise one or more peptides having at least 80% sequence identity to a polypeptide having signal peptidase activity.

Embodiment 25. The isolated polynucleotide of embodiment 24, wherein a polypeptide having signal peptidase activity
(i) is membrane bound;
(ii) has the ability to cleave one or more signal peptides;
(iii) plays a role in converting a secretory protein to a mature form (e.g., from a non-secretory form to a secretory form); and/or
(iv) or any combination thereof.

Embodiment 26. The isolated polynucleotide of any one of embodiments 1-2 or 6-25, wherein the one or more methanogen antigens comprise one or more peptides having: (1) at least 80% sequence identity to an Immunoglobulin (Ig)-like polypeptide, or (2) substantially similar function to an Ig-like domain-containing polypeptide.

Embodiment 27. The isolated polynucleotide of embodiment 26, wherein an Ig-like domain-containing polypeptide comprises a polypeptide characterized as a cell-surface protein that facilitates binding to other cells and/or cell surfaces.

Embodiment 28. The isolated polynucleotide of any one of embodiments 1-2 or 6-27, wherein the one or more methanogen antigens comprises an adhesin or fragment or variant thereof.

Embodiment 29. The isolated polynucleotide of embodiment 28, wherein the one or more methanogen antigens comprises an adhesin protein provided in Table 1 or a sequence with at least 85% identity thereto.

Embodiment 30. The isolated polynucleotide of any one of embodiments 1-2 or 6-29, wherein the one or more methanogen antigens comprises mru1499, or a fragment or variant thereof.

Embodiment 31. The isolated polynucleotide of any one of embodiments 1-2 or 6-30, wherein the one or more methanogen antigens comprises an antigen sequence provided in any one of SEQ ID NOs: 2, 6, or 8 or a sequence with at least 85% identity thereto, or is encoded by a sequence provided in any one of SEQ ID NOs: 1, 5, or 7 or a sequence with at least 85% identity thereto.

Embodiment 32. The isolated polynucleotide of any one of embodiments 1-2 or 6-31, wherein the methanogen antigen comprises a pili protein or fragment or variant thereof.

Embodiment 33. The isolated polynucleotide of any one of embodiments 1-2 or 6-32, wherein the methanogen antigen comprises a flagellin protein, or fragment or variant thereof.

Embodiment 34. The isolated polynucleotide of any one of embodiments 1-2 or 6-33, wherein the one or more methanogen antigens comprise one or more peptides having: (1) at least 80% sequence identity to an archaeal oligosaccharyltransferase (AglB) polypeptide, or (2) substantially similar function to an AglB polypeptide.

Embodiment 35. The isolated polynucleotide of embodiment 34, wherein an AglB polypeptide is characterized as having N-glycosylation activity.

Embodiment 36. The isolated polynucleotide of any one of the preceding embodiments, wherein the one or more methanogen antigens comprise:
(i) one or more peptides having at least 80% sequence identity to a methanogen protein;
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to a polypeptide having signal peptidase activity;
(vi) or any combination thereof.

Embodiment 37. The isolated polynucleotide of embodiment 36, wherein the polynucleotide comprises three methanogen antigens.

Embodiment 38. The isolated polynucleotide of embodiment 37, wherein the three methanogen antigens are associated with at least three different methanogen species.

Embodiment 39. The isolated polynucleotide of any one of embodiments 1-35, wherein the one or more methanogen antigens comprise:
(i) one or more peptides having at least 80% sequence identity to a methanogen protein;
(ii) one or more secreted antigens comprising a signal peptide;
(iii) a plurality of peptides associated with at least 2 different, at least 3 different, at least 4 different or at least 5 different methanogen species;
(iv) one or more peptides having at least 80% sequence identity to an AglB polypeptide or substantially similar function to an AglB polypeptide;
(v) or any combination thereof.

Embodiment 40. The isolated polynucleotide of embodiment 39, wherein the polynucleotide comprises five methanogen antigens.

Embodiment 41. The isolated polynucleotide of embodiment 40, wherein the five methanogen antigens are associated with at least five different methanogen species.

Embodiment 42. The isolated polynucleotide of any one of embodiments 1-35, wherein the one or more methanogen antigens comprise.
(i) one or more peptides having at least 80% sequence identity to a methanogen protein;
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to an Ig-like domain-containing polypeptide or substantially similar function to an Ig-like domain-containing polypeptide; and/or
(vi) any combination thereof.

Embodiment 43. The isolated polynucleotide of embodiment 42, wherein the polynucleotide comprises eight methanogen antigens.

Embodiment 44. The isolated polynucleotide of embodiment 43, wherein the eight methanogen antigens are associated with at least three different methanogen species.

Embodiment 45. The isolated polynucleotide of any one of the preceding embodiments, wherein the one or more ruminal antigens and/or the one or more methanogen antigens are each situated on a separate nucleotide sequence.

Embodiment 46. The isolated polynucleotide of any one of embodiments 1-2 and 6-45, wherein the polynucleotide comprises at least 2 methanogen antigens and the at least 2 methanogen antigens are situated on separate nucleotide sequences.

Embodiment 47. The isolated polynucleotide of any one of embodiments 1-2 and 6-45, wherein the polynucleotide comprises at least 3 methanogen antigens and the at least 3 methanogen antigens are situated on separate nucleotide sequences.

Embodiment 48. The isolated polynucleotide of any one of embodiments 1-2 and 6-45, wherein the polynucleotide comprises at least 4 methanogen antigens and the at least 4 methanogen antigens are situated on separate nucleotide sequences.

Embodiment 49. The isolated polynucleotide of any one of embodiments 1-2 and 6-45, wherein the polynucleotide comprises at least 5 methanogen antigens and the at least 5 methanogen antigens are situated on separate nucleotide sequences.

Embodiment 50. The isolated polynucleotide of any one of embodiments 1-2 and 6-45, wherein the polynucleotide comprises at least 6 methanogen antigens and the at least 6 methanogen antigens are situated on separate nucleotide sequences.

Embodiment 51. The isolated polynucleotide of any one of embodiments 1-2 and 6-45, wherein the polynucleotide comprises at least 7 methanogen antigens and the at least 7 methanogen antigens are situated on separate nucleotide sequences.

Embodiment 52. The isolated polynucleotide of any one of embodiments 1-2 and 6-45, wherein the polynucleotide comprises at least 8 methanogen antigens and the at least 8 methanogen antigens are situated on separate nucleotide sequences.

Embodiment 53. The isolated polynucleotide of any one of embodiments 1-2 and 6-45, wherein the polynucleotide comprises at least 9 methanogen antigens and the at least 9 methanogen antigens are situated on separate nucleotide sequences.

Embodiment 54. The isolated polynucleotide of any one of embodiments 1-2 and 6-45, wherein the polynucleotide comprises at least 10 methanogen antigens and the at least 10 methanogen antigens are situated on separate nucleotide sequences.

Embodiment 55. The isolated polynucleotide of any one of embodiments 1-45, wherein the one or more ruminal antigens and/or the one or more methanogen antigens are situated on the same nucleotide sequence.

Embodiment 56. The isolated polynucleotide of any one of embodiments 1-2 and 6-55, wherein the one or more methanogen antigens comprises a polypeptide antigen sequence provided in Table 2, or a variant and/or fragment thereof.

Embodiment 57. The isolated polynucleotide of any one of embodiments 1-2 and 6-55, wherein the one or more methanogen antigens comprises a polypeptide antigen sequence having at least 85%, at least 90%, at least 91%, at least 92%, at least 93%, at least 94%, at least 95%, at least 96%, at least 97%, at least 98%, or at least 99% identity to an antigen sequence provided in Table 2.

Embodiment 58. The isolated polynucleotide of any one of the preceding embodiments, wherein the polynucleotide comprises a signal peptide.

Embodiment 59. The isolated polynucleotide of embodiment 58, wherein the signal peptide has at least 85% homology to an archaeal signal peptide.

Embodiment 60. The isolated polynucleotide of embodiment 58, wherein the signal peptide has at least 85% homology to a bacterial signal peptide.

Embodiment 61. The isolated polynucleotide of any one of embodiments 58-60, wherein the signal peptide is or comprises a PPA2 signal peptide, or a fragment or variant thereof.

Embodiment 62. The isolated polynucleotide of any one of embodiments 58-60, wherein the signal peptide is or comprises an SSP signal peptide, or a fragment or variant thereof.

Embodiment 63. The isolated polynucleotide of embodiment 58, wherein the signal peptide is or comprises a SARS-CoV-2 Spike secretion signal, or a fragment or variant thereof.

Embodiment 64. The isolated polynucleotide of any one of embodiments 58-63, wherein the signal peptide is situated at the N terminal of the ruminal-associated antigen sequence.

Embodiment 65. The isolated polynucleotide of embodiment 2, wherein the polynucleotide comprises:
(i) a first nucleotide sequence encoding one or more ruminal antigens,
(ii) a second nucleotide sequence encoding one or more methanogen antigens; and/or
(iii) a third nucleotide sequence encoding a chemokine and/or cytokine.

Embodiment 66. The isolated polynucleotide of embodiment 65, wherein the first nucleotide sequence comprises at least 2, at least 3, at least 4, at least 5, at least 6, at least 7, at least 8, at least 9, or at least 10 ruminal antigens.

Embodiment 67. The isolated polynucleotide of embodiment 65 or 66, wherein the second nucleotide sequence comprises at least 2, at least 3, at least 4, at least 5, at least 6, at least 7, at least 8, at least 9, or at least 10 methanogen antigens.

Embodiment 68. The isolated polynucleotide of any one of embodiments 65-67, wherein the first nucleotide sequence, the second nucleotide sequence and/or the third nucleotide sequence are situated on one polynucleotide.

Embodiment 69. The isolated polynucleotide of any one of embodiments 65-67, wherein the first nucleotide sequence, the second nucleotide sequence and/or the third nucleotide sequence are situated on different polynucleotides.

Embodiment 70. The isolated polynucleotide of any one of the preceding embodiments, wherein the polynucleotide comprises a transmembrane domain.

Embodiment 71. The isolated polynucleotide of any one of the preceding embodiments, further comprising a complement C3d-binding polypeptide from an immunoglobulin-binding protein (Sbi) of Staphylococcus aureus.

Embodiment 72. The isolated polynucleotide of embodiment 71, wherein the complement C3d-binding polypeptide is or comprises:
(i) domain III of the Sbi of Staphylococcus aureus, or a functional fragment or a variant thereof;
(ii) domain IV of the Sbi of Staphylococcus aureus, or a functional fragment or a variant thereof; or
(iii) both (i) and (ii).

Embodiment 73. The isolated polynucleotide of any one of the preceding embodiments, wherein the polynucleotide is or comprises DNA.

Embodiment 74. The isolated polynucleotide of any one of the preceding embodiments wherein the polynucleotide is or comprises RNA.

Embodiment 75. The isolated polynucleotide of embodiment 74, wherein the RNA comprises a 5′ cap.

Embodiment 76. The isolated polynucleotide of embodiment 74 or 75, wherein the RNA comprises a polyA tail.

Embodiment 77. The isolated polynucleotide of any one of embodiments 74-76, wherein the polynucleotide sequence comprises one or more ribonucleotides comprising a nucleoside comprising an acetyl group, wherein the nucleoside is N4-acetylcytidine and the modified ribonucleotide has a structure of

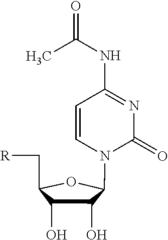

wherein R is a 5′ monophosphate, a 5′ diphosphate, or a 5′ triphosphate.

Embodiment 78. The isolated polynucleotide of embodiment 77, wherein the polyribonucleotide further comprises one or more modified ribonucleotides other than N4-acetylcytidine, optionally wherein the nucleoside is chosen from: an adenosine, an inosine, a guanosine, a cytidine or a uridine, or any combination thereof.

Embodiment 79. The isolated polynucleotide of embodiment 78, wherein the nucleoside of the one or more modified ribonucleotides is 5-hydroxymethyluridine, and the modified ribonucleotide and has a structure of:

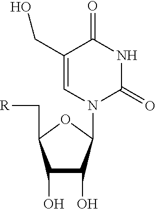

wherein R is a 5′ monophosphate, a 5′ diphosphate, or a 5′ triphosphate.

Embodiment 80. The isolated polynucleotide of any one of embodiments 74-76, wherein the polynucleotide sequence comprises one or more ribonucleotides comprising a nucleoside comprising a hydroxymethyl group, wherein the nucleoside is 5-hydroxymethyluridine and has a structure of:

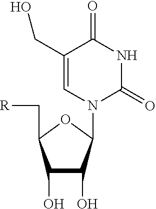

wherein R is a 5′ monophosphate, a 5′ diphosphate, or a 5′ triphosphate.

Embodiment 81. The isolated polynucleotide of embodiment 80, wherein the polyribonucleotide further comprises one or more modified ribonucleotides other than 5-hydroxymethyluridine, wherein the one or more modified ribonucleotides comprises a nucleoside chosen from: an adenosine, an inosine, a guanosine, a cytidine or a uridine, or any combination thereof.

Embodiment 82. The isolated polynucleotide of embodiment 80 or 81, wherein the nucleoside of the one or more modified ribonucleotides is N4-acetylcytidine and the modified ribonucleotide has a structure of:

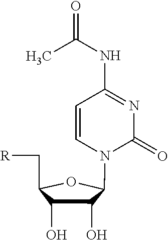

wherein R is a 5′ monophosphate, a 5′ diphosphate, or a 5′ triphosphate.

Embodiment 83. A polypeptide encoded by the polynucleotide of any one of the preceding embodiments.

Embodiment 84. A composition comprising one or more polyribonucleotides of any one of embodiments 1-82, or a polypeptide of embodiment 83.

Embodiment 85. The composition of embodiment 84, wherein the composition is a pharmaceutical composition.

Embodiment 86. The composition of embodiment 84, wherein the composition is an immunogenic composition.

Embodiment 87. The composition of embodiment 84, wherein the composition is a vaccine composition.

Embodiment 88. The composition of any one of embodiments 83-87, wherein the composition is formulated for delivery with a carrier.

Embodiment 89. The composition of 88, wherein the carrier is a lipid nanoparticle, a cationic lipid, a polymeric particle.

Embodiment 90. The composition of any one of embodiments 83-87, wherein the composition is formulated for delivery without a carrier.

Embodiment 91. A method comprising administering a composition according to any one of embodiments 84-90, to a cell, tissue or an animal.

Embodiment 92. The method of embodiment 91, wherein the method is a vaccination method.

Embodiment 93. The method of embodiment 90 or 91, wherein the animal is a ruminant.

Embodiment 94. The method of any one of embodiments 90-92, wherein the ruminant is a cattle, sheep, goats, buffalo, moose, antelope, caribou, or deer.

Embodiment 95. The method of any one of embodiments 90-92, wherein the animal is a domestic animal.

Embodiment 96. The method of any one of embodiments 90-95, wherein the composition is characterized in that administration of the composition to the animal reduces methane emissions from the animal as compared to an otherwise comparable animal not administered the composition or administered a different composition.

Embodiment 97. The method of embodiment 96, wherein the reduction in methane emissions is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to an animal not administered the composition or administered a different composition.

Embodiment 98. The method of embodiment 97, wherein the reduction in methane emissions is about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to an otherwise comparable animal not administered the composition or administered a different composition.

Embodiment 99. The method of any one of embodiments 90-98, wherein the composition is characterized in that administration of the composition to the animal reduces a population of microorganisms in the animal, as compared to an animal not administered the composition or administered a different composition.

Embodiment 100. The method of embodiment 99, wherein the microorganisms is a methanogen.

Embodiment 101. The method of embodiment 100, wherein the methanogen comprises a methanogen from one or more of the following clades: Methanobrevibacter, Methanosphaera, Methanobacterium, Methanosarcinales, Methanonicrobiales, Methanothermobacter, Candidatus Methanomethylophilus, Thermoplasmatales.

Embodiment 102. The method of embodiment 100, wherein the methanogen comprises Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

Embodiment 103. The method of any one of embodiments 100-102, wherein the methanogen abundance is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to methanogen abundance in an animal not administered the composition or administered a different composition.

Embodiment 104. The method of any one of embodiments 100-102, wherein the methanogen abundance is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to abundance of the methanogen in an otherwise comparable animal not administered the composition or administered a different composition.

Embodiment 105. The method of any one of embodiments 100-104, wherein the composition is characterized in that administration of the composition to the animal reduces the abundance of all or substantially all methanogens (e.g., total methanogen abundance) in the animal as compared to methanogen abundance in an otherwise comparable animal not administered the composition or administered a different composition.

Embodiment 106. The method of embodiment 105, wherein total methanogen abundance is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to methanogen abundance in an otherwise comparable animal not administered the composition or administered a different composition.

Embodiment 107. The method of embodiment 105, wherein total methanogen abundance is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to methanogen abundance in an otherwise comparable animal not administered the composition or administered a different composition.

Embodiment 108. The method of any one of embodiments 90-107, wherein the composition is characterized in that administration of the composition to the animal increases a growth rate of the animal as compared to the growth rate of an otherwise comparable animal not administered the composition or administered a different composition.

Embodiment 109. The method of embodiment 108, wherein growth rate is assessed by measuring the weight of the animal at one or more timepoints.

Embodiment 110. The method of embodiment 109, wherein the weight of the animal is measured daily.

Embodiment 111. The method of any one of embodiments 108-110, wherein the growth rate of the animal increases over a period of time.

Embodiment 112. The method of claim 11, wherein the period of time comprises at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months or at least 12 months.

Embodiment 113. The method of any one of embodiments 108-110, wherein the increase in growth rate comprises a daily increase in weight of the animal.

Embodiment 114. The method of embodiment 113, wherein the daily increase in weight of the animal is an increase of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to the daily increase in weight of an otherwise comparable animal not administered the composition or administered a different composition.

Embodiment 115. The method of embodiment 113, wherein the daily increase in weight of the animal is an increase of about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to daily increase in weight of an otherwise comparable animal not administered the composition or administered a different composition.

Embodiment 116. The method of any one of embodiments 91-115, wherein the composition modifies outputs of cellulose fermentation.

Embodiment 117. The method of any one of embodiments 91-116, wherein the composition is delivered with a carrier.

Embodiment 118. The method of any one of embodiments 91-117, wherein the composition is delivered without a carrier.

Embodiment 119. The method of any one of embodiments 91-118, comprising administering one dose of the composition to the animal.

Embodiment 120. The method of any one of embodiments 91-119, comprising administering a plurality of doses of the composition to the animal.

Embodiment 121. The method of embodiment 120, wherein the animal is administered a first dose of the composition followed by one or more subsequent doses of the composition.

Embodiment 122. The method of embodiment 121, wherein the first dose and the one or more subsequent doses of the composition comprise the same methanogen antigens and/or ruminal antigens.

Embodiment 123. The method of embodiment 121, wherein the first dose and the one or more subsequent doses of the composition comprise different methanogen antigens and/or ruminal antigens.

Embodiment 124. The method of any one of embodiments 91-123, wherein the one or more methanogen antigens and/or the one or more ruminal antigens are specific to the animal.

Embodiment 125. The method of embodiment 124, wherein the one or more methanogen antigens and/or the one or more ruminal antigens that are specific to the animal are obtained by a method comprising:
(i) identifying one or more methanogen antigens and/or one or more ruminal antigens that are expressed in the animal; and
(ii) responsive to the identification, selecting one or more methanogen antigens and/or the one or more ruminal antigens to be included in the composition.

Embodiment 126. The method of embodiment 124 or 125, wherein the one or more methanogen antigens and/or the one or more ruminal antigens that are specific to the animal is different from the one or more methanogen antigens and/or the one or more ruminal antigens that are specific to a different animal.

Embodiment 127. The method of embodiment 126, wherein the different animal comprises:
(i) a different species of animal,
(ii) an animal of a different sex,
(iii) an animal of a different age, or
(iv) an animal located in a different geographical location;
(v) the same animal but at a different timepoint; and/or
(vi) any combination thereof.

Embodiment 128. The method of any one of embodiments 120-127, wherein the composition is administered 1, 2, 3, 4, 5, 6, 7, 8, 9, 10, 20 or 30 times to the animal.

Embodiment 129. The method of any one of embodiments 120-128, wherein the composition is administered once every 1 week, 2 weeks, 3 weeks, 1 month, 2 months, 3 months, 4 months, 5 months or 6 months.

Embodiment 130. The method ofany one of embodiments 91-129, wherein the composition is administered in combination with one or more additional agents.

Embodiment 131. The method of embodiment 130, wherein the one or more additional agents comprises a chemical additive, a biological feed additive.

Embodiment 132. The method of any one of embodiments 91-131, wherein the composition is administered in combination with one or more additional compositions.

Embodiment 133. The method of embodiment 132, wherein the additional composition immunizes the animal from a disease, e.g., an infectious disease.

Embodiment 134. A method of manufacturing a composition comprising one or more polyribonucleotides of any one of embodiments 1-82.

Embodiment 135. The method of embodiment 134, wherein the composition is an RNA composition.

Embodiment 136. The method of embodiment 134 or 135, wherein:
(i) the method is an in vitro transcription reaction method; and/or
(ii) the method produces a plurality of polyribonucleotides.

Embodiment 137. The isolated polynucleotide of any one of embodiments 1-5 or 58-81, wherein the ruminal antigen comprises an antigen expressed in any one or all of: (a) Rumen ciliates symbiotic with methanogens; (b) Hydrogen-producing organisms which supply methanogens; (c) Taxa associated with low feed efficiency.

Embodiment 138. The isolated polynucleotide of embodiment 137, wherein Rumen ciliates symbiotic with methanogens comprises one or more, or any combination of: Diplodinium deniatum; Diploplastron affine, Enoploplastron triloricatum; Entodinium simplex; Entodinium caudatum; Entodinium longinucleatum; Epidinium ecaudatum; Eremoplastron bovis; Eudiplodinium maggii; Ostracodinium obtusum; or Polyplastron multivesiculatum.

Embodiment 139. The isolated polynucleotide of embodiment 137, wherein Hydrogen-producing organisms comprise one or more, or any combination of: Butyrivibrio proteoclasticus; Bacteroides thetaiotaomicron; Ruminococcus flavefaciens; Ruminococcus albus.

Embodiment 140. The isolated polynucleotide of embodiment 137, wherein taxa associated with low feed efficiency comprise one or more of: Veillonellaceae; Prevotellaceae; Lachnospiraceae; Succinivibrionaceae; Fibrobacteraceae; Anaerovibrio; Clostridiales; Prevotella; Ruminococcaceae.

Embodiment 141. The method of any one of embodiments 100-104, wherein the composition is characterized in that administration of the composition to the animal reduces the abundance of at least one methanogen species in the animal as compared to abundance of the same methanogen species in an otherwise comparable animal not administered the composition or administered a different composition.

Embodiment 142. The method of embodiment 141, wherein abundance of at least one methanogen species is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to abundance of the same methanogen species in an otherwise comparable animal not administered the composition or administered a different composition.

Embodiment 143. The method of embodiment 141, wherein abundance of at least one methanogen species is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to abundance of the same methanogen species in an otherwise comparable animal not administered the composition or administered a different composition.

Embodiment 144. The method of any one of embodiments 91-123, wherein the one or more methanogen antigens and/or the one or more ruminal antigens are present and/or expressed in the animal and one or more additional animals.

Embodiment 145. The method of embodiment 144, wherein the animal and the one or more additional animals are or comprise:
(i) a same or different species of animal,
(ii) an animal of the same or different sex,
(iii) an animal of substantially the same age or development stage, or
(iv) an animal located in the same or a substantially similar geographical location; or
(v) any combination of (i)-(iv).

Embodiment 146. The method of embodiment 144 or 156, wherein the composition is administered 1, 2, 3, 4, 5, 6, 7, 8, 9, 10, 20 or 30 times to the animal.

Embodiment 147. The method of any one of embodiments 144-146, wherein the composition is administered once every 1 week, 2 weeks, 3 weeks, 1 month, 2 months, 3 months, 4 months, 5 months or 6 months.

Embodiment 148. The method of any one of embodiments 144-147, wherein the composition is administered in combination with one or more additional agents.

Embodiment 149. The method of embodiment 148, wherein the one or more additional agents comprises a chemical additive, a biological feed additive.

Embodiment 150. The method of any one of embodiments 144-149, wherein the composition is administered in combination with one or more additional compositions.

Embodiment 151. The method of embodiment 150, wherein the additional composition immunizes the animal from a disease, e.g. an infectious disease.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. An isolated polynucleotide encoding one or more ruminal-associated antigens, fragments thereof, variants thereof or variant fragments thereof.

2. The isolated polynucleotide of claim 1, wherein the one or more ruminal-associated antigens comprise one or more ruminal antigens and/or one or more methanogen antigens.

3. The isolated polynucleotide of claim 1 or 2, wherein the one or more ruminal-associated antigens comprise one or more ruminal antigens.

4. The isolated polynucleotide of any one of the preceding claims, wherein the one or more ruminal antigens are derived from a polypeptide that is involved in attachment to fermenting bacteria, or a fragment or variant or variant fragment thereof.

5. The isolated polynucleotide of claim 3 or 4, wherein the one or more ruminal antigens comprises at least 2, at least 3, at least 4, at least 5, at least 6, at least 7, at least 8, at least 9, or at least 10 ruminal antigens.

6. The isolated polynucleotide of claim 1 or 2, wherein the one or more ruminal-associated antigens comprise one or more methanogen antigens.

7. The isolated polynucleotide of claim 6, wherein the one or more methanogen antigens comprises at least 2, at least 3, at least 4, at least 5, at least 6, at least 7, at least 8, at least 9, at least 10, at least 15, or at least 20 methanogen antigens.

8. The isolated polynucleotide of claim 6 or 7, wherein the one or more methanogen antigens are the same.

9. The isolated polynucleotide of claim 6 or 7, wherein the one or more methanogen antigens are different.

10. The isolated polynucleotide of any one of claims 6-9, wherein the one or more methanogen antigens comprise:
(i) one or more peptides having at least 80% sequence identity to a methanogen protein;
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to a polypeptide having signal peptidase activity;
(vi) one or more peptides having at least 80% sequence identity to an AglB polypeptide or substantially similar function to an AglB polypeptide;
(vii) one or more peptides having at least 80% sequence identity to an Ig-like domain-containing polypeptide or substantially similar function to an Ig-like domain-containing polypeptide; or
(viii) or any combination thereof.

11. The isolated polynucleotide of any one of claims 6-9, wherein the one or more methanogen antigens comprise:
(i) one or more peptides having at least 80% sequence identity to a methanogen protein;
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to a polypeptide having signal peptidase activity;
(vi) or any combination thereof.

12. The isolated polynucleotide of claim 11, wherein the polynucleotide comprises three methanogen antigens, optionally wherein the three methanogen antigens are associated with at least three different methanogen species.

13. The isolated polynucleotide of any one of claims 6-9, wherein the one or more methanogen antigens comprise:
(i) one or more peptides having at least 80% sequence identity to a methanogen protein;
(ii) one or more secreted antigens comprising a signal peptide;
(iii) a plurality of peptides associated with at least 2 different, at least 3 different, at least 4 different or at least 5 different methanogen species;
(iv) one or more peptides having at least 80% sequence identity to an AglB polypeptide or substantially similar function to an AglB polypeptide; or
(v) any combination thereof.

14. The isolated polynucleotide of claim 13, wherein the polynucleotide comprises five methanogen antigens, optionally wherein the five methanogen antigens are associated with at least five different methanogen species.

15. The isolated polynucleotide of any one of claims 6-9, wherein the one or more methanogen antigens comprise:
(i) one or more peptides having at least 80% sequence identity to a methanogen protein;
(ii) one or more secreted antigens comprising a signal peptide;
(iii) a plurality of peptides having at least 80% sequence identity to each other;
(iv) a plurality of peptides associated with at least 2 different, at least 3 different, at least 4 different or at least 5 different methanogen species;
(v) one or more peptides having at least 80% sequence identity to an Ig-like domain-containing polypeptide or substantially similar function to an Ig-like domain-containing polypeptide; or
(vi) any combination thereof.

16. The isolated polynucleotide of claim 15, wherein the polynucleotide comprises eight methanogen antigens, optionally wherein the eight methanogen antigens are associated with at least three different methanogen species.

17. The isolated polynucleotide of any one of claims 1-2 or 6-16, wherein the one or more methanogen antigens are derived from a polypeptide found on the cell surface of a wild-type methanogen, or a fragment or variant thereof.

18. The isolated polynucleotide of any one of claims 1-2 or 6-17, wherein the methanogen antigen is secreted.

19. The isolated polynucleotide of any one of claims 1-2 or 6-18, wherein the methanogen antigen comprises a peptide that is involved in adhesion, attachment, or mobility, or a fragment or variant of a peptide that is involved in adhesion, attachment, mobility.

20. The isolated polynucleotide of any one of claims 1-2 or 6-18, wherein the methanogen antigen comprises: an adhesin or fragment or variant thereof; a pili protein or fragment or variant thereof; and/or a flagellin protein, or fragment or variant thereof.

21. The isolated polynucleotide of claim 20, wherein the methanogen antigen comprises an adhesin protein provided in Table 1 or a sequence with at least 85% identity thereto.

22. The isolated polynucleotide of any one of claims 1-2 or 6-21, wherein the methanogen antigen comprises: an antigen provided in Table 2 or a fragment or a variant or a variant fragment thereof; or a sequence with at least 85% identity to an antigen sequence provided in Table 2.

23. The isolated polynucleotide of any one of the preceding claims, wherein the one or more ruminal antigens and/or the one or more methanogen antigens are situated on different polynucleotides.

24. The isolated polynucleotide of any one of claims 1-23, wherein the one or more ruminal antigens and/or the one or more methanogen antigens are situated on one polynucleotide.

25. The isolated polynucleotide of any one of the preceding claims, wherein the polynucleotide comprises a signal peptide or a variant or fragment thereof.

26. The isolated polynucleotide of claim 25, wherein the signal peptide has at least 85% homology to an archaeal signal peptide or to bacterial signal peptide.

27. The isolated polynucleotide of claim 25 or 26, wherein the signal peptide is or comprises:
(i) a PPA2 signal peptide, or a fragment or variant thereof;
(ii) an SSP signal peptide, or a fragment or variant thereof;
(iii) a SARS-CoV-2 Spike secretion signal, or a fragment or variant thereof; or
(iv) any combination of (i)-(iii).

28. The isolated polynucleotide of any one of claims 25-27, wherein the signal peptide is situated at the N terminal of the ruminal-associated antigen sequence.

29. The isolated polynucleotide of any one of the preceding claims, wherein the polynucleotide comprises:
(i) a first nucleotide sequence encoding one or more ruminal antigens,
(ii) a second nucleotide sequence encoding one or more methanogen antigens; and/or
(iii) a third nucleotide sequence encoding a chemokine and/or cytokine.

30. The isolated polynucleotide of claim 29, wherein:
the first nucleotide sequence comprises at least 2, at least 3, at least 4, at least 5, at least 6, at least 7, at least 8, at least 9, or at least 10 ruminal antigens; and
the second nucleotide sequence comprises at least 2, at least 3, at least 4, at least 5, at least 6, at least 7, at least 8, at least 9, or at least 10 methanogen antigens.

31. The isolated polynucleotide of any one of the preceding claims, wherein the polynucleotide comprises a transmembrane domain.

32. The isolated polynucleotide of any one of the preceding claims, further comprising a complement C3d-binding polypeptide from an immunoglobulin-binding protein (Sbi) of Staphylococcus aureus.

33. The isolated polynucleotide of claim 32, wherein the complement C3d-binding polypeptide is or comprises
(i) domain III of the Sbi of Staphylococcus aureus, or a functional fragment or a variant thereof;
(ii) domain IV of the Sbi of Staphylococcus aureus, or a functional fragment or a variant thereof; or
(iii) both (i) and (ii).

34. The isolated polynucleotide of any one of the preceding claims, wherein the polynucleotide is or comprises DNA.

35. The isolated polynucleotide of any one of claims 1-34, wherein the polynucleotide is or comprises RNA.

36. The isolated polynucleotide of claim 35, wherein the polynucleotide sequence comprises one or more ribonucleotides comprising a nucleoside comprising an acetyl group, wherein the nucleoside is N4-acetylcytidine and the modified ribonucleotide has a structure of: 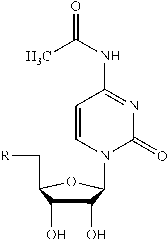
wherein R is a 5′ monophosphate, a 5′ diphosphate, or a 5′ triphosphate.

37. The isolated polynucleotide of claim 36, wherein the polyribonucleotide further comprises one or more modified ribonucleotides other than N4-acetylcytidine, optionally wherein the nucleoside is chosen from: an adenosine, an inosine a guanosine, a cytidine or a uridine, or any combination thereof.

38. The isolated polynucleotide of claim 36, wherein the nucleoside of the one or more modified ribonucleotides is 5-hydroxymethyluridine, and the modified ribonucleotide has a structure of; 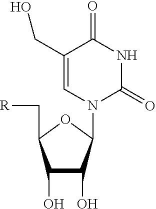
wherein R is a 5′ monophosphate, a 5′ diphosphate, or a 5′ triphosphate.

39. The isolated polynucleotide of any one of claims 33-35, wherein the polynucleotide sequence comprises one or more ribonucleotides comprising a nucleoside comprising a hydroxymethyl group, wherein the nucleoside is 5-hydroxymethyluridine and the modified ribonucleotide has a structure of 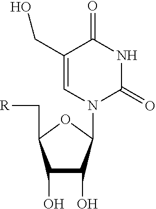
wherein R is a 5′ monophosphate, a 5′ diphosphate, or a 5′ triphosphate.

40. The isolated polynucleotide of claim 39, wherein the polyribonucleotide further comprises one or more modified ribonucleotides other than 5-hydroxymethyluridine, wherein the one or more modified ribonucleotides comprises a nucleoside chosen from: an adenosine, an inosine, a guanosine, a cytidine or a uridine, or any combination thereof.

41. The isolated polynucleotide of claim 39 or 40, wherein the nucleoside of the one or more modified ribonucleotides is N4-acetylcytidine and the modified ribonucleotide has a structure of: 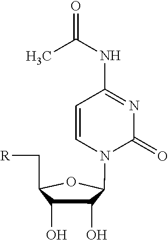
wherein R is a 5′ monophosphate, a 5′ diphosphate, or a 5′ triphosphate.

42. A polypeptide encoded by the polynucleotide of any one of the preceding claims.

43. A composition comprising one or more polyribonucleotides of any one of claims 1-41, or a polypeptide of claim 42.

44. The composition of claim 43, wherein the composition is a pharmaceutical composition.

45. The composition of claim 44, wherein the composition is an immunogenic composition and/or a vaccine composition.

46. A method comprising administering a composition according to any one of claims 43-45, to a cell, tissue or an animal.

47. The method of claim 46, wherein the method is a vaccination method, and the animal is a ruminant or a domestic animal.

48. The method of claim 46 or 47, wherein the composition is characterized in that administration of the composition to the animal reduces methane emissions from the animal as compared to an otherwise comparable animal not administered the composition or administered a different composition.

49. The method of claim 48, wherein the reduction in methane emissions is at least 5%, at least 10%, at least 20%, at least 30%, at least 40%, at least 50%, at least 60%, at least 70%, at least 80%, or at least 90% as compared to an otherwise comparable animal not administered the composition or administered a different composition.

50. The method of any one of claims 46-49, wherein the composition is characterized in that administration of the composition to the animal reduces a population of microorganisms in the animal, as compared to an otherwise comparable animal not administered the composition or administered a different composition.

51. The method of claim 50, wherein the microorganisms is a methanogen, optionally wherein the methanogen comprises a methanogen from one or more of the following clades: Methanobrevibacter, Methanosphaera, Methanobacterium, Methanosarcinales, Methanonicrobiales, Methanothermobacter, Candidatus Methanomethylophilus, Thermoplasmatales.

52. The method of claim 51, wherein the methanogen Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methancaldoococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.

53. The method of any one of claims 46-52, wherein the composition is characterized in that administration of the composition to the animal increases a growth rate of the animal as compared to the growth rate of an otherwise comparable animal not administered the composition or administered a different composition.

54. The method of claim 53, wherein the increase in growth rate comprises a daily increase in weight of the animal, optionally wherein the daily increase in weight of the animal is an increase of at least 5%, at least 10%, at least 20%, at least 30%, at least 40%, at least 50%, at least 60%, at least 70%, at least 80%, or at least 90% of weight as compared to an otherwise comparable animal not administered the composition or administered a different composition.

55. The method of any one of claims 46-54, comprising administering one dose of the vaccine composition to the animal.

56. The method of any one of claims 46-54, comprising administering a plurality of doses of the vaccine composition to the animal.

57. The method of claim 56, wherein the animal is administered a first dose of the composition followed by one or more subsequent doses of the composition.

58. The method of claim 56, wherein the first dose and the one or more subsequent doses of the composition comprise the same methanogen antigens and/or ruminal antigens.

59. The method of claim 56, wherein the first dose and the one or more subsequent doses of the composition comprise different methanogen antigens and/or ruminal antigens.

60. The method of any one of claims 57-59, wherein the composition is administered at least 1, at least 2, at least 3, at least 4, at least 5, at least 6, at least 7, at least 8, at least 9, at least 10, at least 20 or at least 30 times to the animal.

61. The method of any one of claims 46-60, wherein the composition is administered in combination with one or more additional agents, optionally wherein the one or more additional agents comprises a chemical additive, a biological feed additive.

62. The method of any one of claims 46-61, wherein the composition is administered in combination with one or more additional compositions, optionally wherein the additional composition immunizes the animal from a disease, e.g., an infectious disease.

63. A composition comprising the isolated polynucleotide of any one of claims 1-41, or the pharmaceutical composition of claim 43 or 44, for use in administration to (e.g., vaccination of) an animal.

64. Use of a composition comprising the isolated polynucleotide of any one of claims 1-41, or the pharmaceutical composition of claim 43 or 44, in the preparation of a medicament for administration to (e.g., vaccination of) an animal.

65. The composition of claim 63, or the use of claim 64, wherein the isolated polynucleotide or pharmaceutical composition is administered to the animal.

66. The composition of claim 65, or the use of claim 65, wherein administration of the isolated polynucleotide or pharmaceutical composition results in:
(i) reduced methane emissions,
(ii) reduced abundance of one or more microorganisms (e.g., methanogens) in a digestive tract of the animal, and/or
(iii) increased growth rate of the animal,
as compared to an otherwise comparable animal not administered the composition, or administered a different composition.

67. The composition of claim 65 or 66, or the use of claim 65 or 66, wherein the animal is a ruminant or a domestic animal.

68. The composition of any one of claims 65-67, or the use of any one of claims 65-67, methanogen comprises Methanobrevibacter ruminantium, Methanobrevibacter smithii, Methanobrevibacter oralis, Methanomicrobium mobile, Methanobrevibacter wolinii, Methanobrevibacter arboriphilus, Methanobrevibacter boviskoreani, Methanosphaera stadtmanae, Methanosarcina mazei, Methanobrevibacter thaueri, Methanobrevibacter sp. UBA188, Methanosarcina soligelidi, Methanothermobacter thermautotrophicus, Methanococcus aeolicus, Methanocaldococcus jannaschii, Methanococcus voltae, Methanococcus vannielii, Methanococcus maripaludis, Methanopyrus kandleri, Methanocorpusculum labreanum, Methanococcoides burtonii, Methanosaete thermophilia, Methanoregula boonei, Methanosphaerula palustris, Methanoculleus marisnigri, Methanospirillim hungatei, Mathanosarcina acetivorans, or any combination thereof.


SEQUENCE LISTING (122 sequences)

SEQ ID NO: 1 - DNA, 3562 bp, synthetic construct
  source 1..3562: mol_type = other DNA
cttgttctttttgcagaagctcagaataaacgctcaactttggccaccatgctcctggcggtcttgctgatgggttttgtgctcattagtagcgtctctgctatcgatatagatgaggcatcaagtagcagcgatttgtccgattctagtatctctaatgactatcttgttgctaacagcggcgatgactctgtcgcgagctcatcagcctcctcttctattgccgcggatgactcagacctctctaataatgcttcttcatctaatgtcaatttcgagaacgaagtgttgtctactaacaacaacgaagacacagaaagcgaaatcgttaaggattctaaaaatcaactttccagcagctcattgcaggcttctaccaagacaaagacgaccctgaagggtagcggctcatctgtctataggggcaacccttactatgttacactcacggacagcaacgggaaggttctggcttcacagaaagttacgttcaatatcctcggaaaaaattatacacgaacaactgattctaagggggtcgcctctatcaacataaacttggcgaaggggaagtataatatcgcctgtctgtatgctggtacagagaattatgccagttccaaactttctgtggcgcttacagtcaacttgatgtctacaaaaattaacactggaggctcaacggtcaaaaaagggaatgcatacagtgtgactcttacggatggaaacggtaaagccttgagcagtcaaaaagtgacactgaacatactgggtaaaaattatacaaggacgacggatagcaagggcgttgcgagcatagcaataaatctcgctgcaggaaaaaaattcacacttacagccagttacgcagggtccgcaaattacctcagcagcaaagtgtcagctaccgtgacggttcagaaaggggacacatcaatcaagcctagcgggacctcaattgttaaaggtaattcctattcctttacactcgttgacgggtctggaaagggacttgcaaaccaaaaggtggccatcaagatctctggaaagtcatactcccgcacaacaaacagtaatggcgtcgcatccatagctatcaatttggcagccgggaagaaatatagtattgtgtgttcttatgcaggttcctctaattataaagcgagctcctcaacggtgtccctctccgtgacaaatccgtcaactaattctaaaacattctccattgcgaagattgaggccgcagcgacaaatctcaaagcttatgtcaacaagaataaggctgtccctacaacggtttctgttggcggcactaatctgaagatttccgagttctcctatctgatgtctaaggccatagtgaatctgaactctaacaacaccaacgcgatcactctgccgagtggcatttataatggcgcttccgcatccaactcccttaatgccactgtttacaaggcccaatatgttgatttgtccaaacgggtgtataactatatagacaagaataaggttcctgccgcttatggtacggtgtacaatgccaatggagcgtcactgggcaacgcagggtttaacctctacacattcgcgttcgctaagattcttgacttccacaaaacaaataaatatcttccaaattattgttccttcgatagctctgttttcaaggcatccaatggcagtagcagtagcaatagtagtagctccactaactcatcatcatctactaattccagcagtggaagtagcaattcaagcggttctggctctagtacccctgcggtcacagttaaagctaccagtttgaaggcagccagcacaagtgtcataaggggggacgactatagcgtgacgctcacagattccagcggtaatgccctcgccaaccagaaaataacgttcgcgctttcctctagtagttatacccggacaacgaacagcaagggggtcgctagcttgaccctgaacctcgcaggaggcaaatactccatcaccacttcctacgccggaacgtctgcctacaaagcaagcaaacttactaacaccgttacgatatctaactcatcatccaggttttttttgaacgatatagaaaccgctgcggaaaacgttaaaacgtatgtgactaaaaacaaagcactgcctaatactgtcactgtcgccggaacccaactgaccctttctcagtttagttacgtcatggcaaaggcgatccacaatattaacgcttccaattcaaactatatttctctgaagtctgtcgcttccagtaactctactggtgactatttggataccaccgtgtaccgggcacagtacatgaacttgacaaatcgagtgataagcttcgttgaatctgataagatcacacctactttcgcaacggtgtataattctaatggtaaaagcgtcggtaaagccgaattcaaactgtatacattcgcatttgctaaaatactcgctttttacaagactaataattatttgccgacctattgcaccttccagtcaagtgccatcggggttgtcccagacgtcgccaccaacgtgacgataaatagcaagataaatgccaatatgaatcaattcaaagtcgggttgaacgaaaagaatactgtttctaatttgtctgcatatctggttggtacaggacagtccacgatcacgactaatataaaaaacgtcgcggcgcaactgacgaaaggtctcaattccaccgccacaaaggcgctcgcgatctataactttgtgagagacgacatttcctattcctattatagtgattcacggaaaggggcagatgggacgctctcttccggtagcgggaattgcgtggatcaagctagccttgtcgtggcattgtgcagggctgccggtattcctgcaaggtacagccatgctcaggggtgcacattttcctctggcctggtcacaggccacgtgtgggctcaaattttggttgatggagtttggtactctgcggacgcaacgtcagttcggaactcccttggtaacatagttaattggaacaccaatagctatcacagcatgaaacagtatgccgctgttccatttggcggcggcggttctggaggcgggggttccgggggcggtggctccatcgaaaacgctgacaaggccattaaggatttccaggataataaagcgccacatgataagtctgcggcctatgaagctaatagtaaactgccgaaagatctgcgggacaagaataaccgctttgttgaaaaggtgtccatcgaaaaggcaatagtccgccacgatgaaagagtgaaatcagctaacgatgccataagtaaattgaacgagaaggattctatcgagaatcgacggttggcacagagagaggtgaacaaagctccaatggatgtcaaggagcaccttcagaaacaactcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 2 - protein, 1125 aa, synthetic construct
MLLAVLLMGFVLISSVSAIDIDEASSSSDLSDSSISNDYLVANSGDDSVASSSASSSIAADDSDLSNNASSSNVNFENEVLSTNNNEDTESEIVKDSKNQLSSSSLQASTKTKTTLKGSGSSVYRGNPYYVTLTDSNGKVLASQKVTFNILGKNYTRTTDSKGVASININLAKGKYNIACLYAGTENYASSKLSVALTVNLMSTKINTGGSTVKKGNAYSVTLTDGNGKALSSQKVTLNILGKNYTRTTDSKGVASIAINLAAGKKFTLTASYAGSANYLSSKVSATVTVQKGDTSIKPSGTSIVKGNSYSFTLVDGSGKGLANQKVAIKISGKSYSRTTNSNGVASIAINLAAGKKYSIVCSYAGSSNYKASSSTVSLSVTNPSTNSKTFSIAKIEAAATNLKAYVNKNKAVPTTVSVGGTNLKISEFSYLMSKAIVNLNSNNTNAITLPSGIYNGASASNSLNATVYKAQYVDLSKRVYNYIDKNKVPAAYGTVYNANGASLGNAGFNLYTFAFAKILDFHKTNKYLPNYCSFDSSVFKASNGSSSSNSSSSTNSSSSTNSSSGSSNSSGSGSSTPAVTVKATSLKAASTSVIRGDDYSVTLTDSSGNALANQKITFALSSSSYTRTTNSKGVASLTLNLAGGKYSITTSYAGTSAYKASKLTNTVTISNSSSRFFLNDIETAAENVKTYVTKNKALPNTVTVAGTQLTLSQFSYVMAKAIHNINASNSNYISLKSVASSNSTGDYLDTTVYRAQYMNLTNRVISFVESDKITPTFATVYNSNGKSVGKAEFKLYTFAFAKILAFYKTNNYLPTYCTFQSSAIGVVPDVATNVTINSKINANMNQFKVGLNEKNTVSNLSAYLVGTGQSTITTNIKNVAAQLTKGLNSTATKALAIYNFVRDDISYSYYSDSRKGADGTLSSGSGNCVDQASLVVALCRAAGIPARYSHAQGCTFSSGLVTGHVWAQILVDGVWYSADATSVRNSLGNIVNWNTNSYHSMKQYAAVPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 3 - DNA, 1690 bp, synthetic construct
  source 1..1690: mol_type = other DNA
cttgttctttttgcagaagctcagaataaacgctcaactttggccaccatggggtccataggggccgcgtcaatggagttttgcttcgacgtcttcaaagagttgaaggtccaccacgcgaatgaaaatatcttctattgtcctattgcaattatgagtgcccttgcaatggtttaccttggggcaaaagacagtacaaggacacaaatcaacaaagtggtccgatttgataaattgccgggatttggggatagtatcgaagctcagtgtgggacgagtgtcaacgtgcattcttctcttcgcgatatcctcaatcagatcaccaagcctaacgacgtgtactccttttccctcgcttctcgactttatgcggaagagcggtatcctatattgcctgagtatcttcagtgcgttaaagagctgtatcgcggcggtctcgaaccaatcaattttcaaacggcggccgaccaagctcgagagctgattaattcttgggtggaatctcagaccaacgggattatccgcaacgttcttcagccgtcttctgtcgactcacagaccgctatggttttggtgaacgcaatcgtctttaaagggttgtgggagaaagcgtttaaggacgaagatactcaggcaatgccatttagagttacagaacaagaatccaaacctgtccagatgatgtaccaaattggcttgttcagggtggcgtcaatggcttcagagaaaatgaagatcttggagcttccttttgcttccggtactatgtccatgctggtcctgctcccggatgaagtctcaggtctggaacaactggagagcattataaattttgagaaactcacggaatggacctcctcaaatgttatggaagaaagaaagatcaaggtctatctgccgcggatgaagatggaggaaaaatacaaccttacttctgtgttgatggctatgggcataactgatgtgttcagcagttctgctaatcttagcgggatatcaagcgcagaatctttgaagatctctcaagctgttcacgctgctcacgctgaaataaacgaggctgggcgagaagtggtcggttccgcggaggccggggtggatgcggcgtccgtttcagaagagttccgagcggaccatcctttcctgttttgtatcaagcatattgcgactaatgctgtgctctttttcgggcgctgcgtgtctcctggcggcggcggttctggaggcgggggttccgggggcggtggctccatcgaaaacgctgacaaggccattaaggatttccaggataataaagcgccacatgataagtctgcggcctatgaagctaatagtaaactgccgaaagatctgcgggacaagaataaccgctttgttgaaaaggtgtccatcgaaaaggcaatagtccgccacgatgaaagagtgaaatcagctaacgatgccataagtaaattgaacgagaaggattctatcgagaatcgacggttggcacagagagaggtgaacaaagctccaatggatgtcaaggagcaccttcagaaacaactcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 4 - protein, 501 aa, synthetic construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 5 - DNA, 3553 bp, synthetic construct
  source 1..3553: mol_type = other DNA
cttgttctttttgcagaagctcagaataaacgctcaactttggccaccatgttcgtgttcctggtgctcctgcctctggtgtcatcagccgccatcgacatagatgaagcttcctcctccagcgatctgtccgattctagcatcagcaatgactacctggtcgctaacagcggggacgactcagtcgcttccagtagtgcaagctcctccatagcagctgacgacagcgatctcagcaataatgccagctccagcaatgtgaatttcgagaacgaggtcctcagcaccaacaataacgaggacacagagagtgaaatcgtgaaagatagcaagaaccaattgagcagctccagtctgcaggccagcacaaagactaagaccactttgaaggggtccggaagcagcgtctaccgggggaacccatactacgtcaccctgaccgattcaaatggaaaggttctggcctcccagaaagtgactttcaacatcctgggcaaaaattacacccggaccactgatagtaaaggcgtggcctccatcaacatcaatctggccaaaggcaaatataatatcgcttgtctgtacgccggtacagagaattacgcatcatctaagttgagtgtggctctgaccgtcaaccttatgtccaccaaaattaataccggtggatctaccgtcaagaagggtaatgcatattccgttaccctgaccgatggaaacggaaaggccctctccagccagaaggtcaccttgaacatcctcggtaaaaactatacccgcactaccgactctaaaggcgtggccagcatcgccatcaatcttgctgcaggcaagaaattcacactgactgctagctatgcaggctctgcaaactatcttagtagtaaggtgtctgccacagtgaccgtccagaagggcgatactagcatcaaaccaagcggaacctccatcgtgaaaggaaactcatatagcttcaccttggtggacggcagtgggaagggcctcgcaaatcaaaaggtcgccatcaaaatcagcggtaagagctactccaggaccaccaactctaacggtgtggccagtatcgccatcaacctggccgctggcaagaagtactctatcgtgtgttcctatgccggcagcagcaactacaaggcttcctctagcaccgttagtctgagtgtcaccaacccatccactaatagcaagaccttctccatagccaaaattgaagctgctgccactaacctcaaagcctatgtgaacaagaacaaagcagtgccaacaaccgtgagcgtcggaggcactaacttgaagatcagtgagttttcatacttgatgtctaaggccattgtgaatctgaatagcaataataccaacgcaatcaccctgccttcaggtatctacaatggggcaagcgcatctaactcactgaacgcaactgtctataaagcacagtacgtcgacctgtcaaagagagtttacaattatatagataaaaacaaggtgccagccgcttacggaaccgtttataacgctaacggtgcctccctggggaacgctggctttaatctgtatacctttgcctttgccaagatcctggacttccacaaaacaaacaagtacctgccgaactactgttccttcgacagttccgtctttaaggcctctaatggctcaagtagctccaatagttcaagttccactaactctagtagttctaccaattcaagctccggctccagcaactctagtggctctgggtcctctacaccggccgtcaccgtgaaggctacctcactgaaagccgcctcaaccagcgttatccggggcgacgactactctgttactctgactgactccagcgggaatgcccttgctaatcagaaaattaccttcgcactgtccagcagctcatatacacgcaccacaaacagcaagggcgtggcttcactcacactcaatctcgctggagggaagtattcaatcacaaccagctacgctggaacatccgcttacaaggcttccaagcttaccaacaccgtgactatcagcaattcttcttccagattctttttgaacgacattgagacagcagctgagaatgttaagacttatgtcacaaaaaacaaggcacttccaaatactgtcaccgtggctggaacccagctgacactgtcacagttctcttacgtcatggcaaaagccattcacaacataaacgccagtaatagcaactatatatccctgaaaagcgtggctagctctaactctactggagactaccttgacaccaccgtgtaccgggcccagtacatgaatctcacaaaccgagtgatttcttttgtggaaagcgataagattacacctacctttgcaaccgtttataacagcaatggcaagagcgtgggcaaggctgagttcaagctgtacaccttcgcctttgccaaaattctggccttttacaagaccaataactacctgcctacctattgcacattccagtctagcgccattggggtggtgcctgacgtggccaccaacgtgactatcaactccaagattaacgctaacatgaatcagttcaaggtggggctgaatgagaagaacaccgtgtcaaacttgtccgcctacctggtgggcacaggccaatccaccataacaaccaatatcaagaacgttgctgcacagctcaccaaaggcctgaattccaccgcaaccaaggccctcgctatatacaactttgtgagagacgacatctcctactcatactacagtgactctaggaagggtgcagacggtaccctgtccagtggaagtggtaactgtgttgaccaggccagtctcgtcgtggctctgtgccgcgccgccgggatcccagctcgatatagtcacgcacagggatgtacattttcaagcgggctggttacagggcatgtgtgggctcaaattctggtcgatggcgtctggtactcagccgacgcaacatccgtgcggaattcactggggaacattgtgaattggaacaccaactcctaccatagcatgaaacagtatgctgctgtcccttttggtggcggcggttctgggggaggcggcagcggaggtggcggctctatcgaaaacgctgataaggccatcaaggatttccaagataataaggccccgcatgacaagtccgctgcctacgaggccaactccaaactgccgaaggacctccgggacaagaataaccgctttgtggagaaagtgtccattgaaaaggccatcgtccgccacgacgagagggtcaagagcgccaacgatgctatttccaagcttaacgaaaaggacagcattgagaaccgccggctggcacagcgagaagtgaataaagcacctatggatgtgaaggagcacctgcagaaacaactggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 6 - protein, 1122 aa, synthetic construct
MFVFLVLLPLVSSAAIDIDEASSSSDLSDSSISNDYLVANSGDDSVASSSASSSIAADDSDLSNNASSSNVNFENEVLSTNNNEDTESEIVKDSKNQLSSSSLQASTKTKTTLKGSGSSVYRGNPYYVTLTDSNGKVLASQKVTFNILGKNYTRTTDSKGVASININLAKGKYNIACLYAGTENYASSKLSVALTVNLMSTKINTGGSTVKKGNAYSVTLTDGNGKALSSQKVTLNILGKNYTRTTDSKGVASIAINLAAGKKFTLTASYAGSANYLSSKVSATVTVQKGDTSIKPSGTSIVKGNSYSFTLVDGSGKGLANQKVAIKISGKSYSRTTNSNGVASIAINLAAGKKYSIVCSYAGSSNYKASSSTVSLSVTNPSTNSKTFSIAKIEAAATNLKAYVNKNKAVPTTVSVGGTNLKISEFSYLMSKAIVNLNSNNTNAITLPSGIYNGASASNSLNATVYKAQYVDLSKRVYNYIDKNKVPAAYGTVYNANGASLGNAGFNLYTFAFAKILDFHKTNKYLPNYCSFDSSVFKASNGSSSSNSSSSTNSSSSTNSSSGSSNSSGSGSSTPAVTVKATSLKAASTSVIRGDDYSVTLTDSSGNALANQKITFALSSSSYTRTTNSKGVASLTLNLAGGKYSITTSYAGTSAYKASKLTNTVTISNSSSRFFLNDIETAAENVKTYVTKNKALPNTVTVAGTQLTLSQFSYVMAKAIHNINASNSNYISLKSVASSNSTGDYLDTTVYRAQYMNLTNRVISFVESDKITPTFATVYNSNGKSVGKAEFKLYTFAFAKILAFYKTNNYLPTYCTFQSSAIGVVPDVATNVTINSKINANMNQFKVGLNEKNTVSNLSAYLVGTGQSTITTNIKNVAAQLTKGLNSTATKALAIYNFVRDDISYSYYSDSRKGADGTLSSGSGNCVDQASLVVALCRAAGIPARYSHAQGCTFSSGLVTGHVWAQILVDGVWYSADATSVRNSLGNIVNWNTNSYHSMKQYAAVPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 7 - DNA, 3832 bp, synthetic construct
  source 1..3832: mol_type = other DNA
cttgttctttttgcagaagctcagaataaacgctcaactttggccaccatgcggttcccttctattttcaccgcagtcttgttcgctgctagtagcgctttggctatgccactgagcggaggcggatctggggggtcaggctctatgcctctgaacactaccacagaggacgagactgcacagatacctgcagaggcagtcatcgggtacctcgacctcgagggtgactttgatgtggccgttttgcctttctctaattccactaacaacggcctgctgttcatcaacaccaccatagccagtatcgccgcaaaagaagagggagtcagccggaggttgttgcggaggttgctgagaaggttgctggccgcaatcgacatagatgaagcttcctcctccagcgatctgtccgattctagcatcagcaatgactacctggtcgctaacagcggggacgactcagtcgcttccagtagtgcaagctcctccatagcagctgacgacagcgatctcagcaataatgccagctccagcaatgtgaatttcgagaacgaggtcctcagcaccaacaataacgaggacacagagagtgaaatcgtgaaagatagcaagaaccaattgagcagctccagtctgcaggccagcacaaagactaagaccactttgaaggggtccggaagcagcgtctaccgggggaacccatactacgtcaccctgaccgattcaaatggaaaggttctggcctcccagaaagtgactttcaacatcctgggcaaaaattacacccggaccactgatagtaaaggcgtggcctccatcaacatcaatctggccaaaggcaaatataatatcgcttgtctgtacgccggtacagagaattacgcatcatctaagttgagtgtggctctgaccgtcaaccttatgtccaccaaaattaataccggtggatctaccgtcaagaagggtaatgcatattccgttaccctgaccgatggaaacggaaaggccctctccagccagaaggtcaccttgaacatcctcggtaaaaactatacccgcactaccgactctaaaggcgtggccagcatcgccatcaatcttgctgcaggcaagaaattcacactgactgctagctatgcaggctctgcaaactatcttagtagtaaggtgtctgccacagtgaccgtccagaagggcgatactagcatcaaaccaagcggaacctccatcgtgaaaggaaactcatatagcttcaccttggtggacggcagtgggaagggcctcgcaaatcaaaaggtcgccatcaaaatcagcggtaagagctactccaggaccaccaactctaacggtgtggccagtatcgccatcaacctggccgctggcaagaagtactctatcgtgtgttcctatgccggcagcagcaactacaaggcttcctctagcaccgttagtctgagtgtcaccaacccatccactaatagcaagaccttctccatagccaaaattgaagctgctgccactaacctcaaagcctatgtgaacaagaacaaagcagtgccaacaaccgtgagcgtcggaggcactaacttgaagatcagtgagttttcatacttgatgtctaaggccattgtgaatctgaatagcaataataccaacgcaatcaccctgccttcaggtatctacaatggggcaagcgcatctaactcactgaacgcaactgtctataaagcacagtacgtcgacctgtcaaagagagtttacaattatatagataaaaacaaggtgccagccgcttacggaaccgtttataacgctaacggtgcctccctggggaacgctggctttaatctgtatacctttgcctttgccaagatcctggacttccacaaaacaaacaagtacctgccgaactactgttccttcgacagttccgtctttaaggcctctaatggctcaagtagctccaatagttcaagttccactaactctagtagttctaccaattcaagctccggctccagcaactctagtggctctgggtcctctacaccggccgtcaccgtgaaggctacctcactgaaagccgcctcaaccagcgttatccggggcgacgactactctgttactctgactgactccagcgggaatgcccttgctaatcagaaaattaccttcgcactgtccagcagctcatatacacgcaccacaaacagcaagggcgtggcttcactcacactcaatctcgctggagggaagtattcaatcacaaccagctacgctggaacatccgcttacaaggcttccaagcttaccaacaccgtgactatcagcaattcttcttccagattctttttgaacgacattgagacagcagctgagaatgttaagacttatgtcacaaaaaacaaggcacttccaaatactgtcaccgtggctggaacccagctgacactgtcacagttctcttacgtcatggcaaaagccattcacaacataaacgccagtaatagcaactatatatccctgaaaagcgtggctagctctaactctactggagactaccttgacaccaccgtgtaccgggcccagtacatgaatctcacaaaccgagtgatttcttttgtggaaagcgataagattacacctacctttgcaaccgtttataacagcaatggcaagagcgtgggcaaggctgagttcaagctgtacaccttcgcctttgccaaaattctggccttttacaagaccaataactacctgcctacctattgcacattccagtctagcgccattggggtggtgcctgacgtggccaccaacgtgactatcaactccaagattaacgctaacatgaatcagttcaaggtggggctgaatgagaagaacaccgtgtcaaacttgtccgcctacctggtgggcacaggccaatccaccataacaaccaatatcaagaacgttgctgcacagctcaccaaaggcctgaattccaccgcaaccaaggccctcgctatatacaactttgtgagagacgacatctcctactcatactacagtgactctaggaagggtgcagacggtaccctgtccagtggaagtggtaactgtgttgaccaggccagtctcgtcgtggctctgtgccgcgccgccgggatcccagctcgatatagtcacgcacagggatgtacattttcaagcgggctggttacagggcatgtgtgggctcaaattctggtcgatggcgtctggtactcagccgacgcaacatccgtgcggaattcactggggaacattgtgaattggaacaccaactcctaccatagcatgaaacagtatgctgctgtcccttttggtggcggcggttctgggggaggcggcagcggaggtggcggctctatcgaaaacgctgataaggccatcaaggatttccaagataataaggccccgcatgacaagtccgctgcctacgaggccaactccaaactgccgaaggacctccgggacaagaataaccgctttgtggagaaagtgtccattgaaaaggccatcgtccgccacgacgagagggtcaagagcgccaacgatgctatttccaagcttaacgaaaaggacagcattgagaaccgccggctggcacagcgagaagtgaataaagcacctatggatgtgaaggagcacctgcagaaacaactggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 8 - protein, 1215 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSRRLLRRLLRRLLAAIDIDEASSSSDLSDSSISNDYLVANSGDDSVASSSASSSIAADDSDLSNNASSSNVNFENEVLSTNNNEDTESEIVKDSKNQLSSSSLQASTKTKTTLKGSGSSVYRGNPYYVTLTDSNGKVLASQKVTFNILGKNYTRTTDSKGVASININLAKGKYNIACLYAGTENYASSKLSVALTVNLMSTKINTGGSTVKKGNAYSVTLTDGNGKALSSQKVTLNILGKNYTRTTDSKGVASIAINLAAGKKFTLTASYAGSANYLSSKVSATVTVQKGDTSIKPSGTSIVKGNSYSFTLVDGSGKGLANQKVAIKISGKSYSRTTNSNGVASIAINLAAGKKYSIVCSYAGSSNYKASSSTVSLSVTNPSTNSKTFSIAKIEAAATNLKAYVNKNKAVPTTVSVGGTNLKISEFSYLMSKAIVNLNSNNTNAITLPSGIYNGASASNSLNATVYKAQYVDLSKRVYNYIDKNKVPAAYGTVYNANGASLGNAGFNLYTFAFAKILDFHKTNKYLPNYCSFDSSVFKASNGSSSSNSSSSTNSSSSTNSSSGSSNSSGSGSSTPAVTVKATSLKAASTSVIRGDDYSVTLTDSSGNALANQKITFALSSSSYTRTTNSKGVASLTLNLAGGKYSITTSYAGTSAYKASKLTNTVTISNSSSRFFLNDIETAAENVKTYVTKNKALPNTVTVAGTQLTLSQFSYVMAKAIHNINASNSNYISLKSVASSNSTGDYLDTTVYRAQYMNLTNRVISFVESDKITPTFATVYNSNGKSVGKAEFKLYTFAFAKILAFYKTNNYLPTYCTFQSSAIGVVPDVATNVTINSKINANMNQFKVGLNEKNTVSNLSAYLVGTGQSTITTNIKNVAAQLTKGLNSTATKALAIYNFVRDDISYSYYSDSRKGADGTLSSGSGNCVDQASLVVALCRAAGIPARYSHAQGCTFSSGLVTGHVWAQILVDGVWYSADATSVRNSLGNIVNWNTNSYHSMKQYAAVPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 9 - DNA, 760 bp, synthetic construct
  source 1..760: mol_type = other DNA
cttgttctttttgcagaagctcagaataaacgctcaactttggccaccatgcggttcccttctattttcaccgcagtcttgttcgctgctagtagcgctttggctatgccactgagcggaggcggatctggggggtcaggctctatgcctctgaacactaccacagaggacgagactgcacagatacctgcagaggcagtcatcgggtacctcgacctcgagggtgactttgatgtggccgttttgcctttctctaattccactaacaacggcctgctgttcatcaacaccaccatagccagtatcgccgcaaaagaagagggagtcagccggaggttgttgcggaggttgctgagaaggttgctggccgcagtgcctttgtcccggaatacacggtgctcttgcattgagatttccaacggtagtgtgaaccctcggtcccttgagaaactcgaggtgatccctgcctcacagtcttgtcctagagtggagattatcgctaccatgaaaaagaatggagagaagcgatgtcttaaccctgaatccaagactatcaagaatctgctgaaggctatcaataagcagcggaccaagcgctcaccacggacacggaaagaggcctaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 10 - protein, 191 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSRRLLRRLLRRLLAAVPLSRNTRCSCIEISNGSVNPRSLEKLEVIPASQSCPRVEIIATMKKNGEKRCLNPESKTIKNLLKAINKQRTKRSPRTRKEA

SEQ ID NO: 11 - DNA, 595 bp, synthetic construct
  source 1..595: mol_type = other DNA
cttgttctttttgcagaagctcagaataaacgctcaactttggccaccatgcggttcccttctattttcaccgcagtcttgttcgctgctagtagcgctttggctatgccactgagcggaggcggatctggggggtcaggctctatgcctctgaacactaccacagaggacgagactgcacagatacctgcagaggcagtcatcgggtacctcgacctcgagggtgactttgatgtggccgttttgcctttctctaattccactaacaacggcctgctgttcatcaacaccaccatagccagtatcgccgcaaaagaagagggagtcagccggaggttgttgcggaggttgctgagaaggttgctggccgcacatagtgatgccgtctttaccgacaactacactaggttgcgaaagcagatggccgtgaagaagtacctgaacagtattttgaactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 12 - protein, 136 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSRRLLRRLLRRLLAAHSDAVFTDNYTRLRKQMAVKKYLNSILN

SEQ ID NO: 13 - DNA, 949 bp, synthetic construct
  source 1..949: mol_type = other DNA
cttgttctttttgcagaagctcagaataaacgctcaactttggccaccatgcggttcccttctattttcaccgcagtcttgttcgctgctagtagcgctttggctatgccactgagcggaggcggatctggggggtcaggctctatgcctctgaacactaccacagaggacgagactgcacagatacctgcagaggcagtcatcgggtacctcgacctcgagggtgactttgatgtggccgttttgcctttctctaattccactaacaacggcctgctgttcatcaacaccaccatagccagtatcgccgcaaaagaagagggagtcagccggaggttgttgcggaggttgctgagaaggttgctggccgcagcagtgctgactcggaagcacaagaagaaacgaagcgtgcttcacctcgtgccgatcaacattacaagcaaggaagatagtgacgtcaccgaagtgatgtggcagccagcactccagcggggccggggacttgaggctcagggctacgtggtgagggtttgggacgcaggggtctatctgctgtatagccaggtgctgtttcacgatgaaactttcaccatgggccagatggtgtcccgggagggccaggggaggcaggaaacactgttccggtgcattcagagcatgccatctaaccctgactgggcctacaatagttgctactccgccggggtgtttcaccttcaccagggggatatactgagcgttgttatcccacgggccagagcaaagctgtctctgagcccgcacggcacctttttggggctcgtgaagctctaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 14 - protein, 254 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSRRLLRRLLRRLLAAAVLTRKHKKKRSVLHLVPINITSKEDSDVTEVMWQPALQRGRGLEAQGYVVRVWDAGVYLLYSQVLFHDETFTMGQMVSREGQGRQETLFRCIQSMPSNPDWAYNSCYSAGVFHLHQGDILSVVIPRARAKLSLSPHGTFLGLVKL

SEQ ID NO: 15 - protein, 1523 aa, synthetic construct
MFVFLVLLPLVSSAAENTDNALSTDTHSNDNVLSTDSRSNENALTNENTLLTDTHSNENALTTDPLTKENTHSYKDSEKSLSSDAFNKTIYVNKTGSDEGDGSEANPYATLKKSISQLDDSDNAVIYIGPGNYTGENNSALEINLDHKDHDGSLSIIGDSNGGTVFDGENLNPIIISISEDSIVTLINITFTHGKNNMGSAIRSSGNLTIDNCIFTENYATNLAALYVDKHSPLTVMNSKFLENRAKQCADIYFSQNSEIILLNNLFEGSTAEYSYAYSPSVSLQTGKSLVKGNTFKNLTGAYYKGALYIAYNNGINIANITDNTFINCNYTGTDGAILFFQNAYLKNNKFIDCHSSTAFLYSNTEFNAYLSFEDAEIDGTTFFLKANVTDDMGNKVKNAKVIFYLNGENVGSASSDNNGVAMISIKKLLENGEYVISGTQSYSEINPFGVNVKNATARVNYDHSSLEVWVSTDGDDGSGNGSEDNPFKTLRKALDYGTASAVNLTVHVKNGIYNGDDNRDLSYSTLGKITIVGESYSNVVIDGENITKSIFAFSSTLDVTLINLTLINCPSTLINAYTLSMMDNIVINSGTIRAQTGNNGVTIDNLRVINGTDQAITGYNLRLTNSRFENCDGLTHTGLIWLSTNNNKVTYLENNTFFNNTIAGSAGGGAAYYIQSDLISINNTFDSNWITESRGENVAYAGGRHIISINDKFINNEVPKYVAQYRSIGNEECEIIVENITFINNKASGNGAGLATTGAIVKGGKFINNSASGNGGAIYLLNHDNTSSYCQMSLEDVIFENNSATCGKDIFIEGSSGNNIFTYLNNLTIVANDLNVTSLSDNLTVSVFHPSGAIIGGGEISFYLDGEYIGKSTLVNQNASLEYVGFKNNTIYEFTSIYEYASLNDTYIDGIVSTKIPYALENIELYVSDGSGDDENGNGSISNPFKSISKALSEGYQKSTNITVHILEGTYTGSLNSNLRIPTTVNILLIGEGAAKTIISDSSSDYFITALKGKCELRISQMTLNRAARDTQSAIYIEEESNVAIDNVTFIGGQGNYGGAINTAGNLSIRNSYFHDNGYADRTLRANAYYGGAICNDGTLIIDNTIFESDHAGRLSEIANQGTLYMNNSKVIDSINAYSINMDLVAIGAYGGQKGEITIENSQFIVSSKTINELNDRIYDPTRALTCLGIGSCQHAILINSTFIGEGAVFSPYVFGGINSNNLASGGCTMIPGDLEVYNSTFRNVQAVNIIYSRTDNVRYHSQRVFEGCLFDNVEYIIAVLNTGNFSVEMHDCVILSDDLAKIGFGSEKTMKMDISNNWWSSNDGSYDNATLGTTNYISNCVVKLKEISSETVHPESYLILTLNSSNRTGLLQDAILAFKAYDGENISDYDGALYPRNFEMSAINATGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 16 - DNA, 4756 bp, synthetic construct
  source 1..4756: mol_type = other DNA
cttgttctttttgcagaagctcagaataaacgctcaactttggccaccatgttcgtcttcttggtgcttctgcctctcgtgtctagtgctgccgagaacacagacaacgctctgtctacggacacccattctaacgataacgtgctttctacagactctcggtctaacgagaacgccttgaccaacgagaacaccctcctcaccgacacccactctaacgagaacgccctgacgaccgaccctctgaccaaggagaacacccactcctacaaggactcagaaaaaagtctctcttctgacgccttcaacaagaccatctacgtgaacaagactggttctgacgagggcgacgggtccgaagcgaacccttacgcgaccttgaaaaagagtatttctcagctcgacgattctgacaacgccgtcatctacatcgggcctggcaactacaccggggagaacaattcagccttggagatcaacctggaccacaaggaccacgacgggtccctgagcatcatcggggactcaaacggcgggacggtgttcgacggcgagaacctcaaccctatcatcatctcaatctccgaggactctatcgtgaccttgatcaacatcacgttcacccacggcaagaacaacatgggcagtgccatccggtcttctggcaaccttacgattgacaactgcatcttcaccgagaactatgccacgaacttggcggccctctacgtggacaagcactcccctctgacggtcatgaactctaaattcctcgagaaccgcgcgaagcagtgcgccgacatctacttcagtcagaactctgagatcatcctcctcaacaaccttttcgagggctctacagcggagtactcttacgcctactctccttctgtctctcttcagaccgggaagtctctggtgaaggggaataccttcaagaacctgaccggggcgtactacaagggcgccctgtacatagcgtacaacaacggcatcaatatcgccaacatcacggacaacaccttcatcaactgcaactacaccggcaccgacggagccatcttgttcttccagaacgcctacctgaagaacaacaagttcatcgactgccactcatctaccgcattcctgtactcaaacacggagttcaacgcctacctgtctttcgaggatgccgagatcgatggcaccaccttttttctgaaggccaacgtgaccgacgacatgggtaacaaggtcaagaacgccaaggtcatcttctacctgaacggggagaacgttggcagtgcctcttctgacaacaacggcgtcgccatgatctctatcaagaaactgctggagaacggagagtatgtcatctctggaactcagtcctactcagagatcaaccctttcggcgtgaacgtgaagaacgccaccgcccgagtcaattacgaccatagcagtttggaggtctgggtgagcaccgacggcgacgacggttctggcaacggctctgaggacaaccctttcaagaccctgcggaaggcccttgactacgggacggcctctgccgtgaacctcaccgtgcacgtgaagaacggcatctacaatggcgatgacaacagagacctgtcttactccacgctgggaaagattaccatagttggcgagagttacagcaacgttgtgatcgacggtgagaacatcaccaagtctatattcgcattctcttccaccctggatgtgaccttgatcaacctcaccctcatcaactgtcctagcacccttatcaacgcctacaccctgtctatgatggacaatatcgtcataaactctgggaccatccgcgcccagaccggcaacaatggggtgactatcgataacctgcgggtgattaacggcacggaccaggcgataaccggttataacctccgcctgaccaactctcgcttcgagaactgcgacggccttacacacaccgggctgatctggctgtctaccaacaacaacaaagtgacttacctggagaataacaccttcttcaacaacacgatcgcgggcagtgcaggcgggggcgccgcgtactacatccagtccgaccttatcagtattaacaatacgttcgactctaactggatcaccgagagtcgaggcgagaacgtggcgtacgcgggcggacggcacatcataagcatcaacgataagttcataaacaacgaggtgcctaaatacgtcgcacagtaccgatcaatcgggaacgaggagtgtgagatcatcgtggaaaacatcaccttcatcaacaacaaggcctccgggaacggcgccgggctggccaccaccggggccatcgtcaagggggggaagttcattaacaactctgcctctgggaatggtggggccatctacctgctgaaccacgacaacacgtcttcgtactgccagatgtctctggaagacgtgatcttcgagaacaactctgccacctgcggcaaggacatcttcatcgagggctcatctgggaacaacatcttcacctacctgaacaacctgacgatcgtggctaacgacctgaacgtgacgtcactctcagacaacctcaccgtctctgtgttccaccctagcggcgccatcatcgggggcggggagatatctttctacctggacggcgagtacataggaaagtctactctcgtcaaccagaacgcatccctcgagtacgtggggttcaagaacaacactatctacgaattcacgtctatctacgagtacgcgtctctcaacgacacgtatatcgacggcatcgtctctacgaagatcccttacgctctcgagaacattgagctgtacgtgtcagacggcagcggcgacgacgagaacggcaacgggagtatctctaaccctttcaagtccatctctaaggccctgtctgagggttatcagaagtccaccaacatcacagtgcacatcttggaaggtacatacacaggctctctgaactcaaacctccgcatccctaccactgtcaacatcctgctgatcggcgagggcgccgccaagacaatcatctctgactcttcaagcgactacttcatcacagccctgaaggggaagtgcgagctgcggatctctcagatgaccctgaaccgcgccgcccgggacactcagtctgctatctacatcgaggaggagagtaacgtggcaatcgacaacgtcaccttcataggtggccagggcaactacgggggagccatcaacaccgctgggaacctcagcatacgcaacagctacttccacgacaatggctacgccgaccggactctccgggccaatgcttactacggaggggcaatctgcaacgacggcaccctgatcatcgacaacacgatcttcgagagcgaccacgcaggtagactgtctgagatcgctaaccaaggcactctgtacatgaacaacagcaaggtgattgactctatcaacgcctattctatcaacatggaccttgtggccatcggggcgtacgggggccaaaagggcgagatcaccatcgagaatagtcaattcatcgtcagctctaagaccatcaacgagctgaacgataggatttacgaccctacccgggcactcacgtgcctgggaatcggctcttgccagcacgcgattctcatcaactctaccttcattggagagggggccgtcttctctccttacgtcttcggagggataaattcaaacaacctggcctctgggggttgcaccatgattcctggggacctcgaggtgtacaactctacgtttaggaacgttcaggccgtgaacataatctactctcgcactgacaacgtgaggtaccactctcagcgagtctttgagggttgcctgttcgacaacgttgagtacattatcgccgttctgaacacaggcaatttctccgttgagatgcacgactgcgtgatcctcagtgacgatctggccaagatcggcttcgggagcgagaagaccatgaagatggacatctctaacaactggtggtcttccaacgacggctcttacgacaacgctaccctcgggaccaccaactatatctccaactgcgtggtgaagcttaaggagatctctagtgagaccgtgcaccctgagtcttacctgatactgacgctcaactcttctaatagaaccggacttctgcaggacgcgatcctggcctttaaggcctacgacggcgagaacatctctgactacgacggcgccctgtaccctcggaatttcgagatgtctgccatcaacgctacaggcggcggcggctctggtggggggggctctggcggcggagggtctatcgaaaacgccgacaaggcaatcaaggacttccaggacaacaaggcccctcacgacaaatctgccgcctacgaggccaactctaagctgcctaaggacctgcgcgacaagaacaatcggtttgtggaaaaggtgtctatcgagaaggccatcgtgaggcacgacgagcgcgtgaagtcagccaacgacgcgatttctaagctgaacgagaaggacagcatcgagaaccggcgcctggcccagcgcgaggtgaacaaggcgcctatggacgtcaaggagcacttgcagaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 17 - protein, 968 aa, synthetic construct
MFVFLVLLPLVSSAALDDLEGTIINKAINPFEGVENSGYYIEAIVDNQKVNLTVHDSLSIGNAYILAENISINYNETQINVTVLEENGKRADGGNVSLKLHDKTYISEIINGTAIFDIDVLPKGDYLLNYSLNRPKVYHSISNSSNLTVIPFKINAYANASNIKVGEDAIVIAYLDKDAGGNVTLGEEIQKVNDGTATFIISNLAKGDYTYQLSYSGDEKYDNETFHVSFSVNLKDASISVENDTLDLFVGSNETIVATISPIGLAVNYSCSNESVAMVDENGVVSAVGAGMAVITLTVGDDVTYSKNSSSVTVIVSKIPTIIEIVNDTISLEVTDSLDSIASLNPEEGGNLNYAISDDLIAKIDNGQITALSEGSAIITVSFDGNDKYTKAQNKSIKIIVNLKEASVSAFSNIDLLVGENDTLSPKTSPEGLDVRYESNDTSVVLVDNGQAIAVGEGNATITLTVGGDGVYAENTTTVKVSVSRIATMIEIEKDTIELKVNEESPIGAILEPDVGNLTYSISDESIAKVENGKIIALAEGNASLSISFAGDERYIGTNASVEIRVNKINTILTETDITTTYKEEGYLIATLKDSQNNPISGAVLTVDLDGIKNYTTDSNGQIKIATNNLIPDTYTARISFAGNENYSSSNGNASVTVKRIGTKLNFNDMNTTAFDSNIEGRIGEYFYFQLVDCDGNPLANKKVIIGFNGVKYNRQTNETGWAKIQINLKYANSYTFAIAFLGDDNYSGSFNFAVIRVSQQTPKLTASSKTYKSSAKTKTLTATLKSSSGKAIAGKKISFRLNGKVYTANTNSLGVATVKVSLNKKGTYSFTAQFSGDSTYKKVTKTAKLTIKGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 18 - DNA, 3091 bp, synthetic construct
  source 1..3091: mol_type = other DNA
cttgttctttttgcagaagctcagaataaacgctcaactttggccaccatgtttgtcttcttggtcctgctccctttggttagcagtgctgccctggatgacctcgaaggaacaattatcaataaggccatcaatccttttgagggcgtcgagaattccggatactatatcgaggccattgtggataatcagaaggtgaacttgacagtgcatgatagcctgtcaatcggcaatgcctacatcctggcagagaatatctcaatcaattacaacgagactcagatcaacgtgaccgtgctggaagaaaacggcaagagggcggacgggggtaatgtgagcctgaagctccatgacaagacctacatcagtgagattattaacgggaccgccatattcgacattgacgtgctgcctaagggggactacttgctcaactattctttgaaccgacctaaagtgtaccacagtatatccaattcctcaaaccttaccgttatacctttcaaaatcaatgcatacgccaacgccagcaacattaaagtgggcgaggatgccattgtgattgcttatctggacaaggacgcaggagggaacgttacactcggcgaggaaatccagaaagttaatgatggtacggcaacatttataattagcaatttggctaaaggcgactacacctaccagctgagttattccggcgacgagaagtacgacaatgagacctttcatgtctcattctcagtcaatctgaaagacgcttcaatcagcgtggagaatgacacattggacctctttgtgggatctaacgagactatcgtcgctactatctctcctatcggccttgcagtgaactacagctgctccaatgaatccgtggcgatggttgacgaaaatggcgtggtgagcgcagtgggtgcgggaatggcagtgattaccctgacagtgggagacgatgtgacctactccaagaacagcagctctgtcaccgtgatagtcagcaagatccctaccattatcgaaattgttaatgacaccatttctcttgaggtcaccgattcactggacagcattgcatctctcaatcctgaggagggaggtaaccttaattatgctatctccgacgatctgattgcaaaaattgacaatgggcagataacggccctgtctgaggggagtgcaatcatcaccgtcagctttgacgggaatgacaaatataccaaggcccaaaacaagtccattaaaattatcgtgaacctcaaagaggcctcagttagtgctttctcaaacatcgacctgctcgtgggagagaacgataccctgtcccctaagacgagccctgagggcctggatgtccggtacgagagtaatgatactagtgttgtgttggtcgacaacggccaagcaatcgccgtgggcgaaggcaacgccacgattacactcacggtgggaggtgatggggtctacgccgaaaacacgacgactgttaaggtgagcgttagtcggatagctacgatgatcgagatcgaaaaggataccatcgaactgaaagtgaacgaagaatcccctatcggtgcgatcctggaacctgatgtggggaaccttacctacagtatctctgatgaatctattgccaaggtggagaatggcaagatcatcgcgcttgcagagggcaatgcctccctttccatctcattcgccggcgacgaacggtatattgggacaaatgcttccgtggagatacgagtgaataagatcaacacaatcctgaccgaaactgacattactacaacatataaagaggaagggtacctgattgccacgctgaaggactcacaaaataaccctatatccggtgccgtgctcacagtggacctcgacggtatcaagaactataccacggatagcaacggccagattaagatcgccactaacaacctgattcctgacacttacaccgccaggatttcctttgccgggaatgaaaattattcttccagcaacggaaacgcaagcgtcacagtgaagcgcataggcaccaaactgaacttcaacgatatgaacacaactgcctttgacagcaacatcgagggcaggatcggggagtatttctacttccagctggtggactgtgatggaaaccctcttgccaacaagaaggttataataggattcaacggagtgaagtacaaccggcagacaaatgagactggctgggcgaagattcagatcaatttgaaatatgccaacagttacacattcgctatcgcttttctgggcgatgataactacagtggcagttttaacttcgcggtcatcagggtctctcagcagactcctaaactgaccgccagctctaaaacatataaaagtagcgctaagaccaagacactgacagcgaccctgaagtcctcaagcggtaaggcaattgctggcaagaaaatctccttcagactcaatgggaaagtctataccgccaacactaattctctgggggtcgccactgtcaaagtgtcccttaataaaaagggtacctactccttcactgcgcagttctctggagactctacctacaaaaaggtgactaaaaccgctaagctgaccattaagggcggcggaggatccgggggagggggtagtggagggggtggctccattgagaacgccgataaggccatcaaggatttccaggataacaaagcccctcacgataagtctgccgcttatgaggctaactctaagctgcctaaagacctcagagacaagaataacagatttgttgagaaagtgtcaatcgagaaggctatagtccgccacgatgaaagagttaagagcgctaacgacgcaatcagcaaacttaacgaaaaagacagcatcgagaaccgccggctcgcccagcgcgaagtgaacaaggctcctatggatgtcaaggagcacctccaaaagcagctggattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 19 - protein, 1447 aa, synthetic construct
MFVFLVLLPLVSSAAFDNSNDTIGNNIALDGSDEMLNADESNGNILASASYNKTIYVNYTGDDSGAGSQNSPYASLNKGISSVNASDNAVIYLSAGRFTGENNTDLSINLAHKNYNGSLTIIGASNGQTILDGNGETCIFKSISADSIVTLINITFTHGKAEFGSAIRTSGNLTIDNCTFDSNYATTLAAIYAEDMEDVKITNSVFKNNYGFEGYADLYCTGTESITIDIINSTFINSTTENLYSLPSFSIQNTNANIIGNKFENMTGLYESGTFELRYGYGKRKVIDNIFVNCNYIGNRKGGIIYISNTYLKNNQFINCTSTNALICSVTEFNAYLKFKNLTVNGTEFKLICEVTDDLNNSVSTHGKVHFFIDGKGIGSDNANNGIASITVTKLLDNGDYTLSGTYYYSENPFEVNVKNATIHVDFNHDPLELWVSNDGNDTTGNGSKNNPFKTLRHALNYGFENTIDLTIHMKDGIYTGNDNKDLSYSNIGKLTIFGESYRNTIIDAEYNNTIFTFGKYLNVTLTNMTLRNTSGNVINAYILKICDSFIEHVDFLNGGYSDTSKIIFNNLTYTNSGGIYIYNAEIYNSKFKNCNNTKNNCLLQIISLENNIIHIENTTIINNTVNGQHGSSIVYISGNSILINNNYINNTVISQNAMYIFQSYANKLTSANETFIGNNVSSYIAYYDFNGNNAEIIFENITFKNNYAKIDGSGLVIKTGRIKGAKFINNTALNNGGAIIILPHYGNSPFPKCILEDVFFENNNANNGKDIFIEKANNGYKNGQLDNITITFNNLITHNLQDIVTANISHPSGAVIGGGMIKFYLNGSYMGVAEVVNGVAKLNYLGFTKDGNYTLSGSYNYETNNTIYNNATVNVMLSPLKENITLYVSDSRGDDENGNGSYENPYKTIENALNKGCKQSKVIFIKVLEGNYTDKFNNNITLFDSLNITIIGEGIDKTIITGNNTKNNWFISVLHAGNGFLKLVNMTISEINYNYKNMNSQKSAVVIEDGANVMIDSVKFTKNRGFNGGAINNKGKLNIVNSIFYNNGDSSYGGSIYNTGITIIDNSSFIANHAKFCADIYNEGILNIYNSTIQDSMRTNGWTGTPLVIGGLGNISIINSKIFRTGKTPLELINPGDTYADNPGFTISIGTTGSIILINTTIDGHDAKYTGPSIYSTNNAAISWHVSSNIKIYNTSFLNLDSILNNQRGNITINSSFIKNVSNLIKSTSLYNLTVINSYFADGTISTEKYSNSNICLNNNWWGSNSKPTYKVANVDTNPETWLILTLNQTNQSVLSKNIILAFKVSDGENITDYTGQLYPRQFTMSSINGTGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 20 - DNA, 4528 bp, synthetic construct
  source 1..4528: mol_type = other DNA
cttgttctttttgcagaagctcagaataaacgctcaactttggccaccatgttcgtgttcctcgtgctgcttcctttggtctcctctgccgcgttcgacaactctaacgacaccatagggaacaacatcgcacttgacggcagtgacgagatgctcaatgccgacgagagtaacgggaacattctggcttccgcctcttacaacaagaccatctacgtgaactacaccggagacgactctggggctggctctcagaactctccttacgccagcctgaacaagggcatctctagtgtgaacgcttcagacaacgccgtgatctacctgtcagcgggccggttcaccggcgagaataacaccgacctctctatcaacctggcccataagaactataacgggtctcttacgatcatcggcgcctctaacggccagacgatactcgacgggaacggggagacctgcatcttcaagtctatctctgccgactctatcgtgacccttatcaacatcacattcacgcacggcaaggccgagttcggctctgccatccggacatctgggaacctgacgatagacaactgcacattcgactctaactacgccaccaccctggccgccatctacgcggaggacatggaggacgttaagatcaccaactcagtcttcaaaaacaactacggcttcgaggggtacgccgacctgtactgcaccggcaccgagtctatcacgatcgacattatcaactcaacttttatcaactctacgaccgagaacctgtactctctcccttctttctctatccaaaacacaaacgccaacattatcggaaacaagttcgagaacatgaccggtctctacgagagtgggaccttcgagctgcggtacgggtacggaaaaaggaaagtgatcgacaacatcttcgtgaactgcaactacatcggcaaccgaaaaggcggcatcatctacatctctaacacctacctgaagaataatcaattcatcaactgcaccagcacgaacgccttgatctgctctgtcaccgagttcaacgcgtacctgaagttcaagaacttgaccgtgaacgggaccgagttcaagctgatctgtgaggttaccgatgacctgaacaactctgtgtccacccatggcaaggtgcacttcttcatcgacggaaagggcatcgggagcgacaacgccaacaacggcatcgcctctatcaccgtgaccaagttgctggacaacggcgactacacactctccggcacctactattactccgagaaccctttcgaggtcaacgtgaagaacgccacgatccacgtggacttcaaccacgaccctctggagctttgggtgtctaacgacggtaacgacaccacgggcaacggctctaaaaataaccctttcaagacacttcggcacgcactgaactacggctttgagaacaccatcgacctcaccatccacatgaaggacgggatctacacaggcaatgacaataaggacctgtcctacagcaacatcggaaagctgacgatcttcggggagtcttacaggaacaccattatcgacgccgagtacaacaacactatcttcaccttcggcaagtacctcaacgtgaccctcacgaacatgaccctgcgcaacacaagtggcaacgtcattaacgcctacattctgaagatctgcgactcattcattgaacacgtcgacttcctcaacggtggctactctgacacctccaagatcatcttcaacaacctgacctacaccaattctggaggaatttacatctataacgccgagatctacaactctaagttcaagaactgcaacaatacgaagaacaattgcctgctgcagatcatcagcctcgagaacaacatcatccacatcgagaacactacgatcatcaacaacacggtgaacgggcagcacggctcttctatcgtctacatctctggcaactctatccttatcaacaacaactacatcaataacacggtgataagtcagaacgccatgtacatcttccagagctacgcgaacaagctgacgtctgccaacgagacgttcatcgggaacaatgtctcatcctacatcgcatattacgacttcaacggaaacaacgcagagatcatctttgagaacatcaccttcaaaaacaactatgccaagatcgacgggtctgggctcgtcatcaagaccgggcgcatcaagggggctaagttcatcaataacactgctctgaacaatggcggcgccatcatcatactgcctcactacggtaactctcctttccctaagtgcatcttggaggacgtgttcttcgagaacaacaacgcgaacaacggaaaggacatattcatagagaaggcgaacaacggctacaagaacggccagttggacaacatcaccatcaccttcaataacctcataacccacaacctgcaggacatcgtcaccgccaacatctcccaccctagtggcgcagttatcgggggtggcatgataaagttctacttgaacgggagttacatgggcgtggccgaggtggtgaacggcgtggccaagctgaactacctcggattcaccaaggacggcaactacaccctgtccggctcctacaactacgaaaccaacaacacgatctacaacaacgccaccgttaacgtcatgctcagccctctgaaggagaacatcaccctgtacgtgtctgactcacggggagacgacgagaacgggaacgggagttacgagaacccttacaagactatcgagaacgccctgaataagggctgcaagcagtctaaggtgatcttcatcaaggttcttgagggcaactacactgacaagtttaacaacaacatcaccctgttcgactctctgaatatcactatcatcggggaaggcatagacaagactatcatcaccggcaacaacacaaagaacaactggttcatcagcgttctgcacgcgggcaacggcttcctcaagctggtgaacatgacaatctctgagatcaactacaactacaagaacatgaactctcagaagagcgccgtggtcatcgaggatggggccaacgtgatgatcgactctgtcaagttcacgaagaaccgcggcttcaacgggggcgccatcaacaataagggaaagctgaacattgtcaactccatcttctacaacaacggggactcttcatacggaggctctatttacaacacggggatcactataattgacaactcttctttcatcgctaaccacgcgaagttctgcgccgacatctacaacgagggcatactcaacatctacaatagtaccatccaggattctatgagaaccaacgggtggaccggcacccctctcgtgatcgggggcctggggaacatatctatcatcaactctaagatcttcagaaccggcaagacccctctcgagctcatcaaccctggtgatacttacgccgacaaccctggcttcactatttctatcgggacaaccggctctatcattctgatcaacacgaccatcgatgggcacgacgccaagtataccgggccttctatttattctacgaacaacgccgccatctcttggcacgtgtcttctaacataaagatttacaacacctctttccttaacctggactccatcttgaacaaccagcgaggtaacatcaccatcaacagcagtttcattaagaacgtctcaaacctgataaagagtacctcactttacaacctgacggtcatcaactcttacttcgcggacgggacgatatctactgagaagtactctaactctaacatctgcctcaacaacaactggtgggggagtaactctaagcctacctacaaggtggccaacgtggacaccaaccctgagacctggctgatcctcaccctcaaccagaccaatcagtctgtcctctctaagaacatcatcctggcttttaaggtgtcagacggcgagaacatcaccgactacaccggccagctgtaccctcggcagttcacaatgagctcaatcaatggtaccggtggcgggggttctggggggggtggctctggcgggggtggctctatcgagaacgcagacaaagcgatcaaggactttcaggacaacaaagcccctcacgacaagtcagccgcctacgaggcgaactctaagctgcctaaggatctgcgggacaagaacaaccgcttcgtggagaaagtgagcatcgagaaggccatcgtgcgccacgacgaacgcgtgaagtctgcaaacgacgccatcagcaagttgaacgagaaggattctatcgagaaccggcgcctggcgcagcgcgaagtcaacaaggcccctatggacgtcaaggaacacctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 21 - protein, 575 aa, synthetic construct
MFVFLVLLPLVSSAALKSANGNITNHITNLINISKNATIYYVEATVDNQTVNLTQNITHNVTIIAKDIIFDYGTTTINVNVLFDNLPADSETITLNLNNKKYTAKTKNGTATFNIDIIPHGTYTLKYIINATKLHGEVLNSSILTVNRINANINATSHSVIVGNDVKVEINLPKDLTGTINVVLNNETYPVEIINNKAIVIIPNLAQGEYIAKITYSGDNKYLPTNTTVNIKVLGINITAPDVEKYYKGSEKLQIFIKDSEGNAIIGQNIQIKLNGKNYTATTNNEGIASIELDLNIGKYSATIIFNDKKINASITIKSTIHAPNMIRGYNSGLDYQTTLLNVDGTPLANTHITLKIGDKKYSLTTDANGVAKLNKKIAIGTYNILIINPTNLEKQTKTLKIISRINNNKNLAGDYLSGINYKIRIIDDNGKTAGTGKIVKITINKKHTTYLPIKMDTQHGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 22 - DNA, 1912 bp, synthetic construct
  source 1..1912: mol_type = other DNA
cttgttctttttgcagaagctcagaataaacgctcaactttggccaccatgttcgtcttcctggtgttgctccctcttgtctcctccgcggccctgaagtccgccaacgggaacataaccaaccacatcaccaacctgatcaacatttcaaagaacgccactatctactacgtcgaggcaacggttgacaaccagaccgtgaaccttactcaaaacattacccacaacgtcaccatcatcgccaaggacattatcttcgactacggaaccacgaccatcaacgtcaatgtgttgttcgataacctgcctgccgacagcgaaaccatcacgctcaacctgaacaataagaagtatacagccaagactaagaacggcaccgcaacattcaatatagacataattcctcatggcacttacacccttaagtacatcatcaacgctaccaaactccacggtgaggtgctgaactcaagtattctgacggtgaacaggataaacgcaaacataaacgcgaccagtcactccgtgatcgttggcaacgatgtgaaggtcgagatcaacctgcctaaggacctgactggcacaattaacgtggtgctcaacaacgagacataccctgttgagatcattaacaacaaggccatcgttataatccctaacttggcccagggtgagtacatcgccaagatcacctacagtggagacaataagtaccttcctacaaacaccaccgtgaacatcaaggtgctcgggatcaacatcaccgcacctgacgtcgagaaatactacaagggttctgagaagctgcagatcttcatcaaggacagcgagggcaacgcgatcatcggtcagaacatacagatcaagctgaacggcaagaactacacggccaccaccaacaacgagggaatcgcgtcaatagagttggacctgaatattggcaagtacagcgccacgattatcttcaacgacaagaagatcaatgccagcatcaccatcaagtcaaccatccacgcgcctaacatgatacggggctacaactctggactggattaccagactaccctgctcaacgtggacgggacccctctcgccaacacccacataacactgaagatcggggacaagaagtacagtcttactacggacgctaacggggtggccaaactgaacaaaaaaatcgccattggcacgtacaacatcctgatcatcaatcctaccaacctggagaaacagacgaagacgctcaagatcatatcacgaatcaacaacaacaagaacctggcgggcgactatttgagtgggatcaactacaagatccgaatcatcgacgacaacgggaagacagccgggaccggcaagatcgtgaagatcaccatcaataaaaagcacaccacttatcttcctatcaaaatggacacacagcacggcgggggtggctccggagggggcgggtctggcggaggtggcagcattgaaaacgccgacaaggctatcaaggatttccaggacaacaaggcacctcacgacaagtccgccgcatacgaggctaactctaagctccctaaggacctccgcgacaagaacaatcgctttgtggagaaggtgtcaatcgagaaggctatcgtgcgccacgacgagagggtcaaatctgctaacgatgccatcagcaagctgaacgagaaggacagtatcgagaatcggcggctggcccagagagaagtcaacaaggcccctatggacgttaaggagcatttgcaaaagcagcttgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 23 - protein, 169 aa, synthetic construct
MFVFLVLLPLVSSAAAEDLESDIGSQSNPNSQVQLAPQMGHLHRMINKAASGEPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 24 - DNA, 649 bp, synthetic construct
  source 1..649: mol_type = other DNA
cttgttctttttgcagaagctcagaataaacgctcaactttggccaccgccgaggatctcgagtcagacatcggaagccagtcaaaccctaactctcaggtccagctggcccctcagatggggcatcttcaccgcatgatcaacaaagccgccagtggcgagcctggcggcggcgggagtggtggcggggggtctggtggagggggcagcatagagaacgccgacaaggctatcaaggacttccaggacaacaaggctcctcacgacaagtccgcagcatacgaggccaactcaaagctgcctaaggatcttcgggacaagaataaccggttcgtggagaaagtgtccattgaaaaggcgatcgtccgccacgacgagcgagtgaagtccgccaatgacgccatctctaagctgaacgagaaggacagtatcgagaacagacggctggcgcaaagggaagtgaacaaggcgcctatggacgttaaggagcacctccagaagcagttggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 25 - protein, 157 aa, synthetic construct
MFVFLVLLPLVSSAASHMGRIVGQSQFEQPLFMDVLTQSLGPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 26 - DNA, 658 bp, synthetic construct
  source 1..658: mol_type = other DNA
cttgttctttttgcagaagctcagaataaacgctcaactttggccaccatgtttgtcttcctggtgctgctccctttggtgagttcagccgcctcccacatggggcgcatcgtggggcagtcacaattcgagcagcctctgttcatggacgtgctcacccagtcccttggccctgggggagggggcagcgggggcggcggatctggtggcggcggcagcatcgagaatgcagacaaggccataaaggacttccaggacaacaaagcccctcacgacaagtctgccgcatacgaggcgaactctaagctgcctaaggacctcagagacaagaacaaccggttcgtggagaaggtcagcatcgagaaggccatcgtgcggcacgacgagcgagtgaagagtgccaacgatgccatttctaagctgaacgagaaggactctatcgaaaacaggcgcctggcgcagcgggaggttaacaaggctcctatggacgtcaaggagcacctccagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 27 - protein, 181 aa, synthetic construct
MFVFLVLLPLVSSAASSTGDVHYGAESEYQKFEFGGGTPVAIQGDIVTNAPMGAKNSMDVVNFCAKGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 28 - DNA, 730 bp, synthetic construct
  source 1..730: mol_type = other DNA
cttgttctttttgcagaagctcagaataaacgctcaactttggccaccatgttcgtcttcctggtgttgctgcctctggtctcttctgccgccagtagcacgggcgatgtgcactacggtgcggagtctgagtaccaaaagttcgagttcggcgggggcacccctgttgctattcagggggacatcgtgaccaacgcacctatgggggcgaagaacagtatggacgtggtcaatttttgcgccaagggcggcggcgggtcagggggcggcgggagcggtggcggaggatctatagagaacgccgacaaggccatcaaggacttccaggacaacaaggcccctcacgacaagtccgcagcctacgaggccaactctaagctccctaaagaccttcgcgacaaaaacaacaggttcgttgagaaggtgtctatcgagaaggccatcgtgagacacgacgagcgcgtgaagtctgccaacgacgcgatctccaagctcaacgagaaggactcaatcgagaaccggcgactggcccagcgggaagtgaacaaggctcctatggacgtcaaggagcacctgcagaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 29 - protein, 183 aa, synthetic construct
MFVFLVLLPLVSSAASSTGDVHYGAESEYQKFEFGGGTPVAIQGDIVTKAPIGAKNSMDVVNFCAKFGGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 30 - DNA, 736 bp, synthetic construct
  source 1..736: mol_type = other DNA
cttgttctttttgcagaagctcagaataaacgctcaactttggccaccatgttcgtgttccttgttctgctgcctctggtgtctagcgccgccagctctacgggagacgtgcattacggtgctgagtccgaataccagaagtttgagttcggaggcggcacccctgtcgccatccagggggacatcgtgaccaaggcacctatcggcgctaaaaacagtatggacgtcgtgaacttctgcgccaagttcggagggggcgggggctccggggggggtggcagtggcggcgggggtagtatagagaatgccgacaaggccatcaaggacttccaggacaacaaggcgcctcacgacaagtcagcggcctacgaggcaaactccaagctgcctaaagatctccgggataagaacaacagattcgttgagaaggtgtcaatagagaaggcaatcgtgaggcacgacgagcgagtcaagtctgccaacgacgcgattagcaaacttaatgagaaggactcaatcgaaaaccggcgcctggcccagcgcgaggtgaacaaggcccctatggacgtcaaggagcacttgcaaaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 31 - protein, 162 aa, synthetic construct
MFVFLVLLPLVSSAATSHMGRIVGQSQFEQPLFMDVLTQSLGPIAGHGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 32 - DNA, 672 bp, synthetic construct
  source 1..672: mol_type = other DNA
cttgttctttttgcagaagctcagaataaacgctcaactttggccaccatgttcgtttttctggtgttgcttcctctggttagctccgccgccacgtctcacatggggcgcattgtgggccaatctcagttcgagcagcctctgttcatggacgtgttgacccagagtctcggccctatcgccggacatggcggcgggggcagcggtgggggcgggtcagggggtggcggatctatcgagaacgcagacaaggccatcaaggacttccaggacaataaggcgcctcacgacaagtcagccgcttacgaggcaaacagtaagctccctaaggacctgcgggacaaaaacaaccgcttcgtcgagaaggtgtccatcgagaaggccatagtcagacacgatgagcgggtcaagtcagctaacgacgcgatctccaagctgaacgagaaggacagtatcgagaaccggaggctggcccagcgagaagtgaacaaggcccctatggacgtgaaagagcaccttcagaagcagctcgattaatgatagaccagcctcaagaacacccgaatggagtctctaagctacataataccaacttacactttacaaaatgttgtcccccaaaatgtagccattcgtatctgctcctaataaaaagaaagtttcttcacattc

SEQ ID NO: 33 - protein, 183 aa, synthetic construct; identical to SEQ ID NO: 29

SEQ ID NO: 34 - DNA, 736 bp, synthetic construct
  source 1..736: mol_type = other DNA
cttgttctttttgcagaagctcagaataaacgctcaactttggccaccatgttcgtgttcttggtgcttctgcctcttgtgagctccgccgctagttcaaccggcgacgtccattacggggccgaaagtgagtaccagaagttcgagttcggaggcgggacgcctgtggcgatccagggagacatcgttaccaaggcacctataggtgccaagaactctatggatgtggtcaatttttgcgcgaagttcggcggtgggggagggagcggcggggggggctccggcggcggcggttcaatcgagaatgctgacaaggcaataaaggacttccaggacaacaaagcccctcacgacaagtctgccgcctacgaggcaaactcaaagctgcctaaggacctgcgggacaaaaacaaccgattcgttgagaaggtgtctatcgagaaggccattgtgagacacgacgagcgggtcaagtccgccaacgatgccatcagcaagctgaacgagaaggacagtatcgaaaacaggcgcctcgcccaacgcgaggtgaacaaagcgcctatggacgtcaaggagcacctccagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 35 - protein, 249 aa, synthetic construct
MFVFLVLLPLVSSAAQHLNVVVSGSMEPVMYRGDIVVLQKANLFGIHEFDPHDVQVGDIVVYNAAWYDSPVIHRVINTAEINGTTCFEIKGDNNNKSDPYWVTPEQITDRVITINGQPLVIPKIGYITLWVKGLGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 36 - DNA, 934 bp, synthetic construct
  source 1..934: mol_type = other DNA
cttgttctttttgcagaagctcagaataaacgctcaactttggccaccatgttcgtgttcctggtccttctccctctcgtttcctctgccgcacagcacctgaacgttgtcgtgagcggctctatggaacctgtgatgtaccgcggcgacatcgtggtgctccagaaggccaacctttttggtatccacgagttcgaccctcacgacgtccaggtgggagacatcgtcgtgtacaacgccgcctggtacgactctcctgtgatccacagggtcattaacaccgcggagataaacggcacaacttgcttcgagatcaagggggacaacaataataagtcagatccttattgggtgacccctgagcaaatcaccgaccgggtgatcacgatcaacgggcagcctctggtcatccctaagatcgggtacatcaccctgtgggtgaagggcctcgggggcggcggttctggcgggggcgggtcaggagggggcggcagtatcgagaacgccgacaaggctatcaaggacttccaggacaacaaggcccctcacgataagtctgccgcttacgaggccaactccaagttgcctaaagacctgcgggacaagaacaaccggttcgtggagaaggtctctattgagaaagccatcgtgcggcacgacgagcgcgtcaagagcgcgaacgacgcgatctctaagctgaacgagaaggacagtatagagaaccgaagactggcacagcgcgaggtgaacaaggcccctatggacgtgaaggagcatctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 37 - protein, 247 aa, synthetic construct
MFVFLVLLPLVSSAALNVVVSGSMEPVFYRGDIVAVEKADFLGIHEFDPSDVRVGDIVVYDATWYNEPVIHRVINITQINGTTYYMIKGDHNSHPDPYYATADQINERVLTWDGHPIVIPYIGNISLWLRGLGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 38 - DNA, 928 bp, synthetic construct
  source 1..928: mol_type = other DNA
cttgttctttttgcagaagctcagaataaacgctcaactttggccaccatgttcgtgttcctcgttttgctgcctctggtttcttctgccgccctgaacgtggtcgtgtcaggatctatggagcctgtgttctaccgcggcgacattgtggccgtggagaaagctgacttcctgggcatccatgagttcgatcctagcgatgtcagggtgggggacatcgtggtctatgacgccacgtggtacaacgagcctgtgatacacagagtcatcaacataacccagatcaacggaacaacttactacatgatcaagggcgaccacaattcacaccctgacccttactacgcgaccgccgaccagatcaacgagcgggtcttgacctgggacggtcaccctatcgtgatcccttacatcgggaacatttctctttggcttcgagggctggggggtggtggctcaggcgggggcggcagtgggggcggaggctccatcgagaacgctgacaaggccattaaggactttcaggacaacaaggcccctcacgacaagagcgcagcgtacgaggcaaactccaaactccctaaggacctcagagacaagaataaccgcttcgtcgagaaagttagtatcgaaaaggcaatagtgcggcacgacgaacgagtcaagagtgccaacgatgccatcagcaagctgaacgagaaggactccatcgagaacaggcgcctggcgcagcgggaggtgaacaaggcccctatggacgtgaaggagcacctgcaaaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 39 - protein, 247 aa, synthetic construct
MFVFLVLLPLVSSAALNVVVSGSMEPAFYRGDIVVIEKSDFLGIHEFNPTDVKVGDVVVYDAAWYDQPVIHRVINITQINGSTYYVIKGDNNDSPDPYYVSPNQINERVVTFGDNLCVIPYVGYLSLWLRGLGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 40 - DNA, 928 bp, synthetic construct
  source 1..928: mol_type = other DNA
cttgttctttttgcagaagctcagaataaacgctcaactttggccaccatgttcgtcttcctcgtgcttctccctctggtcagctcagctgcattgaacgtcgtggtctcagggagcatggaacctgcgttctaccgcggagacattgttgtgatcgagaagtccgattttttgggcatccacgagttcaaccctaccgacgtgaaggtcggtgacgtggtggtgtacgacgcagcgtggtatgaccagcctgtgatccaccgggtgataaacatcacacagatcaacggcagtacctactacgttatcaagggggacaataacgactctcctgacccttactacgttagccctaaccaaatcaacgagagagtggtcacgttcggcgacaacctgtgcgtgatcccttacgtggggtacctgtccctctggctgaggggacttggcggcggtggttctggaggcggcgggagtgggggcgggggctctatcgagaacgccgataaggccataaaggacttccaggacaataaagcccctcacgataagtctgcggcttacgaggccaactctaaactccctaaggacctgcgggacaagaacaaccgattcgtggagaaggtcagtatcgagaaggccattgtccgccacgacgagagggtgaagtccgccaatgacgccatatctaagctgaacgagaaggactcaatcgaaaaccgacggctggcccagcgcgaggtgaacaaggcccctatggacgttaaagagcatctgcagaagcagcttgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 41 - protein, 1180 aa, synthetic construct
MFVFLVLLPLVSSAARGNNFESIQEYIDNEGSSFNSIFNNLVLYKDINDKANLKNELLYILYLLGHGCSYESANFTGVQASGIFRDRWYSFDEYVLTFFNESRNRTIGILESTMYIQSQQLDLVNEITERLESKGYNVIPIYCPAGNAEQLNIMVKYWTSACSNISGFLENPQDFDIYVDGIISMVAYGVGGENFTNATKFFEDANVPIFRAVHSEYITNEQWELSPVGLSTTKSDKWWHVTIAESQGIFDATYVGGVDSYISNRTGAIILTFVPVHENIELLTDRVDAWVDLKYTPNEDKNISIVYYNYPPGKQNIGASYLDAITSVYNMLYTLKDEGYYLTDLPNNVSELEDMMIACGINVANWAPGEVEKLANRSGVALLPVDEYLEWFDSLDDIVKVQITEGPVAYIGQMVRRAVLINYTDEVETMVNDWYNQIKALLPENQTVAATNILDKLVNSLKLYANASSDGDENASLYYDEFLRYYDEFKSLNVSGLNGWGEAPGNIMLVNRNGTDYFVIPGLTFGNVFIGPEPQRGWEADIENLYHCTAVAPTHQYLAAYYYMQTRQSNAMVFVGRHATHEWLPGKEVLLSYNDYGSIVVGKVPQVYFYITDGLAEAIQAKRRGFAVLISHLDSPKSYTHLYGNLTVLATLLEEYDNNHIIIESDSDKDNQAITYQVIKDNQTITYQVINQELEDNLTRAIKDLVIANNYYLTIGFTAEELNNTDMFSLSSTLNAFLKNTQNTLYPLGLHAIGQKWTDEDLANTVAIIVSHDFEYGGKKTNLFDQLSLYYYGEKYSNLTPLKRDYILNRSVDVCKALIYWDTETVSDTIGIGSPEFIESLNIAKKYIDLYNQCISLELEEMVSALNGGYVPVNIGGESVTVPQVLPTGANMYQDQSSELPTQKAWDYAKTLSLLTLADLNDTTEKIIMGIWCVETARDDGALVSTVLYLLGMEPVWHNSSSAGFDEEGIPTGKKVEDLPNVIALENLTRPDGWAKKRIDVTVITSGLFRDLYSSQARLMDNAYRMALACSYYTIVNNKTIMDSEYGPQVYDALRSIMRSISFKGGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 42 - DNA, 3727 bp, synthetic construct
  source 1..3727: mol_type = other DNA
cttgttctttttgcagaagctcagaataaacgctcaactttggccaccatgttcgtcttcctggtgctcttgcctctggtgtctagtgccgcgcggggcaacaacttcgagtctatccaggagtacatcgacaacgagggcagttccttcaactctatcttcaacaacctggtgctttataaggacatcaacgacaaggccaacctgaagaatgagttgctgtacatcctctacctcctgggacacggttgctcatacgagagtgccaacttcaccggggtccaggcctctgggatcttccgggaccgctggtactctttcgacgaatacgtcctcaccttcttcaatgagtctcggaaccggacgatcgggatcctggagtccacgatgtacatccagtctcagcagctggacctcgtgaacgagatcacagagcgactggagtctaagggctacaacgttatccctatctactgtcctgcagggaacgcggagcagctgaacatcatggtgaagtactggacctctgcctgctcaaatatatctggattcctcgagaatcctcaagacttcgacatatacgtggacggcatcatatctatggtggcgtacggcgtggggggggagaactttaccaacgcgactaagttcttcgaagacgcaaacgttcctatcttcagggccgtgcatagtgagtacataacgaacgagcagtgggaactctctcctgtcggcctgtccacaaccaagtctgacaagtggtggcacgtgaccatcgccgagtctcaaggcatcttcgacgccacctacgtcggcggcgtcgactcttacatttctaaccgaaccggcgctatcatcttgacctttgtccctgtgcacgagaacattgagctgctgaccgaccgcgtcgacgcatgggtggacctgaagtacacacctaacgaggacaagaacatctctatcgtgtactacaactaccctcctgggaagcagaacataggggcctcataccttgacgccatcacctctgtttataacatgctgtacaccctgaaggatgagggatactacctgaccgacctgcctaacaacgtgtctgagctggaggacatgatgatcgcctgcggcatcaatgtggccaactgggcccctggggaggttgaaaaattggccaatcggtctggcgtggcgctcctgcctgtggacgagtacctggagtggttcgactcactggacgatatcgtgaaggtgcagatcactgagggccctgttgcctacatcgggcagatggtccggagggcggtgctcatcaactacaccgacgaggtcgagaccatggtcaacgattggtacaaccagatcaaggctctgctgcctgagaaccagaccgtcgccgccaccaacatcctggacaaactcgttaactcccttaagctgtacgccaacgcatcctctgacggcgacgagaacgccagcctctactacgacgagttcctgcggtactacgacgagttcaagtctctgaacgtgtctggactgaacgggtggggcgaggcacctgggaacatcatgctggtcaacagaaacggcacggactacttcgtgatccctggcctgacattcgggaacgtgttcatcggacctgaacctcagaggggctgggaggccgacatcgagaacttgtaccactgcaccgcggtggcgcctacccaccagtacctggccgcctactattacatgcagacccgccagtctaacgccatggtgttcgtggggcggcacgccacccacgagtggctgcctggcaaggaggtcctcctgtcttacaacgactacggctctattgttgtgggcaaggtgcctcaggtctacttttacatcaccgacggcctcgcggaggctatccaggccaagcgccgcggcttcgccgtgctcatctcacacctggactctcctaagtcttacacgcacctgtacggcaacctgacggtgcttgccactcttctcgaggagtacgacaacaatcatatcataattgagtctgattctgacaaagacaaccaggccatcacataccaagttatcaaggacaaccagacaatcacgtaccaggtcattaaccaggagttggaggacaacctgacgcgcgccatcaaggacctggtcatcgccaacaactactacctgactatcggcttcaccgccgaggagctgaacaacaccgacatgttctcactttcttcaactctgaacgctttcctgaagaatacccagaacaccctctaccctctcggcctgcacgccatcgggcagaagtggaccgatgaggacctcgccaacaccgtggcgataatcgtcagccacgacttcgagtacgggggcaagaagacgaaccttttcgaccaactgtctttgtactattacggggagaagtactcaaacttgacccctctgaagcgggactacatactcaaccgctctgtcgacgtgtgcaaggcccttatctactgggacacggagactgtgtccgacacgattggcatcggtagccctgagttcatcgagtctctgaacatcgccaagaagtatattgacctgtacaaccagtgcatcagcctcgagctggaagagatggtctccgcactcaacgggggttatgtgcctgtgaacatcgggggcgagagtgtgactgtccctcaggtgctgcctaccggcgccaacatgtaccaggaccagtcttctgagttgcctacgcagaaggcttgggactacgccaaaaccctcagcctgctgacactggcggacctgaatgataccaccgagaaaatcatcatgggcatctggtgcgtggagaccgcgcgcgacgacggagctctcgtgagtaccgtcttgtacctgctcggcatggagcctgtctggcacaactcctcttctgccggcttcgacgaagagggtatccctaccgggaagaaggtggaggacctccctaacgtgatagcccttgagaacctgacgcgccctgacggttgggcaaagaagagaatcgacgtgacggtgatcacctctggtctgttcagggacttgtactcctctcaggcacggctgatggataacgcctacagaatggccctcgcctgcagttattacaccatcgtcaacaacaagaccatcatggactctgagtacggccctcaggtgtacgacgctcttcgcagtatcatgcgctctatcagctttaaggggggcggagggggctcagggggtgggggcagcgggggtgggggatctatcgagaacgccgacaaggccatcaaggacttccaggacaacaaggcccctcacgacaagtctgccgcgtacgaggccaacagtaagctgcctaaggaccttcgggacaaaaacaaccgattcgtggagaaggtcagcattgagaaggccatcgtgcgccacgacgagcgggtgaagtctgccaacgacgcgatctctaagctgaacgagaaggacagcatcgagaaccggcgactcgctcagagagaagtgaacaaggcccctatggatgtgaaggagcacctccagaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 43 - protein, 1144 aa, synthetic construct
MFVFLVLLPLVSSAARGQNYDIYYEYITNGDGKLVNAEFNKAVLYKNYNNKENQINEILWALNITGYECKYSDPRFSKTYEYGIFREQYMTLEEYKKKYFDSSRPYTVGLLESNMYVSNGQLQPYYALIKSLEAKGCNVIPVVAAGGSENQLKVMVKYFTNAPSYEAYLNNPLKYTNNVNAIISMPAYGIGGNLFDNTTKYFETAGVPVFRAVHSDYVSNEEWELSATGLPGNRSDKWWHVAIGEAQGIIEATFVGGVTHEISSKTGAQLSGFKAHEKNIDLFTKRIVSWINLQYTVNSDKKISLVYFNYPPGKQNIGSSYLDSITSVYNLLYELKSQGYNVGKLPTTVKELEDMMIKSGINVATWAPGELEKLSNQPNIVLLPVAEYENWFNSLEPISKVQVIEGPVAYIGQLARNAIAINYTSPMKDIISDWYNGVKSLLPENYTESGVMLLDKIVAALNKYLQSGNDSDYQEYLSLKSQWKALNIPGLNGWGKAPGNIMTVTKNGVAYFVIPGLKFGNIFIAPEPQRGWEAKSDLLYHSSAVAPTHQYLAAYYYMQKEYSSAMVFIGRHATHEWLPGKEVLLSTTDYGSIVVGDVPQIYFYISDGLGEGLEAKRRGFAVMITHLTSPLAYTSLYGNLTAIANLINKYENTTDKTQKDTIASNIKLLIEKNNYIQSMGLTQEEFEKLNLNEVVKAADKFLFEVQNTLYPLGLHAIGQNWTVTDISRSVVAALSQEFTYDGITTTIFDEVAKYLFSKKYSELNALERDKVLNTSEQIVAALIFSNSTTVANVLGSDNPSLIAAMNYARYYISLIYASINNELTSFINGLNGKYIPVVSDGDVININSLPTGGNFFHDQSQELPTEEAYNYAKTLTLLTLSSLNEKTQKIAMGIWCVETARDNGALISVVLYLLGMQPVYTSSPSAGGKTEDGDSVGTKTKIMPKFVGLKDLVRPEGWAKKRIDIVVITSGNFRDLYSTQVSLLDNAFRVALARSYLTIINNKTLMESKYGKDMKEALDKVMEGIGYYGGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 44 - DNA, 3619 bp, synthetic construct
  source 1..3619: mol_type = other DNA
cttgttctttttgcagaagctcagaataaacgctcaactttggccaccatgttcgtgttcctggtcctgctgcctctggtgtcttctgccgctagaggacagaactacgacatctactacgagtacatcaccaacggcgacggcaagcttgtcaatgccgagttcaacaaggccgtgctttacaagaactataacaacaaggagaaccagatcaacgagatcctgtgggctctgaacattaccggctacgagtgcaagtactcagaccctcggttctctaagacctacgagtacggcatcttccgcgagcagtacatgaccctcgaggagtacaagaagaagtacttcgactcttctcggccttacactgtcgggcttctcgaatctaatatgtacgtgtctaacgggcagctgcagccttactacgccctcatcaagagcctcgaggccaagggctgcaacgttatccctgtggtggcggcaggaggctctgagaaccagctcaaggtcatggtgaagtactttacgaacgcgccttcttacgaggcctacctcaacaatcctctgaagtacaccaataatgtgaacgccatcatctctatgcctgcctacggcataggtggcaaccttttcgacaacaccacgaagtacttcgagaccgccggggtgcctgtgttccgggccgtgcactccgactatgtgtctaacgaggagtgggagctctctgccaccgggctgcctggcaaccggtctgacaagtggtggcacgtcgccatcggcgaggcccagggcatcatcgaagcgaccttcgtgggaggtgtgacccacgagatctcttccaagacgggcgcgcaattgagcggtttcaaggcccatgagaagaacatagacctgttcacgaaacggatcgtctcttggatcaatctgcagtacaccgtgaacagcgacaagaagatctctctggtgtacttcaactaccctcctgggaagcagaacattggctctagctacctcgactctatcacctccgtctacaacctgctttacgagcttaagtctcagggttacaacgtggggaagctgcctacaaccgtgaaggagttggaggacatgatgattaaatccgggataaacgttgccacatgggcccctggggaactcgagaagctgagtaaccagcctaacatcgttctcctgcctgtggccgagtacgagaactggttcaactccttggagcctatctcaaaggtccaggtgatcgagggacctgtggcctacatcgggcagctggcccggaacgccatcgctatcaactacacctctcctatgaaggacatcatctctgactggtacaacggagtgaagtctcttctgcctgagaactacaccgagtccggggtgatgctcctggacaagatcgtcgctgctctcaacaagtacctgcagtctgggaacgactccgattaccaagagtacctgtctctgaagtctcagtggaaggccctcaacattcctgggctgaacgggtggggcaaggcccctggcaacataatgactgtcaccaagaacggcgtggcctactttgtgatccctggcttgaagtttgggaacatcttcatcgcccctgagcctcagagaggctgggaggctaagtctgatttgctgtaccactcttctgcggtggctcctactcaccagtaccttgcggcatattactacatgcaaaaggagtacagtagtgccatggtcttcatcggccgccacgccacgcacgagtggctgcctgggaaggaggtgctcctgagtaccaccgactatggctctatcgtggtcggggatgtccctcagatctacttctacatctctgacgggctgggggaaggtcttgaggccaagcgacgcgggttcgccgtgatgatcacgcacctgacttctcctctggcctatacatcactgtacggcaacctgaccgccatcgccaacctcattaacaagtacgagaacacgacagacaagacgcagaaggataccatcgcgtctaacatcaagctgctgatcgaaaaaaacaactacatccagtctatggggctgactcaggaggagtttgagaagctgaaccttaacgaggttgtgaaggccgccgacaagttcctcttcgaagtccagaacacgctgtaccctctgggcttgcacgcgataggccagaactggaccgtcaccgacatcagccgcagcgttgtggccgcgctctctcaggagttcacctacgacgggatcaccaccactattttcgacgaagttgcgaaatacctgttctctaagaagtactcagagctgaacgccctggagcgagacaaagtccttaacacctctgagcagatagtggcggccctcattttctcaaacagcaccacggtggccaacgtgctcggctctgacaacccttctctgatcgccgccatgaactacgcccgctactacatctctctgatctacgcatctatcaacaacgagctgacctctttcattaatgggctgaacggcaagtacatccctgtggtcagtgacggagacgttatcaatatcaactcactgcctacgggcgggaacttcttccacgaccagagccaggagctccctaccgaggaggcctataactacgcgaagaccctcacactgctgaccctctcttccttgaacgagaaaacccagaagatcgcgatgggaatctggtgcgtcgagacagcacgggacaacggcgccctcatctctgtggtgctctacctgttgggaatgcagcctgtgtacacgtcatctccttctgccggcgggaagacggaggacggcgactccgtgggcacgaagaccaagataatgcctaagttcgtcgggctgaaggacctggtccggcctgagggctgggccaagaagcgcatcgacatcgtggtgatcacctctggcaacttccgcgacctgtatagtacccaggtcagtctcctcgacaacgccttccgggtggccctggcccggtcatacctgaccatcatcaacaacaagaccctgatggagagcaagtacgggaaagacatgaaggaggcgctggacaaggttatggagggcatcggttactacggcggcggtggaggcagtggtggcgggggctctggcggcgggggctctatcgagaacgctgacaaggccatcaaggacttccaggacaacaaggcccctcacgacaaatctgccgcatacgaggcgaactctaagctccctaaggacttgagagacaagaacaaccgcttcgtggagaaggtgtcaatcgagaaggccatcgtgaggcacgacgagagggtcaagagtgcaaacgatgcaatatctaagctgaatgagaaggactctatcgagaacaggcgactcgcccagcgcgaggtcaacaaggcacctatggacgtgaaggagcacctgcagaagcaattggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 45 - protein, 355 aa, synthetic construct
MFVFLVLLPLVSSAAYTGTGFSHDIPFSKYSSQSNSDILNKYNNTDCHSEIKGICTYVADGDTIDVEGVGRVRFVGVNTPERGVTAYICSKRFVQKFCLNKEVSLDVDDSKRNDRYGRTLAVVIVDGKNLNEMLLKEGLAEIMYIPPSEFYPYDWSSDSTTSSSYTSGSSSSNSGGSYSSSSFTSGSTVSAPYVGSANSHKFHYSTCKWGKKISDKNRVTFNSRSDAISQGYAPCKACQPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 46 - DNA, 1252 bp, synthetic construct
  source 1..1252: mol_type = other DNA
cttgttctttttgcagaagctcagaataaacgctcaactttggccaccatgttcgtcttccttgtgctgctgcctttggtcagttccgccgcttacaccgggaccggcttctcccacgacatccctttctccaagtacagtagccagtctaactctgatatcctgaacaagtacaacaacacagactgccacagcgaaatcaaagggatctgcacctacgtcgccgacggagacaccatagatgttgagggggtgggacgcgtccggtttgtgggagtgaacacccctgagagaggtgtcaccgcgtacatctgcagtaagcggttcgtgcagaagttctgccttaacaaggaggtgtcactcgacgttgacgactccaagcgaaacgaccgctacggcaggacgctggcggttgtgatcgtggacggtaagaatcttaacgagatgttgctcaaggagggtctggcagagatcatgtacatccctccttcggagttctacccttatgactggagctccgactctaccacgtctagtagctacacttctggctcttcttcttcaaactctggcggctcatatagttcatcttcattcacgagcggcagtaccgtgtccgccccttacgtggggtctgcaaactcacacaagttccactacagcacttgcaagtgggggaagaagatctctgacaagaaccgggtgacattcaactcaagatcagacgcaatcagtcagggatacgcgccttgtaaggcctgccaacctgggggaggcggctctggcgggggcggtagcggcgggggcgggagtatagaaaacgccgacaaagctatcaaggatttccaggacaacaaggcccctcatgacaagtctgccgcctacgaggccaatagcaagctgcctaaggacctccgagacaaaaataacaggtttgtcgagaaggtctctatcgaaaaggccattgtgcggcacgacgagcgcgtgaagtccgcgaacgacgctatatctaaattgaacgagaaggactccattgagaaccggcgcctggcccagcgagaggttaacaaggcccctatggacgtgaaagagcacctccagaagcagctggattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 47 - protein, 343 aa, synthetic construct
MFVFLVLLPLVSSAAYTGTGFSHDIPLSKYSDSSYKNILDKYNDTECEAEVSGICTRVVDGDTIYVDGVGKVRFVGVNTPENGVEGGDVSKYFVQKLCMNQEVGLDIDDSKQQDKYGRTLAVVIIDDKNLNEMLLKEGLAEIMYIPPSEFDPYSWSNGSTDINEHAHSKSTDTSSDSSGKYVASMNSDKFHKPSCRWAEKIYEQNKISFNSRESAINNGYQPCKVCNPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 48 - DNA, 1216 bp, synthetic construct
  source 1..1216: mol_type = other DNA
cttgttctttttgcagaagctcagaataaacgctcaactttggccaccatgttcgtgttccttgttctgctccctttggtgagttctgccgcctacacgggcaccggctttagccatgacatccctctgagtaagtactccgactcttcctacaagaacattcttgataagtacaacgacacagagtgtgaggcagaggtgtccgggatctgcaccagagtggtggacggtgacactatctatgtggacggcgtcggtaaggtgcgcttcgtcggcgtgaacacacctgagaacggcgtcgagggtggcgacgttagtaagtattttgtccagaagctgtgcatgaaccaggaggtgggccttgatatcgacgactctaagcagcaagacaagtacgggaggaccctggccgtggtgatcatcgacgacaagaacctcaacgagatgctgctcaaggaggggctcgccgagatcatgtacatccctccttctgagttcgacccttactcctggagcaacgggtctacggacataaacgagcacgctcactctaagtcaaccgatacctccagcgactcatctggtaaatacgtcgccagtatgaattcagacaagttccacaaaccttcttgcaggtgggccgagaagatctacgaacagaacaagataagtttcaactctcgggagtcagccatcaacaatggataccagccttgcaaggtttgcaaccctggcggagggggatccggcggcggcggatcaggcgggggggggagcatcgagaacgcggacaaggcaattaaggacttccaggacaataaggcccctcacgacaaaagtgccgcttacgaagcaaacagcaagctgcctaaggacctgcgggacaagaacaaccgattcgtggagaaggtgagcattgagaaggcgatcgttcggcacgatgagcgcgtgaagtctgccaacgacgctatatctaaattgaacgaaaaggactcaatcgagaatagacgattggcgcagcgcgaggtcaacaaggcccctatggatgtcaaggagcacctgcagaaacagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 49 - protein, 399 aa, synthetic construct
MFVFLVLLPLVSSAAAEDLESDIGSQSNPNSQVQLAPQMGHLHRMINKAASGEPVAYGCWCGISGAIAALAMGMGIIPIVAIAMGSTVAALVHAIYTVTSHMGRIVGQSQFEQPLFMDVLTQSLGPIAAHGFIASFGIVGIAYLMTLPLDGLGHPFPLPLLAVLWGITIGAIGSSTGDVHYGAESEYQKFDYGGGTPVAIQGDIVTKAPLGAKNSIDVGNFCAKYGGPLTGFCFGLIVFVSFWITVVFGALGGQIVGIVIVILLIAANYLLEKSTRAKFGPYEEGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 50 - DNA, 1384 bp, synthetic construct
  source 1..1384: mol_type = other DNA
cttgttctttttgcagaagctcagaataaacgctcaactttggccaccatgttcgttttcctggtgctcttgcctctggtctcttcagcagccgccgaggacctggaaagcgacatcggctcccagagtaaccctaactcacaggtgcagctggcccctcagatgggccacctgcaccggatgatcaacaaggccgcctctggggagcctgtcgcgtacggctgctggtgcgggatctctggagccatcgcggctctggccatgggcatgggcatcatccctatcgtggccatcgccatgggctctaccgtggccgccctggtgcacgccatatacacggtcacctctcacatgggcaggatcgtgggccagagccagttcgagcagcctctgttcatggacgtgcttacgcagagtctcggccctatcgccgcccatgggttcattgcgtctttcggcatcgtcggtatcgcctaccttatgaccctgcctctggacggcctcgggcaccctttccctcttcctctgctggccgtgctctggggaattaccataggcgctatagggtcctctaccggcgacgtgcactacggggctgagtctgagtaccagaagttcgactacggcggaggtacgcctgtggccatccagggtgacatcgttaccaaagcacctcttggcgccaagaacagtatcgacgtgggcaacttctgcgccaagtacggcgggcctctcactgggttttgcttcgggctcatcgtcttcgtgtctttctggatcacagtggtcttcggtgcactggggggccagatcgtcgggattgtcatcgttatcctgctcatcgcggccaactacctgttggaaaagtctacccgagcgaagttcggcccttatgaggagggaggcgggggatctggcgggggcgggtcaggcggtggggggtcgatcgagaacgccgacaaggcgatcaaggacttccaagacaacaaggcccctcacgacaagtctgccgcgtacgaggctaacagcaagctccctaaggacctgcgcgataagaacaacagatttgtggagaaggtgtctatcgagaaggccatcgtgcggcacgacgagcgcgtgaagtctgcgaacgacgccatctccaagctgaacgagaaggactctatcgagaatcggcggttggcccagcgcgaggtgaacaaggcacctatggacgtcaaggagcacctgcagaaacagctcgattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 51 - protein, 401 aa, synthetic construct
MFVFLVLLPLVSSAAAEDLESDIGSQSNPNSQVQLAPQMGHLHRMINKAASGEPVAYGVWCGVAGSIAYVLIMLGFVPIISIAMGSCVAAFVHAIYTVTSHMGRIVGQSQFEQPLFMDVLTQSLGPIVGHGFITSFCIVGISYLMIIPLNGTTLHVFPLPLLAVLWGIALGAIGSSTGDVHYGAESEYQKFEFGGGTPVAIQGDIVTNAPMGAKNSMDVVNFCAKFGGPLTGFCFGLVVFFSFWNTVVFGIYGGLVVGFIIVILLIIMNDRLEVFARNRYGPYEEDGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 52 - DNA, 1390 bp, synthetic construct
  source 1..1390: mol_type = other DNA
cttgttctttttgcagaagctcagaataaacgctcaactttggccaccatgttcgtgttcctggttctgctgcctctggtttcatctgcggccgcggaggacctcgagtctgacatcggaagccagtcaaaccctaactctcaagtgcagcttgcgcctcagatgggccacctccaccggatgatcaataaggcagcctctggcgagcctgtggcttacggggtttggtgcggggtggcaggatccatcgcttacgtcctgatcatgttgggtttcgtgcctatcatctctatcgccatggggagttgcgtcgccgccttcgtccacgcgatctacactgtgaccagccacatgggcagaatcgtcggccagtctcagttcgaacagcctctgttcatggacgtcttgacccagtcactcgggcctatcgtgggccacgggttcatcacctctttctgcatcgtggggatatcctacctgatgatcatccctctgaacgggacgaccctgcacgtgttccctctgcctctgctcgctgtgctgtggggtatcgcgttgggcgccatcgggtcttctacaggagacgtgcactacggcgccgagtctgagtaccagaagtttgagttcggcggcgggacgcctgtggccattcagggggacatcgtgaccaacgcccctatgggcgccaagaactctatggacgtggtgaacttctgcgccaagttcggcggccctctcaccggcttctgcttcggtctggtcgtgttcttctctttttggaacaccgtggtcttcgggatttacggcggcctcgtcgtgggattcatcatcgtgatcctgcttatcataatgaacgaccgccttgaagttttcgccaggaaccgctacggcccttatgaggaggacggagggggcggctctgggggcggcgggtctggcggcggtgggtctatcgagaacgccgacaaggcgatcaaggacttccaggataacaaggcccctcacgacaagtctgccgcctacgaggccaactctaagctgcctaaggatctgcgggacaagaacaaccggttcgtcgagaaagtgtccatcgagaaggccatagtccgccatgacgagcgcgtgaagagtgccaacgacgccatcagcaagctcaacgagaaagacagtattgagaaccggcgactcgcccagcgggaggtcaataaggcacctatggacgtgaaggagcacctgcagaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 53 - protein, 400 aa, synthetic construct
MFVFLVLLPLVSSAAAEDLESDIGSQSNPNSQVQLAPQMGHLHRMINKAASGEPVAYGVWCGVAGAIAYILIHFGIFPIVGIAIGASVAAFVHSIYTVTSHMGRIVGQSQFEQPLFMDVLTQSLGPIAGHGFITSFCIVGISYLMTIPLNGTALHVFPLPLLAMLWGIALGAIGSSTGDVHYGAESEYQKFEFGGGTPVAIQGDIVTKAPIGAKNSMDVVNFCAKFGGPLTGFCFGLVVFFSFWNTVVFGIYGGIIVGLIIVVLLGVMNDRLEVFAREKYGPYEEGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 54 - DNA, 1387 bp, synthetic construct
  source 1..1387: mol_type = other DNA
cttgttctttttgcagaagctcagaataaacgctcaactttggccaccatgttcgtgttcctcgtgctcctgcctctggtgtcttccgcagcggccgaggacttggagagcgacatcggcagtcagtcaaaccctaactctcaagtgcagctggcccctcagatgggccacctccaccgcatgatcaataaggccgcttctggcgagcctgttgcctacggcgtctggtgcggggtcgccggcgccatcgcttacatcctgatacacttcggcatttttcctatcgtgggcatcgccatcggcgcaagcgtggccgccttcgttcactctatctacaccgttacgagtcacatgggtaggatcgtcggacaatcacagttcgagcagcctctgttcatggacgtgcttactcagtctctgggccctatcgccgggcacggcttcatcacctctttctgcatcgtgggcatcagctacctcatgaccatccctctgaatggcacagccctgcacgtcttccctctgcctctcttggcgatgctctgggggattgcccttggggccatcgggtcctctaccggagacgtccactacggggccgagtccgagtaccagaagttcgagttcggtggtgggacacctgtggcaatccagggcgacatcgtgaccaaggcccctatcggagcgaagaactcaatggatgtggtgaacttctgtgctaagttcggcggccctctgacggggttctgcttcgggctcgtcgtttttttcagtttttggaacaccgtggtcttcggcatctacggggggatcatagtcggtcttatcatagtggtgctgctgggcgttatgaatgaccggcttgaagtgttcgctcgagagaagtatggcccttacgaggaggggggtgggggaagtggaggcggaggttccggcgggggaggtagcatagagaacgccgataaggccataaaagacttccaggacaacaaggcgcctcatgacaaaagcgctgcgtacgaggcaaactcaaaattgcctaaggatcttcgcgacaagaacaacaggttcgtcgagaaggtgtcaatcgaaaaggccattgtgcggcacgacgagcgcgtcaagagtgcgaacgacgccatttccaagttgaacgagaaggactctattgagaaccgacggctggcacagagagaggtgaacaaggcccctatggacgtgaaggaacatttgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 55 - protein, 455 aa, synthetic construct
MFVFLVLLPLVSSAAATTVFLTSDNIMGTNDDADMLNSIKTYIEEISNGKINVIVDSQSPGPGEGTRAIEADSNVSVVFAAVDPGNFLVLSKYSTATTDKQIIFVNTGDYDLDTAESLRRAWDDNYSKTIFAGINNPGTFLNDGGISYIQPLKEYHDAGSDGIINQNNDDVNKYIAQEIVNNINNYNNTKHYDNNLVITHKLAPSNMAHGSQSLLESNDNEMNGTYNSYSAPQLLYLTSSYLNGNGLENPGDYKAPDSPLKYSILTKDSYSIYDYIKMGGIVKNYMDENGQAPNYINYEGAYISYYDLQYNFAKITANHTDGSHMDFDREYHFDKVNDSIGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 56 - DNA, 1552 bp, synthetic construct
  source 1..1552: mol_type = other DNA
cttgttctttttgcagaagctcagaataaacgctcaactttggccaccatgtttgtgttcctggtgttgctccctctggtgtctagcgcggccgccacgaccgtgttccttacctccgacaacatcatgggcacgaacgacgacgccgacatgctgaatagcatcaagacctacatcgaggaaatttcaaacgggaagatcaacgtcattgtggactcccagtcacctggacctggggagggaacccgcgccatcgaagccgacagcaacgtgagtgtcgttttcgccgccgtggaccctggcaacttcctggtgctgtctaagtactctaccgccaccaccgacaagcagatcatcttcgtgaacacgggcgactatgacctcgacactgccgagtctctgcggcgggcctgggacgacaactacagcaaaaccatcttcgcagggataaacaaccctggcacattcctgaacgacgggggcatatcttacatccagcctctgaaggagtaccacgacgccggctctgacggcattatcaaccagaacaacgacgacgtgaacaagtacatcgcccaggagatcgtcaacaatatcaacaactacaacaacaccaagcactacgacaacaaccttgtcatcacccacaagctggctcctagcaacatggcccacggctctcagtccctcctggagtctaacgacaacgagatgaatgggacctacaactcttactctgcgcctcagttgttgtaccttacttcgagttacctgaacgggaacgggctggagaaccctggagactataaggcgcctgacagtcctctgaagtacagtatcctcacgaaggactcttactctatctacgattacatcaagatgggcggcatcgtcaagaactacatggatgagaacggccaagcccctaactacatcaactacgagggggcctacatctcttattacgacctccagtacaacttcgcgaagatcaccgcaaaccacacagacgggtctcacatggacttcgaccgcgagtaccatttcgacaaggtcaacgactctatcggcggtggtggatcaggcgggggcggttccgggggcggcggctctatcgagaacgcggacaaggccatcaaagacttccaggacaacaaggcccctcacgataagtctgccgcctacgaggctaactctaagctccctaaggacctccgcgacaagaacaaccgattcgtcgagaaagtgtctatcgagaaggccatagtgcggcacgatgagcgggtgaagtctgcaaatgacgctatctcaaagctcaacgagaaggactcaatcgagaataggagactggcgcagcgcgaggtgaacaaggcccctatggacgttaaggagcacctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 57 - protein, 455 aa, synthetic construct
MFVFLVLLPLVSSAAAQTVFITSDNIIDHDTDVRMLNSIKEHIEELSNGELQVIVDNQAPGPGEGYRSIQVTSDICVNIAASDAGNYLQLANYTVYNDKHIIFVNSGSYDLDNSSNYLRRAWDDNYSNQYFAGVHDPGTLLKNSGVTYIQPLKEYPDAGDGDNIDKYDEEMNKYIAQEVVNQVKNYNYESKILSDSLIVYHHIEPSIMASASKELVKSNDTEMNGTYGGYSAPQLLYQTSSYLNGNGLDNPKNFTAPESPMKFSLLTKGSYTINDYIKMGGIVKTYMDENGQAPDYINYEGAYISYYDLVYNFAKITQNHTNTQHMGFDSEYEFEKTNDSGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 58 - DNA, 1552 bp, synthetic construct
  source 1..1552: mol_type = other DNA
cttgttctttttgcagaagctcagaataaacgctcaactttggccaccatgttcgtgtttctcgtgcttctgcctctcgtgtcaagtgccgccgcccagacagtgttcatcacctccgacaacataatcgaccacgataccgacgttaggatgttgaacagtatcaaggaacacatcgaggagctgtcaaacggggagctgcaggttatcgtggataaccaggcccctgggcctggggagggttaccgaagtatccaagtcacttccgacatctgcgtcaatatcgctgctagcgacgctggcaactatctgcaactcgccaactatactgtctacaacgacaagcatataatcttcgtcaactcaggcagctacgacctggacaactcttccaattacctgagacgagcatgggatgacaactactctaaccagtactttgcaggagtgcacgaccctggcacattgctgaagaactcaggtgtcacctacatccagcctctgaaagagtaccctgacgccggggacggagacaacatcgacaagtacgacgaggagatgaacaagtacatcgctcaggaagttgtgaaccaggtgaagaactacaactacgagtctaagatcctgtctgacagtctgatcgtgtaccaccacattgagcctagcataatggcctctgcatctaaggagttggtcaagagtaacgacacggagatgaatggaacctacgggggctactcagcccctcagctcctgtaccagactagctcttaccttaatggcaacggccttgacaaccctaagaacttcaccgcccctgagtcacctatgaagttctctctcctgacgaaaggaagctacaccatcaatgactacatcaagatgggcgggatagtgaaaacgtatatggacgagaacggccaggcgcctgactacatcaactatgagggggcctacatcagttactatgatctggtgtacaacttcgccaagatcacccagaaccacaccaacacccagcacatgggcttcgactctgagtacgagttcgagaaaacaaacgactctgggggtggtggctctggtgggggcggaagcgggggcggcggctctatcgagaacgcggacaaggccattaaggacttccaagataacaaggcccctcacgacaagtccgcagcctacgaggcgaattctaagctgcctaaggacttgcgcgacaagaacaaccgcttcgtggagaaggtgtccatagaaaaggccatcgtgcggcatgacgagcgggtgaagtctgcgaacgatgcaatttccaagcttaacgagaaggactctattgagaaccggcgcctcgcccagagggaggttaacaaggcgcctatggacgtcaaggaacacctccagaaacagcttgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 59 - protein, 846 aa, synthetic construct
MFVFLVLLPLVSSAANDLSTLGENTTVPNIIIVGDAPEVPDVPDIPDIPDFPVDPDNPDIDDQNDSDTVNLTIFNIDEYFVDGTLGVEHSNTKFVLTQNFDNLGLLKIEANNVTILGNNFTLQNVAFLINGKDVTLANFTLVNDFDFKDADGAAILTLANNTHIRDCVINYNVPRDSEGYGISAVGRRIAPISGLEVINCIINFEGHNYKANTYNYALKVSNCPNALIANNSIYTQLPLRDVNFGAVGADLNSNYVASVGIEYSNNLTFIGNIVASIVNKRPGSPFPTLDGIIIADSNDCLVKNNTLYMEDFLTFPGLNNYLYGIDVWRVNSLTLDSNNIAILTTGGMLSAGTAYPIQITGPSKKINITNNDLYSISNGPNIGIYSQNYYGDTQLYIAHNKINVTGLAGNDSWALVAGIEVQDSNDTIINNTIEVHSVAEVKDNDNMYGISYSQSTKGNHTFVIKNNTVTSDAKYAISLISAENSVIVDNLLISTRKDAKASYDAFNTKGKFYNTSYYNNRVVNAFDYYAEIYNHVDGGSEFNYTTPTNVNNLTNKVDGSKIKPWFPDFPNRNPLLPKPGDGSSVIVTPDDGDDKNPNVPDRPDGDAGYVDVPDLSGDDGGSKNPSNGTSSTDKNSNKLGVSLLDALINFLNSNTDTGDSRSNSYGGTVRTNSSTSSSDSPSLDGNPSPASSTKSSSGSNAKSAGSSAGDDSKSVKAYEIDKNIMKSNPNTGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 60 - DNA, 2725 bp, synthetic construct
  source 1..2725: mol_type = other DNA
cttgttctttttgcagaagctcagaataaacgctcaactttggccaccatgttcgtgttcctggtgctgttgcctctcgtttccagtgcggccaacgacctcagcacgctgggtgaaaacacgaccgtccctaacatcatcatcgtcggagacgcccctgaggtgcctgatgtgcctgacatccctgacatccctgatttccctgtggatcctgataatcctgacatcgacgaccagaacgacagtgacacagttaacctgacgatctttaatatagacgagtactttgttgacggcactctgggagtggagcacagtaatacaaagttcgtgcttacgcagaacttcgacaacctgggcctgttgaaaatagaggcgaacaacgtcactatactgggaaacaacttcaccctgcagaacgtggcgttcctcattaacggcaaggatgttacacttgccaacttcacccttgtgaacgacttcgacttcaaggacgccgacggagccgcaatcctgacgctcgccaacaacacacacatacgcgattgcgtgatcaattacaacgtgcctagggatagcgaggggtacggcatctccgctgtgggcaggcgaatcgcccctatcagtggtctggaggtcataaactgcatcattaacttcgagggtcacaactataaggcaaacacttacaactacgctttgaaagtgtcaaactgtcctaacgccctcatcgctaacaactctatctatacccagctccctttgagagacgtgaacttcggtgccgtgggcgccgatttgaactccaactacgtggcctctgtcgggatcgagtattcaaataacctcaccttcatcggtaacattgtggcctcaatcgtgaacaagcggcctggatcccctttccctacactggacggaatcatcatcgcagacagcaacgactgcctggtgaagaacaacactctctacatggaggactttctcacctttcctgggctgaacaactacctgtatggcatcgacgtctggcgggtgaattcccttacactggacagtaacaacatcgcgattctgaccaccgggggaatgttgtccgccggcaccgcttaccctatccaaattacaggcccttcaaaaaagataaacattaccaacaacgacctttactctatcagcaacgggcctaacatcggaatatacagccagaattactacggagacactcaactgtacatcgctcacaataaaatcaacgtcaccggccttgccggcaacgactcttgggctctggtggccggtatcgaggtccaggactcaaacgacaccattattaacaacactattgaggttcactccgtcgcagaggtcaaggacaacgacaacatgtacggtatctcatactctcagtcaactaaaggtaaccatacgttcgtcataaagaacaacacagttacgtctgacgccaagtacgctatcagccttatatccgccgagaactctgtgattgtcgacaacctgctcatcagtacaagaaaagacgccaaggcatcctatgacgccttcaacaccaaggggaagttctacaataccagttattacaataaccgagtcgtgaatgccttcgactactacgccgagatctacaatcacgtcgacggagggagcgagtttaactatactacccctaccaacgttaacaacctgaccaataaagtggacggctccaagataaagccttggttccctgatttccctaacaggaaccctcttcttcctaagcctggggatgggagcagcgtgatagtcacccctgacgatggcgatgacaagaaccctaacgtgcctgaccggcctgacggcgacgcagggtacgtcgacgttcctgacctgagtggggacgacgggggctcaaagaacccttcaaatggcacctcctccaccgacaagaattccaacaagctgggcgtgagtcttttggacgctctgataaactttcttaactcaaacaccgatacaggcgacagccgcagcaatagttacggcggaactgttagaaccaatagttctacctcttctagcgactccccttcacttgacggaaatcctagccctgcgtcttctacgaagtcctctagcggcagcaacgccaagtctgccgggtcatcagccggcgacgactctaagtcagtgaaggcctacgagatcgacaagaacattatgaaaagtaaccctaacactggtggagggggttcaggcgggggcggtagtggtgggggcggtagtatcgaaaacgctgacaaggcaatcaaggacttccaggacaataaggcacctcacgacaaatctgctgcctacgaggcaaactccaagttgcctaaggatttgcgcgacaaaaacaaccgcttcgttgaaaaggtgagtatcgagaaggcgatagttcgacacgacgagcgggttaagagtgcgaatgatgcaatctcaaagctcaacgaaaaggacagcattgaaaaccgaaggttggcacagagagaggtgaataaggctcctatggacgtgaaggagcatttgcaaaagcagttggattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 61 - protein, 830 aa, synthetic construct
MFVFLVLLPLVSSAASNNLIESLSTNNNGSEAIYIPNSDLNLLSGNGNDVETPDIPDLIVNDTFYVTSDDIEDYFPNRQLESKYYNKTLIFTGNFENVGKLIIDVNNVTLKGVGSYLKNTVFDLRADNITLDSFNMDLDSEFEDNDGAAIEFIANNIVLSNLRINYIVPRNVEAYAIYGIGQPYRSIKNFKMFNSIINFEGHNDYVNKYNYAVKLVDCVDSVMENNSLVTSLPLRNVVFGAMGASLDSDFVLSVGVENCHNFTFIGNSIIANVNNRPAFTYPTLDCFLISKCDNSSILNNSIYMTDFLTFPGVENYLYGIDVYNLNNLTIAYNNISIITTGGKLAAGTAYPIQITGPISAVNITYNDIYTFSNGPNIGIYSQNYYGNTSLSITHNRINVTGLAGSHEWALVAGIETQDSNSTIQNNTIEVHSVAPVDIGDNIYGISYRQKTSGNHTYNIQNNTVFSDGFYSVYLLSSVNSNVINNLLVSYNDKVKAGLNGFNYNEFSSHIGINFYNNKVINAFDYFANKYNNIDGGEGFNYTNPTNINSISNNIDGSSVIAIPSNNHYNYNPLIPGNSNNQGNPNNQGSNQQNNQGNSNGNSSQGGSGNGVNDNNSGEGNSSGNKLSLRDLLANFFNSNSDKGKVNRSNYNSNANHEITSNNTDQTPSTEGEDALMSDVKSVESTSESPSASDSASKKAYELDDLVKENKLFIPSGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 62 - DNA, 2677 bp, synthetic construct
  source 1..2677: mol_type = other DNA
cttgttctttttgcagaagctcagaataaacgctcaactttggccaccatgttcgtgtttctggttcttctgcctctcgtgtcttctgccgctagcaacaacttgattgagtccctgtcaacaaacaacaatggtagcgaggccatttatatccctaacagcgatcttaacctgttgtctggcaatgggaacgatgtcgaaacacctgacatccctgacctcattgtcaacgacaccttctacgtgacgtctgacgatatcgaggattacttccctaacaggcagctcgagtctaaatactataacaaaaccttgatctttacaggtaactttgagaatgtcgggaagcttattattgacgttaataacgtcactctgaagggggttgggtcctatctgaagaacacagtctttgacctgcgagccgacaatattacactggacagcttcaacatggatctggattctgaattcgaggacaacgatggagctgccatcgaatttatcgcaaataatattgtgctgagcaaccttagaattaactacattgttcctcgcaacgtcgaagcttacgccatatacggaataggtcaaccttacagatccattaaaaacttcaagatgttcaactcaatcattaactttgagggtcataatgactatgtgaacaagtacaactatgcagtgaagttggtggactgtgtggattcagttatggagaataacagcctggtcaccagcctccctctcagaaacgtggtgtttggtgccatgggcgcttcactggatagcgacttcgtccttagcgtgggcgtcgagaactgccacaacttcaccttcatagggaattctatcatagcaaacgtgaataacaggcctgcgtttacatatcctaccctggattgtttcttgatctcaaagtgcgataatagcagtatcctcaacaatagcatttatatgacggacttcctgaccttccctggggtcgaaaattatttgtatggcatcgacgtgtacaatctcaacaaccttactatcgcttacaataacatttcaattattacaacaggcggcaaactggccgccggcaccgcataccctatacagatcacggggcctatcagcgctgtgaatattacatacaacgatatatataccttcagtaatggacctaacatcggcatttattcccagaattactacgggaacaccagcctcagcatcactcataatagaatcaacgtcaccgggctggccggaagtcatgagtgggccctggtcgccggcatcgagacacaggatagtaatagcaccatccagaataacactatcgaggttcacagcgtggctcctgtggacataggtgataacatctacggtatctcataccgccagaaaacctctggtaatcacacctacaacatccaaaacaacaccgtgttttcagacggcttctactccgtgtatctgctcagtagtgtgaacagcaacgtgattaataacctgctcgtcagctataacgacaaggtgaaagcaggactgaacgggtttaattacaacgaattctcttcccacatcggaattaacttttacaacaacaaagtgatcaacgcattcgactactttgcgaacaagtacaataacatcgacggcggcgagggctttaactacactaatcctactaacatcaattcaatcagtaacaatattgatgggtcatccgttatcgccataccttctaataatcactataattacaatcctcttatccctggcaactctaacaatcagggcaatcctaacaaccaaggaagtaatcaacagaataatcagggaaacagcaatggaaatagctctcagggaggatccggaaacggggtgaatgacaataactccggggaggggaactcctctggcaataagctgtccttgcgcgacctcctggctaacttctttaacagtaattccgataagggaaaagtgaaccggagcaactataattcaaatgcgaaccatgaaataacttccaacaacactgaccagacgcctagtacggaaggcgaggatgccctgatgagcgatgtgaagtccgttgagtccacctccgagtctcctagcgcctctgacagcgcctccaagaaagcatatgagctggatgatctggtcaaagagaataagctcttcatcccttccggaggcggtggtagtgggggcggaggcagtgggggcggcggctccatcgagaatgcagacaaagcgataaaggactttcaggataataaagctcctcacgacaaaagtgcggcttacgaagccaattccaagctgcctaaggatttgcgagataagaacaataggttcgtggaaaaagtgtcaatcgaaaaggccatcgttcggcatgacgaacgggtgaagagtgccaacgacgctatttccaaactgaacgaaaaggactctattgaaaacaggcgcctcgcacagcgggaggtcaataaggcccctatggacgtgaaggagcacctgcagaagcagcttgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 63 - protein, 922 aa, synthetic construct
MFVFLVLLPLVSSAAIDVDTNDNLDDGSSSNSDLISSSSLDSSSSDDVSSGSSEVSSSDESLDGNNLSDGNVSSSDESVGADNLSDGNVSSSDESVGADNLSDDESSSDALSEELPKTETVIKADPINYNYASVKGLTINLTDSAGLALSNKTLTVKVSALNKTSNLTTNSKGQAIFKLSASVGSYDVFISFTGDESYAPSNASSKITIKKSSTKIKLSNIHGYLTISNYVSVTLLDSAGKPIKSKSVTIQVNKAKYNVKTDSKGIAKVKVANKIGTYSVNAKFSGDKNYYASSNSSKLTITKMKVYIKAPSVKYYMTNSSAPYLTINLTNVKGSPLAKKKVSVKIGKKTYTLKTNSQGIAKFKFTKKVSSYNCKINFKATSNFYGASVNSKMTIQKMPTSLKAPSVSINSTNYGKVLISLKDGKGKALKNTTVTVNVTELKKVFTLKTNASGVATFSFNGEKTFNLKIKYAGNKNYAASSVSSKINVKQIKVKLSDVIGASRVLIDYVNRTKDLPSNVQYNNYNFTVTQLTYLASKAVKNINNKNYGDIVLISVPKSYKSSGEIYDTVYKKDFVKIANSVVGSSYNYKNKEYVSYSIYKVPFKVYSISFAKVLNFYGNNKKLPNYSLFTLADFAKVKDNGGYNFYLTTDNIAGKKSDLNMLKSLAKTLKSMGYNAVIVGIGPDIHNVAYRYGCTGNNSVLLACFGGVDVGCIEEWAGDLGDLNGHSFVNSYQGAHVLGLWFTKPYGASVSLNKKVGIAWDADYGFPLNTPAKYMKSHNISYIETGTVANACKLLSEGKMGGPQLISGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 64 - DNA, 2953 bp, synthetic construct
  source 1..2953: mol_type = other DNA
cttgttctttttgcagaagctcagaataaacgctcaactttggccaccatgttcgtcttcctggtcctcctccctctggtgtcttctgcagctatcgacgtcgatacaaatgacaaccttgacgacgggagctcatctaactccgacttgattagctccagttctctggactcttcttcaagtgatgacgtgtcctccgggtccagtgaagttagctctagtgacgagtcactggacggtaacaatcttagcgacggtaatgtgtccagcagcgacgagtccgtgggagcagacaacttgtctgatggcaatgtctcttcatcagatgagagcgtgggagcagataatctctctgatgacgagtcctcctctgacgccctgtctgaggagctgcctaaaactgagaccgttataaaggctgatcctataaattacaattacgcatctgttaaaggtcttacaatcaatctgactgattccgctggcctggccctcagcaacaaaactctgaccgtgaaagtctccgcactgaataagaccagcaacctgactacaaacagtaagggtcaggccatcttcaaactgagcgcaagtgtcggatcatacgatgtgttcatctctttcacgggggacgaaagctacgctccttcaaacgcatcctcaaagataaccattaagaagagctccaccaagatcaagctttcaaacatccatgggtatcttaccatctcaaactatgtttctgtgacccttttggacagcgccggaaagcctattaaatccaagagtgtgacgatccaggtgaataaggccaagtataacgtgaagaccgactctaagggcatcgcgaaagtcaaggtcgcaaataagatagggacatatagcgtcaacgccaagttcagcggcgataaaaattactacgcatccagcaactcttcaaagctgacgataacgaaaatgaaggtgtacattaaagccccttccgtcaaatactacatgaccaacagttctgccccttatctgactatcaacctcactaatgttaagggctcccctctcgccaagaagaaggtctctgtcaagattgggaagaaaacgtataccctgaagacaaacagccaaggaattgctaaatttaaatttacaaagaaagtgtcctcttacaactgtaagatcaactttaaggccaccagcaacttctatggagcatcagtcaattcaaaaatgacgatccaaaagatgcctacatcactgaaggccccttccgtgagcattaacagtaccaactatggcaaggttctgataagtctgaaggatgggaagggcaaggccctcaagaacacgactgtgaccgtgaatgtcaccgagctgaaaaaggtgtttacactcaagaccaacgctagcggcgtcgcgacttttagttttaacggtgaaaaaacttttaacctgaagattaagtacgccggcaacaagaattacgctgcttcttccgttagcagtaaaatcaatgtgaagcagataaaggtgaagctgagcgacgtgatcggcgcgtcccgagtgttgattgactacgtgaataggactaaagacctgcctagtaatgttcagtataataattacaattttacagtgacacagttgacctatctggctagcaaagcggtcaagaatatcaacaacaagaactacggggatatcgttctgattagtgtgcctaaatcctataaaagctctggcgagatttatgataccgtctacaagaaggactttgtgaagattgctaactccgtggttggctcctcatacaattataaaaacaaagaatacgtgtcctatagtatttacaaagtgcctttcaaagtgtactccatctcattcgctaaggtcctgaatttttacggaaacaataagaagctccctaattacagtttgttcacactcgctgatttcgccaaagttaaagataacggaggctataatttctaccttaccacagacaatatcgcgggtaagaagtccgatctgaacatgctcaaatctctggccaagaccctgaagagtatgggatacaacgctgtgattgttggaattggccctgacatccacaacgtggcttatagatatgggtgcacaggaaataattccgttctcctcgcctgcttcggaggggtggatgtcggctgcatcgaggaatgggccggggaccttggcgacctgaacggccatagcttcgtcaattcataccagggtgcgcacgtgctgggcctttggttcactaaaccttacggtgctagtgtgtccctcaataaaaaggtggggatcgcctgggatgccgactatggatttcctttgaacacccctgccaagtatatgaaaagtcataacatcagctacatcgagactggcaccgtggccaacgcctgtaaactcttgagcgaaggcaagatgggaggacctcaactgatatccgggggcggagggagcggtggcgggggtagcggtgggggcggcagcatcgagaacgccgacaaggccatcaaggatttccaggacaacaaagcacctcacgataagagcgcagcctacgaggcgaacagcaaactccctaaggatttgagagataagaacaacaggtttgtggagaaagtgagtattgaaaaagcaatcgtgcgccacgatgaacgggtgaaaagcgccaacgacgctatcagcaagctgaatgaaaaagactccattgagaaccggcgcctggcgcagcgggaagtcaacaaagcccctatggacgtgaaggagcacctccagaaacagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 65 - protein, 779 aa, synthetic construct
MFVFLVLLPLVSSAAQSLDNINYANDGFDSDEMINCDLHKDSSQKSLKSNALSNKKTTNTVKLTDMKKAESNDVKQTGAAKASNTKSTSKSTTKTNATKSNTTKSTATKTTANSSSTKKATQNTTTINTQTLAKSSSSYMAYVEKNAKLQEPITISKKKYKSPEYLYLVSKAVSNISKTKVEIKDKLITNYSNTDCKSVNGTINKTEYVQVAKKTVSFIEKNHRAPNWIASSKGNIPRNQLILVFSKCLDQYNKSGKLPSSIKLNDLDLNKMKQKIDSSKKVNSTSTKKTNTSSTKTNSTSAKKTNTTSTKKTNPTATSTNNNKSLVESTLDSIKSILNNIENKLNPTNKVLSTTGTKKNTVTVNSSKVNVQISSSSTVNVKISAKDNTNSGKNTNSGSAKKTNTTSTKKTNTTSTKKIDTNSTKKTNTTSTKNNTSSAKKTNTTSTKNNTSSAKKTNTTSTKNNTSSAKKVNTSSSKTNTSAKNNTSTTAKSSSNSKYLSTSVLNDKYLGESLKKYLAVGKNCQVTNKAIKTLANTLTSKLKSDYKKGEKIFNWVRDNIGYEKYRNTKKGALKTLQTRGGNCVDHAHLIVALSRAAGLPARYVNANNCKFSSGYVSGHVWAQVLVGNTWVVADATSNRNKFGVVKNWNVNSYKLVGKYSSISFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 66 - DNA, 2524 bp, synthetic construct
  source 1..2524: mol_type = other DNA
cttgttctttttgcagaagctcagaataaacgctcaactttggccaccatgtttgtctttctcgtgttgctccctctggtctcctctgcagcccagagcctggataacattaactacgccaacgacggcttcgattcagatgaaatgattaattgcgatttgcacaaggatagttcccagaagtcccttaagagcaacgctctgtccaacaagaaaacgacaaataccgtgaaactcacggatatgaagaaagcagagtcaaacgacgtgaagcagaccggggcggccaaggccagcaatacaaaaagcacatccaagtctacaacgaaaaccaacgcaaccaaaagcaacacaacgaaaagtactgccaccaaaacgaccgctaactcttcctccacaaaaaaagctacccaaaacactaccactatcaatactcagactctcgccaagtccagttcctcctatatggcctatgtggagaaaaacgcgaagctccaggaacctattaccattagcaagaagaagtacaaatctcctgaatacctgtatctggtgagcaaagcggtgagtaacatcagcaaaaccaaggttgagataaaggacaagctgataacaaactacagtaatacagactgtaagtcagttaatggcaccatcaacaaaaccgagtacgtgcaggtcgcgaagaagaccgtctctttcatagagaagaaccatcgcgcccctaactggatcgcaagcagcaaaggaaacatccctcgcaaccaattgatcctcgtgttcagtaagtgtcttgaccagtacaataaaagcggaaagcttcctagctctatcaagctgaacgatctggacctcaataagatgaagcagaaaatagacagcagtaaaaaggtgaactccacttctactaagaaaacaaatacctcttcaactaagacaaacagcacaagcgctaagaagactaatactaccagtaccaagaagacaaaccctacggccacatctaccaataataacaaatcactggtggaaagtacgttggactctataaagtccattcttaataatattgagaacaagctgaaccctactaataaagttctctcaacaacagggaccaagaagaacactgtgactgtgaattcatccaaagtgaatgttcagatctcctcttcctcaacagttaacgtgaaaatctcagccaaagacaacaccaattctggaaaaaacaccaactccggatccgccaaaaagaccaatacaaccagcaccaagaagacgaacacgacaagtaccaagaagatcgatacaaactccactaaaaagacgaacactactagcactaagaataacacctcttcagcaaaaaaaaccaataccaccagtacgaagaacaacacctccagtgcaaagaagacaaataccacgagtacaaagaataatacatccagcgccaagaaagtgaacacttcctccagtaaaaccaataccagtgccaagaacaacaccagcaccaccgcaaaaagctctagcaacagcaaatatctgagtacctccgttctgaatgacaaatacctcggggagtctctgaagaaataccttgctgtcgggaagaactgccaggtcaccaacaaggctatcaagacgctggccaatacactgacgagcaagttgaagtcagactataagaagggtgaaaagattttcaattgggtccgagataacattggctacgagaagtacaggaacactaaaaagggcgcacttaagactctgcaaacaaggggcggtaattgcgtggaccacgcccaccttattgtcgccctgtccagggctgcgggcctgcctgctcggtacgtgaatgccaataattgtaaattttcatccggctatgtgtctggtcacgtgtgggcccaggtgctcgtcggcaatacttgggtggttgccgacgcaaccagcaaccggaacaaatttggggtcgtgaaaaactggaatgtgaactcatataagctcgtcggaaagtactcctcaatctcattcggtggcggtggcagtgggggaggcggcagcggaggaggggggagcatcgaaaatgcagacaaggctatcaaggacttccaggacaacaaagcccctcatgataaatctgcggcctatgaggctaatagcaaactgcctaaggacctgcgggataagaacaacagatttgtggaaaaggtgagcatcgagaaggctatcgtgcggcacgacgaaagagtcaaatctgctaacgatgctattagcaagttgaatgaaaaagattctatcgagaaccgaagactggcacagcgcgaggtcaacaaggcccctatggacgttaaggagcatctgcagaaacaactggattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 67 - protein, 1110 aa, synthetic construct
MFVFLVLLPLVSSAASNASDNLDDLTISDSNSLDLVSTSNSDILSSDSGVSSDDSSNDASGDVLGSDVSSNESNNQSQSTLDSNNQSQSGLDSDNSTLLDSQSNNQSNSESSDSSDSSETVIKNATSISVSSKTVVRGNSLNITLKDNASTLLSNKTVTFTFNGKTYNKTTNAKGIASLTLTATPKKYLVKIAFVGDELYEASSKSVNVTLSKTPTSISNSGKSIVRGKLYKLTLKDAKGKALSGKKISISFNGKKYTKTTNSNGQVNLTINVNVGKTYKMTYKFAGDSNYLSSSGSVSIKVKMGTSIIGSGSSIVKGKSYTVTLKNANGAVLSNQKIAFTLSGKTYNRTTNAKGQASLKIGLSSGKTYNLTYKYAGNSYYGGSSGKVSLFVKTPTTMKNSGKTIVSGETYKVTLKDADGKSLANKKVSITFNNKTYAKTTNSNGQASLTIKGTFGRSYPLSYKFAGDSKYGPSSGSLCLRVKKATSLKGSASSIVQGKSYTVTLKDSNSTPLANQTIVFTLDTKKYNRTTNAKGQASLKIGLAAGKTYNLAYKYSGTSYYNGSSGSVKLKVKFPTSLTNSGKSVMNGTGYNIVLKDSKSNLVSNKTISIGFNGKTYDEITDANGTVTLLIDANVPKTYKMTYKFAGDSDYGASSGTVNLTVKFKNAFTISQIISASSSLKSYVLKNKKVPATVSVNGVSLNLTSFTYLMAKATISINSNKTSGSILLVPVDSNYTNNGSRINANLYKANYIDLAKKVISSAEANKLVPNSVSTNIGLVSHDLYSFGLAKALVFFNSDHYLPNYLILSSDDVGEKHSTVIPSNARGNASQFKAGLNEAETLTAAQIAKYLVASGHDATNSEIKALAAKLVSGKTSLWDKANAIFTFARDNITYSYYADSKKGAAGTLSSKSGNCCDHSNLIVSLCRAANITARFSHAQGCTFSSGLVAGHVWAQIYIDGVWYTADATSRRNSLGNIVNWNTNHYNTLKQYDHLSFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 68 - DNA, 3517 bp, synthetic construct
  source 1..3517: mol_type = other DNA
cttgttctttttgcagaagctcagaataaacgctcaactttggccaccatgttcgttttcctggtcctgctccctctggtctcctccgcagcgtcaaacgcctctgacaatcttgacgacctcactatctctgactcaaactctctggacctcgtgtctacctctaactctgacatcctttcttctgactctggcgtgagttccgatgacagctctaacgacgcctctggcgacgtgctggggagtgacgtttctagtaacgagagcaacaaccagtctcagtctacgctggattcgaacaaccagtctcagagcgggcttgacagtgacaactctacactgctcgactcccagtctaacaaccagtctaactctgagtcttctgactcttcagactcttctgagaccgtgatcaagaacgccacgtccatctctgtcagttcaaagaccgtggtgcgggggaactctttgaacatcacgcttaaggacaacgcctctaccctgctctctaacaagactgttaccttcacgtttaacggcaagacctacaacaagaccacgaacgccaaggggatcgcctctctcactctgacggccactcctaagaagtacctggttaagatcgccttcgtcggggacgagctctacgaggcatcttctaagtctgtgaacgtcacgctctctaagacccctacctctattagcaactctgggaaatctatcgtccgcggaaagttgtataagctgacgctgaaagacgccaaggggaaggccctgtctggtaaaaagatctctatcagcttcaacggcaagaagtacacaaagaccacaaactctaatggccaggtgaacctgacaatcaatgtcaatgtgggcaagacctacaagatgacctacaagttcgccggcgactccaattatctgtcttcatcaggctcagtgtctatcaaggtcaagatgggcacctccatcatcggctctggctcgtcaatcgtgaagggcaagtcttacaccgtgactctcaagaacgcgaatggcgccgtgctgtctaaccagaaaatcgcattcaccctcagtggcaagacgtacaataggacaaccaacgctaagggccaagcgtctctcaagatcggcctgagtagcggcaagacctacaacctcacatacaagtacgccgggaactcttactacggcggctcttctgggaaagtctctttgttcgtgaagacgcctacgaccatgaagaacagcggcaagacgatcgtgtcaggggagacgtacaaggtgaccctcaaggacgctgacggcaagtctctggcgaacaagaaggtctccatcactttcaacaataagacatacgccaagaccaccaactctaacgggcaggcaagtcttaccattaaagggaccttcggccgctcttaccctctgtcctacaagttcgccggcgactctaagtacggtcctagctctggatctctgtgcctgcgcgtgaagaaggcgacctctctgaaggggtctgcctctagtatcgtccaggggaagtcttacaccgtcaccttgaaggactccaactctacccctctggctaaccagaccatcgtgttcacactggacacgaagaagtacaaccggaccaccaacgccaagggccaagcctctctgaagatcggcctggccgccggtaagacgtacaacttggcctacaagtactctggcacctcttattacaacggcagctctgggagcgtgaagctcaaggtgaagttccctacctctctgaccaacagcggaaagtctgtgatgaacgggaccgggtacaacatcgtgctgaaggactccaagtctaacctggtgagtaacaagaccatatctataggtttcaacgggaagacctacgacgagatcacagatgccaacggcacggtcacccttctcatcgacgctaacgtccctaagacctacaagatgacctacaagttcgccggtgactctgactacggcgcctctagcggaactgtgaacctgacggtcaaatttaagaacgccttcaccatctcacagatcatctctgcaagctctagtctcaagtcttacgtgctcaagaacaaaaaggtgcctgcgactgtgtctgtcaacggcgtgagtctcaatctgaccagtttcacctacctgatggcgaaggccaccatctcgattaactcaaacaagaccagcggttctatcctgctggtgcctgtcgactctaactacaccaacaacggctcaagaatcaatgccaacctgtacaaggccaactacatagacctggccaagaaagtgatctcttctgccgaggccaacaagctggtgcctaactctgtttccaccaacatagggctggtttcccacgacctgtactctttcggactcgccaaggccttggtgttcttcaacagcgaccactacctgcctaactatcttatcctgtctagcgatgacgtgggggagaagcacagtaccgtgatcccttctaacgcccgaggcaacgcctctcagttcaaggccggtctgaacgaggcagagacccttacggctgcccagatagccaagtatctggtggcgtctggccacgatgcaacgaactctgagatcaaggcgctcgcggccaagttggtgtctggcaagacctctctgtgggacaaggccaacgccatcttcaccttcgctagagacaacatcacgtacagttactacgctgactctaagaagggagccgccgggaccctctcttctaagtcaggcaactgctgcgaccactctaacttgatagtgtccctgtgccgcgccgccaacatcactgcgcggtttagtcacgcacagggatgcaccttctcatctggcttggtcgccggccacgtgtgggctcagatttacattgacggcgtctggtacaccgccgacgcaacatcccgccgaaactcacttggaaacatcgttaactggaacaccaaccactacaacaccctcaagcagtacgaccacctgtctttcggcgggggggggtcagggggaggggggtcaggcggcggtgggtctatcgagaacgccgacaaggccatcaaggacttccaggacaacaaagcccctcacgacaagagtgccgcgtacgaggcgaactccaagctgcctaaggacctccgggacaagaacaaccgcttcgtggagaaggtctctatcgagaaggcgatcgtccggcacgacgagcgggtgaagtccgcgaacgacgccatctctaaactcaacgagaaggacagcatcgagaaccgcaggctggcccagcgggaagtgaacaaggcccctatggatgtgaaggaacatctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 69 - protein, 1122 aa, synthetic construct; identical to SEQ ID NO: 6

SEQ ID NO: 70 - DNA, 3553 bp, synthetic construct
  source 1..3553: mol_type = other DNA
cttgttctttttgcagaagctcagaataaacgctcaactttggccaccatgttcgtgttccttgtccttctgcctctcgtttctagcgccgcgatcgacatcgatgaggcctcttcttcctctgacctcagcgactcctctatctctaacgactacctggtggctaacagcggggacgactcagttgccagctcttccgcctctagctctatcgcggcagacgactctgacctgtctaacaacgcctcttcttctaacgtcaacttcgagaacgaggtcctgtccaccaacaataacgaggataccgagtctgagatcgtcaaggactctaagaatcagctgtcctcttctagcctccaggcgtctacaaagaccaagaccaccctcaagggctccggttcttctgtgtaccgggggaacccttactacgttactctcactgactctaacggcaaggtgctggccagtcagaaggtgacgttcaatatactgggcaagaactacaccaggaccaccgacagtaaaggcgtggcaagtatcaacataaatctggccaagggcaagtacaacatcgcttgcctctacgccggaacggagaactacgcatcttctaagctgtctgttgccctgactgtgaacctcatgtctacgaaaattaacacgggcgggtctaccgtgaagaaggggaacgcctactccgtgaccctgactgacggaaacgggaaggctctgtcttctcagaaagtcacccttaacatccttgggaagaactacaccagaactacggactctaagggggttgcttctatcgcgataaacctggccgccggtaagaagttcaccctcaccgcctcttatgccggctctgccaactacctcagctctaaggtgtccgcgaccgtgaccgtgcagaagggcgacacatctattaagcctagtgggaccagcatcgtgaaagggaactcctattctttcacactggtggacggctctgggaagggcctggcgaatcaaaaggtcgcgatcaagatctctggcaaatcctatagccggaccaccaactctaacggggtggcctcaattgccatcaatttggcggccggcaagaagtactctatcgtctgctcttacgcgggttctagtaactataaggcttcttcttcaaccgtgtctctgtctgtgaccaacccttcaaccaacagcaaaaccttctctatcgccaagatcgaggccgccgccaccaacctgaaggcttacgtgaacaagaacaaggccgtgcctaccacagtgtctgtcggcggcaccaacctgaagatctctgaattctcttacttgatgtctaaggccatcgtcaacctgaactctaacaacacaaacgcgataacgctcccttctggcatctacaacggggcctcagcctctaactctctcaacgccaccgtgtacaaggcccagtacgtggacttgagcaagcgagtctacaactacatcgacaagaacaaggtccctgcggcctacggcaccgtctacaacgccaacggcgcttctcttggcaacgccggcttcaacctgtacaccttcgcgttcgccaagatcttggacttccacaagaccaacaagtacctccctaactactgctctttcgactcttctgtcttcaaggcctctaacggctctagctctagtaactcatcttcttctaccaactcatcttcatctacgaactcatcttccggctcttctaacagtagtgggtctgggagttctacgcctgccgttaccgtgaaggcgacctctttgaaggccgccagtacctccgtgatccggggcgatgactactcagtgacgctcacggactcttctgggaacgcactggcgaaccagaagattacgtttgccctctcatctagtagttacacccggaccaccaactctaagggcgttgccagcctcacgctgaacctggccggtggcaagtatagcatcaccacttcatacgccggaacatctgcctacaaggcctctaagctgacaaacaccgtcacgatctctaattcttcatctcgcttcttcctgaacgacattgagaccgccgccgagaacgtgaagacgtacgtcaccaagaacaaggctctccctaacacggtgacggtcgccggcacccagctgaccctgtctcagttctcctacgtgatggcaaaggcgatccataacatcaacgctagtaactctaactacatatctctgaagtctgtggccagttcaaacagtaccggggactacctcgacacgaccgtgtacagggcccagtacatgaacctgaccaaccgcgtcatctctttcgtggagtccgataagatcacccctacctttgccaccgtctacaactcaaacggaaagtccgtcgggaaggccgagttcaagttgtacacattcgctttcgccaagatcctggccttctacaaaaccaataactacctgcctacgtactgcacgtttcagagctctgccatcggcgtggtgcctgacgtggccacgaacgtgaccatcaacagtaagatcaacgccaacatgaaccagttcaaagtcggtctgaacgagaagaacactgtgtctaacctgtctgcatacctggtgggtaccggccagtctactatcacaactaacatcaagaacgtcgccgcccagctgaccaagggactcaactctacagccactaaggccctggctatctacaacttcgtgcgagacgacatctcttactcctactactctgacagccgcaagggagccgacggcaccctgtcttccgggtccgggaactgcgtcgatcaggcatctttggtggtggccctgtgccgggcagccgggatccctgcacgctactcacacgcgcaggggtgtaccttctctagcggccttgtcacgggccacgtgtgggcccaaatcctggtggacggagtctggtactctgcagacgccacctcagtgcggaactctttgggcaacatagtgaactggaataccaactcttaccactcaatgaagcagtacgccgcggtgcctttcggtggcggggggtctggagggggcggttctggcggcgggggctctatcgagaacgcggacaaggcgatcaaggacttccaggacaacaaggcgcctcacgacaagagtgccgcatacgaagccaacagcaagctccctaaggaccttcgcgacaagaataatcggttcgtcgagaaggtgtctatcgagaaggccatcgtgcgccacgacgagagagttaaatctgccaacgacgccatctctaagctgaacgagaaggacagtatcgagaaccgccggcttgcccagcgcgaggtgaacaaggcccctatggacgtgaaggagcacctgcagaagcagctcgactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 71 - protein, 1003 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSAAIDVDTNDNLDDGSSSNSDLISSSSLDSSSSDDVSSGSSEVSSSDESLDGNNLSDGNVSSSDESVGADNLSDGNVSSSDESVGADNLSDDESSSDALSEELPKTETVIKADPINYNYASVKGLTINLTDSAGLALSNKTLTVKVSALNKTSNLTTNSKGQAIFKLSASVGSYDVFISFTGDESYAPSNASSKITIKKSSTKIKLSNIHGYLTISNYVSVTLLDSAGKPIKSKSVTIQVNKAKYNVKTDSKGIAKVKVANKIGTYSVNAKFSGDKNYYASSNSSKLTITKMKVYIKAPSVKYYMTNSSAPYLTINLTNVKGSPLAKKKVSVKIGKKTYTLKTNSQGIAKFKFTKKVSSYNCKINFKATSNFYGASVNSKMTIQKMPTSLKAPSVSINSTNYGKVLISLKDGKGKALKNTTVTVNVTELKKVFTLKTNASGVATFSFNGEKTFNLKIKYAGNKNYAASSVSSKINVKQIKVKLSDVIGASRVLIDYVNRTKDLPSNVQYNNYNFTVTQLTYLASKAVKNINNKNYGDIVLISVPKSYKSSGEIYDTVYKKDFVKIANSVVGSSYNYKNKEYVSYSIYKVPFKVYSISFAKVLNFYGNNKKLPNYSLFTLADFAKVKDNGGYNFYLTTDNIAGKKSDLNMLKSLAKTLKSMGYNAVIVGIGPDIHNVAYRYGCTGNNSVLLACFGGVDVGCIEEWAGDLGDLNGHSFVNSYQGAHVLGLWFTKPYGASVSLNKKVGIAWDADYGFPLNTPAKYMKSHNISYIETGTVANACKLLSEGKMGGPQLISGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 72 - DNA, 3196 bp, synthetic construct
  source 1..3196: mol_type = other DNA
cttgttctttttgcagaagctcagaataaacgctcaactttggccaccatgcgcttcccttctattttcaccgccgtgctcttcgccgcctcttcagcactcgcaatgcctctgtctgggggcgggtctggcggcagcggctctatgcctctgaacaccactaccgaggacgagacggcgcagatacctgcggaggccgtgatcgggtacctcgatctggagggcgacttcgacgtggccgtgctccctttcagtaactcaaccaacaacgggctcctgttcatcaacaccaccatcgctagtatcgccgcaaaggaggagggcgtttcagccgccatcgacgtcgacaccaacgacaacctggatgacggctcttcttctaattctgacctgatcagctcctcttctttggacagctctagcagtgacgacgtgtccagcgggtcaagtgaagtgtcatcctctgacgagtctctggacggcaataacttgagcgacggaaacgtgtcttcatcggacgagagcgttggggccgacaacctgtctgatggcaacgtctcttctagtgacgagtctgtgggcgccgacaacctttctgacgacgagtcatcttctgacgccctttctgaggaactgcctaagaccgagactgtgatcaaggccgaccctataaactacaactacgcgtccgtgaagggtctgaccataaacttgaccgactctgcgggcctggcactgtctaacaagaccctgaccgtgaaggtgtctgcccttaataagacctctaacctgacaacgaactccaaggggcaggccatcttcaagctttctgcctccgttgggtcttacgacgtcttcatctctttcaccggagacgagagctacgcccctagtaacgcatcttctaagatcaccatcaagaagtctagtactaagatcaaactttctaacatccatgggtacctgaccatcagtaactatgtgtctgtgactctgttggactctgccggaaagcctatcaagtccaaatccgtgaccatccaagtgaacaaggcgaagtacaacgtcaagacggactctaaggggatcgccaaggtgaaggtggccaacaagatcgggacctactctgtgaacgctaagttcagcggggacaagaactactacgcgtcttctaactctagtaagctcaccatcacgaagatgaaagtgtacatcaaagccccttctgttaagtactacatgacaaactcttctgccccttacctgacgatcaacctgacgaacgtgaaaggatctcctctggccaagaagaaggttagcgtgaagatagggaagaagacctacacgctgaagaccaactctcagggaatcgccaagttcaagtttacgaagaaggtgtcttcttacaactgcaagatcaacttcaaggccacctctaatttctacggcgcctccgtgaactctaagatgaccatccagaagatgcctacaagtctgaaggccccttctgtgtctatcaactcaacaaactacgggaaggtcctgatctcactgaaggacggcaaggggaaggccctcaagaacacgacagtcacggtcaacgtgaccgagttgaagaaggtcttcaccctgaagacaaacgcctctggtgtggctaccttctcttttaacggcgaaaagaccttcaacctcaagattaagtatgcgggaaacaaaaactacgcggcctcttctgtctcttctaagatcaacgttaagcagattaaggtcaaactctctgacgtcatcggcgcctctcgggtgctcatcgactacgtcaaccggaccaaggatctgccttcaaacgtgcagtacaacaactacaacttcaccgtgacgcagctcacctatctggcttctaaggccgtgaagaacatcaacaacaagaactacggcgacatcgtcctcatatctgtccctaagagttacaagtcatctggcgagatctacgacacggtgtacaagaaggacttcgtgaagatcgctaactccgtggtggggtcttcttacaactacaagaacaaggagtatgtttcttactcaatctacaaggtgcctttcaaggtctactctatttctttcgccaaggtgctgaatttttacggcaacaacaaaaagctgcctaactactccctcttcaccctcgccgacttcgcgaaagtgaaggataacggcggttacaacttctacctgaccaccgacaatatcgccggaaagaagtcagacctgaacatgctgaagtccctcgccaagaccctgaagtctatgggttacaatgccgtcattgtgggcatcgggcctgacatccacaacgtcgcctacagatacgggtgcactgggaacaactctgtcctcctcgcgtgcttcggcggagtcgacgtgggctgcatcgaggagtgggccggcgacctgggcgacctgaacgggcactctttcgtgaactcttatcagggcgcgcacgtcctggggctctggttcactaagccttacggcgcctccgtcagtctgaacaagaaggttggcatcgcctgggacgcggactacggcttccctctcaacacgcctgcaaagtacatgaagagtcacaacatcagctacatcgagaccggtaccgtggcaaacgcatgcaagctgctcagcgaggggaagatgggcggccctcagcttatcagcggcggcggcggctctggcggggggggcagcggtggtgggggttctatagagaatgcggacaaggccatcaaggatttccaggacaacaaggctcctcacgacaagtcagccgcttacgaggccaactctaagttgcctaaagacctgcgcgacaagaacaaccgcttcgtggagaaggtgtctattgagaaagccatcgtgcggcacgacgagcgcgtgaagtctgccaacgacgccatctccaagctgaacgagaaggacagtatcgagaacaggcggttggcccagcgagaggtcaataaggctcctatggacgtcaaggagcaccttcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 73 - protein, 860 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSAAQSLDNINYANDGFDSDEMINCDLHKDSSQKSLKSNALSNKKTTNTVKLTDMKKAESNDVKQTGAAKASNTKSTSKSTTKTNATKSNTTKSTATKTTANSSSTKKATQNTTTINTQTLAKSSSSYMAYVEKNAKLQEPITISKKKYKSPEYLYLVSKAVSNISKTKVEIKDKLITNYSNTDCKSVNGTINKTEYVQVAKKTVSFIEKNHRAPNWIASSKGNIPRNQLILVFSKCLDQYNKSGKLPSSIKLNDLDLNKMKQKIDSSKKVNSTSTKKTNTSSTKTNSTSAKKTNTTSTKKTNPTATSTNNNKSLVESTLDSIKSILNNIENKLNPTNKVLSTTGTKKNTVTVNSSKVNVQISSSSTVNVKISAKDNTNSGKNTNSGSAKKTNTTSTKKTNTTSTKKIDTNSTKKTNTTSTKNNTSSAKKTNTTSTKNNTSSAKKTNTTSTKNNTSSAKKVNTSSSKTNTSAKNNTSTTAKSSSNSKYLSTSVLNDKYLGESLKKYLAVGKNCQVTNKAIKTLANTLTSKLKSDYKKGEKIFNWVRDNIGYEKYRNTKKGALKTLQTRGGNCVDHAHLIVALSRAAGLPARYVNANNCKFSSGYVSGHVWAQVLVGNTWVVADATSNRNKFGVVKNWNVNSYKLVGKYSSISFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 74 - DNA, 2767 bp, synthetic construct
  source 1..2767: mol_type = other DNA
cttgttctttttgcagaagctcagaataaacgctcaactttggccaccatgaggttccctagcatttttaccgcggttctctttgccgcttctagcgctctcgcaatgcctctctccggggggggaagcggagggtcaggctctatgcctttgaacaccaccacagaagacgagactgcgcaaatacctgcggaggccgtgatcggctacctggatctggaaggggatttcgacgtggcagtgctgccttttagcaactctaccaacaatggacttctgttcattaacacaacaatcgcctccatcgcagctaaggaggaaggggtgtcagctgcccagagtctggataacatcaattatgccaacgacggattcgactctgacgaaatgatcaactgcgacctccacaaggactccagtcagaaaagcctcaagtctaacgcactgtccaacaagaagacgaccaataccgtcaagctgacagatatgaaaaaagctgagtcaaacgacgtgaagcaaacaggcgcagctaaggccagcaatactaaaagtacttctaagagtactacaaagaccaacgctactaaatctaataccacaaagtcaactgccaccaagactaccgcaaattcttcaagcacaaagaaagccacgcaaaataccactacaatcaacacccagacgctggccaagtcctcatccagctacatggcatatgtggagaagaatgccaagttgcaagagcctatcaccatttctaagaaaaaatataagtctcctgagtatctgtacctggtgtctaaagccgtgagcaacatcagcaaaacaaaggtggagatcaaggacaaattgattaccaattactccaacacggattgcaaaagcgtgaacggcacaatcaataaaacagaatacgtgcaggtcgccaaaaaaaccgtgagtttcatagagaagaatcatcgggctcctaattggatcgcctcaagcaaggggaacatacctagaaatcagcttattctggtctttagcaaatgtctcgatcagtacaacaaatcaggtaaactcccttcaagtataaaactgaacgatttggacctgaacaagatgaagcagaagatcgactcctccaagaaggttaattccactagcacaaagaagaccaacacctccagcactaaaacaaactccacctcagcaaagaagactaacacgacatccacaaaaaagactaatcctaccgccacgagcacaaataataataagtccctcgtcgagagcactctggatagtatcaagagcattttgaataatattgagaacaagcttaatcctacgaacaaagtcctttccacaaccggaaccaagaaaaacactgttaccgttaacagttctaaagtgaatgttcagattagcagctctagcaccgtgaatgtcaagatcagtgccaaggataacaccaacagcgggaagaacaccaactccggcagcgccaaaaagaccaacactaccagcactaagaaaaccaataccacctcaacaaaaaagattgatacaaattccacgaaaaaaaccaacaccacgagtacaaaaaataacacgtccagcgccaagaagactaatacgacctcaaccaaaaacaatacttccagtgccaagaagacgaacacaacttccactaagaacaacacaagttccgcgaaaaaagtgaacacttcttccagtaaaaccaacacgtccgcaaagaacaatacatctacgacggctaagtcatccagcaattccaagtacctcagcacctccgtgctcaacgacaagtacctgggcgagagtttgaagaaatatctggccgttggaaagaattgtcaggtgaccaataaggctattaaaacacttgccaacaccctgacttccaaactcaagagcgattacaaaaagggggaaaagatctttaactgggtccgggacaacataggctacgaaaagtatagaaacaccaaaaagggagccctgaagacccttcagacacgggggggcaattgtgtcgatcatgcacacttgatagtggcgctgagtcgagccgctggtctgcctgccaggtatgtcaatgccaacaactgcaaattttcaagtggctacgtgtctgggcatgtgtgggctcaggtcctggtcggtaacacttgggttgtggccgacgcaaccagcaaccgcaataagttcggagtggtcaaaaactggaatgtgaactcttacaagctggtgggcaagtattcaagcatcagtttcggtggtggcggttctgggggaggcggctctggtggcggcggaagcatcgaaaacgctgacaaagcgatcaaggacttccaggataacaaggcgcctcacgataagtctgctgcatacgaggctaattctaaactgcctaaggaccttcgcgacaagaataacagattcgttgaaaaggtgtcaatcgagaaggccattgtgaggcacgacgaacgcgttaagagcgccaatgatgcgatcagtaaactgaacgaaaaagattccattgagaaccgacgcctggctcagcgggaggtgaacaaagcacctatggacgtcaaggagcacctccagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 75 - protein, 1191 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSAASNASDNLDDLTISDSNSLDLVSTSNSDILSSDSGVSSDDSSNDASGDVLGSDVSSNESNNQSQSTLDSNNQSQSGLDSDNSTLLDSQSNNQSNSESSDSSDSSETVIKNATSISVSSKTVVRGNSLNITLKDNASTLLSNKTVTFTFNGKTYNKTTNAKGIASLTLTATPKKYLVKIAFVGDELYEASSKSVNVTLSKTPTSISNSGKSIVRGKLYKLTLKDAKGKALSGKKISISFNGKKYTKTTNSNGQVNLTINVNVGKTYKMTYKFAGDSNYLSSSGSVSIKVKMGTSIIGSGSSIVKGKSYTVTLKNANGAVLSNQKIAFTLSGKTYNRTTNAKGQASLKIGLSSGKTYNLTYKYAGNSYYGGSSGKVSLFVKTPTTMKNSGKTIVSGETYKVTLKDADGKSLANKKVSITFNNKTYAKTTNSNGQASLTIKGTFGRSYPLSYKFAGDSKYGPSSGSLCLRVKKATSLKGSASSIVQGKSYTVTLKDSNSTPLANQTIVFTLDTKKYNRTTNAKGQASLKIGLAAGKTYNLAYKYSGTSYYNGSSGSVKLKVKFPTSLTNSGKSVMNGTGYNIVLKDSKSNLVSNKTISIGFNGKTYDEITDANGTVTLLIDANVPKTYKMTYKFAGDSDYGASSGTVNLTVKFKNAFTISQIISASSSLKSYVLKNKKVPATVSVNGVSLNLTSFTYLMAKATISINSNKTSGSILLVPVDSNYTNNGSRINANLYKANYIDLAKKVISSAEANKLVPNSVSTNIGLVSHDLYSFGLAKALVFFNSDHYLPNYLILSSDDVGEKHSTVIPSNARGNASQFKAGLNEAETLTAAQIAKYLVASGHDATNSEIKALAAKLVSGKTSLWDKANAIFTFARDNITYSYYADSKKGAAGTLSSKSGNCCDHSNLIVSLCRAANITARFSHAQGCTFSSGLVAGHVWAQIYIDGVWYTADATSRRNSLGNIVNWNTNHYNTLKQYDHLSFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 76 - DNA, 3760 bp, synthetic construct
  source 1..3760: mol_type = other DNA
cttgttctttttgcagaagctcagaataaacgctcaactttggccaccatgcgcttccctagtatcttcaccgccgtgctgttcgctgcctcttctgccttggccatgcctctgtctggcggaggctctgggggctcaggttctatgcctctcaacacgacgacggaggacgagaccgcccagatacctgccgaggccgtgatcgggtacctcgacctcgaaggtgactttgacgtcgcggtgctccctttctctaactctacaaacaacggcttgctgttcatcaacaccacgatcgcgtcaatcgcggccaaggaggagggcgtctctgccgcctccaatgcctccgataacctggacgatttgaccattagcgacagtaactccctggatcttgtgagcacctctaactctgacatactgtcttctgactctggcgtctcttctgacgactctagtaacgacgccagtggagacgtcctgggcagtgatgtgtcatctaacgagtccaacaaccagtcccagtctaccctcgactctaacaaccagtctcagtctggtctcgactctgacaattctaccctccttgactcacagtctaacaaccagagcaactctgagtcttctgactctagcgactcttcagagactgtcatcaagaatgcaacctctatctctgtgagctctaagaccgtcgtgcggggaaactccctgaacatcaccctgaaggacaacgcttcaaccctgctttccaacaaaaccgtcaccttcacgttcaatggaaagacttacaacaagaccaccaacgcgaaggggatcgcctctctgaccctgactgccacccctaagaagtaccttgtgaagatcgcgttcgtcggggacgagctgtacgaggcgtctagcaagtctgtgaacgtcaccctttctaagacgcctacaagcatatctaactctggcaaaagcatcgtgcgagggaagctgtacaagctcacgctcaaggacgccaaggggaaagccctgtctggaaagaagatctctatctctttcaacggcaagaagtacaccaagaccaccaacagtaacggtcaagttaacctgacgatcaacgtgaacgtgggcaagacatataagatgacctacaagttcgccggtgactctaactacctgtcatctagtggcagcgtgtctatcaaggtcaaaatggggacctctattatcgggtctgggtcttctatcgttaagggtaagtcatacaccgtcacactgaagaacgccaacggggccgtgctgtctaaccagaagatcgcattcacactctccggcaagacgtacaacagaacgaccaacgccaagggccaggcctccctgaagatcgggctgtcttctgggaagacctacaacctgacgtacaagtacgccgggaactcttactacggagggagttctggcaaggtgtctctgttcgtcaagacccctaccacaatgaagaactctgggaagaccatcgtctctggcgagacctacaaggtgaccctcaaggacgccgacggcaagagccttgcaaacaagaaggtgtcaatcaccttcaacaataagacctacgccaagacgaccaacagtaacgggcaggcctctctgacgattaagggaaccttcggacgctcttatcctctgtcttacaagtttgcgggcgacagcaagtacgggccttccagtggcagcctgtgcctgcgcgtcaagaaggcgacgagcctcaaggggtctgcctcctctatcgtgcagggcaagtcttacaccgtgactctgaaggactcaaacagtacgcctctcgccaaccagaccatagtgttcactcttgacaccaagaagtacaacaggaccaccaacgccaagggccaggcctctttgaagatcgggctggccgccgggaagacctacaacctggcctacaagtactctgggacctcttactacaacggctcaagtgggtcagtgaagctgaaggttaagttccctacctctctcaccaactccgggaagtctgtgatgaacggcaccggctacaacatcgtcctcaaggactctaagagcaacctggtttctaataagaccatctccatcggcttcaacggcaagacgtacgacgagatcaccgatgcgaacggcaccgttaccctcctcatcgacgctaacgtccctaagacgtacaagatgacttacaagttcgcgggcgactccgactacggggcgtctagcgggacagtgaacctcactgtgaaattcaagaacgccttcacgatcagtcagatcattagtgccagctcttctctgaagtcttacgtgctcaagaacaagaaggtgcctgccacggtgtcagtcaatggcgtgtctctgaacctgaccagtttcacctacctgatggcgaaggcaaccatctccattaactctaacaagacctctggctctatactgctcgtgcctgtcgactcaaactacaccaacaacggctctcggatcaacgccaatctgtacaaggccaattacatcgacctggccaagaaggtcatctcttctgctgaggccaacaaattggtgcctaacagcgtgtccacaaacatcgggctggtgtctcacgacttgtactccttcggactcgccaaagcgttggtcttcttcaacagtgaccactacttgcctaactacctgatcctttcttctgacgacgtcggggagaaacactctacggtgatcccttctaacgctcgcggcaacgcatctcagttcaaggcgggcctcaacgaggccgagactctcacagccgcacaaatcgccaaatatctggtggcgtctgggcacgacgccacgaactctgagatcaaggccttggccgccaagctggtgtcaggcaagacctctctgtgggacaaggcaaacgccattttcacctttgctcgcgacaacatcacctacagttattacgccgacagtaagaagggcgccgctggcaccttgagctcaaaatctggcaactgctgcgaccactctaaccttatcgtgtctctgtgccgggccgccaacataaccgcccggttctctcacgcacaggggtgcacattctcttctgggctggtggccggccacgtctgggcacagatctacatcgacggcgtgtggtacactgccgacgccacgtctaggcgcaactcccttggtaacatcgtgaattggaacaccaaccactataacactctgaagcagtacgaccacctgtctttcggcggcggcggttctggcggaggtggttctggcggcgggggctctatcgaaaacgccgacaaggccatcaaagacttccaggataacaaggcacctcacgacaagtcagcggcctacgaggctaactctaagctgcctaaggacctccgggacaagaacaaccgcttcgttgagaaggtgtctatcgagaaggctatcgttagacacgacgagcgggtgaagtctgctaacgacgcgatctcaaagctgaacgagaaggactctatcgaaaaccggcggctggcccagcgagaggtgaacaaggcccctatggacgtgaaggagcatctccagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 77 - protein, 1203 aa, synthetic construct
MRFPSIFTAVLFAASSALAMPLSGGGSGGSGSMPLNTTTEDETAQIPAEAVIGYLDLEGDFDVAVLPFSNSTNNGLLFINTTIASIAAKEEGVSAAIDIDEASSSSDLSDSSISNDYLVANSGDDSVASSSASSSIAADDSDLSNNASSSNVNFENEVLSTNNNEDTESEIVKDSKNQLSSSSLQASTKTKTTLKGSGSSVYRGNPYYVTLTDSNGKVLASQKVTFNILGKNYTRTTDSKGVASININLAKGKYNIACLYAGTENYASSKLSVALTVNLMSTKINTGGSTVKKGNAYSVTLTDGNGKALSSQKVTLNILGKNYTRTTDSKGVASIAINLAAGKKFTLTASYAGSANYLSSKVSATVTVQKGDTSIKPSGTSIVKGNSYSFTLVDGSGKGLANQKVAIKISGKSYSRTTNSNGVASIAINLAAGKKYSIVCSYAGSSNYKASSSTVSLSVTNPSTNSKTFSIAKIEAAATNLKAYVNKNKAVPTTVSVGGTNLKISEFSYLMSKAIVNLNSNNTNAITLPSGIYNGASASNSLNATVYKAQYVDLSKRVYNYIDKNKVPAAYGTVYNANGASLGNAGFNLYTFAFAKILDFHKTNKYLPNYCSFDSSVFKASNGSSSSNSSSSTNSSSSTNSSSGSSNSSGSGSSTPAVTVKATSLKAASTSVIRGDDYSVTLTDSSGNALANQKITFALSSSSYTRTTNSKGVASLTLNLAGGKYSITTSYAGTSAYKASKLTNTVTISNSSSRFFLNDIETAAENVKTYVTKNKALPNTVTVAGTQLTLSQFSYVMAKAIHNINASNSNYISLKSVASSNSTGDYLDTTVYRAQYMNLTNRVISFVESDKITPTFATVYNSNGKSVGKAEFKLYTFAFAKILAFYKTNNYLPTYCTFQSSAIGVVPDVATNVTINSKINANMNQFKVGLNEKNTVSNLSAYLVGTGQSTITTNIKNVAAQLTKGLNSTATKALAIYNFVRDDISYSYYSDSRKGADGTLSSGSGNCVDQASLVVALCRAAGIPARYSHAQGCTFSSGLVTGHVWAQILVDGVWYSADATSVRNSLGNIVNWNTNSYHSMKQYAAVPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 78 - DNA, 3796 bp, synthetic construct
  source 1..3796: mol_type = other DNA
cttgttctttttgcagaagctcagaataaacgctcaactttggccaccatgcgcttcccttcaatcttcacagccgtcctgttcgcggcctcttctgccctcgctatgcctttgtctggcggcggctctggcgggtccggctcaatgcctctcaacacgaccacagaagacgagacggcgcagatccctgccgaggcggttatcggatatctggacctggagggagatttcgacgtggcggtgctgcctttcagcaacagcactaacaacgggctcctgttcatcaacaccaccatcgcctctattgcagcgaaggaggagggcgtgtctgccgccatagacatcgacgaggcctcctcctcttctgatctctctgactcttctatctctaacgactacttggtggccaactctggcgacgactccgtcgcgtcttcttctgccagttctagtatcgccgcagacgactcagacctcagtaacaacgcttcttcttctaacgtcaacttcgagaacgaggtcctctctacaaacaacaatgaggacaccgagagtgagatcgtcaaggacagtaagaaccagctgagctcttccagcctgcaagctagcacgaagaccaaaactacccttaagggctccgggtcttctgtgtaccggggtaacccttactacgtgactctgactgactctaacggcaaggtgctcgccagccagaaggtcaccttcaacatcttggggaagaactacactagaacgaccgactctaagggcgtggccagtatcaacatcaaccttgccaagggcaagtacaacattgcctgcctctacgcggggacggagaactacgcctcctctaaactctcagtggctctcaccgtgaacctgatgagcactaagatcaatacggggggttctaccgtcaagaagggcaacgcctactctgtgaccctgacagacggcaacggcaaggcattgtctagccagaaggtcaccctgaacattctggggaagaactacacccggaccaccgacagtaagggcgtggcgtcaatcgccataaacctggcggccggtaagaagttcactctgaccgcctcctacgccgggtctgccaactacctgtcttccaaggtgtcagccaccgttacggtgcagaagggcgacacatctatcaagccttctggcacctcaatcgtcaaggggaattcatactctttcacgcttgtggacggctctgggaagggcctggccaaccaaaaggtcgccatcaagatcagcgggaagtcttactcccggaccacgaattcaaacggggtggcttccatcgccatcaacctggccgccgggaagaagtactcaatcgtgtgcagttacgcgggctcttctaactacaaggcctcttcaagcaccgtgtctctgtctgtgacgaaccctagtaccaactctaagacatttagtatcgcgaagatcgaggccgcagcgaccaacctgaaggcttacgtgaacaagaataaggcagtgcctaccacagtgtctgtggggggcaccaacctgaagatatctgagttctcttacctgatgagtaaggccatcgtcaacctgaactctaacaacaccaacgcgatcacgctgccttctggaatctacaacggggcctctgcctctaactctctcaatgccaccgtgtacaaggcccagtacgtcgacctgtctaagagggtctataactacatcgacaagaacaaggtgcctgccgcctacggcacagtgtacaacgccaacggggcctctctgggcaacgcaggcttcaacctgtacacgttcgccttcgccaagatcctggacttccacaagactaataaatatttgcctaattactgctctttcgattcttcagtgttcaaggcctctaacggctcttcatcttcaaactcttctagcagtaccaactcttcttcttctaccaactctagttctgggagctccaactcttctggatctggctcctctacccctgccgtgaccgtgaaggccaccagtcttaaggcagcttctacctctgtcatccggggcgacgactactcagtgaccctgaccgacagcagcgggaacgccctggccaaccagaagattacgtttgctctgtcttcttcttcctacaccaggacaacgaactctaagggggttgcctctctgaccctcaaccttgcgggcggcaagtattccatcaccacgtcttacgccgggacctcagcgtacaaggccagtaaactgaccaacaccgtcaccatcagcaactcctcttctcgattcttcctcaacgacatcgagaccgcagccgagaacgtgaaaacgtacgtcaccaagaacaaggcgttgcctaacaccgtgactgtcgcggggacccagctgacgctctctcagttctcttatgtgatggccaaggcgatccacaacatcaacgcgtctaactctaactacatctccctgaagtctgtggcttcatctaactccaccggagactacctggacaccactgtttaccgcgcccagtacatgaacctgaccaaccgagtgatcagcttcgttgagtctgacaagatcacccctaccttcgcaacggtctacaactctaacggaaagtctgtcggcaaggccgagttcaaactctatacgttcgccttcgccaagattctcgcgttctacaagacgaacaactaccttcctacctactgcacattccagagttctgccatcggcgtcgtgcctgacgtcgccaccaacgtgaccatcaactctaagatcaacgccaacatgaaccagttcaaggtgggtctcaacgaaaagaacaccgtctctaatctttctgcctacctggtcgggaccggccagtctaccatcacaaccaatattaagaatgtggccgcccagctcacgaagggcctcaacagtaccgccaccaaggccctcgccatatacaacttcgtccgcgacgacatctcttacagttactactctgactctcggaagggtgccgacggtaccctttcttctgggtctggcaactgtgtggaccaggcctcactggttgtggccctgtgccgggccgcgggaatacctgcccgctactctcacgcccagggctgcaccttctcttctgggttggtcaccggccacgtgtgggcccagatcctggtggacggcgtttggtactctgcagacgctacgagcgtgcggaacagcttggggaacatcgtgaactggaacactaactcttaccactctatgaaacagtacgccgccgtgccttttggtggagggggttccggaggcggcgggtctgggggcggtggcagcatcgagaacgctgacaaggccatcaaggacttccaggacaacaaggcacctcacgataagtctgcagcctacgaggccaactctaaactccctaaggacctccgcgacaaaaacaaccgcttcgtggagaaggtcagtatcgagaaggcaatcgttcgccacgacgagcgggtgaagtcagccaacgacgcgatatctaagctgaacgagaaggactctatcgaaaaccggcgcctggctcagagagaggtgaacaaggcccctatggatgtgaaggagcatctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 79 - protein, 473 aa, synthetic construct
MFVFLVLLPLVSSAACGAFQTTAATVPGSSDPMWFAMDYVKENSTNDTVIISWWDFGYLFQVASDHPTSFDGGSQTGDRAYWVGKSLTTSDYAQSKGILQMLATTGSNASMLLSEYTGSNVTAVHALDETLGKSRSEAQKILTSKYNLTNDQAKAVVKQSHPSNPNNVSFVLSSDMIGKAGWWSYFGSWNFDTLNSTNYQYYMANDYVPIKQNTQGNITILNESGIIYQAVVNRGKNGTNETTAQMETIWDNNRSKIDLNGTEYNPLKASNLICIENSYLTVNKTLNKDGNYTLYLLGSGDDYTAILMDNNLKDSVFTRLFLLGGIGQDTFELSNMQDGVSVWTLRDGSSNSDDAGSQGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 80 - DNA, 1605 bp, synthetic construct
  source 1..1605: mol_type = other DNA
cttgttctttttgcagaagctcagaataaacgctcaactttggccaccatgtttgtgttcttggtcctgctccctctggtttcaagcgctgcctgtggggcattccagaccaccgcagccactgtgcctggatcctctgaccctatgtggttcgccatggactacgtcaaggaaaactctactaatgatacagtgatcatttcatggtgggacttcgggtatctgtttcaggtggcgtcagatcatcctacctcatttgatggcgggtctcagacaggcgatagggcttactgggtggggaagagcctgacgacatcagactacgcacagtcaaaggggatcttgcagatgctggctactaccggctccaacgcttccatgctgctgagcgagtataccggaagtaatgtgacggctgtgcacgcccttgatgaaaccctcggcaagagtcgcagtgaggcacaaaagattctcactagcaagtacaaccttaccaatgaccaggccaaagccgtggtcaaacagtctcacccttccaatcctaacaacgtcagcttcgtgctgtccagcgacatgattggcaaagctggctggtggagttacttcggatcttggaatttcgacaccctgaattccaccaattaccagtactacatggccaacgactatgtgcctataaagcaaaacacccagggtaatatcacgatcctcaacgagagcggaatcatttaccaggccgtcgttaatagggggaaaaacggcacaaacgaaacgaccgcccaaatggaaaccatctgggataacaaccggagcaagattgacctcaatgggacagagtataaccctttgaaagccagcaacctgatttgcatcgaaaacagttacctcacagtgaataaaacactcaacaaggatggtaattatactctgtatctgttgggctctggtgatgactacacagcaatcctgatggacaataacctcaaggactctgtgttcactcgcctgtttcttctcggcgggatagggcaagacacctttgagttgtccaacatgcaggatggcgtttccgtgtggactctgagggacggcagttctaacagcgacgacgccggtagccagggtggcggcggttccggcggaggaggaagtggagggggaggatccattgagaatgcggataaagccatcaaggatttccaagacaacaaggcacctcacgacaagtctgccgcatatgaagcgaactccaaacttcctaaggacctgagagacaaaaataatcggtttgtcgagaaggtttccatcgagaaggctatagtgagacatgatgaacgagttaaaagcgccaacgatgctatcagtaagctgaacgagaaggatagcatcgagaatagacgccttgcgcagcgggaggtcaacaaagcccctatggatgtgaaggagcacctgcagaaacagctggactaatgatagaccagcctcaagaacacccgaatggagtctctaagctacataataccaacttacactttacaaaatgttgtcccccaaaatgtagccattcgtatctgctcctaataaaaagaaagtttcttcacattc

SEQ ID NO: 81 - protein, 491 aa, synthetic construct
MFVFLVLLPLVSSAAYQTANQVVPGTSDAMWDSMVWINENTADNTVVASWWDFGYLFEIAADRQVIFDGGSQSGNSRAFWLGQAMTTDNMDLSAGIFRMLGTSGENATNTLTDYTGSPGKATDILIDILPKNAQDAKNALINTYGLTTEQANTIIPLTHPDNPRPVIFVASSDMLQKAGWWSYFGAWDFDKQNSTNFNYYVPSQQVTVEPGSTGRLALINESGLEYDAVITRGTGNNTTTGHTEAVYSNNGSLLKINGSEFNPLNVSRIMVIEGNQLVKNESIAGAPSDSNYTLFLMGENNVYTPIIMHNKLADSMFTRLYLLGGMGQNVFSMVHMENGVSLWQVNYNNTAAGGTSTPAASGATDGNIGTTSPDRAGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 82 - DNA, 1660 bp, synthetic construct
  source 1..1660: mol_type = other DNA
cttgttctttttgcagaagctcagaataaacgctcaactttggccaccatgtttgtgttcctggtccttctgcctctcgtcagtagcgctgcctatcaaactgccaatcaggtggttcctggcacctctgacgctatgtgggattctatggtgtggattaacgagaacacggccgacaacacggtggtggcctcatggtgggatttcgggtacctgtttgagattgccgccgaccgacaggttattttcgatgggggctcccagagcggaaattccagggctttttggctcgggcaggctatgaccacggacaatatggacctctcagccggcattttccggatgctcggtacttctggcgaaaacgccactaacaccctgacagattataccggctcacctggtaaggcgactgatatacttatcgacattctgcctaaaaatgcacaagacgccaagaatgccttgatcaacacatatggcctgaccacagagcaggcaaatacaatcattcctttgacacaccctgataatcctaggcctgtcatcttcgtcgccagctccgacatgcttcaaaaggcaggctggtggtcatacttcggcgcttgggattttgacaaacaaaatagtacgaattttaattactatgtgccttctcagcaggttaccgtggagcctggaagcacggggcgcctggcactgataaatgaaagtggcctcgagtatgatgcagtcatcactaggggtaccgggaacaacaccactaccggccatactgaggcagtgtactccaacaacggcagcctgctgaaaatcaacgggtccgaatttaaccctctgaacgtctcccgaataatggtgattgagggtaaccagctggtgaagaacgaaagcatcgcgggagcacctagcgacagtaattatacactgttcttgatgggtgaaaacaacgtgtacacacctatcatcatgcataacaaacttgcggacagcatgttcacccgcctgtacctgttgggcgggatgggacagaacgttttctctatggtccacatggaaaacggagtctcactgtggcaggtgaactacaataatacagccgccggtggcacctccacacctgcggcttccggagcaaccgatggcaatattggcaccacttcacctgatcgcgctggtgggggaggctctgggggaggaggatccggagggggcgggtccatagagaatgctgataaggctattaaggactttcaggacaacaaagcccctcatgacaagtctgctgcatacgaagcgaacagcaagctccctaaggatcttagagacaagaataaccggttcgtggaaaaagttagtatcgaaaaggccatcgtgcgccacgacgagcgggtcaagagcgccaacgacgctatcagcaaactcaatgagaaggacagtatcgagaacagacggctcgcccagagagaggtgaacaaggcccctatggatgtgaaagagcacctgcagaaacagttggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 83 - protein, 467 aa, synthetic construct
MFVFLVLLPLVSSAAYITSENVIPGTSDPMWNSMEWINEHTSNDTVITSWWDFGYLFEIAADRQVTFDGGSQTGSRAFWLGQAMTTDNLELSAGIFRMLDTSGERAVDALINYTGDTGKTTKILIDILPMTKQNAQKTLIDKYDLSTDQAKEIVGYTHPSKPRPVIFVASSDMLQKAGWWSYFGAWDFKNQSSENYNYYVPTEQVKVESGSSGKLSLIVDGGMTVNAVITRGTGNNTTSAYTEAVYTHNGSQIYVNKTVYNPLNISNLIVVEDGYLMKNESVGNVKDANYTLFLMGEKNKYTPILISNKLANSMFTKLYLLGGAGQDIFTNVHTEEGVMLWQVNFNNTVAGKGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 84 - DNA, 1588 bp, synthetic construct
  source 1..1588: mol_type = other DNA
cttgttctttttgcagaagctcagaataaacgctcaactttggccaccatgttcgtctttctggtgctgcttcctttggtgagtagcgccgcttatatcacttctgagaacgttattcctggtacatctgaccctatgtggaattccatggagtggatcaacgaacatactagtaacgacactgttatcacctcttggtgggactttgggtacctcttcgagatcgcggcagacagacaagtgaccttcgatggcgggagccaaacagggagcagggcattctggttgggccaggccatgactactgataacctcgagctcagcgcaggtatctttcgcatgctcgatacgagtggggaacgggccgtggacgccctgataaactatacgggagacaccggcaaaacaaccaagatcttgattgacatcctgcctatgacgaaacagaacgcccagaaaactctgattgataagtacgatctgagtacagatcaggccaaagagattgtcgggtacacacatcctagcaagcctaggcctgttatatttgtcgcttctagcgacatgctgcagaaggcgggttggtggtcatactttggagcctgggatttcaaaaaccagagttcagaaaattacaactactatgtgcctacagaacaagtcaaggtggagtccggatcatctggcaagctttccctgatcgttgatggcggcatgaccgtgaacgctgtgatcacccgcggcaccggcaacaataccaccagtgcctacactgaggctgtgtatacccataatgggtcccagatctatgtgaataagaccgtttataatcctctgaatatctcaaatcttattgtcgtggaggacggctacctgatgaagaatgagagcgttggtaacgtgaaggacgctaattacaccctgttcttgatgggcgaaaagaacaaatatacgcctatactgatctccaacaagctggccaactctatgttcaccaagctgtacctcctcggaggagctggccaggacattttcacaaatgtccacacagaggaaggagtgatgctctggcaggtgaacttcaacaacacggtggcaggaaaagggggcggaggctctggtggaggggggagcggcggtgggggatcaattgagaacgccgacaaggcgattaaggattttcaggataataaagcacctcacgataaatccgctgcttacgaggccaacagcaagcttcctaaagacctccgagataaaaacaatcggtttgtcgaaaaggtctccatcgaaaaagccatcgtgcggcacgacgagagagtcaagtccgcgaacgacgccataagcaagctgaatgagaaagactccattgaaaatcgcagactggcacagagggaagtgaacaaggcccctatggatgtgaaggagcaccttcagaagcaattggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 85 - protein, 252 aa, synthetic construct
MFVFLVLLPLVSSAASLIDTIDTINRYDHYTDTTMELINNFDNDTIVIYSIEHEFIRFHEQLKGTNEFLVPNKNRLFNPNDYNLSVMPIDKIVNENPHKDIYLILRIQKSNENFGEGIKVNKSAYIGPSFVKLEKINGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 86 - DNA, 943 bp, synthetic construct
  source 1..943: mol_type = other DNA
cttgttctttttgcagaagctcagaataaacgctcaactttggccaccatgtttgtgtttctggtgcttctgcctcttgtgtcaagtgcggcctctctcattgacacaatcgatacaattaaccggtatgaccactacactgacaccaccatggagcttattaacaatttcgataatgacaccatagtgatctactccatcgaacacgaatttatcagattccatgagcaactcaagggcacgaatgagttcctggttcctaataagaacaggttgtttaaccctaacgattataacctgtcagttatgcctatcgacaaaatcgtcaacgagaaccctcacaaagacatctatctcattctgcgcatacagaaatctaacgaaaacttcggggaggggataaaagtcaataagtctgcttacattggccctagctttgtcaaattggagaagatcaacggtgggggaggcagtggaggcgggggatccggtggcggaggcagcattgagaatgctgacaaggccatcaaggacttccaggacaataaagcccctcatgacaaaagcgcggcctacgaagcaaactccaagctgcctaaggatctgcgagataagaacaatagattcgtcgaaaaggtgagcatcgagaaagcaatcgtgcggcatgatgaacgggtgaagagcgctaatgatgccatctccaaactcaatgagaaggatagtattgagaaccgccgcctggcacagagggaggttaacaaggcccctatggacgtgaaggaacacttgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 87 - protein, 250 aa, synthetic construct
MFVFLVLLPLVSSAAYFMGYYTTYTKNDWRGFSGQLSSITNEGDYVVVMPDYITKPLNYYYSNSTDGTIEVGASSAATLEQINAKRLNSTGNPAAYYIITWDISAANPSGDALDWISKNAQFIGQNMGIYVFRSAGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 88 - DNA, 937 bp, synthetic construct
  source 1..937: mol_type = other DNA
cttgttctttttgcagaagctcagaataaacgctcaactttggccaccatgttcgtgttcctggtgcttctgcctttggtcagctccgccgcctatttcatgggttactacaccacctacaccaaaaacgattggcgcgggttcagtgggcaactgtcatccatcacaaacgagggagactacgtggtggttatgcctgactatatcactaagcctcttaactattattattctaacagcacagacggcacgattgaagtgggcgcttctagcgcagccactctcgagcagatcaatgcaaaaaggctcaactctacagggaatcctgccgcgtactacatcattacctgggacatttccgccgctaaccctagtggggatgccctcgattggatcagtaagaatgcacagtttataggacaaaatatgggcatttacgtgttccggagcgccggagggggtggtagcggcggcggaggttccggcggcggaggctcaatcgagaatgctgataaggcgatcaaggactttcaggacaataaagctcctcatgataagtcagcagcctacgaggccaactccaaactgcctaaggatttgagggacaagaacaatcgctttgtcgaaaaggtgagcattgagaaggccatagtcagacacgatgaacgggttaaatccgcaaacgacgcgataagtaaactgaatgaaaaggattctatcgagaaccgaagactcgctcagcgggaagtgaacaaagctcctatggacgtcaaggagcacctgcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 89 - protein, 288 aa, synthetic construct
MFVFLVLLPLVSSAAHTSFTTGDIADPIVQVQNSEELREVFPYMDNTTKIAVSNYVRWPFVWYYQGDYPKRISYYSDPGYNKDIGPENYDLIIVHDGEKVDSIEGFEKHTYKKCYWIDVNTLIQSYGKPIDIFNANDRALLVEDLKRVIHYYFTRDAGLGSINIDVFVKKDRAGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 90 - DNA, 1051 bp, synthetic construct
  source 1..1051: mol_type = other DNA
cttgttctttttgcagaagctcagaataaacgctcaactttggccaccatgtttgtcttcctggttctgctgcctttggtgagcagcgccgctcacacctcctttaccacaggagacatagccgaccctatagtgcaggtccagaactccgaggagctgagagaggtgtttccttacatggataacactaccaagattgcagtgtccaattacgtgcggtggcctttcgtgtggtactatcaaggagactatcctaaacgaatctcttactattccgaccctggctacaataaggatatcgggcctgaaaactatgatctcattatcgtccatgacggggaaaaagtggacagcatcgaggggtttgagaaacatacgtacaagaagtgctattggattgacgtcaacactctcatccagagttacggcaaacctattgacatcttcaacgcaaacgacagggccctgctggtggaagatcttaaacgggtgattcactactacttcacaagggacgcagggctcggatcaatcaacatcgacgttttcgtcaaaaaggaccgggctggaggcggtggctctggcggtggagggtcaggcggtggcggcagcatcgagaatgctgataaggccattaaggacttccaggacaataaagcccctcacgataagagcgctgcgtatgaagcgaactctaagctccctaaagatctgcgcgacaagaacaacagatttgtggaaaaggtctccatcgagaaagcaatagtgaggcatgatgagcgagtgaagagtgccaacgatgccatttcaaagcttaatgaaaaagatagtatcgagaatcgccgcttggctcaaagagaggttaataaggcccctatggatgttaaggaacaccttcagaagcagctggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 91 - protein, 484 aa, synthetic construct
MFVFLVLLPLVSSAAFSATHDIALGNAMKQGGMTPDWEEALSWMGENTPDTGIDYYAIYTRDSYENPEEAYGIMTWWDYGHWITFISKRAPNSNPFQRGVAGPNGAAAYFIQQDEAASNAILDNLDTRYVITDAEMDTAKFWAMCTWYNSSAGTGQYQQTFAMINPDNSISTGNLYSEKYYNTMISRLHNLDGSMVEPTSVYYVEYVDGSLYSIPIPIVTDAREMSYADAKAAVDAYNANARAGYGAGIYSLSLTAPSGEVPALEHYRLIHESPSSPFSAATTLKYVKVFEYVKGAKVKGDGIIEIDLESDQGRKFTYSQRSTDGYFTVPYSTTGNNYGTKVLGDYRIRGTQQTFKVSEDAVMNGLSVNGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 92 - DNA, 1639 bp, synthetic construct
  source 1..1639: mol_type = other DNA
cttgttctttttgcagaagctcagaataaacgctcaactttggccaccatgtttgtgttcctcgtgctcttgcctctggtctcttctgcagcttttagcgcaacacacgacattgccctgggcaatgctatgaaacagggaggcatgacacctgactgggaggaggccctctcctggatgggggagaacactcctgatacgggaatcgattactacgccatatacaccagggactcctacgaaaaccctgaagaggcctatgggattatgacatggtgggactacggtcactggatcactttcatcagcaaacgggcacctaactctaacccttttcaaagaggcgtcgcgggacctaatggcgctgccgcttactttatccagcaagatgaagctgccagcaacgccatcctggacaaccttgacactagatatgtgattactgatgcggagatggacacggccaagttctgggccatgtgcacctggtacaatagcagtgccggaacaggccagtatcagcagacctttgccatgataaaccctgataatagcatcagtacggggaatttgtattccgagaaatattacaacacgatgataagccgcctgcataacctggatggtagcatggtcgagcctacaagtgtttactatgtggagtacgtggatggctccctgtacagtatccctatccctatcgtcacagatgctagggagatgtcatatgcggatgccaaagccgctgtggacgcttacaacgccaacgcgagggccggatatggagccggtatctatagcctgtctctgaccgcaccttccggcgaagttcctgcacttgaacattatcggctcattcatgagagcccttcctcacctttctcagcggcaaccaccctgaaatatgttaaagtgttcgagtacgttaagggcgccaaggtgaagggggatgggatcattgagattgaccttgaaagtgaccagggtcgaaagtttacctattctcagagatccactgacggctacttcaccgtgccttactcaaccacagggaacaattacggcactaaggtgttgggcgactacaggatacgagggacccaacagaccttcaaggtgtctgaagatgccgtcatgaacggcctgtccgtcaatggagggggcggaagtgggggaggcgggtctggaggcggtggtagtattgaaaacgctgacaaggccattaaggacttccaggataataaggctcctcacgataaatccgcggcatacgaagctaattcaaaactgcctaaggacctccgcgacaagaataaccgcttcgtggagaaagtgagcatcgagaaagctattgtgcggcacgacgagagagttaaatcagcaaatgacgcaatctccaagctgaacgagaaggacagcatcgagaaccggcggcttgcccagcgcgaagtcaataaggcccctatggacgtgaaggaacacctgcagaagcagctcgattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 93 - protein, 496 aa, synthetic construct
MFVFLVLLPLVSSAAADHLASSQAVGSTNDDMYNTLTWIKANTSQDTVLASWWDFGHLFTAVADRQVVFDGGSQNNMRAYWIGNALTSTDEAKSAGILRMLANSGEDASNTLDLYTNNTEKTVEILNAILPMDRTEANSALTGTYGLSQQEANSVLDLTHPAKVKPVNLILSSDMLSKAAWWSYFGSWDFKNQNSTHYSYYPSQSNIENINGREFTLGMDNGVIGVSSPTNETNGSTMTFAYVDQSKLNKSLNMSTLEDKQRMSKELSDGTGNTLLKPHKLIVVENNQLTEKIVNNNSNMSIMAIHQNDGSYFTVLFDSHLEESLFTKLYLKSGLNVTRFNMTHSEPGISVWDVSEYANTTANSTTNSSTNGTNVQTNTSTGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 94 - DNA, 1675 bp, synthetic construct
  source 1..1675: mol_type = other DNA
cttgttctttttgcagaagctcagaataaacgctcaactttggccaccatgtttgtgttcctggtgttgcttcctctggtgagtagcgccgcggctgatcacctggcctcctctcaggcggtcggaagtacaaatgacgacatgtacaatacccttacttggatcaaggcaaatactagtcaggacacggtcctcgccagttggtgggactttggtcacctcttcactgcagtcgccgacagacaggtggtttttgatggcggttcacagaacaacatgcgcgcttactggatcggcaacgcactgacgtctacagatgaggctaagagcgctggtatccttagaatgttggcaaattccggcgaggacgcctcaaatacattggatctgtacacaaacaataccgaaaagacggtcgaaattctgaacgccatcttgcctatggatcgcacagaggccaattctgccctgaccggtacatacggactctcccagcaagaagcaaatagcgttctggatctcacccatcctgctaaggttaaacctgtgaacctcattctgtcctctgatatgctgtcaaaagcagcttggtggagctatttcggtagctgggacttcaagaaccagaacagcactcattattcttactatccttcacagtcaaacatagagaacatcaacggcagggagttcacccttgggatggacaatggcgtgattggcgtgtccagtcctaccaatgaaaccaacggaagcaccatgacctttgcctacgttgaccagtccaagttgaataagtccctgaacatgtccaccctcgaggacaagcagcggatgtcaaaagagttgagtgacgggacaggaaacacgctgctgaagcctcacaaactgattgtcgtggagaataaccaacttaccgaaaagatcgtgaacaacaacagcaacatgagcatcatggcgattcatcagaatgatggaagctatttcacagtgctgttcgactcccacctggaggagtccctgtttactaagctgtatctcaagtccggactcaatgtgacccggtttaacatgacccactctgagcctgggattagcgtctgggatgtgagtgagtacgctaacactactgccaatagcaccactaattcctctactaatgggacaaacgtgcaaacaaacacgtcaaccgggggcggcgggagtgggggcggcggaagcggaggcggcgggagtatcgaaaacgccgacaaagccatcaaggacttccaggacaataaggcccctcacgataagtctgctgcatacgaagcgaactccaagctgcctaaggatctgcgggataaaaacaacagattcgtcgagaaggtctctatcgaaaaagctattgtgaggcatgacgagcgagttaaaagcgccaacgacgccatatctaaactcaatgaaaaagatagcatagagaaccgcaggcttgcccagcgagaggtgaataaagcgcctatggacgtgaaagaacacctccagaagcaactggactaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 95 - DNA, 45 bp, synthetic construct
  source 1..45: mol_type = other DNA
gaatttaatacgactcactataaggcttgttctttttgcagaagc

SEQ ID NO: 96 - DNA, 149 bp, synthetic construct
  source 1..149: mol_type = other DNA
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 97 - protein, 483 aa, synthetic construct
MFVFLVLLPLVSSAAAKTVFITSDNIIDTDTDLQMLNSIKNYIEEISGGELQVIVDNQAPSPGEGWRAIEVTSDVSICLAACDAGNFIQLAGASTNSDKQIILVNTGDYDLDNHTNFLRRAWDDNYSDAYLAGLRDPGTFLKNSGVYYIQPVKEFPNNANEGYIDRYDDEMNKQIAQEIVDIVNGHGNDTRIFSDELVSKNIVNPGVMAKSSQALINSGDNEMKGTYGNYTAAQLLYQTSSYLNGNGLDVPKTFSEPDNPMGISFLTRDSYSIYDYFRMGGIVREYMDQNGKAPDSIEYEGAHIGYYDLLYNFAKITQNHTDAKHMGFDSEYHFDKVNDSILLHIFPFVLILLVLFVAYRFYKRLRRYGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 98 - protein, 482 aa, synthetic construct
MFVFLVLLPLVSSAATKTVFITTDNIVDHDFDVGLITAIKNYVQELSGGELQVVIDNQAPAAGEGYRSIEVTSDASIDLAASDAGNYIQLANYSAHSNKQIVFVNIGDYDLDNSSNYLRRAWDDNYTNETIAGVHDPGTLLRNSGIFYVQPAKEFPDKYRNGILDNYDDEMAKQIAQEIVGIINTHDNDTKVFSDDLVVKNKISPAGMANASKELLNSGDKEFNGTFGAYTAPQLLYQTSSYLNGNGLDIPKTFKEPENPMGVSIFAKGSYSISDYFKMGGIVRNYMDEHGQAPDSIEYEGAQISYYDLLYNFAKITQNHTDSAHMGFENEYQFEKVNSSFLLDVFPFILVLFILFLAYLVYKRIRRGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 99 - protein, 482 aa, synthetic construct
MFVFLVLLPLVSSAATKTVFITTDNIVDHDFDVGLITAIKNYVQELSGGELQVVIDNQAPAAGEGYRSIEVTSDASIDLAASDAGNYIQLANYSAHSNKQIVFVNIGDYDLDNSSNYLRRAWDDNYTNETIAGVHDPGTLLRNSGIFYVQPAKEFPDKYKNGILDNYDDEMAKQIAQEIVGIINTHDNDTKVFSEDLVVKNKISPAGMANASKELLNSGDKEFNGTFGAYTAPQLLYQTSSYLNGNGLDIPKTFKEPENPMGVSIFAKGSYSISDYFKMGGIVRNYMDEHGQAPDSIEYEGAQISYYDLLYNFAKITQNHTDSAHMGFENEYQFEKVNSSFLLDVFPFILVLFILFLAYLVYKRIRRGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 100 - protein, 262 aa, synthetic construct
MFVFLVLLPLVSSAASKTVFITSDNIIDTDTDLQMLNSIKNYIEEISGGELQVIVDNQAPSPGEGWRAIEVTSDVSICLAACDAGNFIQLAGASTNSDKQIILVNTGDYDLDNQTNFLRRACDDNYSDAYLAGLRDPGTFLKNSGVYGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 101 - protein, 483 aa, synthetic construct
MFVFLVLLPLVSSAATKTVFLTSDNVIDHDTDLKVLNSLKNYIEEISGGELQVIIDNQAPGPGEGWRAISVTSDVSIALAAADAGNFLQLATSTVDTNKQIILINIGDYDLDNNSNYLRRAWDDNYSNESLAGIRDPGTFLKNAGIYYLQPVKEFPNNAHDGILSNYDEEMNRNLAEQIVKLINAHENDTKVLSDSLIVTNKLSPKGMANASKLLVNSDDKEMKGPYGSYSAPQLLYQTSAYLNGDGIDIPKEYSEPENPMGISFLVKDTYSIYDYMNMAGIVKNYMDENGQAPDSIEYEGAHIGYYDLLYNFAKITQNHTDAKHMGFESEYHFDKVNDSILLHIFPFVLIFLVLLIAYVFLKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 102 - protein, 483 aa, synthetic construct
MFVFLVLLPLVSSAAAKTVFITSDNIIDHDSDVKLLNSLKSYIEELSGGELQVIVDNQSPAPGEGWRSIEVTSDVSVDLAASDAGNYLQLASSTVNSNKQIVFVNIGNYDLDNHTNFLRRAWDDNYSNETLAGMHDPGTFLKNAGIQYVQPAKEFPNNVNDGVISNYDEEMNKQIAQEIVDIINTHGNDEKTLSDGLVTHNIIKPSIMAKASQELIKSNDKEMKGTYGNYSAPQLLYQTSSYLNGNGLDIPKSYDEPENPMGISFMAKDTYSVYDYFKMGGIVREYMDQNGRAPDSIEYDGAQISYYDLLYNFAKITQSHTDVKHMGFESEYHFDRVNDSILLHIFPFILILFVLFLAYLFLKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 103 - protein, 483 aa, synthetic construct
MFVFLVLLPLVSSAAAKTVFITSDNIIDHDNDIKMLNSLKQYIEEISGGELQVIVDNQAPAPGEGWRAIEVTSDVSICLAASDAGNYLQLATATTNSDKQIVFVNTGSYDLDNHSNYLRRAWDDNYSNESLAGLHDPGTFLKNAGIYYIQPSQDFPDNAKDGYLAKYDESMNKQLAQEIVDIIKTHENDTTILSDGLITHNIIKPSIMANASKELIKSGDKEMDGTYGNYSAPQLLYQTSSYLNGNGLDVPKAYDAPENPMGISFMAKDTYSIYDYFKMGGIVRDYMDQNGRAPDSIEYEGAHISYYDLMYNFAKITQNHTNGKNMGFESEYHFDKVNDSILLHIFPFVAILFILFLAYLLYKRLRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 104 - protein, 403 aa, synthetic construct
MFVFLVLLPLVSSAATKTVFITSDNIIDHDTDIEMLNSLKNYIEEISGGELQVIIDNQAPGPGEGWRAINVTSDVCINLAAADAGNYLQLAKATVYSDKQIVLINTGDYDLDNNTNYLRRAWDDNYSNESLAGIRDPGTFLKNAGIYYLQPVKEFPDNAHDGYLDRYDEEMNRKLAEEIVEIVNKHGNDTKILSDSLIVTNKIPPTGMANASKMLVNSEDKEMKGPYGSYSAPQLLYQTSAYLNGDGIDIPKEYTEPDHPMGLSFLVRDSYSCYDYMHMAGLDKNYMVGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 105 - protein, 475 aa, synthetic construct
MFVFLVLLPLVSSAADNIIDTDTDLQMLNSIKNYIEEISGGELQVIVDNQAPSPGEGWRAIEVTSDVSICLAACDAGNFIQLAGASTNSDKQIILVNTGDYDLDNHTNFLRRAWDDNYSDAYLAGLRDPGTFLKNSGVYYIQPVKEFPNNANEGYIDRYDDEMNKQIAQEIVDIVNGHGNDTRIFSDELVSNNIVNPGVMAKSSQALINSGDNEMKGTYGNYTAAQLLYQTSSYLNGNGLDVPKTFSEPDKPMGISFLTRDSYSIYDNFRMGGLVREYMDQNGKAPDSIEYEGAHIGYYDLLYNFAKITQNHTDAKHMGFDSEYHFDKVNDSILLHIFPFVIILLVLFVAYWFYKRLRRYGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 106 - protein, 330 aa, synthetic construct
MFVFLVLLPLVSSAAEEMNKQIAQEIVDIVNTHENDTKILSDGLITHNIIKPAVMANASKALIKSNDTEMKGTYGNYTGPQLLYQTSSYLNGNGLDIPKAYDEPEDPMGISFLAKDTYSVYDYFKMGGIVREYMDANGRAPDSIEYQGAHIGYYDLLYNFAKITQNHTDTAHMGFESEYHFDRVNDSILLHIFPFILILFVLFIAYLFLKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 107 - protein, 1110 aa, synthetic construct
MFVFLVLLPLVSSAASNASDNLDDLTISDSNSLDLVSTSNSDILSSDSGVSSDDSSNDASGDVLGSDVSSNESNNQSQSTLDSNNQSQSGLDSDNSTLLDSQSNNQSNSESSDSSDSSETVIKNATSISVSSKTVVRGNSLNITLKDNASTLLSNKTVTFTFNGKTYNKTTNAKGIASLTLTATPKKYLVKIAFVGDELYEASSKSVNVTLSKTPTSISNSGKSIVRGKLYKLTLKDAKGKALSGKKISISFNGKKYTKTTNSNGQVNLTINVNVGKTYKMTYKFAGDSNYLSSSGSVSIKVKMGTSIIGSGSSIVKGKSYTVTLKNANGAVLSNQKIAFTLSGKTYNRTTNAKGQASLKIGLNSGKTYNLTYKYAGNSYYGGSSGKVSLFVKTPTTMKNSGKTIVSGETYKVTLKDADGKSLANKKVSITFNNKTYAKTTNSNGQASLTIKGTFGRSYPLSYKFAGDSKYGPSSGSLCLRVKKATSLKGSASSIVQGKSYTVTLKDSNSTPLANQTIVFTLDTKKYNRTTNAKGQASLKIGLAAGKTYNLAYKYSGTSYYNGSSGSVKLKVKFPTSLTNSGKSVMNGTGYNIVLKDSKSNLVSNKTISIGFNGKTYDEITDANGTVTLLIDANVPKTYKMTYKFAGDSDYGASSGTVNLTVKFKNAFTISQIISASSSLKSYVLKNKKVPATVSVNGVSLNLTSFTYLMAKATISINSNKTSGSILLVPVDSNYTNNGSRINANLYKANYIDLAKKVISSAEANKLVPNSVSTNIGLVSHDLYSFGLAKALVFFNSDHYLPNYLILSSDDVGEKHSTVIPSNARGNASQFKAGLNEAETLTAAQIAKYLVASGHDATNSEIKALAAKLVSGKTSLWDKANAIFTFARDNITYSYYADSKKGAAGTLSSKSGNCCDHSNLIVSLCRAANITARFSHAQGCTFSSGLVAGHVWAQIYIDGVWYTADATSRRNSLGNIVNWNTNHYNTLKQYDHLSFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 108 - protein, 484 aa, synthetic construct
MFVFLVLLPLVSSAASKTVFITSDNIIDQENDVKVLNSIKEYIEEISDGELQVIVDNEAPAPGEGYRAIQVTSDISICLSAADGGNYLQLAEASADSDKQIILINIGSYDLNNNSNYLRRAWDDNYSNETFMGIHDPGTFLKNAGIYYIQPVEEFPDNFNDDGYLAKYDEEMNKQIAQEIVDIANEHANDTTMLSDNLVVRNTISPAEMAEASKALLNSNDTEMNGEYGNYTAPQLLYQTSSYLNGNGLDMPRSFDAPENPMGISFMVKDSYSIYDYMKMAGIVRNYMDENGQAPDSIEYEGAHIGYYDLVYNFAKITQNHTDAEHMDFEQEYKFDKVNDSILLKAFPFILILFILFLAYLGYQKIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 109 - protein, 1095 aa, synthetic construct
MFVFLVLLPLVSSAAMDIDEVGSSDDISDSGVVLESGADAGAYGSSNADVDSANQLSSNDTAGNVNSESEVLSTDTNENSLESDIIDKDSKNVLSSSSLQAAAKKKTVIKGSGTSITRGSSYYITLTDSNGNVLSNQKVTFSIKGKTYNKTTNSKGVASLKISLAKGKYDFVCAYAGTGAYYSSKLSVSLTVNAMATNIRTSGNSIKRGKSYSITLTDANSNLLANEKVSFNISGHVYNRTTNSKGVASLTMNTAAKKYSLVCSYGGSSYYKASSVALTLTVLKGDTHIKISTDTVKKGNAVVMTLLDVRNKVLSAQKVSFTIKGKTYNLTTNSNGTAKLIINLAAGKYPLVCSYDGSSNFMASKASENLTITDTVKTFSIADIETAATNLKAYVLKNKVLPSTVTVGGASLKISEFSYLMAKAVTNLNSNNKNKITLITGISNGDSATYVLNAKVYKNQYVNVSKRVYSYIDSNKVPATYATVYSSSGANVGKAGFNLYTFAFAKILAFHKTENYLPNYCTFESSALKASTAAAGGSSSTSSANNQSKLKTTSLKAQSTSITRGDSYSVTLKDGSGNALPNQKITFTVSNKQYSDTTDSKGIAYLGADLLSGKYSITASFAGSSAYKSSKLSNTVTVKNSSTRFFLDDIENAAVNVKNYVSKNKALPTTVTVANTKLTIAQFSYIMAKAVHNINAGNKKYISLISISNCKSSGNYLDTTVYKAQYMNLTNRVISFTESNKVPPVYATVYGTNGKSVGNSEFNLYTFAFAKILAFHKTNNYLPNYCTFQSSAIGVKKPSSTTTTVVVNGPIKANSSQFKTGLNEKNTVGNLSAYLVDSGYSKITSSIQNLANQLTKNLNSTASKALAIYNYVRDEISYSYYANSRYGASGTLSVGSGNCVDQANLIVALCRASGIEARYAHAKGCTFSSGLVTGHVWAQILVNGVWYSADATSVRNSLGNIVNWNTNSYYSLKQYTAIPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 110 - protein, 1146 aa, synthetic construct
MFVFLVLLPLVSSAAMDIDDSSSIDDSNDLSGASVSSNSNENLASDSNSNNLESDNAGSSNVNSDYEILNTDNTVEDSDLEHNSNIANTKAMLGASSQSDSAVLQANAKVKTTLKGSSSSIYRGNYYTLTLTDSKGKVLSGQKLSYNINGKTYTLTTDSKGSTYLQINLKEGKYTMVCSYGGSSAYESSRLSVTLSVLKNPNAFTVKEIEDAATNVKNFVLKNKRLPNTVKVGSKTLKISEFSYLSSQLISNLNSNKKGDIILLSGISDGKSSSASLKTTVYKAQYLDLAKNVVSYIGSKKVAPNEILVKDASKRSVGNANFNLYTFAFAKILDFHKSKNYLPNYCTFESSAFNQAVSLKATILKGSSNSIYRGNYYTLTLTDGNGKALSGQKLRYAINGKTYTLTTDSKGSTYLQINLKEGKYPMVCSYAGSKVYKSAKNSVTLTVLKNSNAFSVNEIETAATNVKNYVLKNKRLPNTVKVGSKTLKISEFTYLSSKAVSNLNSNNKKDIVLLNGISNGGSSTYSLKFTVYKAQYVDLAKRSASNIESKKVPANYLSVKDGSNKAGNANYNLYTFAFAKILDFHKSHNNLPNYCTFESSVYAPLKKSTSIKASSNSVNKGDSYSVTLVDNAGNALANQKITFNFSGKLYSQTTNSKGVASLKIGASQGTYSVVSSYAGSSACEASKLSSTVTVKDTNRFSISEIEVAASNVKEYINIKNVRPGTVTVANKRLTISEFSYLMAKAVYNINAGNTNYITLPSGISGGNSEGDSMDATVYKAQYVDLSKRVVSFEESNKVSPVYAKVYSSSGSSLGNAGFDLYTYSFAKILDFHKSHKNLPNYCTFDSSVFKSSVVPAGISSDIPYNSNQFKAGLNEKNTESDLSKYLIGTGQSAITSSISNLANQLTKGLKSNEAKAQAIYNYVRDEIDYSYYANSKYGASGTLSAGSGNCVDQASLVVALCRASGIEARYAHAKGCTFSSGLVTGHVWAQILVNGVWYSADATSVRNKLGNIQNWNTNSYSNLNRYAAVPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 111 - protein, 483 aa, synthetic construct
MFVFLVLLPLVSSAATKTVFLTSDNIIDHEHDLQVLNSLKRHIEEISGGKLQVIVDNQAPAPGEGWRAIEVTSDVSICLAASDAGNYLQLATASTNSNKQIVFVNIGSYDLDNNTNFLRRAWDDNYSPESLAGMNDPGTFLKNAGIYYVQPTKEFPENANDGVLSNYDEAMNEKIAQEIVDIINKHGNDHKILSDGLVTHNNVKPSVMAKASKELIESNDFSMDGKYGRYTGPQLLYQTSSYLNGNGLDIPKDYGEPENPMTTSFLAKDTYSVYDFFKMGGIVRAYMDDTGKAPDSIEYEGAHISYYDLLYNFAKITQTHTSAKHMGFESEYHFDKVNDSILLHIFPFILIIFVLFIAYLFIKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 112 - protein, 483 aa, synthetic construct
MFVFLVLLPLVSSAASKTVYITSDNIVDHDTDVKMLNSIKKYIEEISGGSLQVIVDNQAPAPGEGYRAIQVTSDISICLAAADAGNFYQVSENSADTDKQFVLINTGDYDLDNHSNYLRRAWDDNYSNESLMGIRDPGTLLKNSGIYYIQPLKEFPNNGKNGYIDRYDEEMNKKIAQDIVDIVNGHANDTKILSDNLIVTNKISPSAMANASKALINSNDTEMNGTYGNYTAAQLLYQTSSYLNGNGLDIPHSFNGPEKPMGISFLVKDTYSIYDYMNMAGIVRNYMDANGRAPDSIEYEGAHISYYDLLYNFAKITQNHTDAKHMGFNQEYKFEKVNDSILLHIFPFVLILFVLFLAYLGYQKIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 113 - protein, 483 aa, synthetic construct
MFVFLVLLPLVSSAASKTVFLTSDNIIDQDNDVKMLNSIKNYIEEISGGELQVIVDNQAPSPGEGYRAIQVTSDISICLAAADAGNYLQLGQTSANSNKQIVLINTGDYDLDNHSNYLRRAWDDNYSNETFMGMHDPGTLLKNSGIYYIEPIKEFPDNGKNGYIDRYDEDMNKQIAQEIVDIVNGHGNDTKTFSDNLVVTNTISPKVMADASKELLSSNDTEMNGTYGNYTAGQLLYQTSSYLNGNGLDIPKSYDGPEHPMGISFLVKDTYSIYDYINMAGIVKNYMDENGRAPDSIEYQGAHISYYDLQYNFAKITQNHTDAKHMGFDHEYKFDKVNDSILLHIFPFILILFVLFLAYLGYKKIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 114 - protein, 482 aa, synthetic construct
MFVFLVLLPLVSSAATKTVFITTDNIVDHDFDVGLITAIKNYVQELSGGELQVVIDNQAPAAGEGYRSIEVTSDASIDLAASDAGNYIQLANYSAHSNKQIVFVNIGDYDLDNSSNYLRRAWDDNYTNETIAGVHDPGRLLRNSGIFYVQPAKEYPDKYKNGVLNYYDDAMAKQISQEIVDIINTHDNDTKVFSEDLVVKNKISPAGMANASKELLNSGDKEFNGTFGAYTAPQLLYQTSSYLNGNGLDIPKTFKEPENPMGVSIFAKGSYSISDYFKMGGIVRNYMDEHGQAPDSIEYEGAQISYYDLLYNFAKITQNHTDSAHMGFENEYQFEKVNSSFLLDAFPFILVLFILFLAYLVYKRIRRGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 115 - protein, 483 aa, synthetic construct
MFVFLVLLPLVSSAAAKTVFLTSDNIVDHDTDLQVLNSIKSYIEEISGGELQVIVDNEAPAAGEGWRAIAVTSDVSICLAASDAGNYLQLGTASANSDKQYIFVNVGDYDLDNHTNFLRRAWDDNYSNESLAGMHDPGTFLKNAGVYYIQPTKEFPQNTDDGIMDRYDEGMNRQIAQEIVDIVNSHGGDSKVLSDSLVTHNIVKPAVMAQASKALVESGDKEMQGTYGNYTAAQLLYQTSSYLNGNGLDVPKSYDPPSDPLGISFFTKDTYSVYDYFNMAGIVREYMDQNGKAPDSIEYEGAQISYYDLQYNFAKITQNHTDAEHMGFESEYHFDKVNDSILLHLFPFVVILFVLLIAYRFFKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 116 - protein, 483 aa, synthetic construct
MFVFLVLLPLVSSAAAKTVFITSDNIIDHDSDLKMLNSLKSYIEEISGGSLQVIVDNQAPAAGEGWRSIEVTSDVSIDIAASDAGNYVQLASYTANSDKQIVFVNIGNYDLDNHTNFLRRAWDDNYSNENLAGLHNPGTFLKNAGIYYIQPAKEFPENAKSGILDTNDDEMYKKMAQEIVDIINTHENDEKVLSDGLISRNIVKPSVMANASKELINSNDKEMTGTYGNYTAPQLLYMTSSYLNGNGLDVPKSFEEPENPMGISFMARDKYSVYDYFKMGGIVKEYMDENGRAPDSIEYEGAHISYYDLMYNFAKITQNHTDAKHMGFDSEYHFDKVNDSILLHIFPFILILFVLFIAYLLYKRIRRFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 117 - protein, 484 aa, synthetic construct
MFVFLVLLPLVSSAATKTVFITTDNIVDHDFDVGLINSIKNYVQELSGGELQVVIDNQAPRPGEGYRSIAVTSDVSIDLAASDAGNYLQLANYSANSDKQIVFVNIGDYDLDNSSNYLRRAWDDNYTNESFAGVHDPGTLLRNAGIFYVQPAKEYPDYYHDGILDKYDDEMAKKISQEIVDIINTHDNDTKVFSDELVVKNNIPPKGMADAAKEVVNSDDKELKGPFGAYTGPQLLYQTSSYLNGNGLDVPKTYKDPESPMGISFLTKDSYSIYDYFKMAGIVRNYMDEHGQAPDSIEYEGAHIGYYDLLYNFAKITQNHTDTAHMGFESEYHFDKVNDSFLLHIFPFILILFILFLAYLGYKKIRRFKGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 118 - protein, 482 aa, synthetic construct
MFVFLVLLPLVSSAATKTVFITTDNIVDHDFDVGLITAIKNYVQELSGGELQVVIDNQAPAAGEGYRSIEVTSDASIDLAASDAGNYIQLANYSAHSNKQIVFVNIGDYDLDNSSNYLRRAWDDNYTNETIAGVHDPGTLLRNSGIFYVQPAKEFPDKYRNGILDNYDDEMAKQIAQEIVGIINTHDNDTKVFSDDLIVKNKISPAGMANASKELLNSGDKEFNGTFGAYTAPQLLYQTSSYLNGNGLDIPKTFKEPENPMGVSIFAKGSYSISDYFKMGGIVRNYMDEHGQAPDSIEYEGAQISYYDLLYNFAKITQNHTDSEHMGFEHEYKFEKVNSSFLLDVFPFILVLFVLFLVYLVYKRMRRGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 119 - protein, 648 aa, synthetic construct
MFVFLVLLPLVSSAAQSVNDSDDLDEKILKSCSLSKNATDKNISIKAKANSTKKTSNNKISTSKTKANTTTINRKTLAKTSTSFMNHVEKNGKFPKVKISNKNYSNTEYLYLMTKAVENNSNSKIEIKRNLVKRYNNTNSKSVKGTLNKTEYVKIASKTRKFIDKNKRAPNWVSSSKGNIPYNQLILSYSKCLDYFNKNNRLPNSIRLDDLDLDKINSKLNRNKTKNPSNASVKIKKQTANKTVNKTTSNIVKTNTNKTRNTTTNTAPNKDDKSSKNSSLVERTLNSINNILNNILYRLDPSKYQLDLTKSDEANLKLNTSKINVDINGKSTVNVKVSAKNATKATSKKSTASKAKTNKTKSSVSKAKTKTVNSNKASITESLMKYLSSSKNCQVKNKSIQDLAKTLTSKLKSDYEKGKKLFTWVRDNIQYKKYRNTRRGAVKTLQTKKGNCVDQSHLLIALSRAAGIPARYVKGGNCKFSSGYGSGPIWTQMYINNKWVVADTTSHRNSLGKIKNWNTKNYKLYGQFSSINFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 120 - protein, 647 aa, synthetic construct
MFVFLVLLPLVSSAAQSISADNLDEKTIKSDSSSVKSSTATKVKTSTANKKTTSTDNEKTTSASKKASASATKENKTTVNKKTLAKTSTSFINYVEKNGKFPKVRISNKNYSNTEYLYLMTKAVENNSNPKIEINRNMVKKYNNSNSKSVKGSLNKTEYVKIAGKTRKFIEKNRRAPNWVSSSKGNIPYNQLILSYSKCLDFFNKNSRLPNTVRLDDLDLDKINDKLNKNKSESAVNASSKTSKQTSNKTVNKTTGNIAKTNANKTKNTTTKTASNKEDDSSKNASLVERTLNNIHNILNNILDRLDPSKYQADVTKTDEANVKLNTSKINVDINGKSTVNIKVSAKNTTKATAKKTATKSTVKKTASKTVSSNKNSINEVLRKCLASSKNCQVYKRQIQDLAKTLTSKLKSDYEKGKKLFTWVRDNIQYKKYRNTRRGAVKTLQTKKGNCVDQSHLLIALSRASGIPARYVKGGNCKFSNGYVSGHIWAQMYINKKWVVADTTSHRNTLGNIRNWNTKNYQLCGQFSSINFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 121 - protein, 1075 aa, synthetic construct
MASVSASDVNDINDGNDHISLSEDSASSDLANDDLALSSADADSIDDDLDLASSSNEAYEDFSANPSNGESSYGPQSNALSESGDSNSQTTTNTSIESSTSNVVKGENYSVTLKDKDGNVLSGKNIIFTLNGTNFTRNTNSNGIASLTLNTRVGNYLIVVSFLGDDSYANSSFSQQLTVSKIPTAIENSTGLAVIGRAYSVVLKDKKGNPLSSKAVTLTFNGKTYKRTTNSQGIVSITLDGKRGNSYNLTYKFAGDSSYMASSGSVSLKLKMSTKIVGTDARIVQGKVFTVTLKDASNKLLANKKVTVLHNGKTYNRTSNSKGIVSLTLSQTPGKYYNISYRFAGDSTYTGSSKKLSVFIKTPTKFVNSGSFVCKGNVYYVTLKDSNDNVLANKTVKVTYGTKNYTLTTNSKGKVGVKINSAKGKVYKFTYKFNGNSLYGPSSGSLNLRTKLATSLIGSSATIIKGNPYKVTLKDSDGAVIANQNITFNFSGAKYVRTTNSKGIASLVINPSALKTYNLSYSYAGNSLYNKSSGNVSLAVKLGTTIKNSGTTVANNSSYEVTLKDSNNNALASKVIIFTLDGKTYRNTTNSKGVASLFISEKNFTTVNLTYKFAGDSMYVASSGSVKVRVVSDKVFTFNQIVAASKALRKYVEKNSELPSTVTVNGLKVNITSFAYLMSKSIVNVNNGKKSSVEVVSVSSNYSNSGSGLINANLYKAGYLNLSNYLISYTKSNHKIPNHINTRIGDLSPNLYIFGLSKALDFYSNNSYLPNYLILNSEDVCGKSNASIKHGNASQNKKGLNEAQSLNATQLAVYLKSSGNDALNDAIRNLAKQLTSGKSTTLAKANAIFTYVRDNVAYEYYADTKYKATGTLSAKRGNCCDHANLVVALCRAANIPARYSHAQGCTFSSGLVTGHVWAQIYIEGVWYSADATSKRNGLGNFHIWITNYFNSLKQYVHLPFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 122 - protein, 704 aa, synthetic construct
MFVFLVLLPLVSSAAQSLDDLNYGDSDIGLNDSGNPELNRDSTFKSLQSTVIESSNTDKTEKTNTNESLGAKAISSNCEKTDTYEDKSVKTSTAKASNTTQVKENKTSMKITTLAKSASSYFAYVKKNGKLPNPVSISNKKYQSAQYLYLVSKAVSNISASTVEIKDSLVNNFSYINYKTANGTLSKKECEELANKTARFIEKNHRAPTWIGSDKGNIPCNQLILLFTKCLDYYNNNSKLPSSVKLSDVDLDKISGKSNVSSSKASNSSKNAGNSEILLNSTLNAISSILTDLNNILNPSISTTTTITISSNSKTNTTTATIKTNTTVSTKSNTSAKTNSSAKTNTSAKTNNSTKTNNSTKTNTNTKVNTTAKTNSSTKTNTNTKVNTTAKTNTNTATSSSSKNSLSSWVDKAITAVGKSLIKFIDSSILNDKYNGESLKKYLSASKNCQSTSSAIKSLAKKLTSSLTSDYDKGEKIFNWVRDNIEYQKYSNTKKGALNTLNSKSANCVDQAHLVVALARAAGLPARYVNANNCKFTSGYVSGHVWAQILVGITWVVADTTSSRNSFGIVKNWNVKSYKLVGKYSSISFGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD